SEQUENCE LISTING (68 sequences)

SEQ ID NO: 1 - protein, 232 aa, synthetic construct
MVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYK

SEQ ID NO: 2 - protein, 231 aa, synthetic construct
MSELIKENMHMKLYMEGTVNNHHFKCTSEGEGKPYEGTQTMRIKVVEGGPLPFAFDILATSFMYGSRTFINHTQGIPDFFKQSFPEGFTWERVTTYEDGGVLTATQDTSLQDGCLIYNVKIRGVNFPSNGPVMQKKTLGWEANTEMLYPADGGLEGRTVMALKLVGGGHLICNFKTTYRSKKPAKNLKMPGVYYVDHRLERIKEADKETYVEQHEVAVARYCDLPSKLGHK

SEQ ID NO: 3 - protein, 223 aa, synthetic construct
MVSKGEESVIKPEMKMRYYMDGSVNGHEFTIEGEGTGRPYEGHQEMTLRVTMAEGGPMPFAFDLVSHVFCYGHRVFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGGNHKCQFKTTYKAAKEILEMPGDHYIGHRLVRKTEGNITEQVEDAVAHS

SEQ ID NO: 4 - protein, 237 aa, synthetic construct
MVSKGEELIKENMRMKVVMEGSVNGHQFKCTGEGEGRPYEGVQTMRIKVIEGGPLPFAFDILATSFMYGSRTFIKYPADIPDFFKQSFPEGFTWERVTRYEDGGVVTVTQDTSLEDGELVYNVKVRGVNFPSNGPVMQKKTKGWEPNTEMMYPADGGLRGYTDIALKVDGGGHLHCNFVTTYRSKKTVGNIKMPGVHAVDHRLERIEESDNETYVVQREVAVAKYSNLGGGMDELYK

SEQ ID NO: 5 - protein, 232 aa, synthetic construct
MVSELIKENMPMKLYMEGTVNNHHFKCTSEGEGKPYEGTQTQRIKVVEGGPLPFAFDILATSFMYGSRTFIKHPPGIPDFFKQSFPEGFTWERVTTYEDGGVLTATQDTSLQDGCLIYNVKVRGVNFPANGPVMQKKTLGWEASTETMYPADGGLEGAVDMALKLVGGGHLICNMETTYRSKKPATNLKMPGVYNVDHRLERIKEADDETYVEQHEVAVARYSTGGAGDGGK

SEQ ID NO: 6 - protein, 232 aa, synthetic construct
MVSELIKENMPMKLYMEGTVNNHHFKCTSEGEGKPYEGTQTMRIKVVEGGPLPFAFDILATSFMYGSRTFIKHPPGIPDFFKQSFPEGFTWERVTTYEDGGVLTATQDTSLQDGCLIYNVKVRGVNFPANGPVMQKKTLGWEASTETMYPADGGLEGACDMALKLVGGGHLICNLETTYRSKKPATNLKMPGVYNVDHRLERIKEADDETYVEQHEVAVARYSTGGAGDGGK

SEQ ID NO: 7 - protein, 232 aa, synthetic construct
MVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYK

SEQ ID NO: 8 - protein, 230 aa, synthetic construct
MENVRRKTGIQTEMKTKLHMDGMVNGHSFEIKGEGKGSPYEGVQTMKLKVTKGAPLPFSIDILLPQCMYGSKPFIKYPENIPDYLKLSFPEGITWERTMTFEDGAVCDVSNDSRLVGNCFIYTVKFQGVNFPLDGPVMQKKTRGWEPSTEVLYECDGWMRGLVDIALKLENGGHYMCNFKTTYKSKKGLEVPPYHFVDHKLDLLSHNTDGATFEEFEQGEIAHAHLSKLA

SEQ ID NO: 9 - protein, 232 aa, synthetic construct
MVSELIKENMHMKLYMEGTVNNHHFKCTSEGEGKPYEGTQTMRIKAVEGGPLPFAFDILATSFMYGSKTFINHTQGIPDFFKQSFPEGFTWERVTTYEDGGVLTATQDTSLQDGCLIYNVKIRGVNFPSNGPVMQKKTLGWEASTETLYPADGGLEGRADMALKLVGGGHLICNLKTTYRSKKPAKNLKMPGVYYVDRRLERIKEADKETYVEQHEVAVARYCDLPSKLGHR

SEQ ID NO: 10 - protein, 236 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 11 - protein, 244 aa, synthetic construct [sequence omitted]

SEQ ID NO: 12 - protein, 244 aa, synthetic construct
MVSKGEELIKENMHTKLYMEGTVNNHHFKCTHEGEGKPYEGTQTNRIKVVEGGPLPFAFDILATCFMYGSKTFINHTQGIPDFFKQSFPEGFTWERVTTYEDGGVLTVTQDTSLQDGCLIYNVKLRGVNFPSNGPVMQKKTLGWEASTETLYPADGGLEGRCDMALKLVGGGHLHCNLKTTYRSKKPAKNLKMPGVYFVDRRLERIKEADNETYVEQHEVAVARYCDLPSKLGHKLNGMDELYK

SEQ ID NO: 13 - protein, 239 aa, synthetic construct
MVSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSKLSKDPNEKRDHMVLLEFVTAAGITLGMDELYK

SEQ ID NO: 14 - protein, 244 aa, synthetic construct
GGVQLADHYQQNTPIGDGPVLLPDNHYLSYQSKLSKDPNEKRDHMVLLEFVTAAGITLGMDELYKGGTGGSMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIE

SEQ ID NO: 15 - protein, 237 aa, synthetic construct
MVSKGEGQSKHGLKEEMTVKYHMEGCVNGHKFVITGEGIGNPFKGKQTANLCVIEGGPLPFSEDILSPGFKYGDRIFTEYPQDIVDYFKNSCPAGYTWERSFLFEDGAVCRCNVDITVSEKENCIYHKSIFRGVNFPADGPVMKKMTTNWEASTEKIVPVPKQGILKGKVKMYLLLKDGGRYHCQFDTVYKAKSVPSKMPEWHFIQHKLLREDRSDAKNQKWQLTEHAIAGMDELYK

SEQ ID NO: 16 - protein, 229 aa, synthetic construct
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVMGTGFPADGPVMTNTLTAADLCVSKMTYPNDKTIISTFKWSYTTVNGKRYRSTARTTYTFAKPMAAKYLKNQPMYVLRKTELKHSMTELNFKEWQKAFTDMM

SEQ ID NO: 17 - protein, 316 aa, synthetic construct
MAEGSVARQPDLLTCDDEPIHIPGAIQPHGLLLALAADMTIVAGSDNLPELTGLAIGALIGRSAADVFDSETHNRLTIALAEPGAAVGAPITVGFTMRKDAGFIGSWHRHDQLIFLELEPPQRDVAEPQAFFRRTNSAIRRLQAAETLESACAAAAQEVRKITGFDRVMIYRFASDFSGSVIAEDRCAEVESKLGLHYPASFIPAQARRLYTINPVRIIPDINYRPVPVTPDLNPVTGRPIDLSFAILRSVSPNHLEFMRNIGMHGTMSISILRGERLWGLIVCHHRTPYYVDLDGRQACKRVAERLATQIGVMEE

SEQ ID NO: 18 - protein, 316 aa, synthetic construct
MAEGSVARQPDLLTCDDEPIHIPGAIQPHGLLLALAADMTIVAGSDNLPELTGLAIGALIGRSAADVFDSETHNRLTIALAEPGAAVGAPITVGFTMRKDAGFIGSWHRHDQLIFLELEPPQRDVAEPQAFFRHTNSAIRRLQAAETLESACAAAAQEVRKITGFDRVMIYRFASDFSGEVIAEDRCAEVESKLGQHYPASDIPAQARRLYTINPVRIIPDINYRPVPVTPDLNPVTGRPIDLSFAILRSVSPVHLEFMRNIGMHGTMSISILRGERLWGLIVCHHRTPYYVDLDGRQACELVAQVLARAIGVMEE

SEQ ID NO: 19 - protein, 239 aa, synthetic construct
MVSKGEELFTGVVPILVELDGDVNGHKFSVSGEGEGDATYGKLTLKFICTTGKLPVPWPTLLTTIGYGLMCFARYPDHMKQHDFFKSAMPEGYVQERTIFFKDDGNYKTRAEVKFEGDTLVNRIELKGIDFKEDGNILGHKLEYNHDSHNVYIMADKQKNGIKVNFKIRHNIEDGSVQLADHYQQNTPIGDGPVLLPDNHYLSYQSKLSKDPNEKRDHMVLLEFVTAAGITLGMDELYK

SEQ ID NO: 20 - protein, 226 aa, synthetic construct
MSAIKPDMKIKLRMEGNVNGHHFVIDGDGTGKPFEGKQSMDLEVKEGGPLPFAFDILTTAFSYGNRVFAKYPDNIQDYFKQSFPKGYSWERSLTFEDGGICNARNDITMEGDTFYNKVRFYGTNFPANGPVMQKKTLKWEPSTEKMYVRDGVLTGDVEMALLLEGNAHYRCDFRTTYKAKEKGVKLPGAHFVDHCIEILSHDKDYNKVKLYEHAVAHSGLPDNARR

SEQ ID NO: 21 - protein, 224 aa, synthetic construct
MSVIKPDMKIKLRMEGAVNGHPFAIEGVGLGKPFEGKQSMDLKVKEGGPLPFAYDILTTAFCYGNRVFAKYPENIVDYFKQSFPEGYSWERSMNYEDGGICNATNDITLDGDCYIYEIRFDGVNFPANGPVMQKRTVKWEPSTEKLYVRDGVLKGDVNMALSLEGGGHYRCDFKTTYKAKKVVQLPDYHFVDHHIEIKSHDKDYSNVNLHEHAEAHSELPRQAK

SEQ ID NO: 22 - protein, 239 aa, synthetic construct
MVSKGEELFTGVVPILVELDGDVNGHKFSVSGEGEGDATYGKLTLKFICTTGKLPVPWPTLVTTLAYGVLCFSRYPDHMKQHDFFKSAMPEGYVQERTIFFKDDGNYKTRAEVKFEGDTLVNRIELKGIDFKEDGNILGHKLEYNYNSHNVYIMADKQKNGIKSNFKIRHNIEDGSVQLADHYQQNTPIGDGPVLLPDNHYLSTQSKLSKDPNEKRDHMVLLEFVTAAGITLGMDELYK

SEQ ID NO: 23 - protein, 236 aa, synthetic construct
MVSKGEELFTGVVPILVELDGDVNGHKFSVRGEGEGDATNGKLTLKFICTTGKLPVPWPTLVTTFGYGVACFSRYPDHMKQHDFFKSAMPEGYVQERTISFKDDGTYKTRAEVKFEGDTLVNRIELKGIDFKEDGNILGHKLEYNFNSHYVYITADKQKNCIKANFKIRHNVEDGSVQLADHYQQNTPIGDGPVLLPDNHYLSHQSKLSKDPNEKRDHMVLLEFVTAALESGGEDP

SEQ ID NO: 24 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 25 - protein, 298 aa, synthetic construct
MGSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 26 - protein, 310 aa, synthetic construct
ETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGTGGSGGTGGSGGTGGSMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEW

SEQ ID NO: 27 - protein, 16 aa, synthetic construct
LRRATLVDGGTGGSEL

SEQ ID NO: 28 - protein, 16 aa, synthetic construct
LRRAALVDGGTGGSEL

SEQ ID NO: 29 - protein, 19 aa, synthetic construct
RFRRFQTLKDKAKAGGSEL

SEQ ID NO: 30 - protein, 20 aa, synthetic construct
PRPRSCTWPDPRPEFGGSEL

SEQ ID NO: 31 - protein, 23 aa, synthetic construct
PDVPRTPVDKAKLSFQFPGGSEL

SEQ ID NO: 32 - protein, 17 aa, synthetic construct
MRRVATLVDLGTGGSEL

SEQ ID NO: 33 - protein, 12 aa, synthetic construct
WMEDYDYVHLQG

SEQ ID NO: 34 - protein, 10 aa, synthetic construct
EKIEGTYGVV

SEQ ID NO: 35 - protein, 81 aa, synthetic construct
PSKTSNTIRVFLPNKQRTVVNVRNGMSLHDCLMKALKVRGLQPECCAVFRLLHEHKGKKARLDWNTDAASLIGEELQVDFL

SEQ ID NO: 36 - protein, 147 aa, synthetic construct
DQLTEEQIAEFKEAFSLFDKDGDGTITTKELGTVMRSLGQNPTEAELQDMINEVDADGDGTIDFPEFLTMMARKMKDTDSEEEIREAFRVFDKDGNGYISAAELRHVMTNLGEKLTDEEVDEMIREADIDGDGQVNYEEFVQMMTAK

SEQ ID NO: 37 - protein, 733 aa, synthetic construct
PVGTHEMEEELAEAVALLSQRGPDALLTVALRKPPGQRTDEELDLIFEELLHIKAVAHLSNSVKRELAAVLLFEPHSKAGTVLFSQGDKGTSWYIIWKGSVNVVTHGKGLVTTLHEGDDFGELALVNDAPRAATIILREDNCHFLRVDKQDFNRIIKDVEAKTMRLEEHGKVVLVLERASQGAGPSRPPTPGRNRYTVMSGTPEKILELLLEAMGPDSSAHDQTETFLSDFLLTHRVFMPSAQLCAALLHHFHVEPAGGSEQERSTYVCNKRQQILRLVSQWVALYGSMLHTDPVATSFLQKLSDLVGRDTRLSNLLREQWPERRRCHRLENGCGNASPQMKARNLPVWLPNQDEPLPGSSCAIQVGDKVPYDICRPDHSVLTLQLPVTASVREVMAALAQEDGWTKGQVLVKVNSAGDAIGLQPDARGVATSLGLNERLFVVNPQEAHELIPHPDQLGPTVGSAEGLDLVSAKDLAGQLTDHDWSLFNSIHQVELIHYVLGPQHLRDVTTANLERFMRRFNELQYWVATELCLCPVPGPRAQLLRKFIKLAAHLKEQKNLNSFFAVMFGLSNSAISHLAHTWERLPHKVRKLYSALERLLDPSWNHRVYRLALAKLSPPVIPFMPLLLKDMTFIHEGNHTLVENLINFEKMRMMARVARMLHHCRSHNPVPLSPLRSRVSHLHEDSQVARISTCSEQSLSTRSPASTWAYVQQLKVIDNQRELSRLSRELEP

SEQ ID NO: 38 - protein, 118 aa, synthetic construct
MSGGSSCSQTPSRAIPATRRVVLGDGVQLPPGDYSTTPGGTLFSTTPGGTRIIYDRKFLMECRNSPVTKTPPRDLPTIPGVTSPSSDEPPMEASQSHLRNSPEDKRAGGEESQFEMDI

SEQ ID NO: 39 - protein, 141 aa, synthetic construct
KFSQEQIGENIVCRVICTTGQIPIRDLSADISQVLKEKRSIKKVWTFGRNPACDYHLGNISRLSNKHFQILLGEDGNLLLNDISTNGTWLNGQKVEKNSNQLLSQGDEITVGVGVESDILSLVIFINDKFKQCLEQNKVDR

SEQ ID NO: 40 - protein, 54 aa, synthetic construct
MADEEKLPPGWEKRMSRSSGRVYYFNHITNASQWERPSGNSSSGGKNGQGEPAR

SEQ ID NO: 41 - protein, 98 aa, synthetic construct
WYFGKITRRESERLLLNPENPRGTFLVRESETTKGAYCLSVSDFDNAKGLNVKHYKIRKLDSGGFYITSRTQFSSLQQLVAYYSKHADGLCHRLTNVC

SEQ ID NO: 42 - protein, 17 aa, synthetic construct
QNHFVDEYDPTIGGSEL

SEQ ID NO: 43 - protein, 21 aa, synthetic construct
SSRRKWNKTGHAVRAIGRLSS

SEQ ID NO: 44 - protein, 173 aa, synthetic construct
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNG

SEQ ID NO: 45 - protein, 184 aa, synthetic construct
MREYKLVVLGSGGVGKSALTVQFVQGIFVEKYDPTIEDSYRKQVEVDCQQCMLEILDTAGTEQFTAMRDLYMKNGQGFALVYSITAQSTFNDLQDLREQILRVKDTEDVPMILVGNKCDLEDERVVGKEQGQNLARQWCNCAFLESSAKSKINVNEIFYDLVRQINRKTPVEKKKPKKKSCLLL

SEQ ID NO: 46 - protein, 116 aa, synthetic construct
SAGGSAGGSAGGSAGGSAGGSGSAGGSAGGSTSAGGSAGGSAGGSAGGSAGGSGSAGGSAGGSTSAGGSAGGSAGGSAGGSAGGSGSAGGSAGGSTSAGGSAGGSAGGSAGGSAGG

SEQ ID NO: 47 - protein, 14 aa, synthetic construct
SAGKPGSGEGSTKG

SEQ ID NO: 48 - protein, 20 aa, synthetic construct
SAGGSAGGSAGGSAGGSAGG

SEQ ID NO: 49 - protein, 24 aa, synthetic construct
GTSAGGSAGGSAGGSAGGSAGGSG

SEQ ID NO: 50 - protein, 32 aa, synthetic construct
SAGGSAGGSAGGSAGGSAGGSGSAGGSAGGST

SEQ ID NO: 51 - protein, 40 aa, synthetic construct
SAGGSAGGSGSAGGSAGGSTSAGGSAGGSAGGSAGGSAGG

SEQ ID NO: 52 - protein, 52 aa, synthetic construct
SAGGSAGGSAGGSAGGSAGGSGSAGGSAGGSTSAGGSAGGSAGGSAGGSAGG

SEQ ID NO: 53 - protein, 64 aa, synthetic construct
SAGGSAGGSAGGSAGGSAGGSGSAGGSAGGSTSAGGSAGGSAGGSAGGSAGGSGSAGGSAGGST

SEQ ID NO: 54 - protein, 80 aa, synthetic construct
SAGGSAGGSAGGSAGGSAGGSGSAGGSAGGSTSAGGSAGGSAGGSAGGSGSAGGSAGGSTSAGGSAGGSAGGSAGGSAGG

SEQ ID NO: 55 - protein, 142 aa, synthetic construct
SAGGSAGGSAGGSAGGSAGGSGSAGGSAGGSTSAGGSAGGSAGGSAGGSAGGSGSAGGSAGGSAGGSAGGSGSAGGSAGGSTSAGGSAGGSAGGSAGGSAGGSGSAGGSAGGSTSAGGSAGGSAGGSAGGSAGGSAGGSAGG

SEQ ID NO: 56 - protein, 190 aa, synthetic construct
SAGGSAGGSAGGSAGGSAGGSGSAGGSAGGSTSAGGSAGGSAGGSAGGSAGGSGSAGGSAGGSTSAGGSAGGSAGGSAGGSAGGSGSAGGSAGGSTSAGGSAGGSAGGSAGGSAGGSGSAGGSAGGSTSAGGSAGGSAGGSAGGSAGGSGSAGGSAGGSTSAGGSAGGSAGGSAGGSAGGSGSAGGSAGG

SEQ ID NO: 57 - protein, 220 aa, synthetic construct
SAGGSAGGSAGGSAGGSAGGSGSAGGSAGGSTSAGGSAGGSAGGSAGGSAGGSGSAGGSAGGSTSAGGSAGGSAGGSAGGSAGGSGSAGGSAGGSTSAGGSAGGSAGGSAGGSAGGSGSAGGSAGGSTSAGGSAGGSAGGSAGGSAGGSGSAGGSAGGSTSAGGSAGGSAGGSAGGSAGGSGSAGGSAGGSTSAGGSAGGSAGGSAGGSAGGSAGGSAGG

SEQ ID NO: 58 - protein, 258 aa, synthetic construct
SAGGSAGGSAGGSAGGSAGGSGSAGGSAGGSTSAGGSAGGSAGGSAGGSAGGSGSAGGSAGGSTSAGGSAGGSAGGSAGGSAGGSGSAGGSAGGSTSAGGSAGGSAGGSAGGSAGGSGSAGGSAGGSTSAGGSAGGSAGGSAGGSAGGSGSAGGSAGGSTSAGGSAGGSAGGSAGGSTSAGGSAGGSAGGSAGGSAGGSGSAGGSAGGSTSAGGSAGGSAGGSAGGSGSAGGSAGGSTSAGGSAGGSAGGSAGGSAGG

SEQ ID NO: 59 - protein, 314 aa, synthetic construct
GGTACCAGTGCTGGTGGTAGTGCTGGTGGTAGTGCTGGTGGTTCCGGCAGTGCTGGTGGTAGTGCTGGTGGTAGTACCAGTGCTGGTGGTAGTGCTGGTGGTAGTGCTGGTGGTAGTGCTGGTGGTAGTGCTGGTGGTTCCGGCAGTGCTGGTGGTAGTGCTGGTGGTAGTACCAGTGCTGGTGGTAGTGCTGGTGGTAGTGCTGGTGGTAGTGCTGGTGGTAGTGCTGGTGGTTCCGGCAGTGCTGGTGGTAGTGCTGGTGGTAGTACCAGTGCTGGTGGTAGTGCTGGTGGTAGTGCTGGTGGTAGTGCTGG

SEQ ID NO: 60 - protein, 214 aa, synthetic construct
MSKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSTQTVLSKDPNEK

SEQ ID NO: 61 - protein, 213 aa, synthetic construct
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVMGTGFPADGPVMTNTLTAADWCMSKKTYPNDKTIISTFKWSYTTVNGKRYRSTARTTYTFAKPMAANYLKNQPMYVFRKTELKHSM

SEQ ID NO: 62 - protein, 213 aa, synthetic construct
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVMGTGFPADGPVMTNTLTAADLCVSKMTYPNDKTIISTFKWSYTTVNGKRYRSTARTTYTFAKPMAAKYLKNQPMYVLRKTELKHSM

SEQ ID NO: 63 - protein, 16 aa, synthetic construct
RDHMVLHEYVNAAGIT

SEQ ID NO: 64 - protein, 16 aa, synthetic construct
TELNFKEWQKAFTDMM

SEQ ID NO: 65 - protein, 202 aa, synthetic construct
RDHMVLHEYVNAAGITGGSGSSGGGSGGGSSRDHMVLHEYVNAAGITGSGGSGSGSGSGGSSRDHMVLHEYVNAAGITGSGSGSSGGGSSGSSRDHMVLHEYVNAAGITGSGGSGSGSSGGSGSRDHMVLHEYVNAAGITGSGGSGGSGSGSGGSRDHMVLHEYVNAAGITGGSSGGSSGSGGSSSRDHMVLHEYVNAAGIT

SEQ ID NO: 66 - protein, 58 aa, synthetic construct
TELNFKEWQKAFTDMMGGSGSTELNFKEWQKAFTDMMGSGGSTELNFKEWQKAFTDMM

SEQ ID NO: 67 - protein, 156 aa, synthetic construct
MGSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTT

SEQ ID NO: 68 - protein, 145 aa, synthetic construct
MGSDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

(19) United States
(12) Patent Application Publication
(10) Pub. No.: US 2026/0235593 A1
(43) Pub. Date: Aug. 13, 2026

(54) FRET BIOSENSOR RECOMBINANT PROTEINS

(71) Applicant: The Regents of the University of California, Oakland, CA (US)

(72) Inventors: Xinchang HE, La Jolla, CA (US); Michelle Susanne FREI, La Jolla, CA (US); Jin ZHANG, La Jolla, CA (US)

(21) Appl. No.: 19/474,631

(22) PCT Filed: Apr. 12, 2024
(86) PCT No.: PCT/US2024/024488

§ 371 Date: Oct. 10, 2025

Related U.S. Application Data
(60) Provisional application No. 63/495,948, filed on Apr. 13, 2023.

(51) Int. Cl. G01N 33/542; C07K 14/435; C12N 9/14

(52) U.S. Cl. CPC G01N 33/542; C07K 14/43595; C12N 9/14; C12Y 308/01005; C07K 2319/60; C07K 2319/61

(57) ABSTRACT

Provided herein are, inter alia, FRET biosensor recombinant proteins, which provide quantitative measurement of biomolecule activity in living cells with high spatiotemporal resolution. The FRET biosensor recombinant proteins provided herein are, inter alia, useful for detecting a biomolecule and activity of a biomolecule in a cell.

### RELATED APPLICATION DATA

This application claims the benefit of priority under 35 U.S.C. § 119(e) of U.S. Patent Application No. 63/495,948, filed on Apr. 13, 2023, which is hereby incorporated by reference in its entirety and for all purposes.

### GOVERNMENT SUPPORT CLAUSE

This invention was made with government support under R35 CA197622 awarded by the National Institutes of Health. The government has certain rights in the invention.

### SEQUENCE LISTING

The material in the accompanying Sequence Listing is hereby incorporated by reference in its entirety. The accompanying file, named “037866-727001WO_SL_ST26.xml” was created on Apr. 11, 2024, and is 70,270 bytes in size.

### BACKGROUND

Kinases are key players in cell signaling and thereby regulate almost all aspects of cellular function in both health and disease. Their aberrant function often leads to severe diseases such as cancer. In order to infer the activity of kinases fluorescent kinase activity reporters (KARs) have been developed. The main limitations of current FRET-based biosensors stem from the use of fluorescent proteins (FPs) as FRET pairs. Due to the FP's suboptimal photophysical properties the biosensors show small dynamic ranges, low brightness and low photostability. In addition, their spectral properties are mainly limited to the blue-red region as far-red and NIR fluorescent FPs are rare. This complicates the use of these biosensors in multiplexing experiments or high-throughput measurements. Provided herein, inter alia, are compositions and methods that address these and other problems in the art.

### BRIEF SUMMARY

In an aspect is provided a recombinant protein covalently bound to a chemical fluorophore, the recombinant protein including a fluorescent protein domain bound to a biosensing protein domain and a haloalkane dehalogenase domain bound to the biosensing protein domain, wherein: the chemical fluorophore is covalently bound to the haloalkane dehalogenase domain; and the chemical fluorophore and the fluorescent protein domain are a Forster Resonance Energy Transfer (FRET) pair.

In another aspect is provided a recombinant protein covalently bound to a chemical fluorophore, the recombinant protein including a split fluorescent protein domain bound to a biosensing protein domain and a haloalkane dehalogenase domain bound to the biosensing protein domain, wherein: the chemical fluorophore is covalently bound to the haloalkane dehalogenase domain; the split fluorescent protein domain is one part of a fluorescent protein split pair that forms a reconstituted fluorescent protein upon binding of a cognate split fluorescent protein; and the chemical fluorophore and the reconstituted fluorescent protein domain are a Forster Resonance Energy Transfer (FRET) pair.

In another aspect is provided a kit including the recombinant protein provided herein including embodiments thereof and a second recombinant protein, wherein the second recombinant protein includes a protein of interest domain bound to the cognate split fluorescent protein.

In another aspect is provided a recombinant protein including a fluorescent protein domain bound to a biosensing protein domain and a split haloalkane dehalogenase domain, wherein: the split haloalkane dehalogenase domain is one part of a haloalkane dehalogenase split pair that forms a reconstituted haloalkane dehalogenase protein upon binding of a cognate haloalkane dehalogenase split protein domain.

In another aspect is provided a kit including the recombinant protein provided herein including embodiments thereof and a second recombinant protein, wherein the second recombinant protein includes the cognate haloalkane dehalogenase split protein domain bound to a protein of interest.

In another aspect is provided an isolated nucleic acid encoding the recombinant protein provided herein including embodiments thereof.

In another aspect is provided an expression vector including the isolated nucleic acid provided herein including embodiments thereof.

In another aspect is provided a method for detecting a target biomolecule in a cell, the method including: (a) contacting the cell with an expression vector including a nucleic acid, wherein the nucleic acid encodes a recombinant protein provided herein including embodiments thereof; (b) transducing the cell with the expression vector: (c) allowing the cell to express the recombinant protein and interact with the target biomolecule in the cell; and (d) detecting a change in fluorescent signal from the recombinant protein, thereby detecting the target biomolecule.

In another aspect is provided a method for detecting a target biomolecule in a cell, the method including: (a) contacting the cell with an expression vector including a nucleic acid, wherein the nucleic acid encodes a recombinant protein provided herein including embodiments thereof; (b) transducing the cell with the expression vector; (c) allowing the cell to express the recombinant protein and interact with the target biomolecule in the cell; and (d) detecting a change in fluorescent signal from the recombinant protein, thereby detecting the target biomolecule.

In another aspect is provided a method for detecting a target biomolecule in a cell, the method including: (a) contacting the cell with an expression vector including a nucleic acid, wherein the nucleic acid encodes a recombinant protein provided herein including embodiments thereof; (b) transducing the cell with the expression vector: (c) allowing the cell to express the recombinant protein and interact with the target biomolecule in the cell; and (d) detecting a change in fluorescent signal from the recombinant protein, thereby detecting the target biomolecule.

In some example embodiments, there may be provided biosensors.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIGS. 1A-1D show generation and characterization of different RFP-HaloTag^{(NGD)} FRET-based AKARs. FIG. 1A: Domain structure of the biosensors. FIG. 1B: Schematic diagram of the biosensor's conformational change induced by phosphorylation. FIG. 1C: Average time course of the Azalea-B5-FHA1-EV linker-PKA substrate-HaloTag^{(NGD)} biosensor expressed in HeLa cells in response to 50 μM Fsk, 100 μM IBMX and 20 μM H89 (n=21 cells) [mean±95% CI]. FIG. 1D: Maximum normalized ratio of the different RFP-HaloTag^{(NGD)} biosensors expressed in HeLa cells after stimulation with 50 M Fsk, 100 M IBMX for 8 min (n=5-28 cells) [mean±95% CI].

FIGS. 2A-2C show generation and characterization of different RFP-HaloTag^{(JF669)} FRET-based AKARs using the RFP as FRET donor. FIG. 2A: Schematic diagram of the biosensor's conformational change induced by phosphorylation. FIG. 2B: Average time course of the sTagRFP-FHA1-EV linker-PKA substrate-HaloTag^{(JF669)} biosensor expressed in HeLa cells in response to 50 μM Fsk, 100 μM IBMX and 20 M H89 (n=8 cells) [mean±95% CI]. FIG. 2C: Maximum normalized ratio of the different RFP-HaloTag^{(JF669)} biosensors expressed in HeLa cells after stimulation with 50 M Fsk, 100 M IBMX for 8 min (n=4-15 cells) [mean±95% CI].

FIGS. 3A-3G show generation and characterization of different length EV linker of sTagRFP-HaloTag^{(JF669)} FRET-based AKARs. FIG. 3A: Domain structure of the biosensors. FIGS. 3B-3F: Characterization of the different sTagRFP-FHA1-different length EV linkers-PKA substrate-HaloTag^{(JF669)} biosensors expressed in HeLa cells: basal emission ratio (FIG. 3B), maximum normalized ratio after stimulation for 8 min (FIG. 3C), half-life of the on kinetics (FIG. 3D), half-life of the off kinetics (FIG. 3E), average time course in response to 50 M Fsk, 100 M IBMX and 20 μM H89 (FIG. 3F) (n=8-19 cells) [mean±95% CI]. FIG. 3G: Average time course of the sTagRFP-FHA1-258 aa EV linker-PKA substrate-HaloTag^{(JF669)} (T/A) biosensor expressed in HeLa cells in response to 50 μM Fsk, 100 μM IBMX and 20 M H89.

FIGS. 4A-4H show generation and characterization of new PKC, Akt/PKB, ERK, and cAMP biosensors using the sTagRFP-HaloTag^{(JF669)} FRET pair. FIGS. 4A-4D: Domain structure of the biosensors. PKC biosensor (FIG. 4A). Akt/PKB biosensor (FIG. 4B), ERK biosensor (FIG. 4C), and cAMP biosensor (FIG. 4D). FIG. 4E: Average time course of the PKC biosensor expressed in HeLa cells in response to 50 ng/ml PMA (n=12 cells) [mean±95% CI]. FIG. 4F: Average time course of the Akt biosensor expressed in NIH3T3 cells in response to 100 ng/ml PDGF (n=6 cells) [mean 95% CI]. FIG. 4G: Average time course of the ERK biosensor expressed in HEK293T cells in response to 100 ng/ml hEGF (n=8 cells) [mean±95% CI]. FIG. 4H: Average time course of the cAMP biosensor expressed in HEK293T cells in response to 20 μM isoproterenol (Iso), 50 μM Fsk and 100 μM IBMX (n=9 cells) [mean±95% CI].

FIGS. 5A-5D show generation and characterization of different YFP-HaloTag^{(JF669)} FRET-based AKARs using the YFP as FRET donor. FIG. 5A: Domain structure of the biosensors. FIG. 5B: Schematic diagram of the biosensor's conformational change induced by phosphorylation. FIG. 5C: Average time course of the mCitrine-FAH1-EV linker-PKA substrate-HaloTag biosensor in response to 50 μM Fsk, 100 μM IBMX and 20 μM H89 (n=14 cells) [mean±95% CI]. FIG. 5D: Maximum normalized ratio of the different YFP-HaloTag^{(JF669)} biosensors expressed in HeLa cells after stimulation with 50 uM Fsk and 100 uM IBMX for 8 min (n=4-9 cells) [mean±95% CI].

FIGS. 6A-6G show generation and characterization of different length EV linkers of mCitrine-HaloTag^{(JF669)} FRET-based AKARs. FIG. 6A: Domain structure of the biosensors. FIG. 6B: Average time course of the mCitrine-FHA1-different length EV linkers-PKA substrate-HaloTag^{(JF669)} biosensors expressed in HeLa cells in response to 50 μM Fsk, 100 M IBMX and 20 μM H89 (n=11-43 cells) [mean±95% CI]. FIG. 6C: Average time course of the mCitrine-FHA1-330 aa EV linkers-PKA substrate-HaloTag^{(JF669)} (I/A) biosensor expressed in HeLa cells in response to 50 μM Fsk, 100 μM IBMX and 20 M H89 (n=8-38 cells) [mean±95% CI]. FIGS. 6D-6G: Characterization of the different mCitrine-FHA1-different length EV linkers-PKA substrate-HaloTag^{(JF669)} biosensors expressed in HeLa cells: maximum normalized ratio after stimulation for 8 min (FIG. 6D), basal emission ratio (FIG. 6E), half-life of the on kinetics (FIG. 6F), half-life of the off kinetics (FIG. 6G) (n=11-43 cells) [mean±95% CI].

FIGS. 7A-7H show generation and characterization of PKC, Akt/PKB. ERK, and cAMP biosensors using the mCitrine-HaloTag^{(JF669)} FRET pair. FIGS. 7A-7D: Domain structure of the biosensors: PKC (FIG. 7A), Akt (FIG. 7B), ERK (FIG. 7C), cAMP (FIG. 7D). FIG. 7E: Average time course of the PKC biosensor expressed in HeLa cells in response to 50 ng/ml PMA (n=11 cells) [mean±95% CI]. FIG. 7F: Average time course of the Akt biosensor expressed in NIH3T3 cells in response to 100 ng/ml PDGF (n=6 cells) [mean±95% CI]. FIG. 7G: Average time course of the ERK biosensor expressed in HEK293T cells in response to 100 ng/ml hEGF (n=8 cells) [mean±95% CI]. FIG. 7H: Average time course of the cAMP biosensor expressed in HEK293T cells in response to 50 M Fsk. 100 M IBMX (n=14 cells) [mean±95% CI].

FIGS. 8A-8D show generation and characterization of enhanced FluoSTEPs using HaloTag and three different split donor FPs. FIG. 8A: Domain structure of the biosensors. FIG. 8B: Schematic diagram of the biosensors conformational change induced by phosphorylation. FIG. 8C Maximum normalized ratio of the different split FPs-HaloTag^{(JF669)} FluoSTEP-AKARs expressed in HeLa cells after stimulation with 50 uM Fsk and 100 uM IBMX for 8 min (n=3-20 cells) [mean±95% CI]. FIG. 8D: Average time course of the different split FPs-HaloTag^{(JF669)} FluoSTEP-AKARs in response to 50 μM Fsk, 100 μM IBMX and 20 M H89 (n=3-20 cells) [mean±95% CI].

FIGS. 9A-9E show FluoSTEP-AKAR dynamic range and brightness optimization with multiple mNG11 and longer EV linkers. FIGS. Domain structure of the biosensors. (B) Maximum normalized ratio of the reconstituted mNG2/mNG3A-HaloTag^{(JF669)} FluoSTEP-AKARs (single/triple mNG11-Actin) expressed in HeLa cells after stimulation with 50 uM Fsk and 100 uM IBMX (n=11-20 cells) [mean±95% CI]. (C) Domain structure of the longer EV linker FluoSTEP-AKARs. (D) Maximum normalized ratio of the reconstituted mNG3A-HaloTag^{(JF669)} FluoSTEP-AKARs with different length EV linkers expressed (mNG11x3-Actin) in HeLa cells after stimulation with 50 uM Fsk and 100 uM IBMX (n=10-16 cells) [mean±95% CI]. (E) Average time course of the reconstituted mNG3A-HaloTag^{(JF669)} FluoSTEP-AKARs with different length EV linkers (mNG11x3-Actin) expressed in HeLa cells after stimulation.

FIGS. 10A-10C show generation and characterization of FluoSTEPs using split HaloTag and mScarlet. FIG. 10A: Domain structure of the two biosensors and localization sequences used. FIGS. 10B-10C: Average time course of the different mScarlet-split-HaloTag^{(NGD)} FluoSTEP-AKARs in response to 50 M Fsk and 100 μM IBMX (n=5-8 cells) [mean±95% CI] targeted to the plasma membrane via KRAS (FIG. 10B), and Lyn11 (FIG. 10C).

FIGS. 11A-11C show generation and characterization of different mScarlet-cpHaloTag^{(NGD)} FRET-based AKARs. FIG. 11A: Domain structure of the biosensors. FIG. 11B: Schematic diagram of biosensor conformational change induced by phosphorylation. FIG. 11C: Maximum normalized ratio of the different RFP-HaloTag^{(NGD)} biosensors expressed in HeLa cells after stimulation with 50 M Fsk, 100 M IBMX (n=7-22 cells) [mean±SEM].

FIGS. 12A-12C show generation and characterization of 5 different “shuffling” FRET-based AKARs. FIG. 12A: Domain structure of the biosensors. FIG. 12B: Maximum normalized ratio of 5 different “shuffling” FRET-based AKARs expressed in HeLa cells and labeled by NGD after stimulation with 50 M Fsk, 100 μM IBMX (n=7-12 cells) [mean SEM]. FIG. 12C: Maximum normalized ratio of 5 different “shuffling” FRET-based AKARs expressed in HeLa cells and labeled by JF669 after stimulation with 50 μM Fsk, 100 μM IBMX (n=7-10 cells) [mean±SEM].

FIGS. 13A-13B show the characterization of different length of EV linkers of Azalea B5-FHA1-EV linker-PKA substrate-HaloTag^{(NGD)} FRET-based AKARs. FIG. 13A: Domain structure of the biosensors. FIG. 13B: Maximum normalized ratio of the different RFP-HaloTag^{(NGD)} biosensors expressed in HeLa cells after stimulation with 50 M Fsk and 100 M IBMX (n=6-15 cells)[mean±SEM].

FIG. 14 shows the characterization of mCitrine-FHA1-EV linker-PKA substrate-HaloTag FRET-based AKARs labeled with three different synthetic fluorophores. Maximum normalized ratio of the mCitrine-HaloTag^{(X)} biosensor expressed in HeLa cells after stimulation with 50 M Fsk, 100 M IBMX (n=8-19 cells) [mean±SEM].

FIGS. 15A-15C show the characterization of Dreiklang-FHA1-EV linker-PKA substrate-HaloTag^{(JF669)} photoswitchable-AKARs. FIG. 15A: Domain structure of the biosensor. FIG. 15B: Average time course of the different EV linker Dreiklang-HaloTag^{(JF669)} biosensors expressed in HeLa cells after stimulation with 50 M Fsk, 100 μM IBMX (n=10-12 cells) [mean±SEM]. FIG. 15C: Switching kinetics of the biosensor. Normalized YFP intensity of the Dreiklang biosensor upon irradiation with 405 nm (turn-off) or 365 nm (turn-on) (n=5-14 cells) [mean±SEM].

FIGS. 16A-16C show biosensor multiplexing experiments using HaloTag-based FRET biosensors. FIGS. 16A-16C: Average time courses of co-imaged biosensors. FIG. 16A: Yellow/far-red-HaloTag-EKAR (dashed line) and red/far-red-HaloTag-AKAR (solid black line) in HEK293T cells upon pharmacological stimulation with 100 ng/mL hEGF, 1.7 μM SCH772984, 50 μM Fsk/100 M IBMX, and 20 M H89. FIG. 16B: Yellow/far-red-HaloTag-AKAR (dashed line) and sTagRFP-Epac-HaloTag^{(JF669)} (solid black line) in HeLa cells upon stimulation with 50 μM Fsk and 100 μM IBMX followed by 20 μM H89. FIG. 16C: ExRai-CKAR (dashed line) and red/far-red-HaloTag-AKAR (solid black line) in HeLa cells upon pharmacological stimulation with 50 ng/ml PMA, 1 M G66983, 50 μM Fsk and 100 M IBMX followed by 20 M H89. Mean responses are given.

FIGS. 17A-17C show FluoSTEP-AKAR measurements on the endogenous level. FIGS. 17A-17C: Average time courses of mNG3A₍₁₋₁₀₎-FHA1-116 aa EV linker-PKA substrate-HaloTag^{(JF669)} (solid black line) and mNG3A₍₁₋₁₀₎-FHA1-220 aa EV linker-PKA substrate-HaloTag^{(JF669)} (dashed line) overexpressed in (FIG. 17A) HeLa-mNG₁₁-clathrin, (FIG. 17B) HeLa-mNGn₁₁-β-Actin, or (FIG. 17C) HeLa-mNGn₁₁-ezrin cells upon stimulation with 50 μM Fsk and 100 μM IBMX. Mean responses are given.

FIGS. 18A-18C show universal designs of chemigenetic biosensors. FIGS. 18A-18C: Average time courses of mCitrine-SH2-Src Sub-HaloTag^{(JF669)} (FIG. 18A), mCitrine-SH2-Fyn Sub-HaloTag^{(JF669)} (FIG. 18B), and mCitrine-RBD-PL-HaloTag^{(JF669)} (FIG. 18C) overexpressed in COS7 cells upon stimulation with 100 ng/mL hEGF and 200 nM AG1478 (only in FIG. 18C). Mean responses are given.

### DETAILED DESCRIPTION

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones: non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A): cytosine (C): guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence.

Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified. e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., Ras) in other proteins with different numbering systems. For example, by performing a simple sequence alignment with a protein (e.g., Ras) the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid.

One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments: or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “specifically (or selectively) binds to” when referring to a protein or peptide, refers to a binding reaction that is determinative of the presence of the protein, often in a heterogeneous population of proteins and other biologics. Thus, under designated immunoassay conditions, the specified proteins bind to a particular protein at least two times the background and more typically more than 10 to 100 times background.

A “ligand” refers to an agent, e.g., a polypeptide or other molecule, capable of binding to a specific protein or fragment thereof.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “protein kinase A” or “PKA” as used herein include any of the recombinant or naturally-occurring forms of the protein kinase A (PKA), or variants or homologs thereof that maintain PKA activity (e.g. within at least 50%, 80%, 90%, 95%, 96%, 97%, 98%, 99% or 100% activity compared to PKA). In some aspects, the variants or homologs have at least 90%, 95%, 96%, 97%, 98%, 99% or 100% amino acid sequence identity across the whole sequence or a portion of the sequence (e.g. a 25, 50, 100, 150, 200, 250, or 300 continuous amino acid portion) compared to a naturally occurring PKA protein. In embodiments, the PKA protein is substantially identical to the protein identified by the UniProt reference number P17612 or a variant or homolog having substantial identity thereto.

The term “protein kinase C” or “PKC” as used herein include any of the recombinant or naturally-occurring forms of the protein kinase C (PKC), or variants or homologs thereof that maintain PKC activity (e.g. within at least 50%, 80%, 90%, 95%, 96%, 97%, 98%, 99% or 100% activity compared to PKC). In some aspects, the variants or homologs have at least 90%, 95%, 96%, 97%, 98%, 99% or 100% amino acid sequence identity across the whole sequence or a portion of the sequence (e.g. a 25, 50, 100, 150, 200, 250, 300, 350, 400, 450, 500, 550, or 600 continuous amino acid portion) compared to a naturally occurring PKC protein. In embodiments, the PKC protein is substantially identical to the protein identified by the UniProt reference number P17252 or a variant or homolog having substantial identity thereto.

The term “protein kinase B” or “AKT” as used herein include any of the recombinant or naturally-occurring forms of the protein kinase B (PKB), also known as RAC (Rho family)-alpha serine/threonine-protein kinase (AKT) or variants or homologs thereof that maintain PKB activity (e.g. within at least 50%, 80%, 90%, 95%, 96%, 97%, 98%, 99% or 100% activity compared to PKB). In some aspects, the variants or homologs have at least 90%, 95%, 96%, 97%, 98%, 99% or 100% amino acid sequence identity across the whole sequence or a portion of the sequence (e.g. a 25, 50, 100, 150, 200, 250, 300, 350, 400, or 450 continuous amino acid portion) compared to a naturally occurring PKB protein. In embodiments, the PKB protein is an AKT1 protein. In embodiments, the PKB protein is substantially identical to the protein identified by the UniProt reference number P31749 or a variant or homolog having substantial identity thereto. In embodiments, the PKB protein is an AKT2 protein. In embodiments, the PKB protein is substantially identical to the protein identified by the UniProt reference number P31751 or a variant or homolog having substantial identity thereto. In embodiments, the PKB protein is an AKT3 protein. In embodiments, the PKB protein is substantially identical to the protein identified by the UniProt reference number Q9Y243 or a variant or homolog having substantial identity thereto.

The term “extracellular signal-related kinase” or “MAPK” as used herein include any of the recombinant or naturally-occurring forms of the extracellular signal-related kinase (ERK), also known as mitogen-activated protein kinase (MAPK), or variants or homologs thereof that maintain ERK activity (e.g. within at least 50%, 80%, 90%, 95%, 96%, 97%, 98%, 99% or 100% activity compared to ERK). In some aspects, the variants or homologs have at least 90%, 95%, 96%, 97%, 98%, 99% or 100% amino acid sequence identity across the whole sequence or a portion of the sequence (e.g. a 25, 50, 100, 150, 200, 250, 300, or 350 continuous amino acid portion) compared to a naturally occurring ERK protein. In embodiments, the ERK protein is a ERK1 (MAPK3) protein. In embodiments, the ERK protein is substantially identical to the protein identified by the UniProt reference number P27361 or a variant or homolog having substantial identity thereto. In embodiments, the ERK protein is a ERK2 (MAPK1) protein. In embodiments, the ERK protein is substantially identical to the protein identified by the UniProt reference number P28482 or a variant or homolog having substantial identity thereto.

The term “5′ adenosine monophosphate-activated protein kinase” or “AMPK” as used herein include any of the recombinant or naturally-occurring forms of the 5′ AMP-activated protein kinase (AMPK), or variants or homologs thereof that maintain AMPK activity (e.g. within at least 50%, 80%, 90%, 95%, 96%, 97%, 98%, 99% or 100% activity compared to AMPK). In some aspects, the variants or homologs have at least 90%, 95%, 96%, 97%, 98%, 99% or 100% amino acid sequence identity across the whole sequence or a portion of the sequence (e.g. a 25, 50, 100, 150, 200, 250, 300, or 350 continuous amino acid portion) compared to a naturally occurring AMPK protein. In embodiments, the AMPK protein is substantially identical to the protein identified by the UniProt reference number Q13131 or a variant or homolog having substantial identity thereto In embodiments, the AMPK protein is substantially identical to the protein identified by the UniProt reference number P54646 or a variant or homolog having substantial identity thereto.

The term “mammalian target of rapamycin” or “mTOR” as used herein include any of the recombinant or naturally-occurring forms of the mammalian target of rapamycin (mTOR), also known as FK506-binding protein 12-rapamycin-associated protein 1 (FRAP1), or variants or homologs thereof that maintain mTOR activity (e.g. within at least 50%, 80%, 90%, 95%, 96%, 97%, 98%, 99% or 100% activity compared to mTOR). In some aspects, the variants or homologs have at least 90%, 95%, 96%, 97%, 98%, 99% or 100% amino acid sequence identity across the whole sequence or a portion of the sequence (e.g. a 25, 50, 100, 150, 200.250, 300, 400, 500, 600, 700, 800, 900, 1000, 1100, 1200, 1300, 1400, 1500, 1600, 1700, 1800, 1900, 2000, 2100, 2200, 2300, 2400, or 2500 continuous amino acid portion) compared to a naturally occurring mTOR protein. In embodiments, the mTOR protein is substantially identical to the protein identified by the UniProt reference number P42345 or a variant or homolog having substantial identity thereto.

The term “proto-oncogene tyrosine-protein kinase Fyn” or “Fyn” as used herein include any of the recombinant or naturally-occurring forms of the proto-oncogene tyrosine-protein kinase Fyn (Fyn), or variants or homologs thereof that maintain Fyn activity (e.g. within at least 50%, 80%, 90%, 95%, 96%, 97%, 98%, 99% or 100% activity compared to Fyn). In some aspects, the variants or homologs have at least 90%, 95%, 96%, 97%, 98%, 99% or 100% amino acid sequence identity across the whole sequence or a portion of the sequence (e.g. a 25, 50, 100, 150, 200, 250, 300, 350, 400, 450, or 500 continuous amino acid portion) compared to a naturally occurring Fyn protein. In embodiments, the Fyn protein is substantially identical to the protein identified by the UniProt reference number P06241 or a variant or homolog having substantial identity thereto.

The term “proto-oncogene tyrosine-protein kinase Src” or “Src” as used herein include any of the recombinant or naturally-occurring forms of the proto-oncogene tyrosine-protein kinase Src (Src), also known as proto-oncogene cellular-Src (c-Src), or variants or homologs thereof that maintain Src activity (e.g. within at least 50%, 80%, 90%, 95%, 96%, 97%, 98%, 99% or 100% activity compared to Src). In some aspects, the variants or homologs have at least 90%, 95%, 96%, 97%, 98%, 99% or 100% amino acid sequence identity across the whole sequence or a portion of the sequence (e.g. a 25, 50, 100, 150, 200, 250, 300, 350, 400, 450, or 500 continuous amino acid portion) compared to a naturally occurring Src protein. In embodiments, the Src protein is substantially identical to the protein identified by the UniProt reference number P12931 or a variant or homolog having substantial identity thereto.

The terms “Ras protein” and “Ras” as used herein include any of the recombinant or naturally-occurring forms of the Rat sarcoma virus, also known as Ras GTPase, or variants or homologs thereof that maintain Ras activity (e.g. within at least 50%, 80%, 90%, 95%, 96%, 97%, 98%, 99% or 100% activity compared to Ras). In some aspects, the variants or homologs have at least 90%, 95%, 96%, 97%, 98%, 99% or 100% amino acid sequence identity across the whole sequence or a portion of the sequence (e.g. a 25, 50, 100, or 150 continuous amino acid portion) compared to a naturally occurring Ras protein. In embodiments, the Ras protein is substantially identical to the protein identified by the UniProt reference number P01116 or a variant or homolog having substantial identity thereto.

The terms “Ras-proximate-1” or “Rap1” as used herein include any of the recombinant or naturally-occurring forms of the Ras-proximate-1 protein, also known as Ras-related protein 1 or Rap1 GTPase. or variants or homologs thereof that maintain Rap1 activity (e.g. within at least 50%, 80%, 90%, 95%, 96%, 97%, 98%, 99% or 100% activity compared to Rap1). In some aspects, the variants or homologs have at least 90%, 95%, 96%, 97%, 98%, 99% or 100% amino acid sequence identity across the whole sequence or a portion of the sequence (e.g. a 25, 50, 100, or 150 continuous amino acid portion) compared to a naturally occurring Rap1 protein. In embodiments, the Rap1 protein is substantially identical to the protein identified by the UniProt reference number P62834 or a variant or homolog having substantial identity thereto. In embodiments, the Rap1 protein is substantially identical to the protein identified by the UniProt reference number P61224 or a variant or homolog having substantial identity thereto.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The term “chemical fluorophore” is used herein according to its plain ordinary meaning and refers to a light-sensitive chemical compound that can re-emit light upon light excitation. In embodiments, the chemical fluorophore is one part of a Forster resonance energy transfer (FRET) pair. In embodiments, the chemical fluorophore is a donor chemical fluorophore. In embodiments, the chemical fluorophore is an acceptor chemical fluorophore. In embodiments, the chemical fluorophore is NGD, JF669, JFX646, or JF635. In embodiments, the chemical fluorophore includes the formula of.

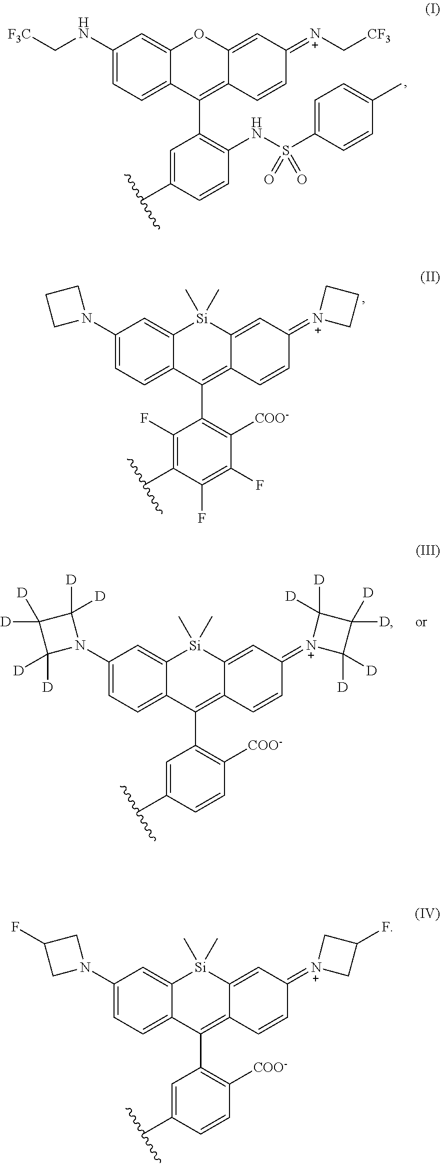

The terms “FRET pair” and “FRET fluorophore pair” are used herein according to their plain ordinary meaning and refer a pair of light-sensitive chemical compounds that are capable of transferring energy through nonradiative dipole coupling with one member being a donor and the other member being an acceptor. In embodiments, the pair of light-sensitive chemical compounds includes a first light-sensitive chemical compound and a second light-sensitive chemical compound. In embodiments, the pair of light-sensitive chemical compounds are capable of emitting light upon light excitation. In embodiments, the pair of light-sensitive chemical compounds absorb light energy of a specific wavelength and emit the light at a specific longer wavelength. In embodiments, the first light-sensitive chemical compound is a fluorescent protein domain or a chemical fluorophore. In embodiments, the second light-sensitive chemical compound is a fluorescent protein domain or a chemical fluorophore. In embodiments, upon excitation by light, the chemical fluorophore transfers energy to the fluorescent protein domain. In embodiments, upon excitation by light, the fluorescent protein domain transfers energy to the chemical fluorophore. In embodiments, this energy transfer between the chemical fluorophore and the fluorescent protein domain is Forster resonance energy transfer (FRET). In embodiments, the chemical fluorophore is a donor fluorophore or an acceptor fluorophore. In embodiments, the fluorescent protein domain is a donor fluorophore or an acceptor fluorophore. In embodiments, the FRET occurs in when the chemical fluorophore and the fluorescent protein domain are in close proximity. In embodiments, the FRET occurs when radius between when the chemical fluorophore and the fluorescent protein domain is smaller than the wavelength of light emitted. In embodiments, the pair of fluorescent chemical compounds are a pair of chemical fluorophores. In embodiments, upon excitation by light, the first fluorophore transfers energy to the second fluorophore. In embodiments, this energy transfer between the pair of fluorophores is Forster resonance energy transfer (FRET). In embodiments, the first fluorophore is a donor fluorophore. In embodiments, the second fluorophore is an acceptor fluorophore. In embodiments, the FRET occurs in when the pair of fluorophores are in close proximity. In embodiments, the FRET occurs when radius between the pair of fluorophores is smaller than the wavelength of light emitted. For the compositions and methods provided herein including embodiments thereof any one of the FRET fluorophore pairs described in Bajar et al., Sensors (Basel), 2016:16(9):1488 which is incorporated herein by reference in its entirety and for all purposes, may be used. In embodiments, the FRET pair is a cyan-yellow or a red-green FRET pair. In embodiments, the FRET pair is a cyan-yellow FRET pair. In embodiments, the FRET pair is a red-green FRET pair.

The term “fluorescent protein domain” is used herein according to its plain ordinary meaning and refers to a light-sensitive peptide or variant or fragment thereof. In embodiments, the fluorescent protein domain absorbs light at a specific wavelength and emits the light at a longer wavelength. In embodiments, the wavelength of light absorbed is the excitation wavelength. In embodiments, the wavelength of light emitted is the emission wavelength. In embodiments, the fluorescent protein domain is one part of a FRET pair. In embodiments, the fluorescent protein domain is an mScarlet domain, a stagRFP domain, an mKOk domain, an mRuby3 domain, a FusionMQV domain, a FusionRed domain, an mScarlet-1 domain, an Azalea-B5 domain, an mKate2 domain, an mCherry domain, an mNeptune2 domain, an mNeptune2.5 domain, an mCitrine domain, a cpVenus domain, an mPapaya domain, a sfGFP domain, an mNG2 domain, an mNG3A domain, an miRFP720 domain, an mRhubarb720 domain, a Dreiklang domain, a Skvlan-S domain, an ffDronpa domain, an rsEGFP2 domain, an mClover3 domain, or an EGFP domain.

The term “haloalkane dehalogenase domain” is used herein according to its plain ordinary meaning and refers to an amino acid sequence of a hydrolase enzyme or variant or fragment thereof that catalyzes a halide bond in a carbon-halide compound. In embodiments, the haloalkane dehalogenase domain is a self-labeling peptide. In embodiments, the haloalkane dehalogenase domain is covalently bound to a chemical fluorophore. In embodiments, the haloalkane dehalogenase domain is a HaloTag domain. In embodiments, the haloalkane dehalogenase domain includes the amino acid sequence of SEQ ID NO:25 or SEQ ID NO:26.

The term “biosensing protein domain” is used according to its plain ordinary meaning and refers to an amino acid sequence that detects the presence or activity of a target biomolecule in a cell. In embodiments, the biosensing protein domain includes a target biomolecule binding domain. In embodiments, the biosensing protein domain includes a target biomolecule substrate domain, a linker domain, and a phosphoamino acid binding domain.

The term “target biomolecule binding domain” is used herein according to its plain ordinary meaning and refers to an amino acid sequence capable of binding a target biomolecule in a cell. In embodiments the target biomolecule is a protein kinase, a second messenger molecule, or a GTPase.

The term “target biomolecule activity sensing domain” is used herein according to its plain ordinary meaning and refers to an amino acid sequence that is capable of detecting activity of a target biomolecule substrate domain. In embodiments, the target biomolecule activity sensing domain is capable of detecting a biomolecule interacting with a target biomolecule substrate domain. In embodiments, the target biomolecule activity sensing domain is capable of detecting a target biomolecule-induced modification of a target biomolecule substrate domain. In embodiments, the target biomolecule sensing domain includes a phosphoamino acid binding domain or an M13 binding domain. In embodiments, the target biomolecule sensing domain includes a phosphoamino acid binding domain. In embodiments, the target biomolecule sensing domain is a phosphoamino acid binding domain. In embodiments, the target biomolecule sensing domain includes a an M13 binding domain. In embodiments, the target biomolecule sensing domain is an M13 binding domain. In embodiments, the target biomolecule activity sensing domain includes a Ras pseudoligand (PL) domain. In embodiments, the Ras pseudoligand domain is capable of binding a Ras binding domain (RBD). In embodiments, the Ras pseudoligand domain includes the amino acid sequence of SEQ ID NO:42. In embodiments, the Ras pseudoligand domain is the amino acid sequence of SEQ ID NO:42.

The term “phosphoamino acid binding domain” is used herein according to its plain ordinary meaning and refers to an amino acid sequence capable of binding a phosphorylated amino acid residue. In embodiments, the target biomolecule substrate domain is capable of binding a phosphoamino acid binding domain. In embodiments, the phosphoamino acid binding domain is capable of binding a target biomolecule substrate domain. In embodiments, a target biomolecule substrate domain undergoes a protein modification in response to cellular signaling activity, thereby enabling the phosphoamino acid binding domain to bind to the target biomolecule substrate domain. In embodiments, the protein modification is phosphorylation of an amino acid residue in the target biomolecule substrate domain. In embodiments, an amino acid in the target biomolecule substrate domain is phosphorylated in response to cell activity, cell signaling, or a cell signal transduction cascade. In embodiments, the phosphorylated amino acid residue is a serine, threonine, or tyrosine. In embodiments, the phosphorylated amino acid residue is a serine or a threonine. In embodiments, the phosphorylated amino acid residue is a serine. In embodiments, the phosphorylated amino acid residue is a threonine. In embodiments, the phosphorylated amino acid residue is a tyrosine.

The term “calmodulin-binding peptide of myosin light chain kinase binding domain” or “M13 binding domain” is used herein according to its plain ordinary meaning and refers to an amino acid sequence capable of binding a calmodulin domain (e.g., CaM), wherein the calmodulin domain is bound to a calcium ion (e.g., calcium-bound calmodulin domain). In embodiments, the M13 binding domain does not bind a calmodulin domain which is not bound to a calcium ion. In embodiments, the M13 binding domain includes the amino acid sequence of SEQ ID NO. 43.

The term “target biomolecule substrate domain” is used herein according to its plain ordinary meaning and refers to an amino acid sequence that is a target on which a biomolecule or enzyme acts. In embodiments, the target biomolecule substrate domain includes an amino acid residue on which a biomolecule or enzyme acts. In embodiments, the target biomolecule substate domain includes a phosphorylatable amino acid residue. In embodiments, the target biomolecule substrate domain is capable of binding a target biomolecule activity sensing domain. In embodiments, the target biomolecule substrate domain is capable of binding a phosphoamino acid binding domain. In embodiments, the target biomolecule substrate domain is capable of binding a target biomolecule domain. In embodiments, the target biomolecule substrate domain undergoes a protein modification in response to cellular signaling activity, thereby enabling a target biomolecule activity sensing domain to bind to the target biomolecule substrate domain. In embodiments, the protein modification is phosphorylation of an amino acid residue in the target biomolecule substrate domain. In embodiments, an amino acid in the target biomolecule substrate domain is phosphorylated in response to cell activity, cell signaling, or a cell signal transduction cascade. In embodiments, the phosphorylated amino acid residue is a serine, threonine, or tyrosine. In embodiments, the phosphorylated amino acid residue is a serine or a threonine. In embodiments, the phosphorylated amino acid residue is a serine. In embodiments, the phosphorylated amino acid residue is a threonine. In embodiments, the phosphorylated amino acid residue is a tyrosine. In embodiments, the target biomolecule substrate domain includes a calmodulin domain. In embodiments, the calmodulin domain includes the amino acid sequence of SEQ ID NO: 36. In embodiments, the calmodulin domain is the amino acid sequence of SEQ ID NO: 36. In embodiments, the target biomolecule substrate domain is a Ras binding domain (RBD). In embodiments, the Ras binding domain is capable of binding a Ras protein or a Ras pseudoligand domain. In embodiments, the Ras binding domain is capable of binding a Ras protein. In embodiments, the Ras binding domain is capable of binding a Ras pseudoligand domain. In embodiments, a Ras protein is activated in response to cellular signaling activity, thereby disrupting or inhibiting the binding of the Ras binding domain. In embodiments, the disrupted binding of the Ras binding domain to the Ras protein, allows the Ras pseudoligand domain to bind the Ras binding domain. In embodiments, the Ras binding domain includes the amino acid sequence of SEQ ID NO:35. In embodiments, the Ras binding domain is the amino acid sequence of SEQ ID NO:35.

The term “linker domain” is used herein according to its plain ordinary meaning and refers to an amino acid sequence between a target biomolecule substrate domain and a target biomolecule activity sensing domain or a target biomolecule domain. In embodiments, the linker domain may be referred to herein as a peptide linker.

The term “protein kinase” is used herein according to its plain ordinary meaning and refers to biomolecule which modifies a target substrate by covalently adding phosphate groups to an amino acid residue within the target substrate. In embodiments, the protein kinase is a protein. In embodiments, the protein kinase phosphorylates the target substrate. In embodiments, the target substate is a target biomolecule. In embodiments, the target substrate is a target protein. In embodiments, the target substate is a target biomolecule substate domain. In embodiments, the protein kinase is a serine/threonine kinase. In embodiments, the protein kinase is a tyrosine kinase.

The term “second messenger molecule” is used herein according to its plain ordinary meaning and refers to an intracellular signaling molecule released within a cell in response to an extracellular signaling molecule. In embodiments, the second messenger molecule is a hydrophobic molecule. In embodiments, the second messenger molecule is diacylglycerol (DAG), or calcium (Ca²⁺). In embodiments, the second messenger molecule is a hydrophilic molecule. In embodiments, the second messenger molecule is cyclic adenosine monophosphate (cAMP), cyclic guanosine monophosphate (cGMP), or inositol triphosphate (IP₃). In embodiments, the second messenger molecule is calcium (Ca²).

The term “GTPase” is used herein according to its plain ordinary meaning and refers to a hydrolase enzyme that catalyzes a hydrolysis reaction. In embodiments, the GTPase binds the nucleotide guanosine triphosphate (GTP) and hydrolyzes it to guanosine diphosphate (GDP). In embodiments, the GTPase is a Ras GTPase. In embodiments, the GTPase is a Rap1 GTPase.

The terms “plasmid”, “vector” or “expression vector” refer to a nucleic acid molecule that encodes for genes, regulatory elements necessary for the expression of genes, proteins, and/or recombinant proteins (e.g., biosensors). Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide specifically reactive with a target peptide. Any appropriate method known in the art for conjugating a peptide to the label may be employed. e.g., using methods described in Hermanson, Bioconjugate Techniques 1996, Academic Press, Inc., San Diego.

When the label or detectable moiety is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA. NOTA, NETA, TETA, porphyrins, polyamines, crown ethers, bis-thiosemicarbazones, polyoximes, and like groups. The chelate is normally linked to the PSMA antibody or functional antibody fragment by a group, which enables the formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antibodies and carriers described herein. Macrocyclic chelates such as NOTA, DOTA, and TETA are of use with a variety of metals and radiometals including, but not limited to, radionuclides of gallium, yttrium and copper, respectively. Other ring-type chelates such as macrocyclic polyethers, which are of interest for stably binding nuclides, such as ²²³Ra for RAIT may be used. In certain embodiments, chelating moieties may be used to attach a PET imaging agent, such as an Al-¹⁸F complex, to a targeting molecule for use in PET analysis.

“Contacting” is used in accordance with its plain ordinary meaning and refers to the process of allowing at least two distinct species (e.g. antibodies and antigens) to become sufficiently proximal to react, interact, or physically touch. It should be appreciated, however, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a pharmaceutical composition as provided herein and a cell. In embodiments contacting includes, for example, allowing a pharmaceutical composition as described herein to interact with a cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

As defined herein, the term “inhibition”. “inhibit”, “inhibiting” and the like in reference to a biomolecule-inhibitor interaction means negatively affecting (e.g., decreasing) the activity or function of the biomolecule (e.g. protein kinase, second messenger molecule, or GTPase) relative to the activity or function of the biomolecule in the absence of the inhibitor. In embodiments inhibition means negatively affecting (e.g., decreasing) the concentration or levels of the biomolecule relative to the concentration or level of the biomolecule in the absence of the inhibitor. In embodiments inhibition refers to reduction of a disease or symptoms of disease. In embodiments, inhibition refers to a reduction in the activity of the biomolecule. Thus, inhibition includes, at least in part, partially or totally blocking stimulation, decreasing, preventing, or delaying activation, or inactivating, desensitizing, or down-regulating signal transduction or enzymatic activity or the amount of the biomolecule. In embodiments, inhibition refers to a reduction of activity of a biomolecule resulting from a direct interaction (e.g., an inhibitor binds to a biomolecule). In embodiments, inhibition refers to a reduction of activity of a biomolecule from an indirect interaction (e.g., an inhibitor binds to a protein that activates a biomolecule, thereby preventing target protein activation).

Thus, the terms “inhibitor,” “repressor” or “antagonist” or “downregulator” interchangeably refer to a substance capable of detectably decreasing the expression or activity of a given gene or biomolecule (e.g., protein kinase, second messenger molecule, or GTPase). The antagonist can decrease the biomolecule expression or activity 10%, 20%, 30%, 40%, 50%, 60%, 70%, 80%, 90% or more in comparison to a control in the absence of the antagonist. In certain instances, biomolecule expression or activity is 1.5-fold, 2-fold, 3-fold, 4-fold, 5-fold, 10-fold or lower than the expression or activity in the absence of the antagonist.

The term “expression” includes any step involved in the production of the biomolecule including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse: rabbit; or a bird; reptile; or fish.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Biosensor Recombinant Proteins

Provided herein are, inter alia, biosensor recombinant proteins which provided quantitative measurement of biomolecule (e.g., protein kinase, second messenger molecule, or GTPase) activity in living cells with high spatiotemporal resolution. The biosensor recombinant proteins provided herein include, for example, a novel combination of fluorescent protein domain, a haloalkane dehalogenase domain, and a chemical, which have increased dynamic range and sensitivity compared to current biosensors and are useful for detecting a biomolecule (e.g., protein kinase, second messenger molecule, or GTPase) or activity of a biomolecule in a cell. Thus, in an aspect is provided a recombinant protein covalently bound to a chemical fluorophore, the recombinant protein including a fluorescent protein domain bound to a biosensing protein domain and a haloalkane dehalogenase domain bound to the biosensing protein domain, wherein: the chemical fluorophore is covalently bound to the haloalkane dehalogenase domain; and the chemical fluorophore and the fluorescent protein domain are a Forster Resonance Energy Transfer (FRET) pair.

In embodiments, the fluorescent protein domain is a red fluorescent protein (RFP) domain, an orange fluorescent protein (OFP) domain, a yellow fluorescent protein (YFP) domain, or a green fluorescent protein (GFP) domain. In embodiments, the fluorescent protein domain is a red fluorescent protein (RFP) domain. In embodiments, the fluorescent protein domain is an orange fluorescent protein (OFP) domain. In embodiments, the fluorescent protein domain is a yellow fluorescent protein (YFP) domain. In embodiments, the fluorescent protein domain is a green fluorescent protein (GFP) domain.

In embodiments, the fluorescent protein domain is an mScarlet domain. In embodiments, the fluorescent protein domain is a stagRFP domain. In embodiments, the fluorescent protein domain is an mKOk domain, an mRuby3 domain, a FusionMQV domain, a FusionRed domain, an mScarlet-1 domain, an Azalea-B5 domain, an mKate2 domain, an mCherry domain, an mNeptune2 domain, an mNeptune2.5 domain, an mCitrine domain, a cpVenus domain, an mPapaya domain, a sfGFP domain, an mNG2 domain, an mNG3A domain, an mIRFP720 domain, an mRhubarb720 domain, a Dreiklang domain, a Skylan-S domain, an ffDronpa domain, an rsEGFP2 domain, an mClover3 domain, or an EGFP domain. In embodiments, the fluorescent protein domain is an mScarlet domain. In embodiments, the fluorescent protein domain is a stagRFP domain. In embodiments, the fluorescent protein domain is an mKOk domain. In embodiments, the fluorescent protein domain is an mRuby3 domain. In embodiments, the fluorescent protein domain is a FusionMQV domain. In embodiments, the fluorescent protein domain is a FusionRed domain. In embodiments, the fluorescent protein domain is an mScarlet-1 domain, an Azalea-B5 domain. In embodiments, the fluorescent protein domain is an mKate2 domain. In embodiments, the fluorescent protein domain is an mCherry domain. In embodiments, the fluorescent protein domain is an mNeptune2 domain. In embodiments, the fluorescent protein domain is an mNeptune2.5 domain. In embodiments, the fluorescent protein domain is an mCitrine domain. In embodiments, the fluorescent protein domain is a cpVenus domain. In embodiments, the fluorescent protein domain is an mPapaya domain. In embodiments, the fluorescent protein domain is a sfGFP domain. In embodiments, the fluorescent protein domain is an mNG2 domain. In embodiments, the fluorescent protein domain is an mNG3A domain. In embodiments, the fluorescent protein domain is an miRFP720 domain. In embodiments, the fluorescent protein domain is an mRhubarb720 domain. In embodiments, the fluorescent protein domain is a Dreiklang domain. In embodiments, the fluorescent protein domain is a Skylan-S domain. In embodiments, the fluorescent protein domain is an ffDronpa domain. In embodiments, the fluorescent protein domain is an rsEGFP2 domain. In embodiments, the fluorescent protein domain is an mClover3 domain. In embodiments, the fluorescent protein domain is an EGFP domain.

In embodiments, the fluorescent protein domain includes the amino acid sequence of any one of SEQ ID NO:1 to SEQ ID NO:24. In embodiments, the fluorescent protein domain includes the amino acid sequence of SEQ ID NO:1. In embodiments, the fluorescent protein domain includes the amino acid sequence of SEQ ID NO:2. In embodiments, the fluorescent protein domain includes the amino acid sequence of SEQ ID NO:3. In embodiments, the fluorescent protein domain includes the amino acid sequence of SEQ ID NO:4. In embodiments, the fluorescent protein domain includes the amino acid sequence of SEQ ID NO:5. In embodiments, the fluorescent protein domain includes the amino acid sequence of SEQ ID NO:6. In embodiments, the fluorescent protein domain includes the amino acid sequence of SEQ ID NO:7. In embodiments, the fluorescent protein domain includes the amino acid sequence of SEQ ID NO:8. In embodiments, the fluorescent protein domain includes the amino acid sequence of SEQ ID NO:9. In embodiments, the fluorescent protein domain includes the amino acid sequence of SEQ ID NO:10. In embodiments, the fluorescent protein domain includes the amino acid sequence of SEQ ID NO:11. In embodiments, the fluorescent protein domain includes the amino acid sequence of SEQ ID NO: 12. In embodiments, the fluorescent protein domain includes the amino acid sequence of SEQ ID NO: 13. In embodiments, the fluorescent protein domain includes the amino acid sequence of SEQ ID NO:14. In embodiments, the fluorescent protein domain includes the amino acid sequence of SEQ ID NO: 15. In embodiments, the fluorescent protein domain includes the amino acid sequence of SEQ ID NO:16. In embodiments, the fluorescent protein domain includes the amino acid sequence of SEQ ID NO: 17. In embodiments, the fluorescent protein domain includes the amino acid sequence of SEQ ID NO:18. In embodiments, the fluorescent protein domain includes the amino acid sequence of SEQ ID NO:19. In embodiments, the fluorescent protein domain includes the amino acid sequence of SEQ ID NO:20. In embodiments, the fluorescent protein domain includes the amino acid sequence of SEQ ID NO:21. In embodiments, the fluorescent protein domain includes the amino acid sequence of SEQ ID NO:22. In embodiments, the fluorescent protein domain includes the amino acid sequence of SEQ ID NO:23. In embodiments, the fluorescent protein domain includes the amino acid sequence of SEQ ID NO:24.

In embodiments, the fluorescent protein domain is the amino acid sequence of any one of SEQ ID NO:1 to SEQ ID NO:24. In embodiments, the fluorescent protein domain is the amino acid sequence of SEQ ID NO: 1. In embodiments, the fluorescent protein domain is the amino acid sequence of SEQ ID NO:2. In embodiments, the fluorescent protein domain is the amino acid sequence of SEQ ID NO: 3. In embodiments, the fluorescent protein domain is the amino acid sequence of SEQ ID NO:4 In embodiments, the fluorescent protein domain is the amino acid sequence of SEQ ID NO: 5. In embodiments, the fluorescent protein domain is the amino acid sequence of SEQ ID NO:6 In embodiments, the fluorescent protein domain is the amino acid sequence of SEQ ID NO: 7. In embodiments, the fluorescent protein domain is the amino acid sequence of SEQ ID NO: 8. In embodiments, the fluorescent protein domain is the amino acid sequence of SEQ ID NO: 9. In embodiments, the fluorescent protein domain is the amino acid sequence of SEQ ID NO: 10. In embodiments, the fluorescent protein domain is the amino acid sequence of SEQ ID NO:11. In embodiments, the fluorescent protein domain is the amino acid sequence of SEQ ID NO: 12. In embodiments, the fluorescent protein domain is the amino acid sequence of SEQ ID NO: 13. In embodiments, the fluorescent protein domain is the amino acid sequence of SEQ ID NO:14. In embodiments, the fluorescent protein domain is the amino acid sequence of SEQ ID NO: 15. In embodiments, the fluorescent protein domain is the amino acid sequence of SEQ ID NO: 16.

In embodiments, the fluorescent protein domain is the amino acid sequence of SEQ ID NO: 17. In embodiments, the fluorescent protein domain is the amino acid sequence of SEQ ID NO:18. In embodiments, the fluorescent protein domain is the amino acid sequence of SEQ ID NO:19. In embodiments, the fluorescent protein domain is the amino acid sequence of SEQ ID NO:20. In embodiments, the fluorescent protein domain is the amino acid sequence of SEQ ID NO:21. In embodiments, the fluorescent protein domain is the amino acid sequence of SEQ ID NO:22. In embodiments, the fluorescent protein domain is the amino acid sequence of SEQ ID NO:23. In embodiments, the fluorescent protein domain is the amino acid sequence of SEQ ID NO:24.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO:1. In embodiments, the fluorescent protein domain includes an amino acid sequence having 70% sequence identity to the sequence of SEQ ID NO:1. In embodiments, the fluorescent protein domain includes an amino acid sequence having 75% sequence identity to the sequence of SEQ ID NO:1. In embodiments, the fluorescent protein domain includes an amino acid sequence having 80% sequence identity to the sequence of SEQ ID NO: 1. In embodiments, the fluorescent protein domain includes an amino acid sequence having 85% sequence identity to the sequence of SEQ ID NO:1. In embodiments, the fluorescent protein domain includes an amino acid sequence having 90% sequence identity to the sequence of SEQ ID NO: 1. In embodiments, the fluorescent protein domain includes an amino acid sequence having 95% sequence identity to the sequence of SEQ ID NO:1. In embodiments, the fluorescent protein domain includes an amino acid sequence having 96% sequence identity to the sequence of SEQ ID NO:1. In embodiments, the fluorescent protein domain includes an amino acid sequence having 97% sequence identity to the sequence of SEQ ID NO:1. In embodiments, the fluorescent protein domain includes an amino acid sequence having 98% sequence identity to the sequence of SEQ ID NO:1. In embodiments, the fluorescent protein domain includes an amino acid sequence having 99% sequence identity to the sequence of SEQ ID NO:1. In embodiments, the fluorescent protein domain includes an amino acid sequence having 100% sequence identity to the sequence of SEQ ID NO:1.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO:2. In embodiments, the fluorescent protein domain includes an amino acid sequence having 70% sequence identity to the sequence of SEQ ID NO:2. In embodiments, the fluorescent protein domain includes an amino acid sequence having 75% sequence identity to the sequence of SEQ ID NO:2. In embodiments, the fluorescent protein domain includes an amino acid sequence having 80% sequence identity to the sequence of SEQ ID NO:2. In embodiments, the fluorescent protein domain includes an amino acid sequence having 85% sequence identity to the sequence of SEQ ID NO:2. In embodiments, the fluorescent protein domain includes an amino acid sequence having 90% sequence identity to the sequence of SEQ ID NO:2. In embodiments, the fluorescent protein domain includes an amino acid sequence having 95% sequence identity to the sequence of SEQ ID NO:2. In embodiments, the fluorescent protein domain includes an amino acid sequence having 96% sequence identity to the sequence of SEQ ID NO:2. In embodiments, the fluorescent protein domain includes an amino acid sequence having 97% sequence identity to the sequence of SEQ ID NO:2. In embodiments, the fluorescent protein domain includes an amino acid sequence having 98% sequence identity to the sequence of SEQ ID NO:2. In embodiments, the fluorescent protein domain includes an amino acid sequence having 99% sequence identity to the sequence of SEQ ID NO:2. In embodiments, the fluorescent protein domain includes an amino acid sequence having 100% sequence identity to the sequence of SEQ ID NO:2.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO:3. In embodiments, the fluorescent protein domain includes an amino acid sequence having 70% sequence identity to the sequence of SEQ ID NO:3. In embodiments, the fluorescent protein domain includes an amino acid sequence having 75% sequence identity to the sequence of SEQ ID NO:3. In embodiments, the fluorescent protein domain includes an amino acid sequence having 80% sequence identity to the sequence of SEQ ID NO:3. In embodiments, the fluorescent protein domain includes an amino acid sequence having 85% sequence identity to the sequence of SEQ ID NO:3. In embodiments, the fluorescent protein domain includes an amino acid sequence having 90% sequence identity to the sequence of SEQ ID NO:3. In embodiments, the fluorescent protein domain includes an amino acid sequence having 95% sequence identity to the sequence of SEQ ID NO:3. In embodiments, the fluorescent protein domain includes an amino acid sequence having 96% sequence identity to the sequence of SEQ ID NO:3. In embodiments, the fluorescent protein domain includes an amino acid sequence having 97% sequence identity to the sequence of SEQ ID NO:3. In embodiments, the fluorescent protein domain includes an amino acid sequence having 98% sequence identity to the sequence of SEQ ID NO:3. In embodiments, the fluorescent protein domain includes an amino acid sequence having 99% sequence identity to the sequence of SEQ ID NO:3. In embodiments, the fluorescent protein domain includes an amino acid sequence having 100% sequence identity to the sequence of SEQ ID NO:3.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO:4. In embodiments, the fluorescent protein domain includes an amino acid sequence having 70% sequence identity to the sequence of SEQ ID NO:4. In embodiments, the fluorescent protein domain includes an amino acid sequence having 75% sequence identity to the sequence of SEQ ID NO:4. In embodiments, the fluorescent protein domain includes an amino acid sequence having 80% sequence identity to the sequence of SEQ ID NO:4. In embodiments, the fluorescent protein domain includes an amino acid sequence having 85% sequence identity to the sequence of SEQ ID NO:4. In embodiments, the fluorescent protein domain includes an amino acid sequence having 90% sequence identity to the sequence of SEQ ID NO:4. In embodiments, the fluorescent protein domain includes an amino acid sequence having 95% sequence identity to the sequence of SEQ ID NO:4. In embodiments, the fluorescent protein domain includes an amino acid sequence having 96% sequence identity to the sequence of SEQ ID NO:4. In embodiments, the fluorescent protein domain includes an amino acid sequence having 97% sequence identity to the sequence of SEQ ID NO:4. In embodiments, the fluorescent protein domain includes an amino acid sequence having 98% sequence identity to the sequence of SEQ ID NO:4. In embodiments, the fluorescent protein domain includes an amino acid sequence having 99% sequence identity to the sequence of SEQ ID NO:4. In embodiments, the fluorescent protein domain includes an amino acid sequence having 100% sequence identity to the sequence of SEQ ID NO:4.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO:5. In embodiments, the fluorescent protein domain includes an amino acid sequence having 70% sequence identity to the sequence of SEQ ID NO:5. In embodiments, the fluorescent protein domain includes an amino acid sequence having 75% sequence identity to the sequence of SEQ ID NO:5. In embodiments, the fluorescent protein domain includes an amino acid sequence having 80% sequence identity to the sequence of SEQ ID NO:5. In embodiments, the fluorescent protein domain includes an amino acid sequence having 85% sequence identity to the sequence of SEQ ID NO:5. In embodiments, the fluorescent protein domain includes an amino acid sequence having 90% sequence identity to the sequence of SEQ ID NO:5. In embodiments, the fluorescent protein domain includes an amino acid sequence having 95% sequence identity to the sequence of SEQ ID NO:5. In embodiments, the fluorescent protein domain includes an amino acid sequence having 96% sequence identity to the sequence of SEQ ID NO:5. In embodiments, the fluorescent protein domain includes an amino acid sequence having 97% sequence identity to the sequence of SEQ ID NO:5. In embodiments, the fluorescent protein domain includes an amino acid sequence having 98% sequence identity to the sequence of SEQ ID NO:5. In embodiments, the fluorescent protein domain includes an amino acid sequence having 99% sequence identity to the sequence of SEQ ID NO:5. In embodiments, the fluorescent protein domain includes an amino acid sequence having 100% sequence identity to the sequence of SEQ ID NO:5.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO:6. In embodiments, the fluorescent protein domain includes an amino acid sequence having 70% sequence identity to the sequence of SEQ ID NO:6. In embodiments, the fluorescent protein domain includes an amino acid sequence having 75% sequence identity to the sequence of SEQ ID NO:6. In embodiments, the fluorescent protein domain includes an amino acid sequence having 80% sequence identity to the sequence of SEQ ID NO:6. In embodiments, the fluorescent protein domain includes an amino acid sequence having 85% sequence identity to the sequence of SEQ ID NO:6. In embodiments, the fluorescent protein domain includes an amino acid sequence having 90% sequence identity to the sequence of SEQ ID NO:6. In embodiments, the fluorescent protein domain includes an amino acid sequence having 95% sequence identity to the sequence of SEQ ID NO:6. In embodiments, the fluorescent protein domain includes an amino acid sequence having 96% sequence identity to the sequence of SEQ ID NO:6. In embodiments, the fluorescent protein domain includes an amino acid sequence having 97% sequence identity to the sequence of SEQ ID NO:6. In embodiments, the fluorescent protein domain includes an amino acid sequence having 98% sequence identity to the sequence of SEQ ID NO:6. In embodiments, the fluorescent protein domain includes an amino acid sequence having 99% sequence identity to the sequence of SEQ ID NO:6. In embodiments, the fluorescent protein domain includes an amino acid sequence having 100% sequence identity to the sequence of SEQ ID NO:6.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO:7. In embodiments, the fluorescent protein domain includes an amino acid sequence having 70% sequence identity to the sequence of SEQ ID NO:7. In embodiments, the fluorescent protein domain includes an amino acid sequence having 75% sequence identity to the sequence of SEQ ID NO:7. In embodiments, the fluorescent protein domain includes an amino acid sequence having 80% sequence identity to the sequence of SEQ ID NO:7. In embodiments, the fluorescent protein domain includes an amino acid sequence having 85% sequence identity to the sequence of SEQ ID NO:7. In embodiments, the fluorescent protein domain includes an amino acid sequence having 90% sequence identity to the sequence of SEQ ID NO:7. In embodiments, the fluorescent protein domain includes an amino acid sequence having 95% sequence identity to the sequence of SEQ ID NO:7. In embodiments, the fluorescent protein domain includes an amino acid sequence having 96% sequence identity to the sequence of SEQ ID NO:7. In embodiments, the fluorescent protein domain includes an amino acid sequence having 97% sequence identity to the sequence of SEQ ID NO:7. In embodiments, the fluorescent protein domain includes an amino acid sequence having 98% sequence identity to the sequence of SEQ ID NO:7. In embodiments, the fluorescent protein domain includes an amino acid sequence having 99% sequence identity to the sequence of SEQ ID NO:7. In embodiments, the fluorescent protein domain includes an amino acid sequence having 100% sequence identity to the sequence of SEQ ID NO:7.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO:8. In embodiments, the fluorescent protein domain includes an amino acid sequence having 70% sequence identity to the sequence of SEQ ID NO:8. In embodiments, the fluorescent protein domain includes an amino acid sequence having 75% sequence identity to the sequence of SEQ ID NO:8. In embodiments, the fluorescent protein domain includes an amino acid sequence having 80% sequence identity to the sequence of SEQ ID NO:8. In embodiments, the fluorescent protein domain includes an amino acid sequence having 85% sequence identity to the sequence of SEQ ID NO:8. In embodiments, the fluorescent protein domain includes an amino acid sequence having 90% sequence identity to the sequence of SEQ ID NO:8. In embodiments, the fluorescent protein domain includes an amino acid sequence having 95% sequence identity to the sequence of SEQ ID NO:8. In embodiments, the fluorescent protein domain includes an amino acid sequence having 96% sequence identity to the sequence of SEQ ID NO:8. In embodiments, the fluorescent protein domain includes an amino acid sequence having 97% sequence identity to the sequence of SEQ ID NO:8. In embodiments, the fluorescent protein domain includes an amino acid sequence having 98% sequence identity to the sequence of SEQ ID NO:8. In embodiments, the fluorescent protein domain includes an amino acid sequence having 99% sequence identity to the sequence of SEQ ID NO:8. In embodiments, the fluorescent protein domain includes an amino acid sequence having 100% sequence identity to the sequence of SEQ ID NO:8.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO:9. In embodiments, the fluorescent protein domain includes an amino acid sequence having 70% sequence identity to the sequence of SEQ ID NO:9. In embodiments, the fluorescent protein domain includes an amino acid sequence having 75% sequence identity to the sequence of SEQ ID NO:9. In embodiments, the fluorescent protein domain includes an amino acid sequence having 80% sequence identity to the sequence of SEQ ID NO:9. In embodiments, the fluorescent protein domain includes an amino acid sequence having 85% sequence identity to the sequence of SEQ ID NO:9. In embodiments, the fluorescent protein domain includes an amino acid sequence having 90% sequence identity to the sequence of SEQ ID NO:9. In embodiments, the fluorescent protein domain includes an amino acid sequence having 95% sequence identity to the sequence of SEQ ID NO:9. In embodiments, the fluorescent protein domain includes an amino acid sequence having 96% sequence identity to the sequence of SEQ ID NO:9. In embodiments, the fluorescent protein domain includes an amino acid sequence having 97% sequence identity to the sequence of SEQ ID NO:9. In embodiments, the fluorescent protein domain includes an amino acid sequence having 98% sequence identity to the sequence of SEQ ID NO:9. In embodiments, the fluorescent protein domain includes an amino acid sequence having 99% sequence identity to the sequence of SEQ ID NO:9. In embodiments, the fluorescent protein domain includes an amino acid sequence having 100% sequence identity to the sequence of SEQ ID NO:9.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO:10. In embodiments, the fluorescent protein domain includes an amino acid sequence having 70% sequence identity to the sequence of SEQ ID NO:10. In embodiments, the fluorescent protein domain includes an amino acid sequence having 75% sequence identity to the sequence of SEQ ID NO: 10. In embodiments, the fluorescent protein domain includes an amino acid sequence having 80% sequence identity to the sequence of SEQ ID NO:10. In embodiments, the fluorescent protein domain includes an amino acid sequence having 85% sequence identity to the sequence of SEQ ID NO:10. In embodiments, the fluorescent protein domain includes an amino acid sequence having 90% sequence identity to the sequence of SEQ ID NO: 10. In embodiments, the fluorescent protein domain includes an amino acid sequence having 95% sequence identity to the sequence of SEQ ID NO:10. In embodiments, the fluorescent protein domain includes an amino acid sequence having 96% sequence identity to the sequence of SEQ ID NO: 10. In embodiments, the fluorescent protein domain includes an amino acid sequence having 97% sequence identity to the sequence of SEQ ID NO:10. In embodiments, the fluorescent protein domain includes an amino acid sequence having 98% sequence identity to the sequence of SEQ ID NO:10. In embodiments, the fluorescent protein domain includes an amino acid sequence having 99% sequence identity to the sequence of SEQ ID NO: 10. In embodiments, the fluorescent protein domain includes an amino acid sequence having 100% sequence identity to the sequence of SEQ ID NO:10.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO:11. In embodiments, the fluorescent protein domain includes an amino acid sequence having 70% sequence identity to the sequence of SEQ ID NO: 11. In embodiments, the fluorescent protein domain includes an amino acid sequence having 75% sequence identity to the sequence of SEQ ID NO: 11. In embodiments, the fluorescent protein domain includes an amino acid sequence having 80% sequence identity to the sequence of SEQ ID NO: 11. In embodiments, the fluorescent protein domain includes an amino acid sequence having 85% sequence identity to the sequence of SEQ ID NO:11. In embodiments, the fluorescent protein domain includes an amino acid sequence having 90% sequence identity to the sequence of SEQ ID NO: 11. In embodiments, the fluorescent protein domain includes an amino acid sequence having 95% sequence identity to the sequence of SEQ ID NO: 11. In embodiments, the fluorescent protein domain includes an amino acid sequence having 96% sequence identity to the sequence of SEQ ID NO:11. In embodiments, the fluorescent protein domain includes an amino acid sequence having 97% sequence identity to the sequence of SEQ ID NO:11. In embodiments, the fluorescent protein domain includes an amino acid sequence having 98% sequence identity to the sequence of SEQ ID NO: 11. In embodiments, the fluorescent protein domain includes an amino acid sequence having 99% sequence identity to the sequence of SEQ ID NO: 11. In embodiments, the fluorescent protein domain includes an amino acid sequence having 100% sequence identity to the sequence of SEQ ID NO:11.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO:12. In embodiments, the fluorescent protein domain includes an amino acid sequence having 70% sequence identity to the sequence of SEQ ID NO:12. In embodiments, the fluorescent protein domain includes an amino acid sequence having 75% sequence identity to the sequence of SEQ ID NO: 12. In embodiments, the fluorescent protein domain includes an amino acid sequence having 80% sequence identity to the sequence of SEQ ID NO:12. In embodiments, the fluorescent protein domain includes an amino acid sequence having 85% sequence identity to the sequence of SEQ ID NO:12. In embodiments, the fluorescent protein domain includes an amino acid sequence having 90% sequence identity to the sequence of SEQ ID NO: 12. In embodiments, the fluorescent protein domain includes an amino acid sequence having 95% sequence identity to the sequence of SEQ ID NO:12. In embodiments, the fluorescent protein domain includes an amino acid sequence having 96% sequence identity to the sequence of SEQ ID NO:12. In embodiments, the fluorescent protein domain includes an amino acid sequence having 97% sequence identity to the sequence of SEQ ID NO:12. In embodiments, the fluorescent protein domain includes an amino acid sequence having 98% sequence identity to the sequence of SEQ ID NO:12. In embodiments, the fluorescent protein domain includes an amino acid sequence having 99% sequence identity to the sequence of SEQ ID NO: 12. In embodiments, the fluorescent protein domain includes an amino acid sequence having 100% sequence identity to the sequence of SEQ ID NO:12.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO:13. In embodiments, the fluorescent protein domain includes an amino acid sequence having 70% sequence identity to the sequence of SEQ ID NO: 1³. In embodiments, the fluorescent protein domain includes an amino acid sequence having 75% sequence identity to the sequence of SEQ ID NO:13. In embodiments, the fluorescent protein domain includes an amino acid sequence having 80% sequence identity to the sequence of SEQ ID NO:13. In embodiments, the fluorescent protein domain includes an amino acid sequence having 85% sequence identity to the sequence of SEQ ID NO:13. In embodiments, the fluorescent protein domain includes an amino acid sequence having 90% sequence identity to the sequence of SEQ ID NO:13. In embodiments, the fluorescent protein domain includes an amino acid sequence having 95% sequence identity to the sequence of SEQ ID NO:13. In embodiments, the fluorescent protein domain includes an amino acid sequence having 96% sequence identity to the sequence of SEQ ID NO:13. In embodiments, the fluorescent protein domain includes an amino acid sequence having 97% sequence identity to the sequence of SEQ ID NO:13. In embodiments, the fluorescent protein domain includes an amino acid sequence having 98% sequence identity to the sequence of SEQ ID NO: 13. In embodiments, the fluorescent protein domain includes an amino acid sequence having 99% sequence identity to the sequence of SEQ ID NO:13. In embodiments, the fluorescent protein domain includes an amino acid sequence having 100% sequence identity to the sequence of SEQ ID NO:13.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO:14. In embodiments, the fluorescent protein domain includes an amino acid sequence having 70% sequence identity to the sequence of SEQ ID NO:14. In embodiments, the fluorescent protein domain includes an amino acid sequence having 75% sequence identity to the sequence of SEQ ID NO: 14. In embodiments, the fluorescent protein domain includes an amino acid sequence having 80% sequence identity to the sequence of SEQ ID NO:14. In embodiments, the fluorescent protein domain includes an amino acid sequence having 85% sequence identity to the sequence of SEQ ID NO:14. In embodiments, the fluorescent protein domain includes an amino acid sequence having 90% sequence identity to the sequence of SEQ ID NO: 14. In embodiments, the fluorescent protein domain includes an amino acid sequence having 95% sequence identity to the sequence of SEQ ID NO:14. In embodiments, the fluorescent protein domain includes an amino acid sequence having 96% sequence identity to the sequence of SEQ ID NO: 14. In embodiments, the fluorescent protein domain includes an amino acid sequence having 97% sequence identity to the sequence of SEQ ID NO: 14. In embodiments, the fluorescent protein domain includes an amino acid sequence having 98% sequence identity to the sequence of SEQ ID NO:14. In embodiments, the fluorescent protein domain includes an amino acid sequence having 99% sequence identity to the sequence of SEQ ID NO:14. In embodiments, the fluorescent protein domain includes an amino acid sequence having 100% sequence identity to the sequence of SEQ ID NO:14.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO:15. In embodiments, the fluorescent protein domain includes an amino acid sequence having 70% sequence identity to the sequence of SEQ ID NO:15. In embodiments, the fluorescent protein domain includes an amino acid sequence having 75% sequence identity to the sequence of SEQ ID NO:15. In embodiments, the fluorescent protein domain includes an amino acid sequence having 80% sequence identity to the sequence of SEQ ID NO:15. In embodiments, the fluorescent protein domain includes an amino acid sequence having 85% sequence identity to the sequence of SEQ ID NO:15. In embodiments, the fluorescent protein domain includes an amino acid sequence having 90% sequence identity to the sequence of SEQ ID NO:15. In embodiments, the fluorescent protein domain includes an amino acid sequence having 95% sequence identity to the sequence of SEQ ID NO: 15. In embodiments, the fluorescent protein domain includes an amino acid sequence having 96% sequence identity to the sequence of SEQ ID NO:15. In embodiments, the fluorescent protein domain includes an amino acid sequence having 97% sequence identity to the sequence of SEQ ID NO:15. In embodiments, the fluorescent protein domain includes an amino acid sequence having 98% sequence identity to the sequence of SEQ ID NO: 15. In embodiments, the fluorescent protein domain includes an amino acid sequence having 99% sequence identity to the sequence of SEQ ID NO: 15. In embodiments, the fluorescent protein domain includes an amino acid sequence having 100% sequence identity to the sequence of SEQ ID NO:15.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO:16. In embodiments, the fluorescent protein domain includes an amino acid sequence having 70% sequence identity to the sequence of SEQ ID NO:16. In embodiments, the fluorescent protein domain includes an amino acid sequence having 75% sequence identity to the sequence of SEQ ID NO: 16. In embodiments, the fluorescent protein domain includes an amino acid sequence having 80% sequence identity to the sequence of SEQ ID NO: 16. In embodiments, the fluorescent protein domain includes an amino acid sequence having 85% sequence identity to the sequence of SEQ ID NO:16. In embodiments, the fluorescent protein domain includes an amino acid sequence having 90% sequence identity to the sequence of SEQ ID NO:16. In embodiments, the fluorescent protein domain includes an amino acid sequence having 95% sequence identity to the sequence of SEQ ID NO:16. In embodiments, the fluorescent protein domain includes an amino acid sequence having 96% sequence identity to the sequence of SEQ ID NO:16. In embodiments, the fluorescent protein domain includes an amino acid sequence having 97% sequence identity to the sequence of SEQ ID NO: 16. In embodiments, the fluorescent protein domain includes an amino acid sequence having 98% sequence identity to the sequence of SEQ ID NO:16. In embodiments, the fluorescent protein domain includes an amino acid sequence having 99% sequence identity to the sequence of SEQ ID NO:16. In embodiments, the fluorescent protein domain includes an amino acid sequence having 100% sequence identity to the sequence of SEQ ID NO: 16.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO:17. In embodiments, the fluorescent protein domain includes an amino acid sequence having 70% sequence identity to the sequence of SEQ ID NO: 17. In embodiments, the fluorescent protein domain includes an amino acid sequence having 75% sequence identity to the sequence of SEQ ID NO:17. In embodiments, the fluorescent protein domain includes an amino acid sequence having 80% sequence identity to the sequence of SEQ ID NO:17. In embodiments, the fluorescent protein domain includes an amino acid sequence having 85% sequence identity to the sequence of SEQ ID NO:17. In embodiments, the fluorescent protein domain includes an amino acid sequence having 90% sequence identity to the sequence of SEQ ID NO:17. In embodiments, the fluorescent protein domain includes an amino acid sequence having 95% sequence identity to the sequence of SEQ ID NO:17. In embodiments, the fluorescent protein domain includes an amino acid sequence having 96% sequence identity to the sequence of SEQ ID NO:17. In embodiments, the fluorescent protein domain includes an amino acid sequence having 97% sequence identity to the sequence of SEQ ID NO:17. In embodiments, the fluorescent protein domain includes an amino acid sequence having 98% sequence identity to the sequence of SEQ ID NO: 17. In embodiments, the fluorescent protein domain includes an amino acid sequence having 99% sequence identity to the sequence of SEQ ID NO: 17. In embodiments, the fluorescent protein domain includes an amino acid sequence having 100% sequence identity to the sequence of SEQ ID NO:17.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO: 18. In embodiments, the fluorescent protein domain includes an amino acid sequence having 70% sequence identity to the sequence of SEQ ID NO: 18. In embodiments, the fluorescent protein domain includes an amino acid sequence having 75% sequence identity to the sequence of SEQ ID NO:18. In embodiments, the fluorescent protein domain includes an amino acid sequence having 80% sequence identity to the sequence of SEQ ID NO: 18. In embodiments, the fluorescent protein domain includes an amino acid sequence having 85% sequence identity to the sequence of SEQ ID NO:18. In embodiments, the fluorescent protein domain includes an amino acid sequence having 90% sequence identity to the sequence of SEQ ID NO:18. In embodiments, the fluorescent protein domain includes an amino acid sequence having 95% sequence identity to the sequence of SEQ ID NO:18. In embodiments, the fluorescent protein domain includes an amino acid sequence having 96% sequence identity to the sequence of SEQ ID NO:18. In embodiments, the fluorescent protein domain includes an amino acid sequence having 97% sequence identity to the sequence of SEQ ID NO: 18. In embodiments, the fluorescent protein domain includes an amino acid sequence having 98% sequence identity to the sequence of SEQ ID NO:18. In embodiments, the fluorescent protein domain includes an amino acid sequence having 99% sequence identity to the sequence of SEQ ID NO:18. In embodiments, the fluorescent protein domain includes an amino acid sequence having 100% sequence identity to the sequence of SEQ ID NO: 18.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO:19. In embodiments, the fluorescent protein domain includes an amino acid sequence having 70% sequence identity to the sequence of SEQ ID NO:19. In embodiments, the fluorescent protein domain includes an amino acid sequence having 75% sequence identity to the sequence of SEQ ID NO:19. In embodiments, the fluorescent protein domain includes an amino acid sequence having 80% sequence identity to the sequence of SEQ ID NO:19. In embodiments, the fluorescent protein domain includes an amino acid sequence having 85% sequence identity to the sequence of SEQ ID NO: 19. In embodiments. the fluorescent protein domain includes an amino acid sequence having 90% sequence identity to the sequence of SEQ ID NO: 19. In embodiments, the fluorescent protein domain includes an amino acid sequence having 95% sequence identity to the sequence of SEQ ID NO:19. In embodiments, the fluorescent protein domain includes an amino acid sequence having 96% sequence identity to the sequence of SEQ ID NO:19. In embodiments, the fluorescent protein domain includes an amino acid sequence having 97% sequence identity to the sequence of SEQ ID NO:19. In embodiments, the fluorescent protein domain includes an amino acid sequence having 98% sequence identity to the sequence of SEQ ID NO:19. In embodiments, the fluorescent protein domain includes an amino acid sequence having 99% sequence identity to the sequence of SEQ ID NO: 19. In embodiments, the fluorescent protein domain includes an amino acid sequence having 100% sequence identity to the sequence of SEQ ID NO:19.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO:20. In embodiments, the fluorescent protein domain includes an amino acid sequence having 70% sequence identity to the sequence of SEQ ID NO:20. In embodiments, the fluorescent protein domain includes an amino acid sequence having 75% sequence identity to the sequence of SEQ ID NO:20. In embodiments, the fluorescent protein domain includes an amino acid sequence having 80% sequence identity to the sequence of SEQ ID NO:20. In embodiments, the fluorescent protein domain includes an amino acid sequence having 85% sequence identity to the sequence of SEQ ID NO:20. In embodiments, the fluorescent protein domain includes an amino acid sequence having 90% sequence identity to the sequence of SEQ ID NO:20. In embodiments, the fluorescent protein domain includes an amino acid sequence having 95% sequence identity to the sequence of SEQ ID NO:20. In embodiments, the fluorescent protein domain includes an amino acid sequence having 96% sequence identity to the sequence of SEQ ID NO:20. In embodiments, the fluorescent protein domain includes an amino acid sequence having 97% sequence identity to the sequence of SEQ ID NO:20. In embodiments, the fluorescent protein domain includes an amino acid sequence having 98% sequence identity to the sequence of SEQ ID NO:20. In embodiments, the fluorescent protein domain includes an amino acid sequence having 99% sequence identity to the sequence of SEQ ID NO:20. In embodiments, the fluorescent protein domain includes an amino acid sequence having 100% sequence identity to the sequence of SEQ ID NO:20.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO:21. In embodiments, the fluorescent protein domain includes an amino acid sequence having 70% sequence identity to the sequence of SEQ ID NO:21. In embodiments, the fluorescent protein domain includes an amino acid sequence having 75% sequence identity to the sequence of SEQ ID NO:21. In embodiments, the fluorescent protein domain includes an amino acid sequence having 80% sequence identity to the sequence of SEQ ID NO:21. In embodiments, the fluorescent protein domain includes an amino acid sequence having 85% sequence identity to the sequence of SEQ ID NO:21. In embodiments, the fluorescent protein domain includes an amino acid sequence having 90% sequence identity to the sequence of SEQ ID NO:21. In embodiments, the fluorescent protein domain includes an amino acid sequence having 95% sequence identity to the sequence of SEQ ID NO:21. In embodiments, the fluorescent protein domain includes an amino acid sequence having 96% sequence identity to the sequence of SEQ ID NO:21. In embodiments, the fluorescent protein domain includes an amino acid sequence having 97% sequence identity to the sequence of SEQ ID NO:21. In embodiments, the fluorescent protein domain includes an amino acid sequence having 98% sequence identity to the sequence of SEQ ID NO:21. In embodiments, the fluorescent protein domain includes an amino acid sequence having 99% sequence identity to the sequence of SEQ ID NO:21. In embodiments, the fluorescent protein domain includes an amino acid sequence having 100% sequence identity to the sequence of SEQ ID NO:21.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO:22. In embodiments, the fluorescent protein domain includes an amino acid sequence having 70% sequence identity to the sequence of SEQ ID NO:22. In embodiments, the fluorescent protein domain includes an amino acid sequence having 75% sequence identity to the sequence of SEQ ID NO:22. In embodiments, the fluorescent protein domain includes an amino acid sequence having 80% sequence identity to the sequence of SEQ ID NO:22. In embodiments, the fluorescent protein domain includes an amino acid sequence having 85% sequence identity to the sequence of SEQ ID NO:22. In embodiments, the fluorescent protein domain includes an amino acid sequence having 90% sequence identity to the sequence of SEQ ID NO:22. In embodiments, the fluorescent protein domain includes an amino acid sequence having 95% sequence identity to the sequence of SEQ ID NO:22. In embodiments, the fluorescent protein domain includes an amino acid sequence having 96% sequence identity to the sequence of SEQ ID NO:22. In embodiments, the fluorescent protein domain includes an amino acid sequence having 97% sequence identity to the sequence of SEQ ID NO:22. In embodiments, the fluorescent protein domain includes an amino acid sequence having 98% sequence identity to the sequence of SEQ ID NO:22. In embodiments, the fluorescent protein domain includes an amino acid sequence having 99% sequence identity to the sequence of SEQ ID NO:22. In embodiments, the fluorescent protein domain includes an amino acid sequence having 100% sequence identity to the sequence of SEQ ID NO:22.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO:23. In embodiments, the fluorescent protein domain includes an amino acid sequence having 70% sequence identity to the sequence of SEQ ID NO:23. In embodiments, the fluorescent protein domain includes an amino acid sequence having 75% sequence identity to the sequence of SEQ ID NO:23. In embodiments, the fluorescent protein domain includes an amino acid sequence having 80% sequence identity to the sequence of SEQ ID NO:23. In embodiments, the fluorescent protein domain includes an amino acid sequence having 85% sequence identity to the sequence of SEQ ID NO:23. In embodiments, the fluorescent protein domain includes an amino acid sequence having 90% sequence identity to the sequence of SEQ ID NO:23. In embodiments, the fluorescent protein domain includes an amino acid sequence having 95% sequence identity to the sequence of SEQ ID NO:23. In embodiments, the fluorescent protein domain includes an amino acid sequence having 96% sequence identity to the sequence of SEQ ID NO:23. In embodiments, the fluorescent protein domain includes an amino acid sequence having 97% sequence identity to the sequence of SEQ ID NO:23. In embodiments, the fluorescent protein domain includes an amino acid sequence having 98% sequence identity to the sequence of SEQ ID NO:23. In embodiments, the fluorescent protein domain includes an amino acid sequence having 99% sequence identity to the sequence of SEQ ID NO:23. In embodiments, the fluorescent protein domain includes an amino acid sequence having 100% sequence identity to the sequence of SEQ ID NO:23.

In embodiments, the fluorescent protein domain includes an amino acid sequence having 70%, 75%, 80%, 85%, 90%, 95%, 96%, 97%, 98%, 99% or 100% sequence identity to the sequence of SEQ ID NO:24. In embodiments, the fluorescent protein domain includes an amino acid sequence having 70% sequence identity to the sequence of SEQ ID NO:24. In embodiments, the fluorescent protein domain includes an amino acid sequence having 75% sequence identity to the sequence of SEQ ID NO:24. In embodiments, the fluorescent protein domain includes an amino acid sequence having 80% sequence identity to the sequence of SEQ ID NO:24. In embodiments, the fluorescent protein domain includes an amino acid sequence having 85% sequence identity to the sequence of SEQ ID NO:24. In embodiments, the fluorescent protein domain includes an amino acid sequence having 90% sequence identity to the sequence of SEQ ID NO:24. In embodiments, the fluorescent protein domain includes an amino acid sequence having 95% sequence identity to the sequence of SEQ ID NO:24. In embodiments, the fluorescent protein domain includes an amino acid sequence having 96% sequence identity to the sequence of SEQ ID NO:24. In embodiments, the fluorescent protein domain includes an amino acid sequence having 97% sequence identity to the sequence of SEQ ID NO:24. In embodiments, the fluorescent protein domain includes an amino acid sequence having 98% sequence identity to the sequence of SEQ ID NO:24. In embodiments, the fluorescent protein domain includes an amino acid sequence having 99% sequence identity to the sequence of SEQ ID NO:24. In embodiments, the fluorescent protein domain includes an amino acid sequence having 100% sequence identity to the sequence of SEQ ID NO:24.

In embodiments, the haloalkane dehalogenase domain includes the amino acid sequence of SEQ ID NO:25 or SEQ ID NO:26. In embodiments, the haloalkane dehalogenase domain includes the amino acid sequence of SEQ ID NO:25. In embodiments, the haloalkane dehalogenase domain includes the amino acid sequence of SEQ ID NO:26.

In embodiments, the haloalkane dehalogenase domain includes an amino acid sequence having 70%, 75%, 80%, 85%, 90%, 95%, 96%, 97%, 98%, 99% or 100% sequence identity to the sequence of SEQ ID NO:25. In embodiments, the haloalkane dehalogenase domain includes an amino acid sequence having 70% sequence identity to the sequence of SEQ ID NO:25. In embodiments, the haloalkane dehalogenase domain includes an amino acid sequence having 75% sequence identity to the sequence of SEQ ID NO:25. In embodiments, the haloalkane dehalogenase domain includes an amino acid sequence having 80% sequence identity to the sequence of SEQ ID NO:25. In embodiments, the haloalkane dehalogenase domain includes an amino acid sequence having 85% sequence identity to the sequence of SEQ ID NO:25. In embodiments, the haloalkane dehalogenase domain includes an amino acid sequence having 90% sequence identity to the sequence of SEQ ID NO:25. In embodiments, the haloalkane dehalogenase domain includes an amino acid sequence having 95% sequence identity to the sequence of SEQ ID NO:25. In embodiments, the haloalkane dehalogenase domain includes an amino acid sequence having 96% sequence identity to the sequence of SEQ ID NO:25. In embodiments, the haloalkane dehalogenase domain includes an amino acid sequence having 97% sequence identity to the sequence of SEQ ID NO:25. In embodiments, the haloalkane dehalogenase domain includes an amino acid sequence having 98% sequence identity to the sequence of SEQ ID NO:25. In embodiments, the haloalkane dehalogenase domain includes an amino acid sequence having 99% sequence identity to the sequence of SEQ ID NO:25. In embodiments, the haloalkane dehalogenase domain includes an amino acid sequence having 100% sequence identity to the sequence of SEQ ID NO:25.

In embodiments, the haloalkane dehalogenase domain includes an amino acid sequence having 70%, 75%, 80%, 85%, 90%, 95%, 96%, 97%, 98%, 99% or 100% sequence identity to the sequence of SEQ ID NO:26. In embodiments, the haloalkane dehalogenase domain includes an amino acid sequence having 70% sequence identity to the sequence of SEQ ID NO:26. In embodiments, the haloalkane dehalogenase domain includes an amino acid sequence having 75% sequence identity to the sequence of SEQ ID NO:26. In embodiments, the haloalkane dehalogenase domain includes an amino acid sequence having 80% sequence identity to the sequence of SEQ ID NO:26. In embodiments, the haloalkane dehalogenase domain includes an amino acid sequence having 85% sequence identity to the sequence of SEQ ID NO:26. In embodiments, the haloalkane dehalogenase domain includes an amino acid sequence having 90% sequence identity to the sequence of SEQ ID NO:26. In embodiments, the haloalkane dehalogenase domain includes an amino acid sequence having 95% sequence identity to the sequence of SEQ ID NO:26. In embodiments, the haloalkane dehalogenase domain includes an amino acid sequence having 96% sequence identity to the sequence of SEQ ID NO:26. In embodiments, the haloalkane dehalogenase domain includes an amino acid sequence having 97% sequence identity to the sequence of SEQ ID NO:26. In embodiments, the haloalkane dehalogenase domain includes an amino acid sequence having 98% sequence identity to the sequence of SEQ ID NO:26. In embodiments, the haloalkane dehalogenase domain includes an amino acid sequence having 99% sequence identity to the sequence of SEQ ID NO:26. In embodiments, the haloalkane dehalogenase domain includes an amino acid sequence having 100% sequence identity to the sequence of SEQ ID NO:26.

In embodiments, the chemical fluorophore is NGD, JF669, JFX646, or JF635. In embodiments, the chemical fluorophore is NGD. In embodiments, the chemical fluorophore is JF669. In embodiments, the chemical fluorophore is JFX646. In embodiments, the chemical fluorophore is JF635.

In embodiments the chemical fluorophore includes the formula of

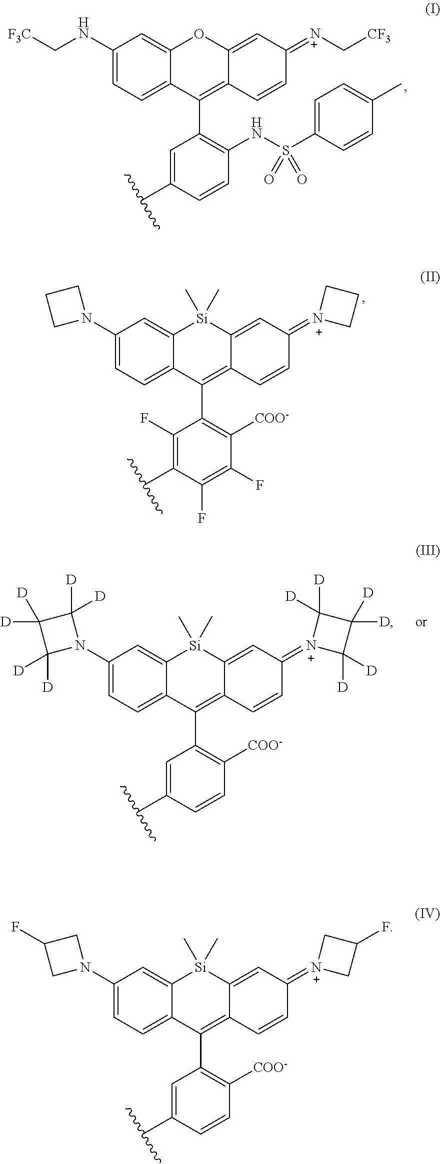

In embodiments, the chemical fluorophore includes the formula of

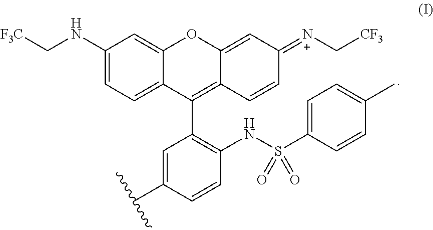 [SMILES: *c1ccc(NS(=O)(=O)c2ccc(C)cc2)c(-c2c3cc/c(=[NH+]\CC(F)(F)F)cc-3oc3cc(NCC(F)(F)F)ccc23)c1]

In embodiments, the chemical fluorophore includes the formula of

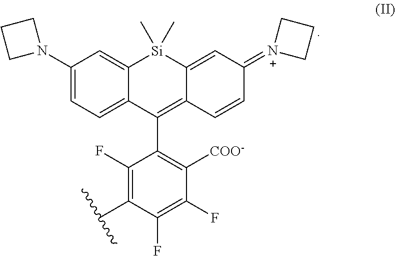

In embodiments, the chemical fluorophore includes the formula of

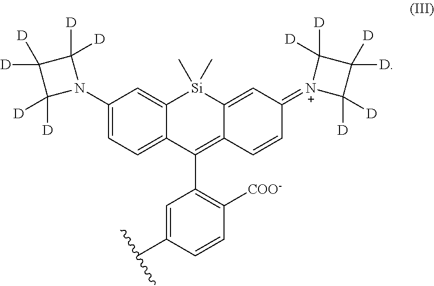

In embodiments, the chemical fluorophore includes the formula of

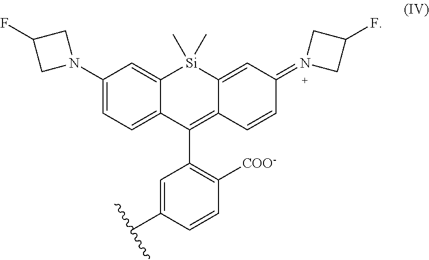

In embodiments, the biosensing protein domain includes: (a) a target biomolecule binding domain; (b) a target biomolecule substrate domain, a linker domain, and a target biomolecule activity sensing domain; or (c) a target biomolecule domain, a linker domain, and a target biomolecule substrate domain. In embodiments, the biosensing protein domain includes a target biomolecule binding domain. In embodiments, the biosensing protein domain includes a target biomolecule substrate domain, a linker domain, and a target biomolecule activity sensing domain. In embodiments, the biosensing protein domain includes a target biomolecule substrate domain. In embodiments, the biosensing protein domain includes a linker domain. In embodiments, the biosensing protein domain includes a target biomolecule activity sensing. In embodiments, the biosensing protein domain includes a target biomolecule domain, a linker domain, and a target biomolecule substrate domain. In embodiments, the biosensing protein domain includes a target biomolecule domain. In embodiments, the biosensing protein domain includes a linker domain. In embodiments, the biosensing protein domain includes a target biomolecule substrate domain.

In embodiments, the target biomolecule binding domain is capable of binding the target biomolecule. In embodiments, the target biomolecule binding domain binds the target biomolecule, thereby inducing a conformational change in the recombinant protein. In embodiments, the target biomolecule binding domain includes a phosphorylatable amino acid residue. In embodiments, the binding of the target biomolecule binding domain to the target biomolecule induces a modification of the target biomolecule binding domain. In embodiments, the modification of the target biomolecule binding domain is phosphorylation of the phosphorylatable amino acid residue. In embodiments, the target biomolecule phosphorylates the phosphorylatable amino acid residue, thereby inducing a conformational change in the recombinant protein. In embodiments, the conformational changes allows: (a) the chemical fluorophore to activate the fluorescent protein domain; or (b) the fluorescent protein domain to activate the chemical fluorophore. In embodiments, the conformational change increases the fluorescent signal from the FRET pair. In embodiments, the conformational change decreases the fluorescent signal from the FRET pair.

In embodiments, the target biomolecule binding domain is capable of interacting with the target biomolecule. In embodiments, the target biomolecule binding domain interacts with the target biomolecule, thereby inducing a conformational change in the recombinant protein. In embodiments, the conformational changes allows: (a) the chemical fluorophore to activate the fluorescent protein domain; or (b) the fluorescent protein domain to activate the chemical fluorophore. In embodiments, the conformational change increases the fluorescent signal from the FRET pair. In embodiments, the conformational change decreases the fluorescent signal from the FRET pair.

In embodiments, the target biomolecule domain is capable of binding the target biomolecule substrate domain. In embodiments, the target biomolecule domain is capable of being activated. In embodiments, the activated target biomolecule domain binds the target biomolecule substrate domain. In embodiments, the target biomolecule domain binds the target biomolecule substrate domain, thereby inducing a conformational change in the recombinant protein. In embodiments, the conformational changes allows: (a) the chemical fluorophore to activate the fluorescent protein domain; or (b) the fluorescent protein domain to activate the chemical fluorophore. In embodiments, the conformational change increases the fluorescent signal from the FRET pair. In embodiments, the conformational change decreases the fluorescent signal from the FRET pair.

In embodiments, the target biomolecule is a protein kinase, a second messenger molecule, or a GTPase. In embodiments, the target biomolecule is a protein kinase. In embodiments, the target biomolecule is a second messenger molecule. In embodiments, the target biomolecule is a GTPase.

In embodiments, the protein kinase is a protein kinase A (PKA), a protein kinase C (PKC), a protein kinase B (PKB), an extracellular signal-regulated kinase (ERK), a 5′ adenosine monophosphate-activated protein kinase (AMPK), a mammalian target of rapamycin (mTOR), a Fyn kinase, or a Src kinase. In embodiments, the protein kinase is a protein kinase A (PKA). In embodiments, the protein kinase is a protein kinase C (PKC). In embodiments, the protein kinase is a protein kinase B (PKB). In embodiments, the protein kinase is an extracellular signal-regulated kinase (ERK). In embodiments, the protein kinase is a 5′ adenosine monophosphate-activated protein kinase (AMPK). In embodiments, the protein kinase is a mammalian target of rapamycin (mTOR). In embodiments, the protein kinase is a Fyn kinase. In embodiments, the protein kinase is a Src kinase.

In embodiments, the second messenger molecule is cyclic adenosine monophosphate (cAMP), cyclic guanosine monophosphate (cGMP), inositol triphosphate (IP₃), diacylglycerol (DAG), or calcium (Ca). In embodiments, the second messenger molecule is cyclic adenosine monophosphate (cAMP). In embodiments, the second messenger molecule is cyclic guanosine monophosphate (cGMP). In embodiments, the second messenger molecule is inositol triphosphate (IP₃). In embodiments, the second messenger molecule is diacylglycerol (DAG). In embodiments, the second messenger molecule is calcium (Ca²⁺).

In embodiments, the GTPase is a Ras GTPase or a Rap1 GTPase. In embodiments, the GTPase is a Ras GTPase. In embodiments, the GTPase is a Rap1 GTPase.

In embodiments, the biomolecule substrate domain includes the amino acid sequence of any one of SEQ ID NO:27 to SEQ ID NO:36. In embodiments, the biomolecule substrate domain includes the amino acid sequence of SEQ ID NO:27. In embodiments, the biomolecule substrate domain includes the amino acid sequence of SEQ ID NO:28. In embodiments, the biomolecule substrate domain includes the amino acid sequence of SEQ ID NO:29. In embodiments, the biomolecule substrate domain includes the amino acid sequence of SEQ ID NO:30. In embodiments, the biomolecule substrate domain includes the amino acid sequence of SEQ ID NO:31. In embodiments, the biomolecule substrate domain includes the amino acid sequence of SEQ ID NO:32. In embodiments, the biomolecule substrate domain includes the amino acid sequence of SEQ ID NO:33. In embodiments, the biomolecule substrate domain includes the amino acid sequence of SEQ ID NO:34. In embodiments, the biomolecule substrate domain includes the amino acid sequence of SEQ ID NO:35. In embodiments, the biomolecule substrate domain includes the amino acid sequence of SEQ ID NO:36.

In embodiments, the biomolecule substrate domain is the amino acid sequence of any one of SEQ ID NO:27 to SEQ ID NO:36. In embodiments, the biomolecule substrate domain is the amino acid sequence of SEQ ID NO:27. In embodiments, the biomolecule substrate domain is the amino acid sequence of SEQ ID NO:28. In embodiments, the biomolecule substrate domain is the amino acid sequence of SEQ ID NO:29. In embodiments, the biomolecule substrate domain is the amino acid sequence of SEQ ID NO:30. In embodiments, the biomolecule substrate domain is the amino acid sequence of SEQ ID NO:31. In embodiments, the biomolecule substrate domain is the amino acid sequence of SEQ ID NO:32. In embodiments, the biomolecule substrate domain is the amino acid sequence of SEQ ID NO:33. In embodiments, the biomolecule substrate domain is the amino acid sequence of SEQ ID NO:34. In embodiments, the biomolecule substrate domain is the amino acid sequence of SEQ ID NO:35. In embodiments, the biomolecule substrate domain is the amino acid sequence of SEQ ID NO:36.

In embodiments, the biomolecule substrate domain includes an amino acid sequence having 70%, 75%, 80%, 85%, 90%, 95%, 96%, 97%, 98%, 99% or 100% sequence identity to 5, 6, 7, 8, 9, 10, 11, 12, 13, 14, 15, or 16 contiguous amino acids of the sequence of SEQ ID NO:27. In embodiments, the biomolecule substrate domain includes an amino acid sequence having 70% sequence identity to 5, 6, 7, 8, 9, 10, 11, 12, 13, 14, 15, or 16 contiguous amino acids of the sequence of SEQ ID NO:27. In embodiments, the biomolecule substrate domain includes an amino acid sequence having 75% sequence identity to 5, 6, 7, 8, 9, 10, 11, 12, 13, 14, 15, or 16 contiguous amino acids of the sequence of SEQ ID NO:27. In embodiments, the biomolecule substrate domain includes an amino acid sequence having 80% sequence identity to 5, 6, 7, 8, 9, 10, 11, 12, 13, 14, 15, or 16 contiguous amino acids of the sequence of SEQ ID NO:27. In embodiments, the biomolecule substrate domain includes an amino acid sequence having 85% sequence identity to 5, 6, 7, 8, 9, 10, 11, 12, 13, 14, 15, or 16 contiguous amino acids of the sequence of SEQ ID NO:27. In embodiments, the biomolecule substrate domain includes an amino acid sequence having 90% sequence identity to 5, 6, 7, 8, 9, 10, 11, 12, 13, 14, 15, or 16 contiguous amino acids of the sequence of SEQ ID NO:27. In embodiments, the biomolecule substrate domain includes an amino acid sequence having 95% sequence identity to 5, 6, 7, 8, 9, 10, 11, 12, 13, 14, 15, or 16 contiguous amino acids of the sequence of SEQ ID NO:27. In embodiments, the biomolecule substrate domain includes an amino acid sequence having 96% sequence identity to 5, 6, 7, 8, 9, 10, 11, 12, 13, 14, 15, or 16 contiguous amino acids of the sequence of SEQ ID NO:27. In embodiments, the biomolecule substrate domain includes an amino acid sequence having 97% sequence identity to 5, 6, 7, 8, 9, 10, 11, 12, 13, 14, 15, or 16 contiguous amino acids of the sequence of SEQ ID NO:27. In embodiments, the biomolecule substrate domain includes an amino acid sequence having 98% sequence identity to 5, 6, 7, 8, 9, 10, 11, 12, 13, 14, 15, or 16 contiguous amino acids of the sequence of SEQ ID NO:27. In embodiments, the biomolecule substrate domain includes an amino acid sequence having 99% sequence identity to 5, 6, 7, 8, 9, 10, 11, 12, 13, 14, 15, or 16 contiguous amino acids of the sequence of SEQ ID NO:27. In embodiments, the biomolecule substrate domain includes an amino acid sequence having 100% sequence identity to 5, 6, 7, 8, 9, 10, 11, 12, 13, 14, 15, or 16 contiguous amino acids of the sequence of SEQ ID NO:27.

In embodiments, the biomolecule substrate domain includes an amino acid sequence having 70%, 75%, 80%, 85%, 90%, 95%, 96%, 97%, 98%, 99% or 100% sequence identity to 5, 6, 7, 8, 9, 10, 11, 12, 13, 14, 15, or 16 contiguous amino acids of the sequence of SEQ ID NO:28. In embodiments, the biomolecule substrate domain includes an amino acid sequence having 70% sequence identity to 5, 6, 7, 8, 9, 10, 11, 12, 13, 14, 15, or 16 contiguous amino acids of the sequence of SEQ ID NO:28. In embodiments, the biomolecule substrate domain includes an amino acid sequence having 75% sequence identity to 5, 6, 7, 8, 9, 10, 11, 12, 13, 14, 15, or 16 contiguous amino acids of the sequence of SEQ ID NO:28. In embodiments, the biomolecule substrate domain includes an amino acid sequence having 80% sequence identity to 5, 6, 7, 8, 9, 10, 11, 12, 13, 14, 15, or 16 contiguous amino acids of the sequence of SEQ ID NO:28. In embodiments, the biomolecule substrate domain includes an amino acid sequence having 85% sequence identity to 5, 6, 7, 8, 9, 10, 11, 12, 13, 14, 15, or 16 contiguous amino acids of the sequence of SEQ ID NO:28. In embodiments, the biomolecule substrate domain includes an amino acid sequence having 90% sequence identity to 5, 6, 7, 8, 9, 10, 11, 12, 13, 14, 15, or 16 contiguous amino acids of the sequence of SEQ ID NO:28. In embodiments, the biomolecule substrate domain includes an amino acid sequence having 95% sequence identity to 5, 6, 7, 8, 9, 10, 11, 12, 13, 14, 15, or 16 contiguous amino acids of the sequence of SEQ ID NO:28. In embodiments, the biomolecule substrate domain includes an amino acid sequence having 96% sequence identity to 5, 6, 7, 8, 9, 10, 11, 12, 13, 14, 15, or 16 contiguous amino acids of the sequence of SEQ ID NO:28. In embodiments, the biomolecule substrate domain includes an amino acid sequence having 97% sequence identity to 5, 6, 7, 8, 9, 10, 11, 12, 13, 14, 15, or 16 contiguous amino acids of the sequence of SEQ ID NO:28. In embodiments, the biomolecule substrate domain includes an amino acid sequence having 98% sequence identity to 5, 6, 7, 8, 9, 10, 11, 12, 13, 14, 15, or 16 contiguous amino acids of the sequence of SEQ ID NO:28. In embodiments, the biomolecule substrate domain includes an amino acid sequence having 99% sequence identity to 5, 6, 7, 8, 9, 10, 11, 12, 13, 14, 15, or 16 contiguous amino acids of the sequence of SEQ ID NO:28. In embodiments, the biomolecule substrate domain includes an amino acid sequence having 100% sequence identity to 5, 6, 7, 8, 9, 10, 11, 12, 13, 14, 15, or 16 contiguous amino acids of the sequence of SEQ ID NO:28.

In embodiments, the biomolecule substrate domain includes an amino acid sequence having 70%, 75%, 80%, 85%, 90%, 95%, 96%, 97%, 98%, 99% or 100% sequence identity to 5, 10, 11, 12, 13, 14, 15, 16, 17, 18, or 19 contiguous amino acids of the sequence of SEQ ID NO:29. In embodiments, the biomolecule substrate domain includes an amino acid sequence having 70% sequence identity to 5, 10, 11, 12, 13, 14, 15, 16, 17, 18, or 19 contiguous amino acids of the sequence of SEQ ID NO:29. In embodiments, the biomolecule substrate domain includes an amino acid sequence having 75% sequence identity to 5, 10, 11, 12, 13, 14, 15, 16, 17, 18, or 19 contiguous amino acids of the sequence of SEQ ID NO:29. In embodiments, the biomolecule substrate domain includes an amino acid sequence having 80% sequence identity to 5, 10, 11, 12, 13, 14, 15, 16, 17, 18, or 19 contiguous amino acids of the sequence of SEQ ID NO:29. In embodiments, the biomolecule substrate domain includes an amino acid sequence having 85% sequence identity to 5, 10, 11, 12, 13, 14, 15, 16, 17, 18, or 19 contiguous amino acids of the sequence of SEQ ID NO:29. In embodiments, the biomolecule substrate domain includes an amino acid sequence having 90% sequence identity to 5, 10, 11, 12, 13, 14, 15, 16, 17, 18, or 19 contiguous amino acids of the sequence of SEQ ID NO:29. In embodiments, the biomolecule substrate domain includes an amino acid sequence having 95% sequence identity to 5, 10, 11, 12, 13, 14, 15, 16, 17, 18, or 19 contiguous amino acids of the sequence of SEQ ID NO:29. In embodiments, the biomolecule substrate domain includes an amino acid sequence having 96% sequence identity to 5, 10, 11, 12, 13, 14, 15, 16, 17, 18, or 19 contiguous amino acids of the sequence of SEQ ID NO:29. In embodiments, the biomolecule substrate domain includes an amino acid sequence having 97% sequence identity to 5, 10, 11, 12, 13, 14, 15, 16, 17, 18, or 19 contiguous amino acids of the sequence of SEQ ID NO:29. In embodiments, the biomolecule substrate domain includes an amino acid sequence having 98% sequence identity to 5, 10, 11, 12, 13, 14, 15, 16, 17, 18, or 19 contiguous amino acids of the sequence of SEQ ID NO:29. In embodiments, the biomolecule substrate domain includes an amino acid sequence having 99% sequence identity to 5, 10, 11, 12, 13, 14, 15, 16, 17, 18, or 19 contiguous amino acids of the sequence of SEQ ID NO:29. In embodiments, the biomolecule substrate domain includes an amino acid sequence having 100% sequence identity to 5, 10, 11, 12, 13, 14, 15, 16, 17, 18, or 19 contiguous amino acids of the sequence of SEQ ID NO:29.

In embodiments, the biomolecule substrate domain includes an amino acid sequence having 70%, 75%, 80%, 85%, 90%, 95%, 96%, 97%, 98%, 99% or 100% sequence identity to 5, 10, 11, 12, 13, 14, 15, 16, 17, 18, 19, or 20 contiguous amino acids of the sequence of SEQ ID NO:30. In embodiments, the biomolecule substrate domain includes an amino acid sequence having 70% sequence identity to 5, 10, 11, 12, 13, 14, 15, 16, 17, 18, 19, or 20 contiguous amino acids of the sequence of SEQ ID NO:30. In embodiments, the biomolecule substrate domain includes an amino acid sequence having 75% sequence identity to 5, 10, 11, 12, 13, 14, 15, 16, 17, 18, 19, or 20 contiguous amino acids of the sequence of SEQ ID NO:30. In embodiments, the biomolecule substrate domain includes an amino acid sequence having 80% sequence identity to 5, 10, 11, 12, 13, 14, 15, 16, 17, 18, 19, or 20 contiguous amino acids of the sequence of SEQ ID NO:30. In embodiments, the biomolecule substrate domain includes an amino acid sequence having 85% sequence identity to 5, 10, 11, 12, 13, 14, 15, 16, 17, 18, 19, or 20 contiguous amino acids of the sequence of SEQ ID NO:30. In embodiments, the biomolecule substrate domain includes an amino acid sequence having 90% sequence identity to 5, 10, 11, 12, 13, 14, 15, 16, 17, 18, 19, or 20 contiguous amino acids of the sequence of SEQ ID NO:30. In embodiments, the biomolecule substrate domain includes an amino acid sequence having 95% sequence identity to 5, 10, 11, 12, 13, 14, 15, 16, 17, 18, 19, or 20 contiguous amino acids of the sequence of SEQ ID NO:30. In embodiments, the biomolecule substrate domain includes an amino acid sequence having 96% sequence identity to 5, 10, 11, 12, 13, 14, 15, 16, 17, 18, 19, or 20 contiguous amino acids of the sequence of SEQ ID NO:30. In embodiments, the biomolecule substrate domain includes an amino acid sequence having 97% sequence identity to 5, 10, 11, 12, 13, 14, 15, 16, 17, 18, 19, or 20 contiguous amino acids of the sequence of SEQ ID NO:30. In embodiments, the biomolecule substrate domain includes an amino acid sequence having 98% sequence identity to 5, 10, 11, 12, 13, 14, 15, 16, 17, 18, 19, or 20 contiguous amino acids of the sequence of SEQ ID NO:30. In embodiments, the biomolecule substrate domain includes an amino acid sequence having 99% sequence identity to 5, 10, 11, 12, 13, 14, 15, 16, 17, 18, 19, or 20 contiguous amino acids of the sequence of SEQ ID NO:30. In embodiments, the biomolecule substrate domain includes an amino acid sequence having 100% sequence identity to 5, 10, 11, 12, 13, 14, 15, 16, 17, 18, 19, or 20 contiguous amino acids of the sequence of SEQ ID NO:30.

In embodiments, the biomolecule substrate domain includes an amino acid sequence having 70%, 75%, 80%, 85%, 90%, 95%, 96%, 97%, 98%, 99% or 100% sequence identity to 5, 10, 15, 16, 17, 18, 19, 20, 21, 22, or 23 contiguous amino acids of the sequence of SEQ ID NO:31. In embodiments, the biomolecule substrate domain includes an amino acid sequence having 70% sequence identity to 5, 10, 15, 16, 17, 18, 19, 20, 21, 22, or 23 contiguous amino acids of the sequence of SEQ ID NO:31. In embodiments, the biomolecule substrate domain includes an amino acid sequence having 75% sequence identity to 5, 10, 15, 16, 17, 18, 19, 20, 21, 22, or 23 contiguous amino acids of the sequence of SEQ ID NO:31. In embodiments, the biomolecule substrate domain includes an amino acid sequence having 80% sequence identity to 5, 10, 15, 16, 17, 18, 19, 20, 21, 22, or 23 contiguous amino acids of the sequence of SEQ ID NO:31. In embodiments, the biomolecule substrate domain includes an amino acid sequence having 85% sequence identity to 5, 10, 15, 16, 17, 18, 19, 20, 21, 22, or 23 contiguous amino acids of the sequence of SEQ ID NO:31. In embodiments, the biomolecule substrate domain includes an amino acid sequence having 90% sequence identity to 5, 10, 15, 16, 17, 18, 19, 20, 21, 22, or 23 contiguous amino acids of the sequence of SEQ ID NO:31. In embodiments, the biomolecule substrate domain includes an amino acid sequence having 95% sequence identity to 5, 10, 15, 16, 17, 18, 19, 20, 21, 22, or 23 contiguous amino acids of the sequence of SEQ ID NO:31. In embodiments, the biomolecule substrate domain includes an amino acid sequence having 96% sequence identity to 5, 10, 15, 16, 17, 18, 19, 20, 21, 22, or 23 contiguous amino acids of the sequence of SEQ ID NO:31. In embodiments, the biomolecule substrate domain includes an amino acid sequence having 97% sequence identity to 5, 10, 15, 16, 17, 18, 19, 20, 21, 22, or 23 contiguous amino acids of the sequence of SEQ ID NO:31. In embodiments, the biomolecule substrate domain includes an amino acid sequence having 98% sequence identity to 5, 10, 15, 16, 17, 18, 19, 20, 21, 22, or 23 contiguous amino acids of the sequence of SEQ ID NO:31. In embodiments, the biomolecule substrate domain includes an amino acid sequence having 99% sequence identity to 5, 10, 15, 16, 17, 18, 19, 20, 21, 22, or 23 contiguous amino acids of the sequence of SEQ ID NO:31. In embodiments, the biomolecule substrate domain includes an amino acid sequence having 100% sequence identity to 5, 10, 15, 16, 17, 18, 19, 20, 21, 22, or 23 contiguous amino acids of the sequence of SEQ ID NO:31.

In embodiments, the biomolecule substrate domain includes an amino acid sequence having 70%, 75%, 80%, 85%, 90%, 95%, 96%, 97%, 98%, 99% or 100% sequence identity to 5, 6, 7, 8, 9, 10, 11, 12, 13, 14, 15, 16, or 17 contiguous amino acids of the sequence of SEQ ID NO:32. In embodiments, the biomolecule substrate domain includes an amino acid sequence having 70% sequence identity to 5, 6, 7, 8, 9, 10, 11, 12, 13, 14, 15, 16, or 17 contiguous amino acids of the sequence of SEQ ID NO:32. In embodiments, the biomolecule substrate domain includes an amino acid sequence having 75% sequence identity to 5, 6, 7, 8, 9, 10, 11, 12, 13, 14, 15, 16, or 17 contiguous amino acids of the sequence of SEQ ID NO:32.

In embodiments, the biomolecule substrate domain includes an amino acid sequence having 80% sequence identity to 5, 6, 7, 8, 9, 10, 11, 12, 13, 14, 15, 16, or 17 contiguous amino acids of the sequence of SEQ ID NO:32. In embodiments, the biomolecule substrate domain includes an amino acid sequence having 85% sequence identity to 5, 6, 7, 8, 9, 10, 11, 12, 13, 14, 15, 16, or 17 contiguous amino acids of the sequence of SEQ ID NO:32. In embodiments, the biomolecule substrate domain includes an amino acid sequence having 90% sequence identity to 5, 6, 7, 8, 9, 10, 11, 12, 13, 14, 15, 16, or 17 contiguous amino acids of the sequence of SEQ ID NO:32. In embodiments, the biomolecule substrate domain includes an amino acid sequence having 95% sequence identity to 5, 6, 7, 8, 9, 10, 11, 12, 13, 14, 15, 16, or 17 contiguous amino acids of the sequence of SEQ ID NO:32. In embodiments, the biomolecule substrate domain includes an amino acid sequence having 96% sequence identity to 5, 6, 7, 8, 9, 10, 11, 12, 13, 14, 15, 16, or 17 contiguous amino acids of the sequence of SEQ ID NO:32. In embodiments, the biomolecule substrate domain includes an amino acid sequence having 97% sequence identity to 5, 6, 7, 8, 9, 10, 11, 12, 13, 14, 15, 16, or 17 contiguous amino acids of the sequence of SEQ ID NO:32. In embodiments, the biomolecule substrate domain includes an amino acid sequence having 98% sequence identity to 5, 6, 7, 8, 9, 10, 11, 12, 13, 14, 15, 16, or 17 contiguous amino acids of the sequence of SEQ ID NO:32. In embodiments, the biomolecule substrate domain includes an amino acid sequence having 99% sequence identity to 5, 6, 7, 8, 9, 10, 11, 12, 13, 14, 15, 16, or 17 contiguous amino acids of the sequence of SEQ ID NO:32. In embodiments, the biomolecule substrate domain includes an amino acid sequence having 100% sequence identity to 5, 6, 7, 8, 9, 10, 11, 12, 13, 14, 15, 16, or 17 contiguous amino acids of the sequence of SEQ ID NO:32.

In embodiments, the biomolecule substrate domain includes an amino acid sequence having 70%, 75%, 80%, 85%, 90%, 95%, 96%, 97%, 98%, 99% or 100% sequence identity to 5, 6, 7, 8, 9, 10, 11, or 12 contiguous amino acids of the sequence of SEQ ID NO:33. In embodiments, the biomolecule substrate domain includes an amino acid sequence having 70% sequence identity to 5, 6, 7, 8, 9, 10, 11, or 12 contiguous amino acids of the sequence of SEQ ID NO:33. In embodiments, the biomolecule substrate domain includes an amino acid sequence having 75% sequence identity to 5, 6, 7, 8, 9, 10, 11, or 12 contiguous amino acids of the sequence of SEQ ID NO:33. In embodiments, the biomolecule substrate domain includes an amino acid sequence having 80% sequence identity to 5, 6, 7, 8, 9, 10, 11, or 12 contiguous amino acids of the sequence of SEQ ID NO:3³. In embodiments, the biomolecule substrate domain includes an amino acid sequence having 85% sequence identity to 5, 6, 7, 8, 9, 10, 11, or 12 contiguous amino acids of the sequence of SEQ ID NO:33. In embodiments, the biomolecule substrate domain includes an amino acid sequence having 90% sequence identity to 5, 6, 7, 8, 9, 10, 11, or 12 contiguous amino acids of the sequence of SEQ ID NO:33. In embodiments, the biomolecule substrate domain includes an amino acid sequence having 95% sequence identity to 5, 6, 7, 8, 9, 10, 11, or 12 contiguous amino acids of the sequence of SEQ ID NO:33. In embodiments, the biomolecule substrate domain includes an amino acid sequence having 96% sequence identity to 5, 6, 7, 8, 9, 10, 11, or 12 contiguous amino acids of the sequence of SEQ ID NO:33. In embodiments, the biomolecule substrate domain includes an amino acid sequence having 97% sequence identity to 5, 6, 7, 8, 9, 10, 11, or 12 contiguous amino acids of the sequence of SEQ ID NO:33. In embodiments, the biomolecule substrate domain includes an amino acid sequence having 98% sequence identity to 5, 6, 7, 8, 9, 10, 11, or 12 contiguous amino acids of the sequence of SEQ ID NO:33. In embodiments, the biomolecule substrate domain includes an amino acid sequence having 99% sequence identity to 5, 6, 7, 8, 9, 10, 11, or 12 contiguous amino acids of the sequence of SEQ ID NO:33. In embodiments, the biomolecule substrate domain includes an amino acid sequence having 100% sequence identity to 5, 6, 7, 8, 9, 10, 11, or 12 contiguous amino acids of the sequence of SEQ ID NO:33.

In embodiments, the biomolecule substrate domain includes an amino acid sequence having 70%, 75%, 80%, 85%, 90%, 95%, 96%, 97%, 98%, 99% or 100% sequence identity to 5, 6, 7, 8, 9, or 10 contiguous amino acids of the sequence of SEQ ID NO:34. In embodiments, the biomolecule substrate domain includes an amino acid sequence having 70% sequence identity to 5, 6, 7, 8, 9, or 10 contiguous amino acids of the sequence of SEQ ID NO:34. In embodiments, the biomolecule substrate domain includes an amino acid sequence having 75% sequence identity to 5, 6, 7, 8, 9, or 10 contiguous amino acids of the sequence of SEQ ID NO:34. In embodiments, the biomolecule substrate domain includes an amino acid sequence having 80% sequence identity to 5, 6, 7, 8, 9, or 10 contiguous amino acids of the sequence of SEQ ID NO:34. In embodiments, the biomolecule substrate domain includes an amino acid sequence having 85% sequence identity to 5, 6, 7, 8, 9, or 10 contiguous amino acids of the sequence of SEQ ID NO:34. In embodiments, the biomolecule substrate domain includes an amino acid sequence having 90% sequence identity to 5, 6, 7, 8, 9, or 10 contiguous amino acids of the sequence of SEQ ID NO:34. In embodiments, the biomolecule substrate domain includes an amino acid sequence having 95% sequence identity to 5, 6, 7, 8, 9, or 10 contiguous amino acids of the sequence of SEQ ID NO:34. In embodiments, the biomolecule substrate domain includes an amino acid sequence having 96% sequence identity to 5, 6, 7, 8, 9, or 10 contiguous amino acids of the sequence of SEQ ID NO:34. In embodiments, the biomolecule substrate domain includes an amino acid sequence having 97% sequence identity to 5, 6, 7, 8, 9, or 10 contiguous amino acids of the sequence of SEQ ID NO:34. In embodiments, the biomolecule substrate domain includes an amino acid sequence having 98% sequence identity to 5, 6, 7, 8, 9, or 10 contiguous amino acids of the sequence of SEQ ID NO:34. In embodiments, the biomolecule substrate domain includes an amino acid sequence having 99% sequence identity to 5, 6, 7, 8, 9, or 10 contiguous amino acids of the sequence of SEQ ID NO:34. In embodiments, the biomolecule substrate domain includes an amino acid sequence having 100% sequence identity to 5, 6, 7, 8, 9, or 10 contiguous amino acids of the sequence of SEQ ID NO:34.

In embodiments, the biomolecule substrate domain includes an amino acid sequence having 70%, 75%, 80%, 85%, 90%, 95%, 96%, 97%, 98%, 99% or 100% sequence identity to 5, 10, 20, 30, 40, 50, 60, 70, 75, 76, 77, 78, 79, 80, or 81 contiguous amino acids of the sequence of SEQ ID NO:35. In embodiments, the biomolecule substrate domain includes an amino acid sequence having 70% sequence identity to 5, 10, 20, 30, 40, 50, 60, 70, 75, 76, 77, 78, 79, 80, or 81 contiguous amino acids of the sequence of SEQ ID NO:35. In embodiments, the biomolecule substrate domain includes an amino acid sequence having 75% sequence identity to 5, 10, 20, 30, 40, 50, 60, 70, 75, 76, 77, 78, 79, 80, or 81 contiguous amino acids of the sequence of SEQ ID NO:35. In embodiments, the biomolecule substrate domain includes an amino acid sequence having 80% sequence identity to 5, 10, 20, 30, 40, 50, 60, 70, 75, 76, 77, 78, 79, 80, or 81 contiguous amino acids of the sequence of SEQ ID NO:35. In embodiments, the biomolecule substrate domain includes an amino acid sequence having 85% sequence identity to 5, 10, 20, 30, 40, 50, 60, 70, 75, 76, 77, 78, 79, 80, or 81 contiguous amino acids of the sequence of SEQ ID NO:35. In embodiments, the biomolecule substrate domain includes an amino acid sequence having 90% sequence identity to 5, 10, 20, 30, 40, 50, 60, 70, 75, 76, 77, 78, 79, 80, or 81 contiguous amino acids of the sequence of SEQ ID NO:35. In embodiments, the biomolecule substrate domain includes an amino acid sequence having 95% sequence identity to 5, 10, 20, 30, 40, 50, 60, 70, 75, 76, 77, 78, 79, 80, or 81 contiguous amino acids of the sequence of SEQ ID NO:35. In embodiments, the biomolecule substrate domain includes an amino acid sequence having 96% sequence identity to 5, 10, 20, 30, 40, 50, 60, 70, 75, 76, 77, 78, 79, 80, or 81 contiguous amino acids of the sequence of SEQ ID NO:35. In embodiments, the biomolecule substrate domain includes an amino acid sequence having 97% sequence identity to 5, 10, 20, 30, 40, 50, 60, 70, 75, 76, 77, 78, 79, 80, or 81 contiguous amino acids of the sequence of SEQ ID NO:35. In embodiments, the biomolecule substrate domain includes an amino acid sequence having 98% sequence identity to 5, 10, 20, 30, 40, 50, 60, 70, 75, 76, 77, 78, 79, 80, or 81 contiguous amino acids of the sequence of SEQ ID NO:35. In embodiments, the biomolecule substrate domain includes an amino acid sequence having 99% sequence identity to 5, 10, 20, 30, 40, 50, 60, 70, 75, 76, 77, 78, 79, 80, or 81 contiguous amino acids of the sequence of SEQ ID NO:35. In embodiments, the biomolecule substrate domain includes an amino acid sequence having 100% sequence identity to 5, 10, 20, 30, 40, 50, 60, 70, 75, 76, 77, 78, 79, 80, or 81 contiguous amino acids of the sequence of SEQ ID NO:35.

In embodiments, the biomolecule substrate domain includes an amino acid sequence having 70%, 75%, 80%, 85%, 90%, 95%, 96%, 97%, 98%, 99% or 100% sequence identity to 5, 10, 25, 50, 75, 100, 125, 135, 140, 141, 142, 143, 144, 145, 146, or 147 contiguous amino acids of the sequence of SEQ ID NO:36. In embodiments, the biomolecule substrate domain includes an amino acid sequence having 70% sequence identity to 5, 10, 25, 50, 75, 100, 125, 135, 140, 141, 142, 143, 144, 145, 146, or 147 contiguous amino acids of the sequence of SEQ ID NO:36. In embodiments, the biomolecule substrate domain includes an amino acid sequence having 75% sequence identity to 5, 10, 25, 50, 75, 100, 125, 135, 140, 141, 142, 143, 144, 145, 146, or 147 contiguous amino acids of the sequence of SEQ ID NO:36. In embodiments, the biomolecule substrate domain includes an amino acid sequence having 80% sequence identity to 5, 10, 25, 50, 75, 100, 125, 135, 140, 141, 142, 143, 144, 145, 146, or 147 contiguous amino acids of the sequence of SEQ ID NO:36. In embodiments, the biomolecule substrate domain includes an amino acid sequence having 85% sequence identity to 5, 10, 25, 50, 75, 100, 125, 135, 140, 141, 142, 143, 144, 145, 146, or 147 contiguous amino acids of the sequence of SEQ ID NO:36. In embodiments, the biomolecule substrate domain includes an amino acid sequence having 90% sequence identity to 5, 10, 25, 50, 75, 100, 125, 135, 140, 141, 142, 143, 144, 145, 146, or 147 contiguous amino acids of the sequence of SEQ ID NO:36. In embodiments, the biomolecule substrate domain includes an amino acid sequence having 95% sequence identity to 5, 10, 25, 50, 75, 100, 125, 135, 140, 141, 142, 143, 144, 145, 146, or 147 contiguous amino acids of the sequence of SEQ ID NO:36. In embodiments, the biomolecule substrate domain includes an amino acid sequence having 96% sequence identity to 5, 10, 25, 50, 75, 100, 125, 135, 140, 141, 142, 143, 144, 145, 146, or 147 contiguous amino acids of the sequence of SEQ ID NO:36. In embodiments, the biomolecule substrate domain includes an amino acid sequence having 97% sequence identity to 5, 10, 25, 50, 75, 100, 125, 135, 140, 141, 142, 143, 144, 145, 146, or 147 contiguous amino acids of the sequence of SEQ ID NO:36. In embodiments, the biomolecule substrate domain includes an amino acid sequence having 98% sequence identity to 5, 10, 25, 50, 75, 100, 125, 135, 140, 141, 142, 143, 144, 145, 146, or 147 contiguous amino acids of the sequence of SEQ ID NO:36. In embodiments, the biomolecule substrate domain includes an amino acid sequence having 99% sequence identity to 5, 10, 25, 50, 75, 100, 125, 135, 140, 141, 142, 143, 144, 145, 146, or 147 contiguous amino acids of the sequence of SEQ ID NO:36. In embodiments, the biomolecule substrate domain includes an amino acid sequence having 100% sequence identity to 5, 10, 25, 50, 75, 100, 125, 135, 140, 141, 142, 143, 144, 145, 146, or 147 contiguous amino acids of the sequence of SEQ ID NO:36.

In embodiments, the target biomolecule substrate domain further includes a phosphorylatable amino acid residue. In embodiments, the phosphorylatable amino acid residue is capable of being phosphorylated by the protein kinase.

In embodiments, the target biomolecule domain includes the amino acid sequence of SEQ ID NO:44 or SEQ ID NO:45. In embodiments, the target biomolecule domain includes the amino acid sequence of SEQ ID NO:44. In embodiments, the target biomolecule domain includes the amino acid sequence of SEQ ID NO:45. In embodiments, the target biomolecule domain is the amino acid sequence of SEQ ID NO:44 or SEQ ID NO:45. In embodiments, the target biomolecule domain is the amino acid sequence of SEQ ID NO:44.

In embodiments, the target biomolecule domain is the amino acid sequence of SEQ ID NO:45.

In embodiments, the target biomolecule domain includes an amino acid sequence having 70%, 75%, 80%, 85%, 90%, 95%, 96%, 97%, 98%, 99% or 100% sequence identity to the sequence of SEQ ID NO:44. In embodiments, the target biomolecule domain includes an amino acid sequence having 70% sequence identity to the sequence of SEQ ID NO:44. In embodiments, the target biomolecule domain includes an amino acid sequence having 75% sequence identity to the sequence of SEQ ID NO:44. In embodiments, the target biomolecule domain includes an amino acid sequence having 80% sequence identity to the sequence of SEQ ID NO:44. In embodiments, the target biomolecule domain includes an amino acid sequence having 85% sequence identity to the sequence of SEQ ID NO:44. In embodiments, the target biomolecule domain includes an amino acid sequence having 90% sequence identity to the sequence of SEQ ID NO:44. In embodiments, the target biomolecule domain includes an amino acid sequence having 95% sequence identity to the sequence of SEQ ID NO:44. In embodiments, the target biomolecule domain includes an amino acid sequence having 96% sequence identity to the sequence of SEQ ID NO:44. In embodiments, the target biomolecule domain includes an amino acid sequence having 97% sequence identity to the sequence of SEQ ID NO:44. In embodiments, the target biomolecule domain includes an amino acid sequence having 98% sequence identity to the sequence of SEQ ID NO:44. In embodiments, the target biomolecule domain includes an amino acid sequence having 99% sequence identity to the sequence of SEQ ID NO:44. In embodiments, the target biomolecule domain includes an amino acid sequence having 100% sequence identity to the sequence of SEQ ID NO:44.

In embodiments, the target biomolecule domain includes an amino acid sequence having 70%, 75%, 80%, 85%, 90%, 95%, 96%, 97%, 98%, 99% or 100% sequence identity to the sequence of SEQ ID NO:45. In embodiments, the target biomolecule domain includes an amino acid sequence having 70% sequence identity to the sequence of SEQ ID NO:45. In embodiments, the target biomolecule domain includes an amino acid sequence having 75% sequence identity to the sequence of SEQ ID NO:45. In embodiments, the target biomolecule domain includes an amino acid sequence having 80% sequence identity to the sequence of SEQ ID NO:45. In embodiments, the target biomolecule domain includes an amino acid sequence having 85% sequence identity to the sequence of SEQ ID NO:45. In embodiments, the target biomolecule domain includes an amino acid sequence having 90% sequence identity to the sequence of SEQ ID NO:45. In embodiments, the target biomolecule domain includes an amino acid sequence having 95% sequence identity to the sequence of SEQ ID NO:45. In embodiments, the target biomolecule domain includes an amino acid sequence having 96% sequence identity to the sequence of SEQ ID NO:45. In embodiments, the target biomolecule domain includes an amino acid sequence having 97% sequence identity to the sequence of SEQ ID NO:45. In embodiments, the target biomolecule domain includes an amino acid sequence having 98% sequence identity to the sequence of SEQ ID NO:45. In embodiments, the target biomolecule domain includes an amino acid sequence having 99% sequence identity to the sequence of SEQ ID NO:45. In embodiments, the target biomolecule domain includes an amino acid sequence having 100% sequence identity to the sequence of SEQ ID NO:45.

In embodiments, the target biomolecule activity sensing domain includes a phosphoamino acid binding domain. In embodiments, the phosphoamino acid binding domain is capable of binding a phosphorylated amino acid residue. In embodiments, the phosphoamino acid binding domain is capable of binding a phosphorylated amino acid residue in the target biomolecule substrate domain. In embodiments, the phosphoamino acid binding domain binds a phosphorylated amino acid residue in the target biomolecule substrate domain. In embodiments, the phosphoamino acid binding domain binds a phosphorylated amino acid residue in the target biomolecule substrate domain, thereby inducing a conformational change in the recombinant protein. In embodiments, the conformational changes allows: (a) the chemical fluorophore to activate the fluorescent protein domain; or (b) the fluorescent protein domain to activate the chemical fluorophore. In embodiments, the conformational change increases the fluorescent signal from the FRET pair. In embodiments, the conformational change decreases the fluorescent signal from the FRET pair.

In embodiments, the phosphoamino acid binding domain includes the amino acid sequence of SEQ ID NO:39. SEQ ID NO:40, or SEQ ID NO:41. In embodiments, the phosphoamino acid binding domain includes the amino acid sequence of SEQ ID NO:39. In embodiments, the phosphoamino acid binding domain includes the amino acid sequence of SEQ ID NO:40. In embodiments, the phosphoamino acid binding domain includes the amino acid sequence of SEQ ID NO:41. In embodiments, the phosphoamino acid binding domain is the amino acid sequence of SEQ ID NO:39, SEQ ID NO:40, or SEQ ID NO:41. In embodiments, the phosphoamino acid binding domain is the amino acid sequence of SEQ ID NO:39. In embodiments, the phosphoamino acid binding domain is the amino acid sequence of SEQ ID NO:40. In embodiments, the phosphoamino acid binding domain is the amino acid sequence of SEQ ID NO:41.

In embodiments, the phosphoamino acid binding domain includes an amino acid sequence having 70%, 75%, 80%, 85%, 90%, 95%, 96%, 97%, 98%, 99% or 100% sequence identity to the sequence of SEQ ID NO:39. In embodiments, the phosphoamino acid binding domain includes an amino acid sequence having 70% sequence identity to the sequence of SEQ ID NO:39. In embodiments, the phosphoamino acid binding domain includes an amino acid sequence having 75% sequence identity to the sequence of SEQ ID NO:39. In embodiments, the phosphoamino acid binding domain includes an amino acid sequence having 80% sequence identity to the sequence of SEQ ID NO:39. In embodiments, the phosphoamino acid binding domain includes an amino acid sequence having 85% sequence identity to the sequence of SEQ ID NO:39. In embodiments, the phosphoamino acid binding domain includes an amino acid sequence having 90% sequence identity to the sequence of SEQ ID NO:39. In embodiments, the phosphoamino acid binding domain includes an amino acid sequence having 95% sequence identity to the sequence of SEQ ID NO:39. In embodiments, the phosphoamino acid binding domain includes an amino acid sequence having 96% sequence identity to the sequence of SEQ ID NO:39. In embodiments, the phosphoamino acid binding domain includes an amino acid sequence having 97% sequence identity to the sequence of SEQ ID NO:39. In embodiments, the phosphoamino acid binding domain includes an amino acid sequence having 98% sequence identity to the sequence of SEQ ID NO:39. In embodiments, the phosphoamino acid binding domain includes an amino acid sequence having 99% sequence identity to the sequence of SEQ ID NO:39. In embodiments, the phosphoamino acid binding domain includes an amino acid sequence having 100% sequence identity to the sequence of SEQ ID NO:39.

In embodiments, the phosphoamino acid binding domain includes an amino acid sequence having 70%, 75%, 80%, 85%, 90%, 95%, 96%, 97%, 98%, 99% or 100% sequence identity to the sequence of SEQ ID NO:40. In embodiments, the phosphoamino acid binding domain includes an amino acid sequence having 70% sequence identity to the sequence of SEQ ID NO:40. In embodiments, the phosphoamino acid binding domain includes an amino acid sequence having 75% sequence identity to the sequence of SEQ ID NO:40. In embodiments, the phosphoamino acid binding domain includes an amino acid sequence having 80% sequence identity to the sequence of SEQ ID NO:40. In embodiments, the phosphoamino acid binding domain includes an amino acid sequence having 85% sequence identity to the sequence of SEQ ID NO:40. In embodiments, the phosphoamino acid binding domain includes an amino acid sequence having 90% sequence identity to the sequence of SEQ ID NO:40. In embodiments, the phosphoamino acid binding domain includes an amino acid sequence having 95% sequence identity to the sequence of SEQ ID NO:40. In embodiments, the phosphoamino acid binding domain includes an amino acid sequence having 96% sequence identity to the sequence of SEQ ID NO:40. In embodiments, the phosphoamino acid binding domain includes an amino acid sequence having 97% sequence identity to the sequence of SEQ ID NO:40. In embodiments, the phosphoamino acid binding domain includes an amino acid sequence having 98% sequence identity to the sequence of SEQ ID NO:40. In embodiments, the phosphoamino acid binding domain includes an amino acid sequence having 99% sequence identity to the sequence of SEQ ID NO:40. In embodiments, the phosphoamino acid binding domain includes an amino acid sequence having 100% sequence identity to the sequence of SEQ ID NO:40.

In embodiments, the phosphoamino acid binding domain includes an amino acid sequence having 70%, 75%, 80%, 85%, 90%, 95%, 96%, 97%, 98%, 99% or 100% sequence identity to the sequence of SEQ ID NO:41. In embodiments, the phosphoamino acid binding domain includes an amino acid sequence having 70% sequence identity to the sequence of SEQ ID NO:41. In embodiments, the phosphoamino acid binding domain includes an amino acid sequence having 75% sequence identity to the sequence of SEQ ID NO:41. In embodiments, the phosphoamino acid binding domain includes an amino acid sequence having 80% sequence identity to the sequence of SEQ ID NO:41. In embodiments, the phosphoamino acid binding domain includes an amino acid sequence having 85% sequence identity to the sequence of SEQ ID NO:41. In embodiments, the phosphoamino acid binding domain includes an amino acid sequence having 90% sequence identity to the sequence of SEQ ID NO:41. In embodiments, the phosphoamino acid binding domain includes an amino acid sequence having 95% sequence identity to the sequence of SEQ ID NO:41. In embodiments, the phosphoamino acid binding domain includes an amino acid sequence having 96% sequence identity to the sequence of SEQ ID NO:41. In embodiments, the phosphoamino acid binding domain includes an amino acid sequence having 97% sequence identity to the sequence of SEQ ID NO:41. In embodiments, the phosphoamino acid binding domain includes an amino acid sequence having 98% sequence identity to the sequence of SEQ ID NO:41. In embodiments, the phosphoamino acid binding domain includes an amino acid sequence having 99% sequence identity to the sequence of SEQ ID NO:41. In embodiments, the phosphoamino acid binding domain includes an amino acid sequence having 100% sequence identity to the sequence of SEQ ID NO:41.

In embodiments, the linker domain is a peptide linker. In embodiments, the linker domain is between about 1 to about 500 amino acids in length. In embodiments, the linker domain is between about 2 to about 500 amino acids in length. In embodiments, the linker domain is between about 3 to about 500 amino acids in length. In embodiments, the linker domain is between about 4 to about 500 amino acids in length. In embodiments, the linker domain is between about 5 to about 500 amino acids in length. In embodiments, the linker domain is between about 6 to about 500 amino acids in length. In embodiments, the linker domain is between about 7 to about 500 amino acids in length. In embodiments, the linker domain is between about 8 to about 500 amino acids in length. In embodiments, the linker domain is between about 9 to about 500 amino acids in length. In embodiments, the linker domain is between about 10 to about 500 amino acids in length. In embodiments, the linker domain is between about 15 to about 500 amino acids in length. In embodiments, the linker domain is between about 20 to about 500 amino acids in length. In embodiments, the linker domain is between about 25 to about 500 amino acids in length. In embodiments, the linker domain is between about 30 to about 500 amino acids in length. In embodiments, the linker domain is between about 35 to about 500 amino acids in length. In embodiments, the linker domain is between about 40 to about 500 amino acids in length. In embodiments, the linker domain is between about 45 to about 500 amino acids in length. In embodiments, the linker domain is between about 50 to about 500 amino acids in length. In embodiments, the linker domain is between about 100 to about 500 amino acids in length. In embodiments, the linker domain is between about 150 to about 500 amino acids in length. In embodiments, the linker domain is between about 200 to about 500 amino acids in length. In embodiments, the linker domain is between about 250 to about 500 amino acids in length. In embodiments, the linker domain is between about 300 to about 500 amino acids in length. In embodiments, the linker domain is between about 350 to about 500 amino acids in length. In embodiments, the linker domain is between about 400 to about 500 amino acids in length. In embodiments, the linker domain is between about 450 to about 500 amino acids in length.

In embodiments, the linker domain is between about 1 to about 450 amino acids in length. In embodiments, the linker domain is between about 1 to about 400 amino acids in length. In embodiments, the linker domain is between about 1 to about 350 amino acids in length. In embodiments, the linker domain is between about 1 to about 300 amino acids in length. In embodiments, the linker domain is between about 1 to about 250 amino acids in length. In embodiments, the linker domain is between about 1 to about 200 amino acids in length. In embodiments, the linker domain is between about 1 to about 150 amino acids in length. In embodiments, the linker domain is between about 1 to about 100 amino acids in length. In embodiments, the linker domain is between about 1 to about 50 amino acids in length. In embodiments, the linker domain is between about 1 to about 45 amino acids in length. In embodiments, the linker domain is between about 1 to about 40 amino acids in length. In embodiments, the linker domain is between about 1 to about 35 amino acids in length. In embodiments, the linker domain is between about 1 to about 30 amino acids in length. In embodiments, the linker domain is between about 1 to about 25 amino acids in length. In embodiments, the linker domain is between about 1 to about 20 amino acids in length. In embodiments, the linker domain is between about 1 to about 15 amino acids in length. In embodiments, the linker domain is between about 1 to about 10 amino acids in length. In embodiments, the linker domain is between about 1 to about 9 amino acids in length. In embodiments, the linker domain is between about 1 to about 8 amino acids in length. In embodiments, the linker domain is between about 1 to about 7 amino acids in length. In embodiments, the linker domain is between about 1 to about 6 amino acids in length. In embodiments, the linker domain is between about 1 to about 5 amino acids in length. In embodiments, the linker domain is between about 1 to about 4 amino acids in length. In embodiments, the linker domain is between about 1 to about 3 amino acids in length. In embodiments, the linker domain is between about 1 to about 2 amino acids in length.

In embodiments, the linker domain is between 1 to 500 amino acids in length. In embodiments, the linker domain is between 2 to 500 amino acids in length. In embodiments, the linker domain is between 3 to 500 amino acids in length. In embodiments, the linker domain is between 4 to 500 amino acids in length. In embodiments, the linker domain is between 5 to 500 amino acids in length. In embodiments, the linker domain is between 6 to 500 amino acids in length. In embodiments, the linker domain is between 7 to 500 amino acids in length. In embodiments, the linker domain is between 8 to 500 amino acids in length. In embodiments, the linker domain is between 9 to 500 amino acids in length. In embodiments, the linker domain is between 10 to 500 amino acids in length. In embodiments, the linker domain is between 15 to 500 amino acids in length. In embodiments, the linker domain is between 20 to 500 amino acids in length. In embodiments, the linker domain is between 25 to 500 amino acids in length. In embodiments, the linker domain is between 30 to 500 amino acids in length. In embodiments, the linker domain is between 35 to 500 amino acids in length. In embodiments, the linker domain is between 40 to 500 amino acids in length. In embodiments, the linker domain is between 45 to 500 amino acids in length. In embodiments, the linker domain is between 50 to 500 amino acids in length. In embodiments, the linker domain is between 100 to 500 amino acids in length. In embodiments, the linker domain is between 150 to 500 amino acids in length. In embodiments, the linker domain is between 200 to 500 amino acids in length. In embodiments, the linker domain is between 250 to 500 amino acids in length. In embodiments, the linker domain is between 300 to 500 amino acids in length. In embodiments, the linker domain is between 350 to 500 amino acids in length. In embodiments, the linker domain is between 400 to 500 amino acids in length. In embodiments, the linker domain is between 450 to 500 amino acids in length.

In embodiments, the linker domain is between 1 to 450 amino acids in length. In embodiments, the linker domain is between 1 to 400 amino acids in length. In embodiments, the linker domain is between 1 to 350 amino acids in length. In embodiments, the linker domain is between 1 to 300 amino acids in length. In embodiments, the linker domain is between 1 to 250 amino acids in length. In embodiments, the linker domain is between 1 to 200 amino acids in length. In embodiments, the linker domain is between 1 to 150 amino acids in length. In embodiments, the linker domain is between 1 to 100 amino acids in length. In embodiments, the linker domain is between 1 to 50 amino acids in length. In embodiments, the linker domain is between 1 to 45 amino acids in length. In embodiments, the linker domain is between 1 to 40 amino acids in length. In embodiments, the linker domain is between 1 to 35 amino acids in length. In embodiments, the linker domain is between 1 to 30 amino acids in length. In embodiments, the linker domain is between 1 to 25 amino acids in length. In embodiments, the linker domain is between 1 to 20 amino acids in length. In embodiments, the linker domain is between 1 to 15 amino acids in length. In embodiments, the linker domain is between 1 to 10 amino acids in length. In embodiments, the linker domain is between 1 to 9 amino acids in length. In embodiments, the linker domain is between 1 to 8 amino acids in length. In embodiments, the linker domain is between 1 to 7 amino acids in length. In embodiments, the linker domain is between 1 to 6 amino acids in length. In embodiments, the linker domain is between 1 to 5 amino acids in length. In embodiments, the linker domain is between 1 to 4 amino acids in length. In embodiments, the linker domain is between 1 to 3 amino acids in length. In embodiments, the linker domain is between 1 to 2 amino acids in length.

In embodiments, the linker domain includes the amino acid sequence of any one of SEQ ID NO:46 to SEQ ID NO:59. In embodiments, the linker domain includes the amino acid sequence of any one of SEQ ID NO: 46. In embodiments, the linker domain includes the amino acid sequence of any one of SEQ ID NO:47. In embodiments, the linker domain includes the amino acid sequence of any one of SEQ ID NO:48. In embodiments, the linker domain includes the amino acid sequence of any one of SEQ ID NO:49. In embodiments, the linker domain includes the amino acid sequence of any one of SEQ ID NO: 50. In embodiments, the linker domain includes the amino acid sequence of any one of SEQ ID NO:51. In embodiments, the linker domain includes the amino acid sequence of any one of SEQ ID NO:52. In embodiments, the linker domain includes the amino acid sequence of any one of SEQ ID NO:53. In embodiments, the linker domain includes the amino acid sequence of any one of SEQ ID NO:54. In embodiments, the linker domain includes the amino acid sequence of any one of SEQ ID NO:55. In embodiments, the linker domain includes the amino acid sequence of any one of SEQ ID NO:56. In embodiments, the linker domain includes the amino acid sequence of any one of SEQ ID NO:57. In embodiments, the linker domain includes the amino acid sequence of any one of SEQ ID NO:58. In embodiments, the linker domain includes the amino acid sequence of any one of SEQ ID NO:59.

In embodiments, the linker domain is the amino acid sequence of any one of SEQ ID NO:46 to SEQ ID NO:59. In embodiments, the linker domain is the amino acid sequence of any one of SEQ ID NO:46. In embodiments, the linker domain is the amino acid sequence of any one of SEQ ID NO:47. In embodiments, the linker domain is the amino acid sequence of any one of SEQ ID NO:48. In embodiments, the linker domain is the amino acid sequence of any one of SEQ ID NO:49. In embodiments, the linker domain is the amino acid sequence of any one of SEQ ID NO:50. In embodiments, the linker domain is the amino acid sequence of any one of SEQ ID NO:51. In embodiments, the linker domain is the amino acid sequence of any one of SEQ ID NO:52. In embodiments, the linker domain is the amino acid sequence of any one of SEQ ID NO:53. In embodiments, the linker domain is the amino acid sequence of any one of SEQ ID NO:54. In embodiments, the linker domain is the amino acid sequence of any one of SEQ ID NO:55. In embodiments, the linker domain is the amino acid sequence of any one of SEQ ID NO:56. In embodiments, the linker domain is the amino acid sequence of any one of SEQ ID NO:57. In embodiments, the linker domain is the amino acid sequence of any one of SEQ ID NO:58. In embodiments, the linker domain is the amino acid sequence of any one of SEQ ID NO:59.

In embodiments, the linker domain includes an amino acid sequence having 70%, 75%, 80%, 85%, 90%, 95%, 96%, 97%, 98%, 99% or 100% sequence identity to the sequence of SEQ ID NO:46. In embodiments, the linker domain includes an amino acid sequence having 70% sequence identity to the sequence of SEQ ID NO:46. In embodiments, the linker domain includes an amino acid sequence having 75% sequence identity to the sequence of SEQ ID NO:46. In embodiments, the linker domain includes an amino acid sequence having 80% sequence identity to the sequence of SEQ ID NO:46. In embodiments, the linker domain includes an amino acid sequence having 85% sequence identity to the sequence of SEQ ID NO:46. In embodiments, the linker domain includes an amino acid sequence having 90% sequence identity to the sequence of SEQ ID NO:46. In embodiments, the linker domain includes an amino acid sequence having 95% sequence identity to the sequence of SEQ ID NO:46. In embodiments, the linker domain includes an amino acid sequence having 96% sequence identity to the sequence of SEQ ID NO:46. In embodiments, the linker domain includes an amino acid sequence having 97% sequence identity to the sequence of SEQ ID NO:46. In embodiments, the linker domain includes an amino acid sequence having 98% sequence identity to the sequence of SEQ ID NO:46. In embodiments, the linker domain includes an amino acid sequence having 99% sequence identity to the sequence of SEQ ID NO:46. In embodiments, the linker domain includes an amino acid sequence having 100% sequence identity to the sequence of SEQ ID NO:46.

In embodiments, the linker domain includes an amino acid sequence having 70%, 75%, 80%, 85%, 90%, 95%, 96%, 97%, 98%, 99% or 100% sequence identity to the sequence of SEQ ID NO:47. In embodiments, the linker domain includes an amino acid sequence having 70% sequence identity to the sequence of SEQ ID NO:47. In embodiments, the linker domain includes an amino acid sequence having 75% sequence identity to the sequence of SEQ ID NO:47. In embodiments, the linker domain includes an amino acid sequence having 80% sequence identity to the sequence of SEQ ID NO:47. In embodiments, the linker domain includes an amino acid sequence having 85% sequence identity to the sequence of SEQ ID NO:47. In embodiments, the linker domain includes an amino acid sequence having 90% sequence identity to the sequence of SEQ ID NO:47. In embodiments, the linker domain includes an amino acid sequence having 95% sequence identity to the sequence of SEQ ID NO:47. In embodiments, the linker domain includes an amino acid sequence having 96% sequence identity to the sequence of SEQ ID NO:47. In embodiments, the linker domain includes an amino acid sequence having 97% sequence identity to the sequence of SEQ ID NO:47. In embodiments, the linker domain includes an amino acid sequence having 98% sequence identity to the sequence of SEQ ID NO:47. In embodiments, the linker domain includes an amino acid sequence having 99% sequence identity to the sequence of SEQ ID NO:47. In embodiments, the linker domain includes an amino acid sequence having 100% sequence identity to the sequence of SEQ ID NO:47.

In embodiments, the linker domain includes an amino acid sequence having 70%, 75%, 80%, 85%, 90%, 95%, 96%, 97%, 98%, 99% or 100% sequence identity to the sequence of SEQ ID NO:48. In embodiments, the linker domain includes an amino acid sequence having 70% sequence identity to the sequence of SEQ ID NO:48. In embodiments, the linker domain includes an amino acid sequence having 75% sequence identity to the sequence of SEQ ID NO:48. In embodiments, the linker domain includes an amino acid sequence having 80% sequence identity to the sequence of SEQ ID NO:48. In embodiments, the linker domain includes an amino acid sequence having 85% sequence identity to the sequence of SEQ ID NO:48. In embodiments, the linker domain includes an amino acid sequence having 90% sequence identity to the sequence of SEQ ID NO:48. In embodiments. the linker domain includes an amino acid sequence having 95% sequence identity to the sequence of SEQ ID NO:48. In embodiments, the linker domain includes an amino acid sequence having 96% sequence identity to the sequence of SEQ ID NO:48. In embodiments, the linker domain includes an amino acid sequence having 97% sequence identity to the sequence of SEQ ID NO:48. In embodiments, the linker domain includes an amino acid sequence having 98% sequence identity to the sequence of SEQ ID NO:48. In embodiments, the linker domain includes an amino acid sequence having 99% sequence identity to the sequence of SEQ ID NO:48. In embodiments, the linker domain includes an amino acid sequence having 100% sequence identity to the sequence of SEQ ID NO:48.

In embodiments, the linker domain includes an amino acid sequence having 70%, 75%, 80%, 85%, 90%, 95%, 96%, 97%, 98%, 99% or 100% sequence identity to the sequence of SEQ ID NO:49. In embodiments, the linker domain includes an amino acid sequence having 70% sequence identity to the sequence of SEQ ID NO:49. In embodiments, the linker domain includes an amino acid sequence having 75% sequence identity to the sequence of SEQ ID NO:49. In embodiments, the linker domain includes an amino acid sequence having 80% sequence identity to the sequence of SEQ ID NO:49. In embodiments, the linker domain includes an amino acid sequence having 85% sequence identity to the sequence of SEQ ID NO:49. In embodiments, the linker domain includes an amino acid sequence having 90% sequence identity to the sequence of SEQ ID NO:49. In embodiments, the linker domain includes an amino acid sequence having 95% sequence identity to the sequence of SEQ ID NO:49. In embodiments, the linker domain includes an amino acid sequence having 96% sequence identity to the sequence of SEQ ID NO:49. In embodiments, the linker domain includes an amino acid sequence having 97% sequence identity to the sequence of SEQ ID NO:49. In embodiments, the linker domain includes an amino acid sequence having 98% sequence identity to the sequence of SEQ ID NO:49. In embodiments, the linker domain includes an amino acid sequence having 99% sequence identity to the sequence of SEQ ID NO:49. In embodiments, the linker domain includes an amino acid sequence having 100% sequence identity to the sequence of SEQ ID NO:49.

In embodiments, the linker domain includes an amino acid sequence having 70%, 75%, 80%, 85%, 90%, 95%, 96%, 97%, 98%, 99% or 100% sequence identity to the sequence of SEQ ID NO:50. In embodiments, the linker domain includes an amino acid sequence having 70% sequence identity to the sequence of SEQ ID NO:50. In embodiments, the linker domain includes an amino acid sequence having 75% sequence identity to the sequence of SEQ ID NO:50. In embodiments, the linker domain includes an amino acid sequence having 80% sequence identity to the sequence of SEQ ID NO:50. In embodiments, the linker domain includes an amino acid sequence having 85% sequence identity to the sequence of SEQ ID NO:50. In embodiments, the linker domain includes an amino acid sequence having 90% sequence identity to the sequence of SEQ ID NO:50. In embodiments, the linker domain includes an amino acid sequence having 95% sequence identity to the sequence of SEQ ID NO:50. In embodiments, the linker domain includes an amino acid sequence having 96% sequence identity to the sequence of SEQ ID NO:50. In embodiments, the linker domain includes an amino acid sequence having 97% sequence identity to the sequence of SEQ ID NO:50. In embodiments, the linker domain includes an amino acid sequence having 98% sequence identity to the sequence of SEQ ID NO:50. In embodiments, the linker domain includes an amino acid sequence having 99% sequence identity to the sequence of SEQ ID NO:50. In embodiments, the linker domain includes an amino acid sequence having 100% sequence identity to the sequence of SEQ ID NO:50.

In embodiments, the linker domain includes an amino acid sequence having 70%, 75%, 80%, 85%, 90%, 95%, 96%, 97%, 98%, 99% or 100% sequence identity to the sequence of SEQ ID NO:51. In embodiments, the linker domain includes an amino acid sequence having 70% sequence identity to the sequence of SEQ ID NO:51. In embodiments, the linker domain includes an amino acid sequence having 75% sequence identity to the sequence of SEQ ID NO:51. In embodiments, the linker domain includes an amino acid sequence having 80% sequence identity to the sequence of SEQ ID NO:51. In embodiments, the linker domain includes an amino acid sequence having 85% sequence identity to the sequence of SEQ ID NO:51. In embodiments, the linker domain includes an amino acid sequence having 90% sequence identity to the sequence of SEQ ID NO:51. In embodiments, the linker domain includes an amino acid sequence having 95% sequence identity to the sequence of SEQ ID NO:51. In embodiments, the linker domain includes an amino acid sequence having 96% sequence identity to the sequence of SEQ ID NO:51. In embodiments, the linker domain includes an amino acid sequence having 97% sequence identity to the sequence of SEQ ID NO:51. In embodiments, the linker domain includes an amino acid sequence having 98% sequence identity to the sequence of SEQ ID NO:51. In embodiments, the linker domain includes an amino acid sequence having 99% sequence identity to the sequence of SEQ ID NO:51. In embodiments, the linker domain includes an amino acid sequence having 100% sequence identity to the sequence of SEQ ID NO:51.

In embodiments, the linker domain includes an amino acid sequence having 70%, 75%, 80%, 85%, 90%, 95%, 96%, 97%, 98%, 99% or 100% sequence identity to the sequence of SEQ ID NO:52. In embodiments, the linker domain includes an amino acid sequence having 70% sequence identity to the sequence of SEQ ID NO:52. In embodiments, the linker domain includes an amino acid sequence having 75% sequence identity to the sequence of SEQ ID NO:52. In embodiments, the linker domain includes an amino acid sequence having 80% sequence identity to the sequence of SEQ ID NO:52. In embodiments, the linker domain includes an amino acid sequence having 85% sequence identity to the sequence of SEQ ID NO:52. In embodiments, the linker domain includes an amino acid sequence having 90% sequence identity to the sequence of SEQ ID NO:52. In embodiments, the linker domain includes an amino acid sequence having 95% sequence identity to the sequence of SEQ ID NO:52. In embodiments, the linker domain includes an amino acid sequence having 96% sequence identity to the sequence of SEQ ID NO:52. In embodiments, the linker domain includes an amino acid sequence having 97% sequence identity to the sequence of SEQ ID NO:52. In embodiments, the linker domain includes an amino acid sequence having 98% sequence identity to the sequence of SEQ ID NO:52. In embodiments, the linker domain includes an amino acid sequence having 99% sequence identity to the sequence of SEQ ID NO:52. In embodiments, the linker domain includes an amino acid sequence having 100% sequence identity to the sequence of SEQ ID NO:52.

In embodiments, the linker domain includes an amino acid sequence having 70%, 75%, 80%, 85%, 90%, 95%, 96%, 97%, 98%, 99% or 100% sequence identity to the sequence of SEQ ID NO:53. In embodiments, the linker domain includes an amino acid sequence having 70% sequence identity to the sequence of SEQ ID NO:53. In embodiments, the linker domain includes an amino acid sequence having 75% sequence identity to the sequence of SEQ ID NO:53. In embodiments, the linker domain includes an amino acid sequence having 80% sequence identity to the sequence of SEQ ID NO:53. In embodiments, the linker domain includes an amino acid sequence having 85% sequence identity to the sequence of SEQ ID NO:53. In embodiments, the linker domain includes an amino acid sequence having 90% sequence identity to the sequence of SEQ ID NO:53. In embodiments, the linker domain includes an amino acid sequence having 95% sequence identity to the sequence of SEQ ID NO:53. In embodiments, the linker domain includes an amino acid sequence having 96% sequence identity to the sequence of SEQ ID NO:53. In embodiments, the linker domain includes an amino acid sequence having 97% sequence identity to the sequence of SEQ ID NO:53. In embodiments, the linker domain includes an amino acid sequence having 98% sequence identity to the sequence of SEQ ID NO:53. In embodiments, the linker domain includes an amino acid sequence having 99% sequence identity to the sequence of SEQ ID NO:53. In embodiments, the linker domain includes an amino acid sequence having 100% sequence identity to the sequence of SEQ ID NO:53.

In embodiments, the linker domain includes an amino acid sequence having 70%, 75%, 80%, 85%, 90%, 95%, 96%, 97%, 98%, 99% or 100% sequence identity to the sequence of SEQ ID NO:54. In embodiments, the linker domain includes an amino acid sequence having 70% sequence identity to the sequence of SEQ ID NO:54. In embodiments, the linker domain includes an amino acid sequence having 75% sequence identity to the sequence of SEQ ID NO:54. In embodiments, the linker domain includes an amino acid sequence having 80% sequence identity to the sequence of SEQ ID NO:54. In embodiments, the linker domain includes an amino acid sequence having 85% sequence identity to the sequence of SEQ ID NO:54. In embodiments, the linker domain includes an amino acid sequence having 90% sequence identity to the sequence of SEQ ID NO:54. In embodiments, the linker domain includes an amino acid sequence having 95% sequence identity to the sequence of SEQ ID NO:54. In embodiments, the linker domain includes an amino acid sequence having 96% sequence identity to the sequence of SEQ ID NO:54. In embodiments, the linker domain includes an amino acid sequence having 97% sequence identity to the sequence of SEQ ID NO:54. In embodiments, the linker domain includes an amino acid sequence having 98% sequence identity to the sequence of SEQ ID NO:54. In embodiments, the linker domain includes an amino acid sequence having 99% sequence identity to the sequence of SEQ ID NO:54. In embodiments, the linker domain includes an amino acid sequence having 100% sequence identity to the sequence of SEQ ID NO:54.

In embodiments, the linker domain includes an amino acid sequence having 70%, 75%, 80%, 85%, 90%, 95%, 96%, 97%, 98%, 99% or 100% sequence identity to the sequence of SEQ ID NO:55. In embodiments, the linker domain includes an amino acid sequence having 70% sequence identity to the sequence of SEQ ID NO:55. In embodiments, the linker domain includes an amino acid sequence having 75% sequence identity to the sequence of SEQ ID NO:55. In embodiments, the linker domain includes an amino acid sequence having 80% sequence identity to the sequence of SEQ ID NO:55. In embodiments, the linker domain includes an amino acid sequence having 85% sequence identity to the sequence of SEQ ID NO:55. In embodiments, the linker domain includes an amino acid sequence having 90% sequence identity to the sequence of SEQ ID NO:55. In embodiments, the linker domain includes an amino acid sequence having 95% sequence identity to the sequence of SEQ ID NO:55. In embodiments, the linker domain includes an amino acid sequence having 96% sequence identity to the sequence of SEQ ID NO:55. In embodiments, the linker domain includes an amino acid sequence having 97% sequence identity to the sequence of SEQ ID NO:55. In embodiments, the linker domain includes an amino acid sequence having 98% sequence identity to the sequence of SEQ ID NO:55. In embodiments, the linker domain includes an amino acid sequence having 99% sequence identity to the sequence of SEQ ID NO:55. In embodiments, the linker domain includes an amino acid sequence having 100% sequence identity to the sequence of SEQ ID NO:55.

In embodiments, the linker domain includes an amino acid sequence having 70%, 75%, 80%, 85%, 90%, 95%, 96%, 97%, 98%, 99% or 100% sequence identity to the sequence of SEQ ID NO:56. In embodiments, the linker domain includes an amino acid sequence having 70% sequence identity to the sequence of SEQ ID NO:56. In embodiments, the linker domain includes an amino acid sequence having 75% sequence identity to the sequence of SEQ ID NO:56. In embodiments, the linker domain includes an amino acid sequence having 80% sequence identity to the sequence of SEQ ID NO:56. In embodiments, the linker domain includes an amino acid sequence having 85% sequence identity to the sequence of SEQ ID NO:56. In embodiments, the linker domain includes an amino acid sequence having 90% sequence identity to the sequence of SEQ ID NO:56. In embodiments, the linker domain includes an amino acid sequence having 95% sequence identity to the sequence of SEQ ID NO:56. In embodiments, the linker domain includes an amino acid sequence having 96% sequence identity to the sequence of SEQ ID NO:56. In embodiments, the linker domain includes an amino acid sequence having 97% sequence identity to the sequence of SEQ ID NO:56. In embodiments, the linker domain includes an amino acid sequence having 98% sequence identity to the sequence of SEQ ID NO:56. In embodiments, the linker domain includes an amino acid sequence having 99% sequence identity to the sequence of SEQ ID NO:56. In embodiments, the linker domain includes an amino acid sequence having 100% sequence identity to the sequence of SEQ ID NO:56.

In embodiments, the linker domain includes an amino acid sequence having 70%, 75%, 80%, 85%, 90%, 95%, 96%, 97%, 98%, 99% or 100% sequence identity to the sequence of SEQ ID NO:57. In embodiments, the linker domain includes an amino acid sequence having 70% sequence identity to the sequence of SEQ ID NO:57. In embodiments. the linker domain includes an amino acid sequence having 75% sequence identity to the sequence of SEQ ID NO:57. In embodiments, the linker domain includes an amino acid sequence having 80% sequence identity to the sequence of SEQ ID NO:57. In embodiments, the linker domain includes an amino acid sequence having 85% sequence identity to the sequence of SEQ ID NO:57. In embodiments, the linker domain includes an amino acid sequence having 90% sequence identity to the sequence of SEQ ID NO:57. In embodiments, the linker domain includes an amino acid sequence having 95% sequence identity to the sequence of SEQ ID NO:57. In embodiments, the linker domain includes an amino acid sequence having 96% sequence identity to the sequence of SEQ ID NO:57. In embodiments, the linker domain includes an amino acid sequence having 97% sequence identity to the sequence of SEQ ID NO:57. In embodiments, the linker domain includes an amino acid sequence having 98% sequence identity to the sequence of SEQ ID NO:57. In embodiments, the linker domain includes an amino acid sequence having 99% sequence identity to the sequence of SEQ ID NO:57. In embodiments, the linker domain includes an amino acid sequence having 100% sequence identity to the sequence of SEQ ID NO:57.

In embodiments, the linker domain includes an amino acid sequence having 70%, 75%, 80%, 85%, 90%, 95%, 96%, 97%, 98%, 99% or 100% sequence identity to the sequence of SEQ ID NO:58. In embodiments, the linker domain includes an amino acid sequence having 70% sequence identity to the sequence of SEQ ID NO:58. In embodiments, the linker domain includes an amino acid sequence having 75% sequence identity to the sequence of SEQ ID NO:58. In embodiments, the linker domain includes an amino acid sequence having 80% sequence identity to the sequence of SEQ ID NO:58. In embodiments, the linker domain includes an amino acid sequence having 85% sequence identity to the sequence of SEQ ID NO:58. In embodiments, the linker domain includes an amino acid sequence having 90% sequence identity to the sequence of SEQ ID NO:58. In embodiments, the linker domain includes an amino acid sequence having 95% sequence identity to the sequence of SEQ ID NO:58. In embodiments, the linker domain includes an amino acid sequence having 96% sequence identity to the sequence of SEQ ID NO:58. In embodiments, the linker domain includes an amino acid sequence having 97% sequence identity to the sequence of SEQ ID NO:58. In embodiments, the linker domain includes an amino acid sequence having 98% sequence identity to the sequence of SEQ ID NO:58. In embodiments, the linker domain includes an amino acid sequence having 99% sequence identity to the sequence of SEQ ID NO:58. In embodiments, the linker domain includes an amino acid sequence having 100% sequence identity to the sequence of SEQ ID NO:58.

In embodiments, the linker domain includes an amino acid sequence having 70%, 75%, 80%, 85%, 90%, 95%, 96%, 97%, 98%, 99% or 100% sequence identity to the sequence of SEQ ID NO:59. In embodiments, the linker domain includes an amino acid sequence having 70% sequence identity to the sequence of SEQ ID NO:59. In embodiments, the linker domain includes an amino acid sequence having 75% sequence identity to the sequence of SEQ ID NO:59. In embodiments, the linker domain includes an amino acid sequence having 80% sequence identity to the sequence of SEQ ID NO:59. In embodiments, the linker domain includes an amino acid sequence having 85% sequence identity to the sequence of SEQ ID NO:59. In embodiments, the linker domain includes an amino acid sequence having 90% sequence identity to the sequence of SEQ ID NO:59. In embodiments, the linker domain includes an amino acid sequence having 95% sequence identity to the sequence of SEQ ID NO:59. In embodiments, the linker domain includes an amino acid sequence having 96% sequence identity to the sequence of SEQ ID NO:59. In embodiments, the linker domain includes an amino acid sequence having 97% sequence identity to the sequence of SEQ ID NO:59. In embodiments, the linker domain includes an amino acid sequence having 98% sequence identity to the sequence of SEQ ID NO:59. In embodiments, the linker domain includes an amino acid sequence having 99% sequence identity to the sequence of SEQ ID NO:59. In embodiments, the linker domain includes an amino acid sequence having 100% sequence identity to the sequence of SEQ ID NO:59.

In embodiments, the target biomolecule binding domain includes the amino acid sequence of SEQ ID NO:37 or SEQ ID NO:38. In embodiments, the target biomolecule binding domain includes the amino acid sequence of SEQ ID NO:37. In embodiments, the target biomolecule binding domain includes the amino acid sequence of SEQ ID NO:38. In embodiments, the target biomolecule binding domain is the amino acid sequence of SEQ ID NO:37 or SEQ ID NO:38. In embodiments, the target biomolecule binding domain is the amino acid sequence of SEQ ID NO:37. In embodiments, the target biomolecule binding domain is the amino acid sequence of SEQ ID NO:38.

In embodiments, the target biomolecule binding domain includes an amino acid sequence having 70%, 75%, 80%, 85%, 90%, 95%, 96%, 97%, 98%, 99% or 100% sequence identity to the sequence of SEQ ID NO:37. In embodiments, the target biomolecule binding domain includes an amino acid sequence having 70% sequence identity to the sequence of SEQ ID NO:37. In embodiments, the target biomolecule binding domain includes an amino acid sequence having 75% sequence identity to the sequence of SEQ ID NO:37. In embodiments, the target biomolecule binding domain includes an amino acid sequence having 80% sequence identity to the sequence of SEQ ID NO:37. In embodiments, the target biomolecule binding domain includes an amino acid sequence having 85% sequence identity to the sequence of SEQ ID NO:37. In embodiments, the target biomolecule binding domain includes an amino acid sequence having 90% sequence identity to the sequence of SEQ ID NO:37. In embodiments, the target biomolecule binding domain includes an amino acid sequence having 95% sequence identity to the sequence of SEQ ID NO:37. In embodiments, the target biomolecule binding domain includes an amino acid sequence having 96% sequence identity to the sequence of SEQ ID NO:37. In embodiments, the target biomolecule binding domain includes an amino acid sequence having 97% sequence identity to the sequence of SEQ ID NO:37. In embodiments, the target biomolecule binding domain includes an amino acid sequence having 98% sequence identity to the sequence of SEQ ID NO:37. In embodiments, the target biomolecule binding domain includes an amino acid sequence having 99% sequence identity to the sequence of SEQ ID NO:37. In embodiments, the target biomolecule binding domain includes an amino acid sequence having 100% sequence identity to the sequence of SEQ ID NO:37.

In embodiments, the target biomolecule binding domain includes an amino acid sequence having 70%, 75%, 80%, 85%, 90%, 95%, 96%, 97%, 98%, 99% or 100% sequence identity to the sequence of SEQ ID NO:38. In embodiments, the target biomolecule binding domain includes an amino acid sequence having 70% sequence identity to the sequence of SEQ ID NO:38. In embodiments, the target biomolecule binding domain includes an amino acid sequence having 75% sequence identity to the sequence of SEQ ID NO:38. In embodiments, the target biomolecule binding domain includes an amino acid sequence having 80% sequence identity to the sequence of SEQ ID NO:38. In embodiments, the target biomolecule binding domain includes an amino acid sequence having 85% sequence identity to the sequence of SEQ ID NO:38. In embodiments, the target biomolecule binding domain includes an amino acid sequence having 90% sequence identity to the sequence of SEQ ID NO:38. In embodiments, the target biomolecule binding domain includes an amino acid sequence having 95% sequence identity to the sequence of SEQ ID NO:38. In embodiments, the target biomolecule binding domain includes an amino acid sequence having 96% sequence identity to the sequence of SEQ ID NO:38. In embodiments, the target biomolecule binding domain includes an amino acid sequence having 97% sequence identity to the sequence of SEQ ID NO:38. In embodiments, the target biomolecule binding domain includes an amino acid sequence having 98% sequence identity to the sequence of SEQ ID NO:38. In embodiments, the target biomolecule binding domain includes an amino acid sequence having 99% sequence identity to the sequence of SEQ ID NO:38. In embodiments, the target biomolecule binding domain includes an amino acid sequence having 100% sequence identity to the sequence of SEQ ID NO:38.

In embodiments, the fluorescent protein is attached to the N-terminus of the biosensing protein domain and the biosensing protein domain is attached to the N-terminus of the haloalkane dehalogenase domain.

In embodiments, the haloalkane dehalogenase domain is attached to the N-terminus of the biosensing protein domain and the biosensing protein domain is attached to the N-terminus of the fluorescent protein.

In another aspect is provided a recombinant protein covalently bound to a chemical fluorophore, the recombinant protein including a split fluorescent protein domain bound to a biosensing protein domain and a haloalkane dehalogenase domain bound to the biosensing protein domain, wherein: the chemical fluorophore is covalently bound to the haloalkane dehalogenase domain; the split fluorescent protein domain is one part of a fluorescent protein split pair that forms a reconstituted fluorescent protein upon binding of a cognate split fluorescent protein; and the chemical fluorophore and the reconstituted fluorescent protein domain are a Förster Resonance Energy Transfer (FRET) pair.

In embodiments, the recombinant protein is non-covalently bound to a second recombinant protein including the cognate split fluorescent protein domain bound to a protein of interest, wherein the split fluorescent protein domain is non-covalently bound to the cognate split fluorescent protein thereby forming the reconstituted fluorescent protein.

In embodiments, the reconstituted fluorescent protein is a reconstituted red fluorescent protein (RFP), a reconstituted orange fluorescent protein (OFP), a reconstituted yellow fluorescent protein (YFP), or a reconstituted green fluorescent protein (GFP). In embodiments, the reconstituted fluorescent protein is a reconstituted red fluorescent protein (RFP). In embodiments, the reconstituted fluorescent protein is a reconstituted orange fluorescent protein (OFP). In embodiments, the reconstituted fluorescent protein is a reconstituted yellow fluorescent protein (YFP). In embodiments, the reconstituted fluorescent protein is or a reconstituted green fluorescent protein (GFP).

In embodiments, the reconstituted fluorescent protein is a reconstituted mScarlet, a reconstituted stagRFP, a reconstituted mKOk, a reconstituted mRuby3, a reconstituted FusionMQV, a reconstituted FusionRed, a reconstituted mScarlet-1, a reconstituted Azalea-B5, a reconstituted mKate2, a reconstituted mCherry, a reconstituted mNeptune2, a reconstituted mNeptune2.5, a reconstituted mCitrine, a reconstituted cpVenus, a reconstituted mPapaya, a reconstituted sfGFP, a reconstituted mNG2, a reconstituted mNG3A, a reconstituted miRFP720, a reconstituted mRhubarb720, a reconstituted Dreiklang, a reconstituted Skylan-S, a reconstituted ffDronpa, a reconstituted rsEGFP2, a reconstituted mClover3, or a reconstituted EGFP.

In embodiments, the reconstituted fluorescent protein is a reconstituted mScarlet, a reconstituted stagRFP, a reconstituted mKOk, a reconstituted mRuby3, a reconstituted FusionMQV, a reconstituted FusionRed, a reconstituted mScarlet-1, a reconstituted Azalea-B5, a reconstituted mKate2, a reconstituted mCherry, a reconstituted mNeptune2, a reconstituted mNeptune2.5, a reconstituted mCitrine, a reconstituted cpVenus, a reconstituted mPapaya, a reconstituted sfGFP, a reconstituted mNG2, a reconstituted mNG3A, a reconstituted miRFP720, a reconstituted mRhubarb720, a reconstituted Dreiklang, a reconstituted Skylan-S, a reconstituted ffDronpa, a reconstituted rsEGFP2, a reconstituted mClover3, or a reconstituted EGFP. In embodiments, the reconstituted fluorescent protein is a reconstituted mScarlet. In embodiments, the reconstituted fluorescent protein is a reconstituted stagRFP. In embodiments, the reconstituted fluorescent protein is a reconstituted mKOk. In embodiments, the reconstituted fluorescent protein is a reconstituted mRuby3. In embodiments, the reconstituted fluorescent protein is a reconstituted FusionMQV. In embodiments, the reconstituted fluorescent protein is a reconstituted FusionRed. In embodiments, the reconstituted fluorescent protein is a reconstituted mScarlet-1. In embodiments, the reconstituted fluorescent protein is a reconstituted Azalea-B5. In embodiments, the reconstituted fluorescent protein is a reconstituted mKate2. In embodiments, the reconstituted fluorescent protein is a reconstituted mCherry. In embodiments, the reconstituted fluorescent protein is a reconstituted mNeptune2. In embodiments, the reconstituted fluorescent protein is a reconstituted mNeptune2.5. In embodiments, the reconstituted fluorescent protein is a reconstituted mCitrine. In embodiments, the reconstituted fluorescent protein is a reconstituted cpVenus. In embodiments, the reconstituted fluorescent protein is a reconstituted mPapaya. In embodiments, the reconstituted fluorescent protein is a reconstituted sfGFP. In embodiments, the reconstituted fluorescent protein is a reconstituted mNG2. In embodiments, the reconstituted fluorescent protein is a reconstituted mNG3A. In embodiments, the reconstituted fluorescent protein is a reconstituted miRFP720, a reconstituted mRhubarb720.

In embodiments, the reconstituted fluorescent protein is a reconstituted Dreiklang. In embodiments, the reconstituted fluorescent protein is a reconstituted Skylan-S. In embodiments, the reconstituted fluorescent protein is a reconstituted ffDronpa. In embodiments, the reconstituted fluorescent protein is a reconstituted rsEGFP2. In embodiments, the reconstituted fluorescent protein is a reconstituted mClover3. In embodiments, the reconstituted fluorescent protein is a reconstituted EGFP.

In embodiments, the split fluorescent protein domain includes the amino acid sequence of SEQ ID NO:60, SEQ ID NO:61, or SEQ ID NO:62. In embodiments, the split fluorescent protein domain includes the amino acid sequence of SEQ ID NO:60. In embodiments, the split fluorescent protein domain includes the amino acid sequence of SEQ ID NO:61. In embodiments, the split fluorescent protein domain includes the amino acid sequence of SEQ ID NO:62. In embodiments, the split fluorescent protein domain is the amino acid sequence of SEQ ID NO:60. SEQ ID NO:61, or SEQ ID NO:62. In embodiments, the split fluorescent protein domain is the amino acid sequence of SEQ ID NO:60. In embodiments, the split fluorescent protein domain is the amino acid sequence of SEQ ID NO:61. In embodiments, the split fluorescent protein domain is the amino acid sequence of SEQ ID NO:62.

In embodiments, the split fluorescent protein domain includes an amino acid sequence having 70%, 75%, 80%, 85%, 90%, 95%, 96%, 97%, 98%, 99% or 100% sequence identity to the sequence of SEQ ID NO:60. In embodiments, the split fluorescent protein domain includes an amino acid sequence having 70% sequence identity to the sequence of SEQ ID NO:60. In embodiments, the split fluorescent protein domain includes an amino acid sequence having 75% sequence identity to the sequence of SEQ ID NO:60. In embodiments, the split fluorescent protein domain includes an amino acid sequence having 80% sequence identity to the sequence of SEQ ID NO:60. In embodiments, the split fluorescent protein domain includes an amino acid sequence having 85% sequence identity to the sequence of SEQ ID NO:60. In embodiments, the split fluorescent protein domain includes an amino acid sequence having 90% sequence identity to the sequence of SEQ ID NO:60. In embodiments, the split fluorescent protein domain includes an amino acid sequence having 95% sequence identity to the sequence of SEQ ID NO:60. In embodiments, the split fluorescent protein domain includes an amino acid sequence having 96% sequence identity to the sequence of SEQ ID NO:60. In embodiments, the split fluorescent protein domain includes an amino acid sequence having 97% sequence identity to the sequence of SEQ ID NO:60. In embodiments, the split fluorescent protein domain includes an amino acid sequence having 98% sequence identity to the sequence of SEQ ID NO:60. In embodiments, the split fluorescent protein domain includes an amino acid sequence having 99% sequence identity to the sequence of SEQ ID NO:60. In embodiments, the split fluorescent protein domain includes an amino acid sequence having 100% sequence identity to the sequence of SEQ ID NO:60.

In embodiments, the split fluorescent protein domain includes an amino acid sequence having 70%, 75%, 80%, 85%, 90%, 95%, 96%, 97%, 98%, 99% or 100% sequence identity to the sequence of SEQ ID NO:61. In embodiments, the split fluorescent protein domain includes an amino acid sequence having 70% sequence identity to the sequence of SEQ ID NO:61. In embodiments, the split fluorescent protein domain includes an amino acid sequence having 75% sequence identity to the sequence of SEQ ID NO:61. In embodiments, the split fluorescent protein domain includes an amino acid sequence having 80% sequence identity to the sequence of SEQ ID NO:61. In embodiments, the split fluorescent protein domain includes an amino acid sequence having 85% sequence identity to the sequence of SEQ ID NO:61. In embodiments, the split fluorescent protein domain includes an amino acid sequence having 90% sequence identity to the sequence of SEQ ID NO:61. In embodiments, the split fluorescent protein domain includes an amino acid sequence having 95% sequence identity to the sequence of SEQ ID NO:61. In embodiments, the split fluorescent protein domain includes an amino acid sequence having 96% sequence identity to the sequence of SEQ ID NO:61. In embodiments, the split fluorescent protein domain includes an amino acid sequence having 97% sequence identity to the sequence of SEQ ID NO:61. In embodiments, the split fluorescent protein domain includes an amino acid sequence having 98% sequence identity to the sequence of SEQ ID NO:61. In embodiments, the split fluorescent protein domain includes an amino acid sequence having 99% sequence identity to the sequence of SEQ ID NO:61. In embodiments, the split fluorescent protein domain includes an amino acid sequence having 100% sequence identity to the sequence of SEQ ID NO:61.

In embodiments, the split fluorescent protein domain includes an amino acid sequence having 70%, 75%, 80%, 85%, 90%, 95%, 96%, 97%, 98%, 99% or 100% sequence identity to the sequence of SEQ ID NO:62. In embodiments, the split fluorescent protein domain includes an amino acid sequence having 70% sequence identity to the sequence of SEQ ID NO:62. In embodiments, the split fluorescent protein domain includes an amino acid sequence having 75% sequence identity to the sequence of SEQ ID NO:62. In embodiments, the split fluorescent protein domain includes an amino acid sequence having 80% sequence identity to the sequence of SEQ ID NO:62. In embodiments, the split fluorescent protein domain includes an amino acid sequence having 85% sequence identity to the sequence of SEQ ID NO:62. In embodiments, the split fluorescent protein domain includes an amino acid sequence having 90% sequence identity to the sequence of SEQ ID NO:62. In embodiments, the split fluorescent protein domain includes an amino acid sequence having 95% sequence identity to the sequence of SEQ ID NO:62. In embodiments, the split fluorescent protein domain includes an amino acid sequence having 96% sequence identity to the sequence of SEQ ID NO:62. In embodiments, the split fluorescent protein domain includes an amino acid sequence having 97% sequence identity to the sequence of SEQ ID NO:62. In embodiments, the split fluorescent protein domain includes an amino acid sequence having 98% sequence identity to the sequence of SEQ ID NO:62. In embodiments, the split fluorescent protein domain includes an amino acid sequence having 99% sequence identity to the sequence of SEQ ID NO:62. In embodiments, the split fluorescent protein domain includes an amino acid sequence having 100% sequence identity to the sequence of SEQ ID NO:62.

In embodiments, the cognate split fluorescent protein includes the amino acid sequence of SEQ ID NO:63. SEQ ID NO:64, SEQ ID NO:65, or SEQ ID NO:66. In embodiments, the cognate split fluorescent protein includes the amino acid sequence of SEQ ID NO:63. In embodiments, the cognate split fluorescent protein includes the amino acid sequence of SEQ ID NO:64. In embodiments, the cognate split fluorescent protein includes the amino acid sequence of SEQ ID NO:65. In embodiments, the cognate split fluorescent protein includes the amino acid sequence of SEQ ID NO:66. In embodiments, the cognate split fluorescent protein is the amino acid sequence of SEQ ID NO:63, SEQ ID NO:64, SEQ ID NO:65, or SEQ ID NO:66. In embodiments, the cognate split fluorescent protein is the amino acid sequence of SEQ ID NO:63. In embodiments, the cognate split fluorescent protein is the amino acid sequence of SEQ ID NO:64. In embodiments, the cognate split fluorescent protein is the amino acid sequence of SEQ ID NO:65. In embodiments, the cognate split fluorescent protein is the amino acid sequence of SEQ ID NO:66.

In embodiments, the cognate split fluorescent protein includes an amino acid sequence having 70%, 75%, 80%, 85%, 90%, 95%, 96%, 97%, 98%, 99% or 100% sequence identity to the sequence of SEQ ID NO:63. In embodiments, the cognate split fluorescent protein includes an amino acid sequence having 70% sequence identity to the sequence of SEQ ID NO:63. In embodiments, the cognate split fluorescent protein includes an amino acid sequence having 75% sequence identity to the sequence of SEQ ID NO:63. In embodiments, the cognate split fluorescent protein includes an amino acid sequence having 80% sequence identity to the sequence of SEQ ID NO:63. In embodiments, the cognate split fluorescent protein includes an amino acid sequence having 85% sequence identity to the sequence of SEQ ID NO:63. In embodiments, the cognate split fluorescent protein includes an amino acid sequence having 90% sequence identity to the sequence of SEQ ID NO:63. In embodiments, the cognate split fluorescent protein includes an amino acid sequence having 95% sequence identity to the sequence of SEQ ID NO:63. In embodiments, the cognate split fluorescent protein includes an amino acid sequence having 96% sequence identity to the sequence of SEQ ID NO:63. In embodiments, the cognate split fluorescent protein includes an amino acid sequence having 97% sequence identity to the sequence of SEQ ID NO:63. In embodiments, the cognate split fluorescent protein includes an amino acid sequence having 98% sequence identity to the sequence of SEQ ID NO:63. In embodiments, the cognate split fluorescent protein includes an amino acid sequence having 99% sequence identity to the sequence of SEQ ID NO:63. In embodiments, the cognate split fluorescent protein includes an amino acid sequence having 100% sequence identity to the sequence of SEQ ID NO:63.

In embodiments, the cognate split fluorescent protein includes an amino acid sequence having 70%, 75%, 80%, 85%, 90%, 95%, 96%, 97%, 98%, 99% or 100% sequence identity to the sequence of SEQ ID NO:64. In embodiments, the cognate split fluorescent protein includes an amino acid sequence having 70% sequence identity to the sequence of SEQ ID NO:64. In embodiments, the cognate split fluorescent protein includes an amino acid sequence having 75% sequence identity to the sequence of SEQ ID NO:64. In embodiments, the cognate split fluorescent protein includes an amino acid sequence having 80% sequence identity to the sequence of SEQ ID NO:64. In embodiments, the cognate split fluorescent protein includes an amino acid sequence having 85% sequence identity to the sequence of SEQ ID NO:64. In embodiments, the cognate split fluorescent protein includes an amino acid sequence having 90% sequence identity to the sequence of SEQ ID NO:64. In embodiments, the cognate split fluorescent protein includes an amino acid sequence having 95% sequence identity to the sequence of SEQ ID NO:64. In embodiments, the cognate split fluorescent protein includes an amino acid sequence having 96% sequence identity to the sequence of SEQ ID NO:64. In embodiments, the cognate split fluorescent protein includes an amino acid sequence having 97% sequence identity to the sequence of SEQ ID NO:64. In embodiments, the cognate split fluorescent protein includes an amino acid sequence having 98% sequence identity to the sequence of SEQ ID NO:64. In embodiments, the cognate split fluorescent protein includes an amino acid sequence having 99% sequence identity to the sequence of SEQ ID NO:64. In embodiments, the cognate split fluorescent protein includes an amino acid sequence having 100% sequence identity to the sequence of SEQ ID NO:64.

In embodiments, the cognate split fluorescent protein includes an amino acid sequence having 70%, 75%, 80%, 85%, 90%, 95%, 96%, 97%, 98%, 99% or 100% sequence identity to the sequence of SEQ ID NO:65. In embodiments, the cognate split fluorescent protein includes an amino acid sequence having 70% sequence identity to the sequence of SEQ ID NO:65. In embodiments, the cognate split fluorescent protein includes an amino acid sequence having 75% sequence identity to the sequence of SEQ ID NO:65. In embodiments, the cognate split fluorescent protein includes an amino acid sequence having 80% sequence identity to the sequence of SEQ ID NO:65. In embodiments, the cognate split fluorescent protein includes an amino acid sequence having 85% sequence identity to the sequence of SEQ ID NO:65. In embodiments, the cognate split fluorescent protein includes an amino acid sequence having 90% sequence identity to the sequence of SEQ ID NO:65. In embodiments, the cognate split fluorescent protein includes an amino acid sequence having 95% sequence identity to the sequence of SEQ ID NO:65. In embodiments, the cognate split fluorescent protein includes an amino acid sequence having 96% sequence identity to the sequence of SEQ ID NO:65. In embodiments, the cognate split fluorescent protein includes an amino acid sequence having 97% sequence identity to the sequence of SEQ ID NO:65. In embodiments, the cognate split fluorescent protein includes an amino acid sequence having 98% sequence identity to the sequence of SEQ ID NO:65. In embodiments, the cognate split fluorescent protein includes an amino acid sequence having 99% sequence identity to the sequence of SEQ ID NO:65. In embodiments, the cognate split fluorescent protein includes an amino acid sequence having 100% sequence identity to the sequence of SEQ ID NO:65.

In embodiments, the cognate split fluorescent protein includes an amino acid sequence having 70%, 75%, 80%, 85%, 90%, 95%, 96%, 97%, 98%, 99% or 100% sequence identity to the sequence of SEQ ID NO:66. In embodiments, the cognate split fluorescent protein includes an amino acid sequence having 70% sequence identity to the sequence of SEQ ID NO:66. In embodiments, the cognate split fluorescent protein includes an amino acid sequence having 75% sequence identity to the sequence of SEQ ID NO:66. In embodiments, the cognate split fluorescent protein includes an amino acid sequence having 80% sequence identity to the sequence of SEQ ID NO:66. In embodiments, the cognate split fluorescent protein includes an amino acid sequence having 85% sequence identity to the sequence of SEQ ID NO:66. In embodiments, the cognate split fluorescent protein includes an amino acid sequence having 90% sequence identity to the sequence of SEQ ID NO:66. In embodiments, the cognate split fluorescent protein includes an amino acid sequence having 95% sequence identity to the sequence of SEQ ID NO:66. In embodiments, the cognate split fluorescent protein includes an amino acid sequence having 96% sequence identity to the sequence of SEQ ID NO:66. In embodiments, the cognate split fluorescent protein includes an amino acid sequence having 97% sequence identity to the sequence of SEQ ID NO:66. In embodiments, the cognate split fluorescent protein includes an amino acid sequence having 98% sequence identity to the sequence of SEQ ID NO:66. In embodiments, the cognate split fluorescent protein includes an amino acid sequence having 99% sequence identity to the sequence of SEQ ID NO:66. In embodiments, the cognate split fluorescent protein includes an amino acid sequence having 100% sequence identity to the sequence of SEQ ID NO:66.

In embodiments, the haloalkane dehalogenase domain includes the amino acid sequence of SEQ ID NO:25 or SEQ ID NO:26. In embodiments, the haloalkane dehalogenase domain includes the amino acid sequence of SEQ ID NO:25. In embodiments, the haloalkane dehalogenase domain includes the amino acid sequence of SEQ ID NO:26. In embodiments, the haloalkane dehalogenase domain is the amino acid sequence of SEQ ID NO:25 or SEQ ID NO:26. In embodiments, the haloalkane dehalogenase domain is the amino acid sequence of SEQ ID NO:25. In embodiments, the haloalkane dehalogenase domain is the amino acid sequence of SEQ ID NO: 26.

In embodiments, the chemical fluorophore is NGD, JF669, JFX646, or JF635. In embodiments, the chemical fluorophore is NGD. In embodiments, the chemical fluorophore is JF669. In embodiments, the chemical fluorophore is JFX646. In embodiments, the chemical fluorophore is JF635.

In embodiments, the chemical fluorophore includes the formula of

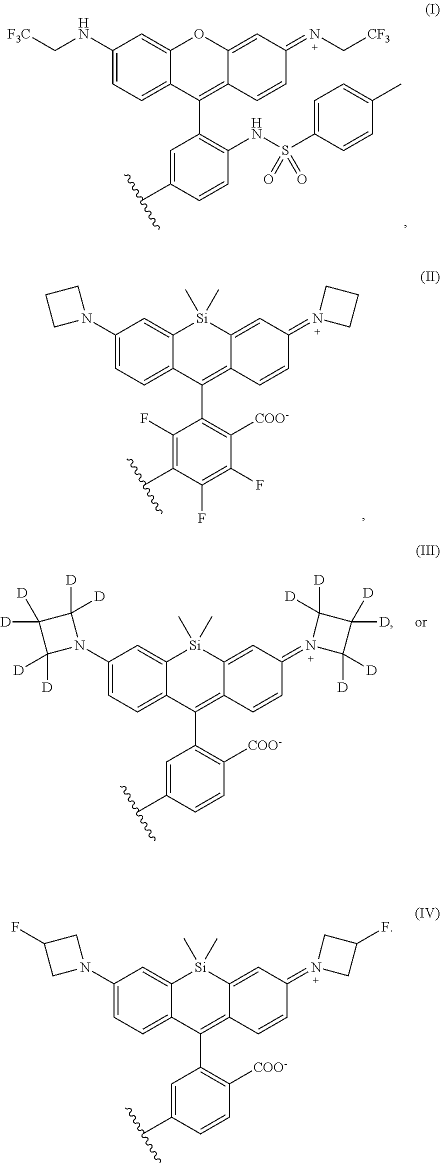

In embodiments, the chemical fluorophore includes the formula of

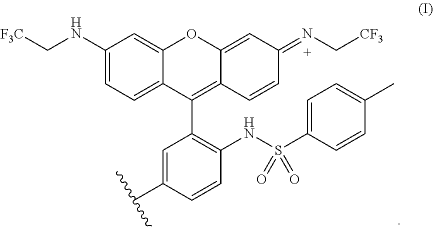 [SMILES: *c1ccc(NS(=O)(=O)c2ccc(C)cc2)c(-c2c3cc/c(=N\CC(F)(F)F)cc-3oc3cc(NCC(F)(F)F)ccc23)c1]

In embodiments, the chemical fluorophore includes the formula of

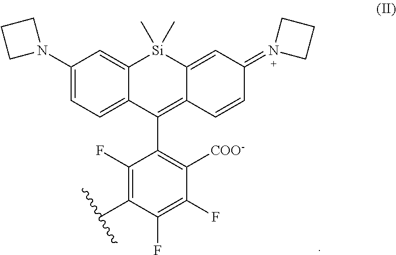

In embodiments, the chemical fluorophore includes the formula of

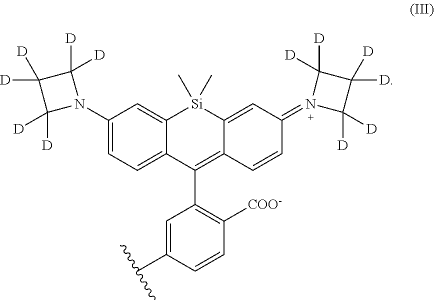

In embodiments, the chemical fluorophore includes the formula of

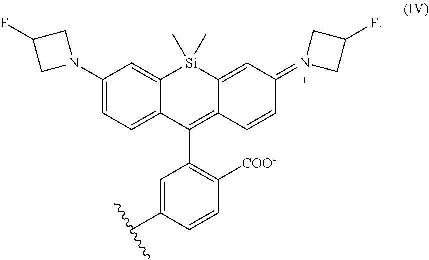

In embodiments, the biosensing protein domain includes: (a) a target biomolecule binding domain; (b) a target biomolecule substrate domain, a linker domain, and a target biomolecule activity sensing domain; or (c) a target biomolecule domain, a linker domain, and a target biomolecule substrate domain. In embodiments, the biosensing protein domain includes a target biomolecule binding domain. In embodiments, the biosensing protein domain includes a target biomolecule substrate domain, a linker domain, and a target biomolecule activity sensing domain. In embodiments, the biosensing protein domain includes a target biomolecule substrate domain. In embodiments, the biosensing protein domain includes a linker domain. In embodiments, the biosensing protein domain includes and a target biomolecule activity sensing domain. In embodiments, the biosensing protein domain includes a target biomolecule domain, a linker domain, and a target biomolecule substrate domain. In embodiments, the biosensing protein domain includes a target biomolecule domain. In embodiments, the biosensing protein domain includes a linker domain. In embodiments, the biosensing protein domain includes a target biomolecule substrate domain.

In embodiments, the target biomolecule binding domain is capable of binding the target biomolecule. In embodiments, the target biomolecule binding domain binds the target biomolecule, thereby inducing a conformational change in the recombinant protein. In embodiments, the target biomolecule binding domain includes a phosphorylatable amino acid residue. In embodiments, the binding of the target biomolecule binding domain to the target biomolecule induces a modification of the target biomolecule binding domain. In embodiments, the modification of the target biomolecule binding domain is phosphorylation of the phosphorylatable amino acid residue. In embodiments, the target biomolecule phosphorylates the phosphorylatable amino acid residue, thereby inducing a conformational change in the recombinant protein. In embodiments, the conformational change allows the split fluorescent protein domain to bind the cognate split fluorescent protein; thereby forming a reconstituted fluorescent protein. In embodiments, the conformational changes allows: (a) the chemical fluorophore to activate the reconstituted fluorescent protein; or (b) the reconstituted fluorescent protein to activate the chemical fluorophore. In embodiments, the conformational change increases the fluorescent signal from the FRET pair. In embodiments, the conformational change decreases the fluorescent signal from the FRET pair.

In embodiments, the target biomolecule binding domain is capable of interacting with the target biomolecule. In embodiments, the target biomolecule binding domain interacts with the target biomolecule, thereby inducing a conformational change in the recombinant protein. In embodiments, the conformational change allows the split fluorescent protein domain to bind the cognate split fluorescent protein; thereby forming a reconstituted fluorescent protein. In embodiments, the conformational changes allows: (a) the chemical fluorophore to activate the reconstituted fluorescent protein; or (b) the reconstituted fluorescent protein to activate the chemical fluorophore. In embodiments, the conformational change increases the fluorescent signal from the FRET pair. In embodiments, the conformational change decreases the fluorescent signal from the FRET pair.

In embodiments, the target biomolecule domain is capable of binding the target biomolecule substrate domain. In embodiments, the target biomolecule domain is capable of being activated. In embodiments, the activated target biomolecule domain binds the target biomolecule substrate domain. In embodiments, the target biomolecule domain binds the target biomolecule substrate domain, thereby inducing a conformational change in the recombinant protein. In embodiments, the conformational change allows the split fluorescent protein domain to bind the cognate split fluorescent protein; thereby forming a reconstituted fluorescent protein. In embodiments, the conformation change In embodiments, the conformational changes allows: (a) the chemical fluorophore to activate the reconstituted fluorescent protein; or (b) the reconstituted fluorescent protein to activate the chemical fluorophore. In embodiments, the conformational change increases the fluorescent signal from the FRET pair. In embodiments, the conformational change decreases the fluorescent signal from the FRET pair.

In embodiments, the target biomolecule is a protein kinase, a second messenger molecule, or a GTPase. In embodiments, the target biomolecule is a protein kinase. In embodiments, the target biomolecule is a second messenger molecule. In embodiments, the target biomolecule is a GTPase.

In embodiments, the protein kinase is a protein kinase A (PKA), a protein kinase C (PKC), a protein kinase B (PKB), an extracellular signal-regulated kinase (ERK), a 5′ adenosine monophosphate-activated protein kinase (AMPK), a mammalian target of rapamycin (mTOR), a Fyn kinase, or a Src kinase. In embodiments, the protein kinase is a protein kinase A (PKA). In embodiments, the protein kinase is a protein kinase C (PKC). In embodiments, the protein kinase is a protein kinase B (PKB). In embodiments, the protein kinase is an extracellular signal-regulated kinase (ERK). In embodiments, the protein kinase is a 5′ adenosine monophosphate-activated protein kinase (AMPK). In embodiments, the protein kinase is a mammalian target of rapamycin (mTOR). In embodiments, the protein kinase is a Fyn kinase. In embodiments, the protein kinase is a Src kinase.

In embodiments, the second messenger molecule is cyclic adenosine monophosphate (cAMP), cyclic guanosine monophosphate (cGMP), inositol triphosphate (IP₃), diacylglycerol (DAG), or calcium (Ca²⁺). In embodiments, the second messenger molecule is cyclic adenosine monophosphate (cAMP). In embodiments, the second messenger molecule is cyclic guanosine monophosphate (cGMP). In embodiments, the second messenger molecule is inositol triphosphate (IP₃). In embodiments, the second messenger molecule is diacylglycerol (DAG). In embodiments, the second messenger molecule is calcium (Ca²⁺).

In embodiments, the GTPase is a Ras GTPase or a Rap1 GTPase. In embodiments, the GTPase is a Ras GTPase. In embodiments, the GTPase is a Rap1 GTPase.

In embodiments, the biomolecule substrate domain includes the amino acid sequence of any one of SEQ ID NO:27 to SEQ ID NO:36. In embodiments, the biomolecule substrate domain includes the amino acid sequence of SEQ ID NO:27. In embodiments, the biomolecule substrate domain includes the amino acid sequence of SEQ ID NO:28. In embodiments, the biomolecule substrate domain includes the amino acid sequence of SEQ ID NO:29. In embodiments, the biomolecule substrate domain includes the amino acid sequence of SEQ ID NO:30. In embodiments, the biomolecule substrate domain includes the amino acid sequence of SEQ ID NO:31. In embodiments, the biomolecule substrate domain includes the amino acid sequence of SEQ ID NO: 32. In embodiments, the biomolecule substrate domain includes the amino acid sequence of SEQ ID NO:33. In embodiments, the biomolecule substrate domain includes the amino acid sequence of SEQ ID NO:34. In embodiments, the biomolecule substrate domain includes the amino acid sequence of SEQ ID NO:35. In embodiments, the biomolecule substrate domain includes the amino acid sequence of SEQ ID NO:36.

In embodiments, the biomolecule substrate domain is the amino acid sequence of any one of SEQ ID NO:27 to SEQ ID NO:36. In embodiments, the biomolecule substrate domain is the amino acid sequence of SEQ ID NO:27. In embodiments, the biomolecule substrate domain is the amino acid sequence of SEQ ID NO:28. In embodiments, the biomolecule substrate domain is the amino acid sequence of SEQ ID NO:29. In embodiments, the biomolecule substrate domain is the amino acid sequence of SEQ ID NO:30. In embodiments, the biomolecule substrate domain is the amino acid sequence of SEQ ID NO:31. In embodiments, the biomolecule substrate domain is the amino acid sequence of SEQ ID NO:32. In embodiments, the biomolecule substrate domain is the amino acid sequence of SEQ ID NO:33. In embodiments, the biomolecule substrate domain is the amino acid sequence of SEQ ID NO:34. In embodiments, the biomolecule substrate domain is the amino acid sequence of SEQ ID NO:35. In embodiments, the biomolecule substrate domain is the amino acid sequence of SEQ ID NO:36.

In embodiments, the target biomolecule substrate domain further includes a phosphorylatable amino acid residue. In embodiments, the phosphorylatable amino acid residue is capable of being phosphorylated by the protein kinase.

In embodiments, the target biomolecule domain includes the amino acid sequence of SEQ ID NO:44 or SEQ ID NO:45. In embodiments, the target biomolecule domain includes the amino acid sequence of SEQ ID NO:44. In embodiments, the target biomolecule domain includes the amino acid sequence of SEQ ID NO:45. In embodiments, the target biomolecule domain is the amino acid sequence of SEQ ID NO:44 or SEQ ID NO:45. In embodiments, the target biomolecule domain is the amino acid sequence of SEQ ID NO:44.

In embodiments, the target biomolecule domain is the amino acid sequence of SEQ ID NO:45.

In embodiments, the target biomolecule activity sensing domain includes a phosphoamino acid binding domain. In embodiments, the phosphoamino acid binding domain is capable of binding a phosphorylated amino acid residue. In embodiments, the phosphoamino acid binding domain is capable of binding a phosphorylated amino acid residue in the target biomolecule substrate domain. In embodiments, the phosphoamino acid binding domain binds a phosphorylated amino acid residue in the target biomolecule substrate domain. In embodiments, the phosphoamino acid binding domain binds a phosphorylated amino acid residue in the target biomolecule substrate domain, thereby inducing a conformational change in the recombinant protein. In embodiments, the conformational changes allows: (a) the chemical fluorophore to activate the fluorescent protein domain; or (b) the fluorescent protein domain to activate the chemical fluorophore. In embodiments, the conformational change increases the fluorescent signal from the FRET pair. In embodiments, the conformational change decreases the fluorescent signal from the FRET pair.

In embodiments, the phosphoamino acid binding domain includes the amino acid sequence of SEQ ID NO:39, SEQ ID NO:40, or SEQ ID NO:41. In embodiments, the phosphoamino acid binding domain includes the amino acid sequence of SEQ ID NO:39. In embodiments, the phosphoamino acid binding domain includes the amino acid sequence of SEQ ID NO:40. In embodiments, the phosphoamino acid binding domain includes the amino acid sequence of SEQ ID NO:41. In embodiments, the phosphoamino acid binding domain is the amino acid sequence of SEQ ID NO:39, SEQ ID NO:40, or SEQ ID NO:41. In embodiments, the phosphoamino acid binding domain is the amino acid sequence of SEQ ID NO:39. In embodiments, the phosphoamino acid binding domain is the amino acid sequence of SEQ ID NO:40. In embodiments, the phosphoamino acid binding domain is the amino acid sequence of SEQ ID NO:41.

In embodiments, the linker domain is peptide linker. In embodiments, the linker domain is between about 1 to about 500 amino acids in length. In embodiments, the linker domain is between about 2 to about 500 amino acids in length. In embodiments, the linker domain is between about 3 to about 500 amino acids in length. In embodiments, the linker domain is between about 4 to about 500 amino acids in length. In embodiments, the linker domain is between about 5 to about 500 amino acids in length. In embodiments, the linker domain is between about 6 to about 500 amino acids in length. In embodiments, the linker domain is between about 7 to about 500 amino acids in length. In embodiments, the linker domain is between about 8 to about 500 amino acids in length. In embodiments, the linker domain is between about 9 to about 500 amino acids in length. In embodiments, the linker domain is between about 10 to about 500 amino acids in length. In embodiments, the linker domain is between about 15 to about 500 amino acids in length. In embodiments, the linker domain is between about 20 to about 500 amino acids in length. In embodiments, the linker domain is between about 25 to about 500 amino acids in length. In embodiments, the linker domain is between about 30 to about 500 amino acids in length. In embodiments, the linker domain is between about 35 to about 500 amino acids in length. In embodiments, the linker domain is between about 40 to about 500 amino acids in length. In embodiments, the linker domain is between about 45 to about 500 amino acids in length. In embodiments, the linker domain is between about 50 to about 500 amino acids in length. In embodiments, the linker domain is between about 100 to about 500 amino acids in length. In embodiments, the linker domain is between about 150 to about 500 amino acids in length. In embodiments, the linker domain is between about 200 to about 500 amino acids in length. In embodiments, the linker domain is between about 250 to about 500 amino acids in length. In embodiments, the linker domain is between about 300 to about 500 amino acids in length. In embodiments, the linker domain is between about 350 to about 500 amino acids in length. In embodiments, the linker domain is between about 400 to about 500 amino acids in length. In embodiments, the linker domain is between about 450 to about 500 amino acids in length.

In embodiments, the linker domain is between about 1 to about 450 amino acids in length. In embodiments, the linker domain is between about 1 to about 400 amino acids in length. In embodiments, the linker domain is between about 1 to about 350 amino acids in length. In embodiments, the linker domain is between about 1 to about 300 amino acids in length. In embodiments, the linker domain is between about 1 to about 250 amino acids in length. In embodiments, the linker domain is between about 1 to about 200 amino acids in length. In embodiments, the linker domain is between about 1 to about 150 amino acids in length. In embodiments, the linker domain is between about 1 to about 100 amino acids in length. In embodiments, the linker domain is between about 1 to about 50 amino acids in length. In embodiments, the linker domain is between about 1 to about 45 amino acids in length. In embodiments, the linker domain is between about 1 to about 40 amino acids in length. In embodiments, the linker domain is between about 1 to about 35 amino acids in length. In embodiments, the linker domain is between about 1 to about 30 amino acids in length. In embodiments, the linker domain is between about 1 to about 25 amino acids in length. In embodiments, the linker domain is between about 1 to about 20 amino acids in length. In embodiments, the linker domain is between about 1 to about 15 amino acids in length. In embodiments, the linker domain is between about 1 to about 10 amino acids in length. In embodiments, the linker domain is between about 1 to about 9 amino acids in length. In embodiments, the linker domain is between about 1 to about 8 amino acids in length. In embodiments, the linker domain is between about 1 to about 7 amino acids in length. In embodiments, the linker domain is between about 1 to about 6 amino acids in length. In embodiments, the linker domain is between about 1 to about 5 amino acids in length. In embodiments, the linker domain is between about 1 to about 4 amino acids in length. In embodiments, the linker domain is between about 1 to about 3 amino acids in length. In embodiments, the linker domain is between about 1 to about 2 amino acids in length.

In embodiments, the linker domain is between 1 to 500 amino acids in length. In embodiments, the linker domain is between 2 to 500 amino acids in length. In embodiments, the linker domain is between 3 to 500 amino acids in length. In embodiments, the linker domain is between 4 to 500 amino acids in length. In embodiments, the linker domain is between 5 to 500 amino acids in length. In embodiments, the linker domain is between 6 to 500 amino acids in length. In embodiments, the linker domain is between 7 to 500 amino acids in length. In embodiments, the linker domain is between 8 to 500 amino acids in length. In embodiments, the linker domain is between 9 to 500 amino acids in length. In embodiments, the linker domain is between 10 to 500 amino acids in length. In embodiments, the linker domain is between 15 to 500 amino acids in length. In embodiments, the linker domain is between 20 to 500 amino acids in length. In embodiments, the linker domain is between 25 to 500 amino acids in length. In embodiments, the linker domain is between 30 to 500 amino acids in length. In embodiments, the linker domain is between 35 to 500 amino acids in length. In embodiments, the linker domain is between 40 to 500 amino acids in length. In embodiments, the linker domain is between 45 to 500 amino acids in length. In embodiments, the linker domain is between 50 to 500 amino acids in length. In embodiments, the linker domain is between 100 to 500 amino acids in length. In embodiments, the linker domain is between 150 to 500 amino acids in length. In embodiments, the linker domain is between 200 to 500 amino acids in length. In embodiments, the linker domain is between 250 to 500 amino acids in length. In embodiments, the linker domain is between 300 to 500 amino acids in length. In embodiments, the linker domain is between 350 to 500 amino acids in length. In embodiments, the linker domain is between 400 to 500 amino acids in length. In embodiments, the linker domain is between 450 to 500 amino acids in length.

In embodiments, the linker domain is between 1 to 450 amino acids in length. In embodiments, the linker domain is between 1 to 400 amino acids in length. In embodiments, the linker domain is between 1 to 350 amino acids in length. In embodiments, the linker domain is between 1 to 300 amino acids in length. In embodiments, the linker domain is between 1 to 250 amino acids in length. In embodiments, the linker domain is between 1 to 200 amino acids in length. In embodiments, the linker domain is between 1 to 150 amino acids in length. In embodiments, the linker domain is between 1 to 100 amino acids in length.

In embodiments, the linker domain is between 1 to 50 amino acids in length. In embodiments, the linker domain is between 1 to 45 amino acids in length. In embodiments, the linker domain is between 1 to 40 amino acids in length. In embodiments, the linker domain is between 1 to 35 amino acids in length. In embodiments, the linker domain is between 1 to 30 amino acids in length. In embodiments, the linker domain is between 1 to 25 amino acids in length. In embodiments, the linker domain is between 1 to 20 amino acids in length. In embodiments, the linker domain is between 1 to 15 amino acids in length. In embodiments, the linker domain is between 1 to 10 amino acids in length. In embodiments, the linker domain is between 1 to 9 amino acids in length. In embodiments, the linker domain is between 1 to 8 amino acids in length. In embodiments, the linker domain is between 1 to 7 amino acids in length. In embodiments, the linker domain is between 1 to 6 amino acids in length. In embodiments, the linker domain is between 1 to 5 amino acids in length. In embodiments, the linker domain is between 1 to 4 amino acids in length. In embodiments, the linker domain is between 1 to 3 amino acids in length. In embodiments, the linker domain is between 1 to 2 amino acids in length.

In embodiments, the linker domain includes the amino acid sequence of any one of SEQ ID NO:46 to SEQ ID NO:59. In embodiments, the linker domain includes the amino acid sequence of any one of SEQ ID NO:46. In embodiments, the linker domain includes the amino acid sequence of any one of SEQ ID NO:47. In embodiments, the linker domain includes the amino acid sequence of any one of SEQ ID NO:48. In embodiments, the linker domain includes the amino acid sequence of any one of SEQ ID NO:49. In embodiments, the linker domain includes the amino acid sequence of any one of SEQ ID NO:50. In embodiments, the linker domain includes the amino acid sequence of any one of SEQ ID NO:51. In embodiments, the linker domain includes the amino acid sequence of any one of SEQ ID NO:52. In embodiments, the linker domain includes the amino acid sequence of any one of SEQ ID NO:53. In embodiments, the linker domain includes the amino acid sequence of any one of SEQ ID NO:54. In embodiments, the linker domain includes the amino acid sequence of any one of SEQ ID NO:55. In embodiments, the linker domain includes the amino acid sequence of any one of SEQ ID NO:56. In embodiments, the linker domain includes the amino acid sequence of any one of SEQ ID NO:57. In embodiments, the linker domain includes the amino acid sequence of any one of SEQ ID NO:58. In embodiments, the linker domain includes the amino acid sequence of any one of SEQ ID NO: 59.

In embodiments, the linker domain is the amino acid sequence of any one of SEQ ID NO:46 to SEQ ID NO:59. In embodiments, the linker domain is the amino acid sequence of any one of SEQ ID NO:46. In embodiments, the linker domain is the amino acid sequence of any one of SEQ ID NO:47. In embodiments, the linker domain is the amino acid sequence of any one of SEQ ID NO:48. In embodiments, the linker domain is the amino acid sequence of any one of SEQ ID NO:49. In embodiments, the linker domain is the amino acid sequence of any one of SEQ ID NO:50. In embodiments, the linker domain is the amino acid sequence of any one of SEQ ID NO:51. In embodiments, the linker domain is the amino acid sequence of any one of SEQ ID NO:52. In embodiments, the linker domain is the amino acid sequence of any one of SEQ ID NO:53. In embodiments, the linker domain is the amino acid sequence of any one of SEQ ID NO:54. In embodiments, the linker domain is the amino acid sequence of any one of SEQ ID NO:55. In embodiments, the linker domain is the amino acid sequence of any one of SEQ ID NO:56. In embodiments, the linker domain is the amino acid sequence of any one of SEQ ID NO:57. In embodiments, the linker domain is the amino acid sequence of any one of SEQ ID NO:58. In embodiments, the linker domain is the amino acid sequence of any one of SEQ ID NO:59.

In embodiments, the target biomolecule binding domain includes the amino acid sequence of SEQ ID NO:37 or SEQ ID NO:38. In embodiments, the target biomolecule binding domain includes the amino acid sequence of SEQ ID NO:37. In embodiments, the target biomolecule binding domain includes the amino acid sequence of SEQ ID NO:38. In embodiments, the target biomolecule binding domain is the amino acid sequence of SEQ ID NO:37 or SEQ ID NO:38. In embodiments, the target biomolecule binding domain is the amino acid sequence of SEQ ID NO:37. In embodiments, the target biomolecule binding domain is the amino acid sequence of SEQ ID NO:38.

In embodiments, the split fluorescent protein domain is attached to the N-terminus of the biosensing protein domain and the biosensing protein domain is attached to the N-terminus of the haloalkane dehalogenase domain.

In embodiments, the haloalkane dehalogenase domain is attached to the N-terminus of the biosensing protein domain and the biosensing protein domain is attached to the N-terminus of the split fluorescent protein domain.

In another aspect is provided a recombinant protein including a fluorescent protein domain bound to a biosensing protein domain and a split haloalkane dehalogenase domain, wherein: the split haloalkane dehalogenase domain is one part of a haloalkane dehalogenase split pair that forms a reconstituted haloalkane dehalogenase protein upon binding of a cognate haloalkane dehalogenase split protein domain.

In embodiments, the split haloalkane dehalogenase domain is bound to a second recombinant protein including the cognate haloalkane dehalogenase split protein domain bound to a protein of interest, wherein the cognate haloalkane dehalogenase split protein domain is covalently bound to a chemical fluorophore, wherein the chemical fluorophore and the fluorescent protein domain are a Forster Resonance Energy Transfer (FRET) pair.

In embodiments, the fluorescent protein domain is a red fluorescent protein (RFP), an orange fluorescent protein (OFP), a yellow fluorescent protein (YFP), or a green fluorescent protein (GFP). In embodiments, the fluorescent protein domain is a red fluorescent protein (RFP). In embodiments, the fluorescent protein domain is an orange fluorescent protein (OFP). In embodiments, the fluorescent protein domain is a yellow fluorescent protein (YFP). In embodiments, the fluorescent protein domain is a green fluorescent protein (GFP).

In embodiments, the fluorescent protein domain is an mScarlet domain, a stagRFP domain, an mKOk domain, an mRuby3 domain, a FusionMQV domain, a FusionRed domain, an mScarlet-1 domain, an Azalea-B5 domain, an mKate2 domain, an mCherry domain, an mNeptune2 domain, an mNeptune2.5 domain, an mCitrine domain, a cpVenus domain, an mPapaya domain, a sfGFP domain, an mNG2 domain, an mNG3A domain, an miRFP720 domain, an mRhubarb720 domain, a Dreiklang domain, a Skylan-S domain, an ffDronpa domain, an rsEGFP2 domain, an mClover3 domain, or an EGFP domain. In embodiments, the fluorescent protein domain is an mScarlet domain. In embodiments, the fluorescent protein domain is a stagRFP domain. In embodiments, the fluorescent protein domain is an mKOk domain. In embodiments, the fluorescent protein domain is an mRuby3 domain. In embodiments, the fluorescent protein domain is a FusionMQV domain. In embodiments, the fluorescent protein domain is a FusionRed domain. In embodiments, the fluorescent protein domain is an mScarlet-1 domain. In embodiments, the fluorescent protein domain is an Azalea-B5 domain. In embodiments, the fluorescent protein domain is an mKate2 domain. In embodiments, the fluorescent protein domain is an mCherry domain. In embodiments, the fluorescent protein domain is an mNeptune2 domain. In embodiments, the fluorescent protein domain is an mNeptune2.5 domain. In embodiments, the fluorescent protein domain is an mCitrine domain. In embodiments, the fluorescent protein domain is a cpVenus domain. In embodiments, the fluorescent protein domain is an mPapaya domain. In embodiments, the fluorescent protein domain is a sfGFP domain. In embodiments, the fluorescent protein domain is an mNG2 domain. In embodiments, the fluorescent protein domain is an mNG3A domain. In embodiments, the fluorescent protein domain is an miRFP720 domain. In embodiments, the fluorescent protein domain is an mRhubarb720 domain. In embodiments, the fluorescent protein domain is a Dreiklang domain. In embodiments, the fluorescent protein domain is a Skylan-S domain. In embodiments, the fluorescent protein domain is an ffDronpa domain. In embodiments, the fluorescent protein domain is an rsEGFP2 domain. In embodiments, the fluorescent protein domain is an mClover3 domain. In embodiments, the fluorescent protein domain is an EGFP domain.

In embodiments, the fluorescent protein domain includes the amino acid sequence of any one of SEQ ID NO:1 to SEQ ID NO:24. In embodiments, the fluorescent protein domain includes the amino acid sequence of SEQ ID NO:1. In embodiments, the fluorescent protein domain includes the amino acid sequence of SEQ ID NO:2. In embodiments, the fluorescent protein domain includes the amino acid sequence of SEQ ID NO:3. In embodiments, the fluorescent protein domain includes the amino acid sequence of SEQ ID NO:4. In embodiments, the fluorescent protein domain includes the amino acid sequence of SEQ ID NO:5. In embodiments, the fluorescent protein domain includes the amino acid sequence of SEQ ID NO:6. In embodiments, the fluorescent protein domain includes the amino acid sequence of SEQ ID NO:7. In embodiments, the fluorescent protein domain includes the amino acid sequence of SEQ ID NO:8. In embodiments, the fluorescent protein domain includes the amino acid sequence of SEQ ID NO:9. In embodiments, the fluorescent protein domain includes the amino acid sequence of SEQ ID NO: 10. In embodiments, the fluorescent protein domain includes the amino acid sequence of SEQ ID NO: 11. In embodiments, the fluorescent protein domain includes the amino acid sequence of SEQ ID NO:12. In embodiments, the fluorescent protein domain includes the amino acid sequence of SEQ ID NO: 13. In embodiments, the fluorescent protein domain includes the amino acid sequence of SEQ ID NO:14. In embodiments, the fluorescent protein domain includes the amino acid sequence of SEQ ID NO: 15. In embodiments, the fluorescent protein domain includes the amino acid sequence of SEQ ID NO:16. In embodiments, the fluorescent protein domain includes the amino acid sequence of SEQ ID NO: 17. In embodiments, the fluorescent protein domain includes the amino acid sequence of SEQ ID NO: 18. In embodiments, the fluorescent protein domain includes the amino acid sequence of SEQ ID NO: 19. In embodiments, the fluorescent protein domain includes the amino acid sequence of SEQ ID NO:20. In embodiments, the fluorescent protein domain includes the amino acid sequence of SEQ ID NO:21. In embodiments, the fluorescent protein domain includes the amino acid sequence of SEQ ID NO: 22. In embodiments, the fluorescent protein domain includes the amino acid sequence of SEQ ID NO:23. In embodiments, the fluorescent protein domain includes the amino acid sequence of SEQ ID NO:24.

In embodiments, the fluorescent protein domain is the amino acid sequence of any one of SEQ ID NO:1 to SEQ ID NO:24. In embodiments, the fluorescent protein domain is the amino acid sequence of SEQ ID NO: 1. In embodiments, the fluorescent protein domain is the amino acid sequence of SEQ ID NO: 2. In embodiments, the fluorescent protein domain is the amino acid sequence of SEQ ID NO:3. In embodiments, the fluorescent protein domain is the amino acid sequence of SEQ ID NO:4 In embodiments, the fluorescent protein domain is the amino acid sequence of SEQ ID NO:5. In embodiments, the fluorescent protein domain is the amino acid sequence of SEQ ID NO: 6. In embodiments, the fluorescent protein domain is the amino acid sequence of SEQ ID NO:7. In embodiments, the fluorescent protein domain is the amino acid sequence of SEQ ID NO: 8. In embodiments, the fluorescent protein domain is the amino acid sequence of SEQ ID NO:9. In embodiments, the fluorescent protein domain is the amino acid sequence of SEQ ID NO: 10. In embodiments, the fluorescent protein domain is the amino acid sequence of SEQ ID NO:11. In embodiments, the fluorescent protein domain is the amino acid sequence of SEQ ID NO: 12. In embodiments, the fluorescent protein domain is the amino acid sequence of SEQ ID NO: 13. In embodiments, the fluorescent protein domain is the amino acid sequence of SEQ ID NO: 14. In embodiments, the fluorescent protein domain is the amino acid sequence of SEQ ID NO: 15. In embodiments, the fluorescent protein domain is the amino acid sequence of SEQ ID NO:16.

In embodiments, the fluorescent protein domain is the amino acid sequence of SEQ ID NO:17. In embodiments, the fluorescent protein domain is the amino acid sequence of SEQ ID NO: 18. In embodiments, the fluorescent protein domain is the amino acid sequence of SEQ ID NO: 19. In embodiments, the fluorescent protein domain is the amino acid sequence of SEQ ID NO:20. In embodiments, the fluorescent protein domain is the amino acid sequence of SEQ ID NO:21. In embodiments, the fluorescent protein domain is the amino acid sequence of SEQ ID NO:22. In embodiments, the fluorescent protein domain is the amino acid sequence of SEQ ID NO: 23. In embodiments, the fluorescent protein domain is the amino acid sequence of SEQ ID NO:24.

In embodiments, the split haloalkane dehalogenase domain includes the amino acid sequence of SEQ ID NO:67 or SEQ ID NO:68. In embodiments, the split haloalkane dehalogenase domain includes the amino acid sequence of SEQ ID NO:67. In embodiments, the split haloalkane dehalogenase domain includes the amino acid sequence of SEQ ID NO:68. In embodiments, the split haloalkane dehalogenase domain is the amino acid sequence of SEQ ID NO:67 or SEQ ID NO:68. In embodiments, the split haloalkane dehalogenase domain is the amino acid sequence of SEQ ID NO:67. In embodiments, the split haloalkane dehalogenase domain is the amino acid sequence of SEQ ID NO:68.

In embodiments, the split haloalkane dehalogenase domain includes an amino acid sequence having 70%, 75%, 80%, 85%, 90%, 95%, 96%, 97%, 98%, 99% or 100% sequence identity to the sequence of SEQ ID NO:67. In embodiments, the split haloalkane dehalogenase domain includes an amino acid sequence having 70% sequence identity to the sequence of SEQ ID NO:67. In embodiments, the split haloalkane dehalogenase domain includes an amino acid sequence having 75% sequence identity to the sequence of SEQ ID NO:67. In embodiments, the split haloalkane dehalogenase domain includes an amino acid sequence having 80% sequence identity to the sequence of SEQ ID NO:67. In embodiments, the split haloalkane dehalogenase domain includes an amino acid sequence having 85% sequence identity to the sequence of SEQ ID NO:67. In embodiments, the split haloalkane dehalogenase domain includes an amino acid sequence having 90% sequence identity to the sequence of SEQ ID NO:67. In embodiments, the split haloalkane dehalogenase domain includes an amino acid sequence having 95% sequence identity to the sequence of SEQ ID NO:67. In embodiments, the split haloalkane dehalogenase domain includes an amino acid sequence having 96% sequence identity to the sequence of SEQ ID NO:67. In embodiments, the split haloalkane dehalogenase domain includes an amino acid sequence having 97% sequence identity to the sequence of SEQ ID NO:67. In embodiments, the split haloalkane dehalogenase domain includes an amino acid sequence having 98% sequence identity to the sequence of SEQ ID NO:67. In embodiments, the split haloalkane dehalogenase domain includes an amino acid sequence having 99% sequence identity to the sequence of SEQ ID NO:67. In embodiments, the split haloalkane dehalogenase domain includes an amino acid sequence having 100% sequence identity to the sequence of SEQ ID NO:67.

In embodiments, the split haloalkane dehalogenase domain includes an amino acid sequence having 70%, 75%, 80%, 85%, 90%, 95%, 96%, 97%, 98%, 99% or 100% sequence identity to the sequence of SEQ ID NO:68. In embodiments, the split haloalkane dehalogenase domain includes an amino acid sequence having 70% sequence identity to the sequence of SEQ ID NO:68. In embodiments, the split haloalkane dehalogenase domain includes an amino acid sequence having 75% sequence identity to the sequence of SEQ ID NO:68. In embodiments, the split haloalkane dehalogenase domain includes an amino acid sequence having 80% sequence identity to the sequence of SEQ ID NO:68. In embodiments, the split haloalkane dehalogenase domain includes an amino acid sequence having 85% sequence identity to the sequence of SEQ ID NO:68. In embodiments, the split haloalkane dehalogenase domain includes an amino acid sequence having 90% sequence identity to the sequence of SEQ ID NO:68. In embodiments, the split haloalkane dehalogenase domain includes an amino acid sequence having 95% sequence identity to the sequence of SEQ ID NO:68. In embodiments, the split haloalkane dehalogenase domain includes an amino acid sequence having 96% sequence identity to the sequence of SEQ ID NO:68. In embodiments, the split haloalkane dehalogenase domain includes an amino acid sequence having 97% sequence identity to the sequence of SEQ ID NO:68. In embodiments, the split haloalkane dehalogenase domain includes an amino acid sequence having 98% sequence identity to the sequence of SEQ ID NO:68. In embodiments, the split haloalkane dehalogenase domain includes an amino acid sequence having 99% sequence identity to the sequence of SEQ ID NO:68. In embodiments, the split haloalkane dehalogenase domain includes an amino acid sequence having 100% sequence identity to the sequence of SEQ ID NO:68.

In embodiments, the cognate haloalkane dehalogenase split protein domain includes the amino acid sequence of SEQ ID NO:67 or SEQ ID NO:68. In embodiments, the cognate haloalkane dehalogenase split protein domain includes the amino acid sequence of SEQ ID NO:67. In embodiments, the cognate haloalkane dehalogenase split protein domain includes the amino acid sequence of SEQ ID NO:68. In embodiments, the cognate haloalkane dehalogenase split protein domain is the amino acid sequence of SEQ ID NO:67 or SEQ ID NO:68. In embodiments, the cognate haloalkane dehalogenase split protein domain is the amino acid sequence of SEQ ID NO:67. In embodiments, the cognate haloalkane dehalogenase split protein domain is the amino acid sequence of SEQ ID NO:68.

In embodiments, the cognate haloalkane dehalogenase split protein domain includes an amino acid sequence having 70%, 75%, 80%, 85%, 90%, 95%, 96%, 97%, 98%, 99% or 100% sequence identity to the sequence of SEQ ID NO:67. In embodiments, the cognate haloalkane dehalogenase split protein domain includes an amino acid sequence having 70% sequence identity to the sequence of SEQ ID NO:67. In embodiments, the cognate haloalkane dehalogenase split protein domain includes an amino acid sequence having 75% sequence identity to the sequence of SEQ ID NO:67. In embodiments, the cognate haloalkane dehalogenase split protein domain includes an amino acid sequence having 80% sequence identity to the sequence of SEQ ID NO:67. In embodiments, the cognate haloalkane dehalogenase split protein domain includes an amino acid sequence having 85% sequence identity to the sequence of SEQ ID NO:67. In embodiments, the cognate haloalkane dehalogenase split protein domain includes an amino acid sequence having 90% sequence identity to the sequence of SEQ ID NO:67. In embodiments, the cognate haloalkane dehalogenase split protein domain includes an amino acid sequence having 95% sequence identity to the sequence of SEQ ID NO:67. In embodiments, the cognate haloalkane dehalogenase split protein domain includes an amino acid sequence having 96% sequence identity to the sequence of SEQ ID NO:67. In embodiments, the cognate haloalkane dehalogenase split protein domain includes an amino acid sequence having 97% sequence identity to the sequence of SEQ ID NO:67. In embodiments, the cognate haloalkane dehalogenase split protein domain includes an amino acid sequence having 98% sequence identity to the sequence of SEQ ID NO:67. In embodiments, the cognate haloalkane dehalogenase split protein domain includes an amino acid sequence having 99% sequence identity to the sequence of SEQ ID NO:67. In embodiments, the cognate haloalkane dehalogenase split protein domain includes an amino acid sequence having 100% sequence identity to the sequence of SEQ ID NO:67.

In embodiments, the cognate haloalkane dehalogenase split protein domain includes an amino acid sequence having 70%, 75%, 80%, 85%, 90%, 95%, 96%, 97%, 98%, 99% or 100% sequence identity to the sequence of SEQ ID NO:68. In embodiments, the cognate haloalkane dehalogenase split protein domain includes an amino acid sequence having 70% sequence identity to the sequence of SEQ ID NO:68. In embodiments, the cognate haloalkane dehalogenase split protein domain includes an amino acid sequence having 75% sequence identity to the sequence of SEQ ID NO:68. In embodiments, the cognate haloalkane dehalogenase split protein domain includes an amino acid sequence having 80% sequence identity to the sequence of SEQ ID NO:68. In embodiments, the cognate haloalkane dehalogenase split protein domain includes an amino acid sequence having 85% sequence identity to the sequence of SEQ ID NO:68. In embodiments, the cognate haloalkane dehalogenase split protein domain includes an amino acid sequence having 90% sequence identity to the sequence of SEQ ID NO:68. In embodiments, the cognate haloalkane dehalogenase split protein domain includes an amino acid sequence having 95% sequence identity to the sequence of SEQ ID NO:68. In embodiments, the cognate haloalkane dehalogenase split protein domain includes an amino acid sequence having 96% sequence identity to the sequence of SEQ ID NO:68. In embodiments, the cognate haloalkane dehalogenase split protein domain includes an amino acid sequence having 97% sequence identity to the sequence of SEQ ID NO:68. In embodiments, the cognate haloalkane dehalogenase split protein domain includes an amino acid sequence having 98% sequence identity to the sequence of SEQ ID NO:68. In embodiments, the cognate haloalkane dehalogenase split protein domain includes an amino acid sequence having 99% sequence identity to the sequence of SEQ ID NO:68. In embodiments, the cognate haloalkane dehalogenase split protein domain includes an amino acid sequence having 100% sequence identity to the sequence of SEQ ID NO:68.

In embodiments, the chemical fluorophore is NGD, JF669, JFX646, or JF635. In embodiments, the chemical fluorophore is NGD. In embodiments, the chemical fluorophore is JF669. In embodiments, the chemical fluorophore is JFX646. In embodiments, the chemical fluorophore is JF635.

In embodiments, the chemical fluorophore includes the formula of

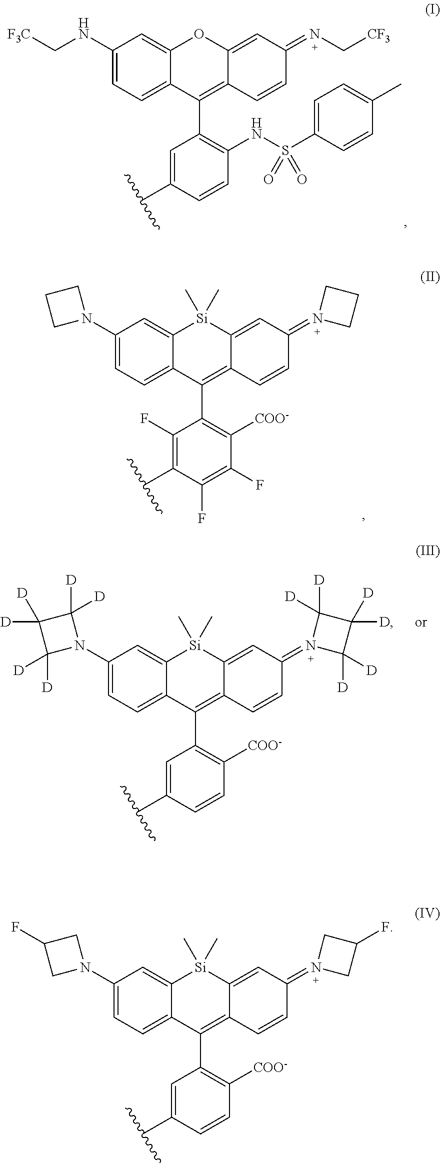

In embodiments, the chemical fluorophore includes the formula of

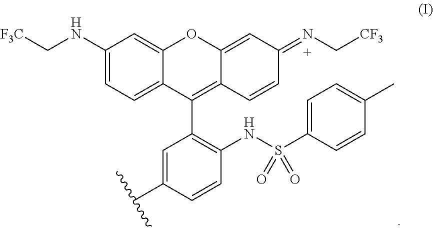 [SMILES: *c1ccc(NS(=O)(=O)c2ccc(C)cc2)c(-c2c3cc/c(=N\CC(F)(F)F)cc-3oc3cc(NCC(F)(F)F)ccc23)c1]

In embodiments, the chemical fluorophore includes the formula of

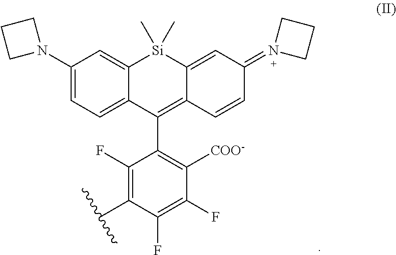

In embodiments, the chemical fluorophore includes the formula of

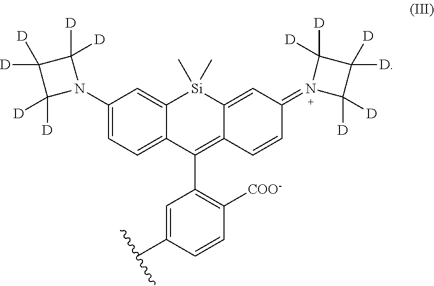

In embodiments, the chemical fluorophore includes the formula of

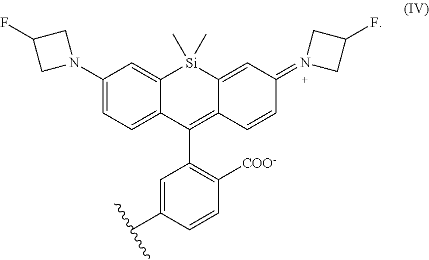

In embodiments, the biosensing protein domain includes: (a) a target biomolecule binding domain; (b) a target biomolecule substrate domain, a linker domain, and a target biomolecule activity sensing domain; (c) a target biomolecule domain, a linker domain, and a target biomolecule substrate domain. In embodiments, the biosensing protein domain includes a target biomolecule binding domain. In embodiments, the biosensing protein domain includes a target biomolecule substrate domain, a linker domain, and a target biomolecule activity sensing domain. In embodiments, the biosensing protein domain includes a target biomolecule substrate domain. In embodiments, the biosensing protein domain includes a linker domain. In embodiments, the biosensing protein domain includes a target biomolecule activity sensing domain. In embodiments, the biosensing protein domain includes a target biomolecule domain, a linker domain, and a target biomolecule substrate domain. In embodiments, the biosensing protein domain includes a target biomolecule domain. In embodiments, the biosensing protein domain includes a linker domain. In embodiments, the biosensing protein domain includes a target biomolecule substrate domain, split haloalkane dehalogenase domain is one part of a haloalkane dehalogenase split pair that forms a reconstituted haloalkane dehalogenase protein upon binding of a cognate haloalkane dehalogenase split protein domain.

In embodiments, the target biomolecule binding domain is capable of binding the target biomolecule. In embodiments, the target biomolecule binding domain binds the target biomolecule, thereby inducing a conformational change in the recombinant protein. In embodiments, the target biomolecule binding domain includes a phosphorylatable amino acid residue. In embodiments, the binding of the target biomolecule binding domain to the target biomolecule induces a modification of the target biomolecule binding domain. In embodiments, the modification of the target biomolecule binding domain is phosphorylation of the phosphorylatable amino acid residue. In embodiments, the target biomolecule phosphorylates the phosphorylatable amino acid residue, thereby inducing a conformational change in the recombinant protein. In embodiments, the conformational change allows the split haloalkane dehalogenase domain to bind the cognate haloalkane dehalogenase split protein domain: thereby forming a haloalkane dehalogenase protein. In embodiments, the conformational changes allows: (a) the chemical fluorophore to activate the fluorescent protein domain; or (b) the fluorescent protein domain to activate the chemical fluorophore. In embodiments, the conformational change increases the fluorescent signal from the FRET pair. In embodiments, the conformational change decreases the fluorescent signal from the FRET pair.

In embodiments, the target biomolecule binding domain is capable of interacting with the target biomolecule. In embodiments, the target biomolecule binding domain interacts with the target biomolecule, thereby inducing a conformational change in the recombinant protein. In embodiments, the conformational change allows the split haloalkane dehalogenase domain to bind the cognate haloalkane dehalogenase split protein domain; thereby forming a haloalkane dehalogenase protein. In embodiments, the conformational changes allows: (a) the chemical fluorophore to activate the fluorescent protein domain; or (b) the fluorescent protein domain to activate the chemical fluorophore. In embodiments, the conformational change increases the fluorescent signal from the FRET pair. In embodiments, the conformational change decreases the fluorescent signal from the FRET pair.

In embodiments, the target biomolecule domain is capable of binding the target biomolecule substrate domain. In embodiments, the target biomolecule domain is capable of being activated. In embodiments, the activated target biomolecule domain binds the target biomolecule substrate domain. In embodiments, the target biomolecule domain binds the target biomolecule substrate domain, thereby inducing a conformational change in the recombinant protein. In embodiments, the conformational change allows the split haloalkane dehalogenase domain to bind the cognate haloalkane dehalogenase split protein domain; thereby forming a haloalkane dehalogenase protein. In embodiments, the conformational changes allows: (a) the chemical fluorophore to activate the fluorescent protein domain; or (b) the fluorescent protein domain to activate the chemical fluorophore. In embodiments, the conformational change increases the fluorescent signal from the FRET pair. In embodiments, the conformational change decreases the fluorescent signal from the FRET pair.

In embodiments, the target biomolecule is a protein kinase, a second messenger molecule, or a GTPase. In embodiments, the target biomolecule is a protein kinase. In embodiments, the target biomolecule is a second messenger molecule. In embodiments, the target biomolecule is a GTPase.

In embodiments, the protein kinase is a protein kinase A (PKA), a protein kinase C (PKC), a protein kinase B (PKB), an extracellular signal-regulated kinase (ERK), a 5′ adenosine monophosphate-activated protein kinase (AMPK), a mammalian target of rapamycin (mTOR), a Fyn kinase, or a Src kinase. In embodiments, the protein kinase is a protein kinase A (PKA). In embodiments, the protein kinase is a protein kinase C (PKC). In embodiments, the protein kinase is a protein kinase B (PKB). In embodiments, the protein kinase is an extracellular signal-regulated kinase (ERK). In embodiments, the protein kinase is a 5′ adenosine monophosphate-activated protein kinase (AMPK). In embodiments, the protein kinase is a mammalian target of rapamycin (mTOR). In embodiments, the protein kinase is a Fyn kinase. In embodiments, the protein kinase is a Src kinase.

In embodiments, the second messenger molecule is cyclic adenosine monophosphate (cAMP), cyclic guanosine monophosphate (cGMP), inositol triphosphate (IP₃), diacylglycerol (DAG), or calcium (Ca²⁺). In embodiments, the second messenger molecule is cyclic adenosine monophosphate (cAMP). In embodiments, the second messenger molecule is cyclic guanosine monophosphate (cGMP). In embodiments, the second messenger molecule is inositol triphosphate (IP₃). In embodiments, the second messenger molecule is diacylglycerol (DAG). In embodiments, the second messenger molecule is calcium (Ca²⁺).

In embodiments, the GTPase is a Ras GTPase or a Rap1 GTPase. In embodiments, the GTPase is a Ras GTPase. In embodiments, the GTPase is a Rap1 GTPase.

In embodiments, the biomolecule substrate domain includes the amino acid sequence of any one of SEQ ID NO:27 to SEQ ID NO:36. In embodiments, the biomolecule substrate domain includes the amino acid sequence of SEQ ID NO:27. In embodiments, the biomolecule substrate domain includes the amino acid sequence of SEQ ID NO:28. In embodiments, the biomolecule substrate domain includes the amino acid sequence of SEQ ID NO:29. In embodiments, the biomolecule substrate domain includes the amino acid sequence of SEQ ID NO:30. In embodiments, the biomolecule substrate domain includes the amino acid sequence of SEQ ID NO:31. In embodiments, the biomolecule substrate domain includes the amino acid sequence of SEQ ID NO:32. In embodiments, the biomolecule substrate domain includes the amino acid sequence of SEQ ID NO:33. In embodiments, the biomolecule substrate domain includes the amino acid sequence of SEQ ID NO:34. In embodiments, the biomolecule substrate domain includes the amino acid sequence of SEQ ID NO:35. In embodiments, the biomolecule substrate domain includes the amino acid sequence of SEQ ID NO:36.

In embodiments, the biomolecule substrate domain is the amino acid sequence of any one of SEQ ID NO:27 to SEQ ID NO:36. In embodiments, the biomolecule substrate domain is the amino acid sequence of SEQ ID NO:27. In embodiments, the biomolecule substrate domain is the amino acid sequence of SEQ ID NO:28. In embodiments, the biomolecule substrate domain is the amino acid sequence of SEQ ID NO:29. In embodiments, the biomolecule substrate domain is the amino acid sequence of SEQ ID NO:30. In embodiments, the biomolecule substrate domain is the amino acid sequence of SEQ ID NO:31. In embodiments, the biomolecule substrate domain is the amino acid sequence of SEQ ID NO:32. In embodiments, the biomolecule substrate domain is the amino acid sequence of SEQ ID NO:33. In embodiments, the biomolecule substrate domain is the amino acid sequence of SEQ ID NO:34. In embodiments, the biomolecule substrate domain is the amino acid sequence of SEQ ID NO:35. In embodiments, the biomolecule substrate domain is the amino acid sequence of SEQ ID NO:36.

In embodiments, the target biomolecule substrate domain further includes a phosphorylatable amino acid residue. In embodiments, the phosphorylatable amino acid residue is capable of being phosphorylated by the protein kinase.

In embodiments, the target biomolecule domain includes the amino acid sequence of SEQ ID NO:44 or SEQ ID NO:45. In embodiments, the target biomolecule domain includes the amino acid sequence of SEQ ID NO:44. In embodiments, the target biomolecule domain includes the amino acid sequence of SEQ ID NO:45. In embodiments, the target biomolecule domain is the amino acid sequence of SEQ ID NO:44 or SEQ ID NO:45. In embodiments, the target biomolecule domain is the amino acid sequence of SEQ ID NO:44. In embodiments, the target biomolecule domain is the amino acid sequence of SEQ ID NO:45.

In embodiments, the target biomolecule activity sensing domain includes a phosphoamino acid binding domain. In embodiments, the phosphoamino acid binding domain is capable of binding a phosphorylated amino acid residue. In embodiments, the phosphoamino acid binding domain is capable of binding a phosphorylated amino acid residue in the target biomolecule substrate domain. In embodiments, the phosphoamino acid binding domain binds a phosphorylated amino acid residue in the target biomolecule substrate domain. In embodiments, the phosphoamino acid binding domain binds a phosphorylated amino acid residue in the target biomolecule substrate domain, thereby inducing a conformational change in the recombinant protein. In embodiments, the conformational changes allows: (a) the chemical fluorophore to activate the fluorescent protein domain; or (b) the fluorescent protein domain to activate the chemical fluorophore. In embodiments, the conformational change increases the fluorescent signal from the FRET pair. In embodiments, the conformational change decreases the fluorescent signal from the FRET pair.

In embodiments, the phosphoamino acid binding domain includes the amino acid sequence of SEQ ID NO:39, SEQ ID NO:40, or SEQ ID NO:41. In embodiments, the phosphoamino acid binding domain includes the amino acid sequence of SEQ ID NO:39. In embodiments, the phosphoamino acid binding domain includes the amino acid sequence of SEQ ID NO:40. In embodiments, the phosphoamino acid binding domain includes the amino acid sequence of SEQ ID NO:41. In embodiments, the phosphoamino acid binding domain is the amino acid sequence of SEQ ID NO:39. SEQ ID NO:40, or SEQ ID NO:41. In embodiments, the phosphoamino acid binding domain is the amino acid sequence of SEQ ID NO:39. In embodiments, the phosphoamino acid binding domain is the amino acid sequence of SEQ ID NO:40. In embodiments, the phosphoamino acid binding domain is the amino acid sequence of SEQ ID NO:41.

In embodiments, the linker domain is a peptide linker. In embodiments, the linker domain is between about 1 to about 500 amino acids in length. In embodiments, the linker domain is between about 2 to about 500 amino acids in length. In embodiments, the linker domain is between about 3 to about 500 amino acids in length. In embodiments, the linker domain is between about 4 to about 500 amino acids in length. In embodiments, the linker domain is between about 5 to about 500 amino acids in length. In embodiments, the linker domain is between about 6 to about 500 amino acids in length. In embodiments, the linker domain is between about 7 to about 500 amino acids in length. In embodiments, the linker domain is between about 8 to about 500 amino acids in length. In embodiments, the linker domain is between about 9 to about 500 amino acids in length. In embodiments, the linker domain is between about 10 to about 500 amino acids in length. In embodiments, the linker domain is between about 15 to about 500 amino acids in length. In embodiments, the linker domain is between about 20 to about 500 amino acids in length. In embodiments, the linker domain is between about 25 to about 500 amino acids in length. In embodiments, the linker domain is between about 30 to about 500 amino acids in length. In embodiments, the linker domain is between about 35 to about 500 amino acids in length. In embodiments, the linker domain is between about 40 to about 500 amino acids in length. In embodiments, the linker domain is between about 45 to about 500 amino acids in length. In embodiments, the linker domain is between about 50 to about 500 amino acids in length. In embodiments, the linker domain is between about 100 to about 500 amino acids in length. In embodiments, the linker domain is between about 150 to about 500 amino acids in length. In embodiments, the linker domain is between about 200 to about 500 amino acids in length. In embodiments, the linker domain is between about 250 to about 500 amino acids in length. In embodiments, the linker domain is between about 300 to about 500 amino acids in length. In embodiments, the linker domain is between about 350 to about 500 amino acids in length. In embodiments, the linker domain is between about 400 to about 500 amino acids in length. In embodiments, the linker domain is between about 450 to about 500 amino acids in length.

In embodiments, the linker domain is between about 1 to about 450 amino acids in length. In embodiments, the linker domain is between about 1 to about 400 amino acids in length. In embodiments, the linker domain is between about 1 to about 350 amino acids in length. In embodiments, the linker domain is between about 1 to about 300 amino acids in length. In embodiments, the linker domain is between about 1 to about 250 amino acids in length. In embodiments, the linker domain is between about 1 to about 200 amino acids in length. In embodiments, the linker domain is between about 1 to about 150 amino acids in length. In embodiments, the linker domain is between about 1 to about 100 amino acids in length. In embodiments, the linker domain is between about 1 to about 50 amino acids in length. In embodiments, the linker domain is between about 1 to about 45 amino acids in length. In embodiments, the linker domain is between about 1 to about 40 amino acids in length. In embodiments, the linker domain is between about 1 to about 35 amino acids in length. In embodiments, the linker domain is between about 1 to about 30 amino acids in length. In embodiments, the linker domain is between about 1 to about 25 amino acids in length. In embodiments, the linker domain is between about 1 to about 20 amino acids in length. In embodiments, the linker domain is between about 1 to about 15 amino acids in length. In embodiments, the linker domain is between about 1 to about 10 amino acids in length. In embodiments, the linker domain is between about 1 to about 9 amino acids in length. In embodiments, the linker domain is between about 1 to about 8 amino acids in length. In embodiments, the linker domain is between about 1 to about 7 amino acids in length. In embodiments, the linker domain is between about 1 to about 6 amino acids in length. In embodiments, the linker domain is between about 1 to about 5 amino acids in length. In embodiments, the linker domain is between about 1 to about 4 amino acids in length. In embodiments, the linker domain is between about 1 to about 3 amino acids in length. In embodiments, the linker domain is between about 1 to about 2 amino acids in length.

In embodiments, the linker domain is between 1 to 500 amino acids in length. In embodiments, the linker domain is between 2 to 500 amino acids in length. In embodiments, the linker domain is between 3 to 500 amino acids in length. In embodiments, the linker domain is between 4 to 500 amino acids in length. In embodiments, the linker domain is between 5 to 500 amino acids in length. In embodiments, the linker domain is between 6 to 500 amino acids in length. In embodiments, the linker domain is between 7 to 500 amino acids in length. In embodiments, the linker domain is between 8 to 500 amino acids in length. In embodiments, the linker domain is between 9 to 500 amino acids in length. In embodiments, the linker domain is between 10 to 500 amino acids in length. In embodiments, the linker domain is between 15 to 500 amino acids in length. In embodiments, the linker domain is between 20 to 500 amino acids in length. In embodiments, the linker domain is between 25 to 500 amino acids in length. In embodiments, the linker domain is between 30 to 500 amino acids in length. In embodiments, the linker domain is between 35 to 500 amino acids in length. In embodiments, the linker domain is between 40 to 500 amino acids in length. In embodiments, the linker domain is between 45 to 500 amino acids in length. In embodiments, the linker domain is between 50 to 500 amino acids in length. In embodiments, the linker domain is between 100 to 500 amino acids in length. In embodiments, the linker domain is between 150 to 500 amino acids in length. In embodiments, the linker domain is between 200 to 500 amino acids in length. In embodiments, the linker domain is between 250 to 500 amino acids in length. In embodiments, the linker domain is between 300 to 500 amino acids in length. In embodiments, the linker domain is between 350 to 500 amino acids in length. In embodiments, the linker domain is between 400 to 500 amino acids in length. In embodiments, the linker domain is between 450 to 500 amino acids in length.

In embodiments, the linker domain is between 1 to 450 amino acids in length. In embodiments, the linker domain is between 1 to 400 amino acids in length. In embodiments, the linker domain is between 1 to 350 amino acids in length. In embodiments, the linker domain is between 1 to 300 amino acids in length. In embodiments, the linker domain is between 1 to 250 amino acids in length. In embodiments, the linker domain is between 1 to 200 amino acids in length. In embodiments, the linker domain is between 1 to 150 amino acids in length. In embodiments, the linker domain is between 1 to 100 amino acids in length. In embodiments, the linker domain is between 1 to 50 amino acids in length. In embodiments, the linker domain is between 1 to 45 amino acids in length. In embodiments, the linker domain is between 1 to 40 amino acids in length. In embodiments, the linker domain is between 1 to 35 amino acids in length. In embodiments, the linker domain is between 1 to 30 amino acids in length. In embodiments, the linker domain is between 1 to 25 amino acids in length. In embodiments, the linker domain is between 1 to 20 amino acids in length. In embodiments, the linker domain is between 1 to 15 amino acids in length. In embodiments, the linker domain is between 1 to 10 amino acids in length. In embodiments, the linker domain is between 1 to 9 amino acids in length. In embodiments, the linker domain is between 1 to 8 amino acids in length. In embodiments, the linker domain is between 1 to 7 amino acids in length. In embodiments, the linker domain is between 1 to 6 amino acids in length. In embodiments, the linker domain is between 1 to 5 amino acids in length. In embodiments, the linker domain is between 1 to 4 amino acids in length. In embodiments, the linker domain is between 1 to 3 amino acids in length. In embodiments, the linker domain is between 1 to 2 amino acids in length.

In embodiments, the linker domain includes the amino acid sequence of any one of SEQ ID NO:46 to SEQ ID NO:59. In embodiments, the linker domain includes the amino acid sequence of any one of SEQ ID NO: 46. In embodiments, the linker domain includes the amino acid sequence of any one of SEQ ID NO:47. In embodiments, the linker domain includes the amino acid sequence of any one of SEQ ID NO:48. In embodiments, the linker domain includes the amino acid sequence of any one of SEQ ID NO:49. In embodiments, the linker domain includes the amino acid sequence of any one of SEQ ID NO:50. In embodiments, the linker domain includes the amino acid sequence of any one of SEQ ID NO:51. In embodiments, the linker domain includes the amino acid sequence of any one of SEQ ID NO:52. In embodiments, the linker domain includes the amino acid sequence of any one of SEQ ID NO: 53. In embodiments, the linker domain includes the amino acid sequence of any one of SEQ ID NO:54. In embodiments, the linker domain includes the amino acid sequence of any one of SEQ ID NO:55. In embodiments, the linker domain includes the amino acid sequence of any one of SEQ ID NO:56. In embodiments, the linker domain includes the amino acid sequence of any one of SEQ ID NO:57. In embodiments, the linker domain includes the amino acid sequence of any one of SEQ ID NO:58. In embodiments, the linker domain includes the amino acid sequence of any one of SEQ ID NO:59.

In embodiments, the linker domain is the amino acid sequence of any one of SEQ ID NO:46 to SEQ ID NO:59. In embodiments, the linker domain is the amino acid sequence of any one of SEQ ID NO:46. In embodiments, the linker domain is the amino acid sequence of any one of SEQ ID NO:47. In embodiments, the linker domain is the amino acid sequence of any one of SEQ ID NO:48. In embodiments, the linker domain is the amino acid sequence of any one of SEQ ID NO:49. In embodiments, the linker domain is the amino acid sequence of any one of SEQ ID NO:50. In embodiments, the linker domain is the amino acid sequence of any one of SEQ ID NO:51. In embodiments, the linker domain is the amino acid sequence of any one of SEQ ID NO:52. In embodiments, the linker domain is the amino acid sequence of any one of SEQ ID NO:53. In embodiments, the linker domain is the amino acid sequence of any one of SEQ ID NO:54. In embodiments, the linker domain is the amino acid sequence of any one of SEQ ID NO:55. In embodiments, the linker domain is the amino acid sequence of any one of SEQ ID NO:56. In embodiments, the linker domain is the amino acid sequence of any one of SEQ ID NO:57. In embodiments, the linker domain is the amino acid sequence of any one of SEQ ID NO:58. In embodiments, the linker domain is the amino acid sequence of any one of SEQ ID NO:59.

In embodiments, the target biomolecule binding domain includes the amino acid sequence of SEQ ID NO:37 or SEQ ID NO:38. In embodiments, the target biomolecule binding domain includes the amino acid sequence of SEQ ID NO:37. In embodiments, the target biomolecule binding domain includes the amino acid sequence of SEQ ID NO:38. In embodiments, the target biomolecule binding domain is the amino acid sequence of SEQ ID NO:37 or SEQ ID NO:38. In embodiments, the target biomolecule binding domain is the amino acid sequence of SEQ ID NO:37. In embodiments, the target biomolecule binding domain is the amino acid sequence of SEQ ID NO:38.

In embodiments, the fluorescent protein domain is attached to the N-terminus of the biosensing protein domain and the biosensing protein domain is attached to the N-terminus of the split haloalkane dehalogenase domain.

In embodiments, the split haloalkane dehalogenase domain is attached to the N-terminus of the biosensing protein domain and the biosensing protein domain is attached to the N-terminus of the fluorescent protein domain.

### Kits

The compositions provided herein include kits including a biosensor recombinant protein or portion thereof provided herein including embodiments thereof. The biosensor recombinant proteins included in the kit provided herein are described in detail throughout this application (including the description above and in the examples section). Thus, in an aspect is provided a kit including the recombinant protein provided herein including embodiments thereof and a second recombinant protein, wherein the second recombinant protein includes a protein of interest domain bound to the cognate split fluorescent protein.

In another aspect is provided a kit including the recombinant protein provided herein including embodiments thereof and a second recombinant protein, wherein the second recombinant protein includes the cognate haloalkane dehalogenase split protein domain bound to a protein of interest.

### Nucleic Acid Compositions

The compositions provided herein include nucleic acid molecules encoding the biosensor recombinant proteins or portions thereof provided herein including embodiments thereof. The biosensor recombinant proteins encoded by the isolated nucleic acid provided herein are described in detail throughout this application (including the description above and in the examples section). Thus, in an aspect is provided an isolated nucleic acid encoding the recombinant protein of provided herein including embodiments thereof.

### Expression Vector Compositions

The compositions provided herein include expression vectors including an isolated nucleic acid encoding biosensor recombinant proteins or portions thereof provided herein including embodiments thereof. The biosensor recombinant proteins encoded by the isolated nucleic acid provided herein are described in detail throughout this application (including the description above and in the examples section). Thus, in an aspect is provided an expression vector including the isolated nucleic acid provided herein including embodiments thereof.

In embodiments, the expression vector is a viral vector. In embodiments, the viral vector is an Adeno-associated viral (AAV) vector, an Adenovirus vector, or a lentiviral vector. In embodiments, the viral vector is an Adeno-associated viral (AAV) vector. In embodiments, the viral vector is an Adenovirus vector. In embodiments, the viral vector is a lentiviral vector.

### Methods

The biosensor recombinant proteins provided herein including embodiments thereof may be used for detecting a biomolecule or the activity of a biomolecule in a cell. Thus, in an aspect is provided a method for detecting a target biomolecule in a cell, the method including: (a) contacting the cell with an expression vector including a nucleic acid, wherein the nucleic acid encodes a recombinant protein provided herein including embodiments thereof; (b) transducing the cell with the expression vector; (c) allowing the cell to express the recombinant protein and interact with the target biomolecule in the cell; and (d) detecting a change in fluorescent signal from the recombinant protein, thereby detecting the target biomolecule.

In embodiments, the change in fluorescent signal is relative to the fluorescent signal of the recombinant protein prior to interaction with the target biomolecule in the cell. In embodiments, the change in fluorescent signal is a decrease in the fluorescent signal of the recombinant protein relative to the fluorescent signal of the recombinant protein prior to interaction with the target biomolecule in the cell. In embodiments, the change in fluorescent signal is an increase in the fluorescent signal of the recombinant protein relative to the fluorescent signal of the recombinant protein prior to interaction with the target biomolecule in the cell. In embodiments, the change in fluorescent signal is a decrease in fluorescent signal of the chemical fluorophore relative to the fluorescent signal of the recombinant protein prior interaction with the target biomolecule in the cell. In embodiments, the change in fluorescent signal is a decrease in fluorescent signal of the fluorescent protein domain relative to the fluorescent signal of the recombinant protein prior interaction with the target biomolecule in the cell. In embodiments, the change in fluorescent signal is an increase in fluorescent signal of the chemical fluorophore relative to the fluorescent signal of the recombinant protein prior interaction with the target biomolecule in the cell. In embodiments, the change in fluorescent signal is an increase in fluorescent signal of the fluorescent protein domain relative to the fluorescent signal of the recombinant protein prior interaction with the target biomolecule in the cell. In embodiments, the change in fluorescent signal is a decrease in fluorescent signal of the FRET pair relative to the fluorescent signal of the recombinant protein prior interaction with the target biomolecule in the cell. In embodiments, the change in fluorescent signal is an increase in fluorescent signal of the FRET pair relative to the fluorescent signal of the recombinant protein prior interaction with the target biomolecule in the cell.

In embodiments, the cell is imaged at a first time point to generate a first image. In embodiments, the cell is imaged at a second time point to generate a second image. In embodiments, step (d) includes comparing the first image to the second image to determine activity of the target biomolecule at the first time point compared to the second time point.

In embodiments, the cell is a living cell. The cell is a mammalian cell.

In embodiments, the cell is imaged using a fluorescence microscope.

In another aspect is provided a method for detecting a target biomolecule in a cell, the method including: (a) contacting the cell with an expression vector including a nucleic acid, wherein the nucleic acid encodes a recombinant protein provided herein including embodiments thereof; (b) transducing the cell with the expression vector; (c) allowing the cell to express the recombinant protein and interact with the target biomolecule in the cell; and (d) detecting a change in fluorescent signal from the recombinant protein, thereby detecting the target biomolecule.

In embodiments, the expression vector further includes a second nucleic acid encoding the second recombinant protein provided herein including embodiments thereof.

In embodiments, step (a) further includes contacting the cell with a second expression vector including a second nucleic acid, wherein the second nucleic acid encodes the second recombinant protein provided herein including embodiments thereof; wherein step (b) further includes transducing the cell with the second expression vector; and wherein step (c) further includes allowing the cell to express the second recombinant protein.

In embodiments, the change in fluorescent signal is relative to the fluorescent signal of the recombinant protein prior to interaction with the target biomolecule in the cell. In embodiments, the change in fluorescent signal is a decrease in the fluorescent signal of the recombinant protein relative to the fluorescent signal of the recombinant protein prior to interaction with the target biomolecule in the cell. In embodiments, the change in fluorescent signal is an increase in the fluorescent signal of the recombinant protein relative to the fluorescent signal of the recombinant protein prior to interaction with the target biomolecule in the cell. In embodiments, the change in fluorescent signal is a decrease in fluorescent signal of the chemical fluorophore relative to the fluorescent signal of the recombinant protein prior interaction with the target biomolecule in the cell. In embodiments, the change in fluorescent signal is a decrease in fluorescent signal of the reconstituted fluorescent protein relative to the fluorescent signal of the recombinant protein prior interaction with the target biomolecule in the cell. In embodiments, the change in fluorescent signal is an increase in fluorescent signal of the chemical fluorophore relative to the fluorescent signal of the recombinant protein prior interaction with the target biomolecule in the cell. In embodiments, the change in fluorescent signal is an increase in fluorescent signal of the reconstituted fluorescent protein relative to the fluorescent signal of the recombinant protein prior interaction with the target biomolecule in the cell. In embodiments, the change in fluorescent signal is a decrease in fluorescent signal of the FRET pair relative to the fluorescent signal of the recombinant protein prior interaction with the target biomolecule in the cell. In embodiments, the change in fluorescent signal is an increase in fluorescent signal of the FRET pair relative to the fluorescent signal of the recombinant protein prior interaction with the target biomolecule in the cell.

In embodiments, the cell is imaged at a first time point to generate a first image. In embodiments, the cell is imaged at a second time point to generate a second image. In embodiments, step (d) includes comparing the first image to the second image to determine activity of the target molecule at the first time point compared to the second time point.

In embodiments, the cell is a living cell. In embodiments, the cell is a mammalian cell.

In embodiments, the cell is imaged using a fluorescence microscope.

In another aspect is provided a method for detecting a target biomolecule in a cell, the method including: (a) contacting the cell with an expression vector including a nucleic acid, wherein the nucleic acid encodes a recombinant protein provided herein including embodiments thereof; (b) transducing the cell with the expression vector; (c) allowing the cell to express the recombinant protein and interact with the target biomolecule in the cell; and (d) detecting a change in fluorescent signal from the recombinant protein, thereby detecting the target biomolecule.

In embodiments, the expression vector further includes a second nucleic acid encoding the second recombinant protein provided herein including embodiments thereof. In embodiments, step (a) further includes contacting the cell with a second expression vector including a second nucleic acid, wherein the second nucleic acid encodes the second recombinant protein provided herein including embodiments thereof; wherein step (b) further includes transducing the cell with the second expression vector; and wherein step (c) further includes allowing the cell to express the second recombinant protein.

In embodiments, the change in fluorescent signal is relative to the fluorescent signal of the recombinant protein prior to interaction with the target biomolecule in the cell. In embodiments, the change in fluorescent signal is a decrease in the fluorescent signal of the recombinant protein relative to the fluorescent signal of the recombinant protein prior to interaction with the target biomolecule in the cell. In embodiments, the change in fluorescent signal is an increase in the fluorescent signal of the recombinant protein relative to the fluorescent signal of the recombinant protein prior to interaction with the target biomolecule in the cell. In embodiments, the change in fluorescent signal is a decrease in fluorescent signal of the chemical fluorophore relative to the fluorescent signal of the recombinant protein prior interaction with the target biomolecule in the cell. In embodiments, the change in fluorescent signal is a decrease in fluorescent signal of the fluorescent protein domain relative to the fluorescent signal of the recombinant protein prior interaction with the target biomolecule in the cell. In embodiments, the change in fluorescent signal is an increase in fluorescent signal of the chemical fluorophore relative to the fluorescent signal of the recombinant protein prior interaction with the target biomolecule in the cell. In embodiments, the change in fluorescent signal is an increase in fluorescent signal of the fluorescent protein domain relative to the fluorescent signal of the recombinant protein prior interaction with the target biomolecule in the cell. In embodiments, the change in fluorescent signal is a decrease in fluorescent signal of the FRET pair relative to the fluorescent signal of the recombinant protein prior interaction with the target biomolecule in the cell. In embodiments, the change in fluorescent signal is an increase in fluorescent signal of the FRET pair relative to the fluorescent signal of the recombinant protein prior interaction with the target biomolecule in the cell.

In embodiments, the cell is imaged at a first time point to generate a first image. In embodiments, the cell is imaged at a second time point to generate a second image. In embodiments, step (d) includes comparing the first image to the second image to determine activity of the target biomolecule at the first time point compared to the second time point.

In embodiments, the cell is a living cell. In embodiments, the cell is a mammalian cell.

In embodiments, the cell is imaged using a fluorescence microscope.

### EXAMPLES

### Example 1: A Fluorescent Biosensor for Measuring Ras Activity in Living Cells

### Introduction

Protein kinases transmit signals through phosphorylation, regulating most aspects of cellular function. Kinases are embedded in complex signaling networks and signaling specificity relies on their tight spatiotemporal regulation. It may therefore crucial to investigate not only the overall activity level of kinases in a cell but to study the spatiotemporal organization on a subcellular level in order to understand differentially regulated signaling pathways. The method of choice to do so is fluorescence microscopy as it is highly compatible with live-cell experiments. In order to infer the activity of kinases fluorescent kinase activity reporters (KARs) have been developed. They consist of a reporting unit that is usually based on fluorescent proteins (FPs) and a sensing unit that undergoes a conformational change upon phosphorylation by the kinase, which leads to a change in fluorescence signal. The main class of kinase biosensors is based on a common sensing unit, which consists of a peptide substrate that can be specifically phosphorylated by the kinase and a phosphoaminoacid binding protein (e.g. FHA1). The reporting unit most often follows a FRET-based design where the distance and orientation between two FPs changes upon sensing kinase activity, leading to a change in FRET efficiency, which can be detected by fluorescence microscopy^{[1]}.

The main limitations of current FRET-based biosensors are small dynamic ranges, low brightness and low photostability as well as their spectral properties mainly being limited to the blue-red region as far-red and near infrared (NIR) fluorescent biosensors are rare. This complicates the use of these biosensors in advanced microscopy applications such as biosensor multiplexing experiments or high-throughput measurements^{[2]}. An alternative to FP-based biosensors are so called hybrid or chemigenetic biosensors. They make use of synthetic fluorophores (chemi) in combination with self-labeling protein tags (genetic)^{[3]}. Self-labeling protein tags can be genetically encoded and react specifically and irreversibly with a ligand carrying a cell-permeable synthetic fluorophore. This renders the self-labeling protein tag fluorescent, which can be used analogously to a FP with an additional labeling step. The most commonly used self-labeling protein tags are SNAP-tag and HaloTag due to their fast-labeling kinetics^{[4, 5]} Synthetic fluorophores are available as cell permeable variants in the form of SNAP-tag and HaloTag substrates and are brighter and more photostable than FPs^{[6]}. FRET-based biosensors were previously built with the use of self-labeling protein tag-synthetic fluorophore couples. The main class of self-labeling protein tag containing biosensors, so called semisynthetic fluorescent sensor proteins (Snifits), are FRET-based biosensors which rely on specialized synthetic ligands complicating the generalization of this concept^{[7]}. More recently, FRET-based biosensors using a combination of FPs and self-labeling protein tags^{[8, 9]} as well as biosensors using two self-labeling protein tags were introduced^{[10]}. The latter study also generated kinase biosensors albeit these biosensors showed limited dynamic ranges (<60%). Here, we present a generalizable strategy to improve the dynamic range and red-shift the emission properties of FRET-based KARs. The presented biosensors will hence enable multiplexing experiments as well as measurements that require more sensitive ratiometric biosensors such as high-throughput measurements. Due to the enhanced properties of the generated biosensors this concept was additionally used to generate new FluoSTEP biosensors^{[11]} that can be expressed at the endogenous level.

### FRET-Based KARs to Measure Protein Kinase a (PKA) Activity Using HaloTag as FRET Donor

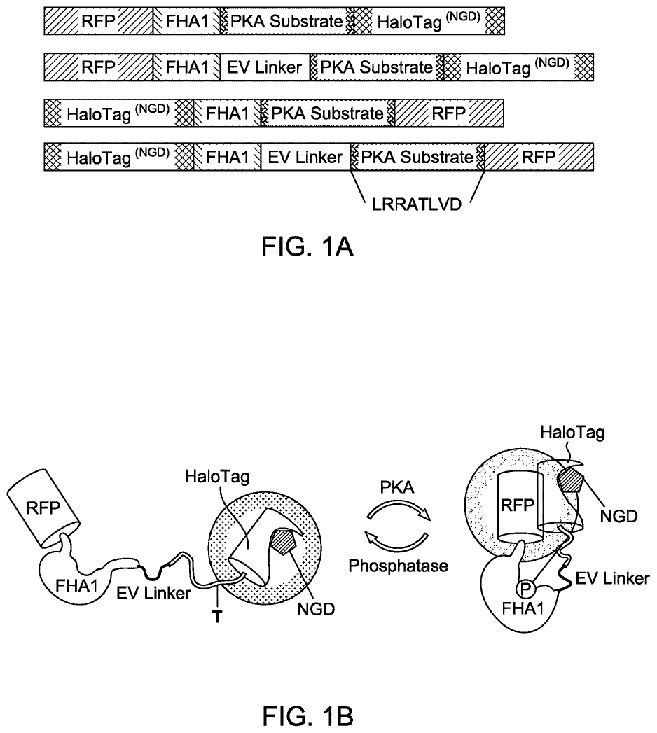

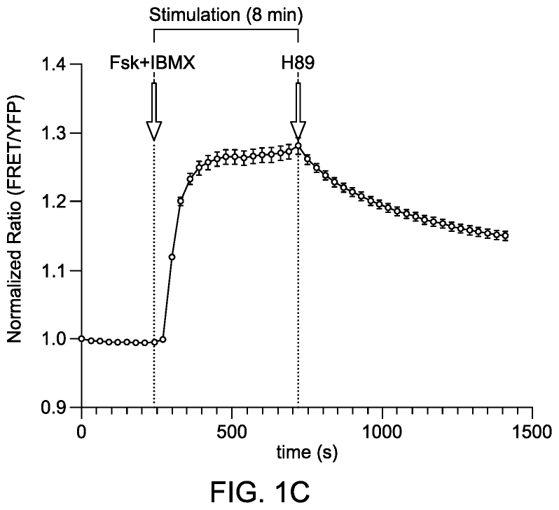

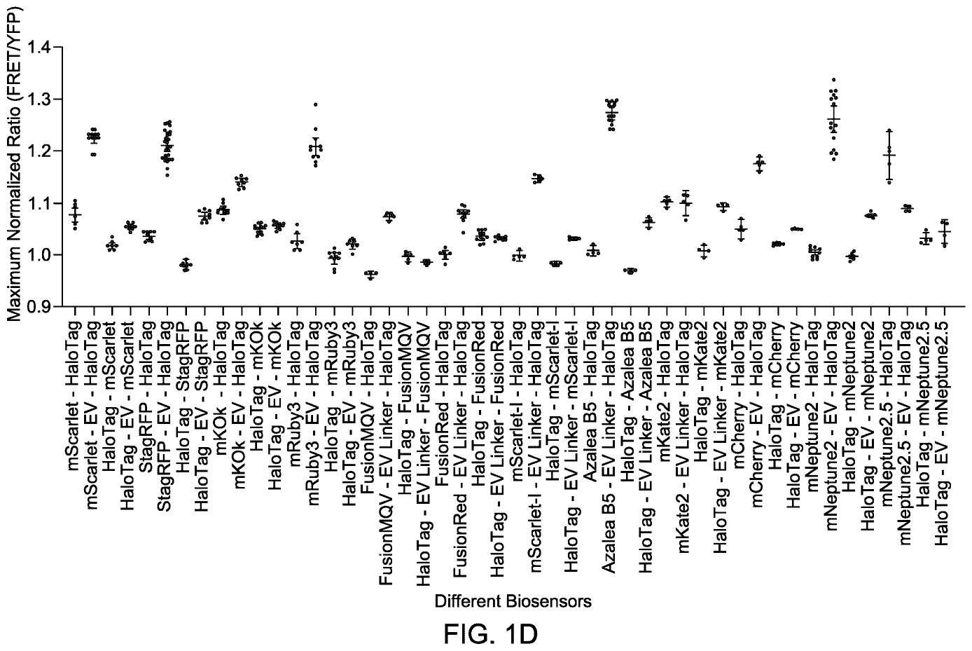

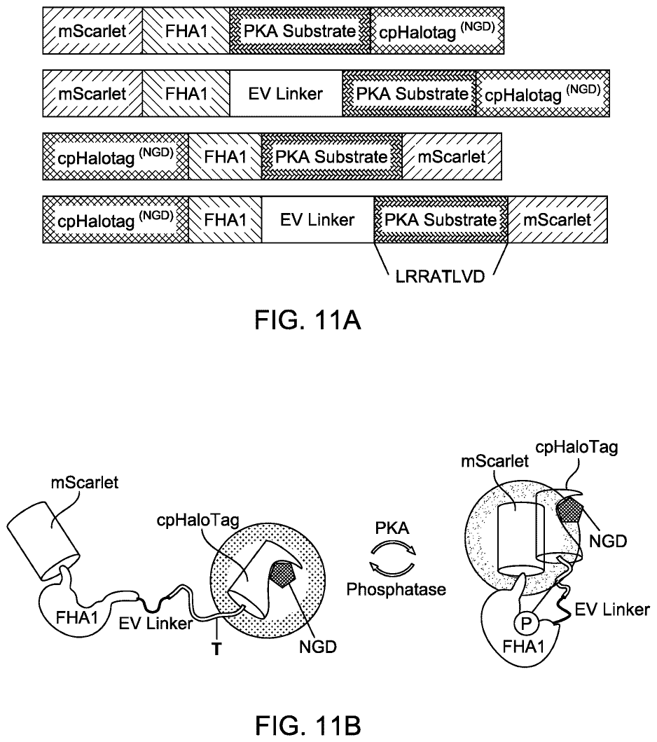

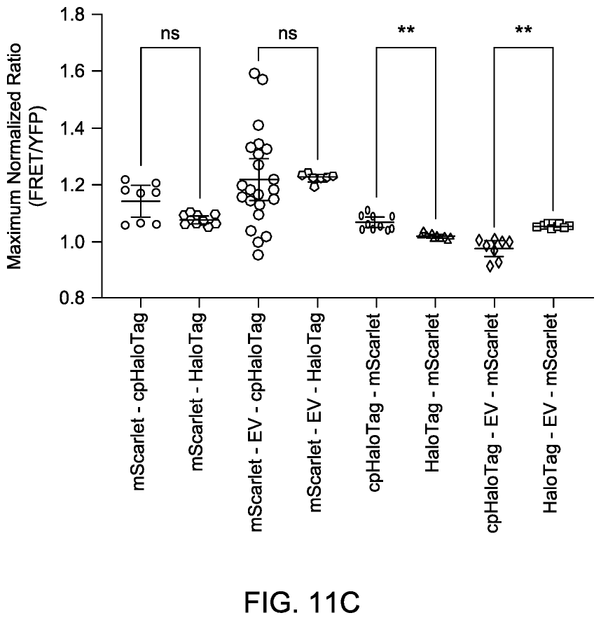

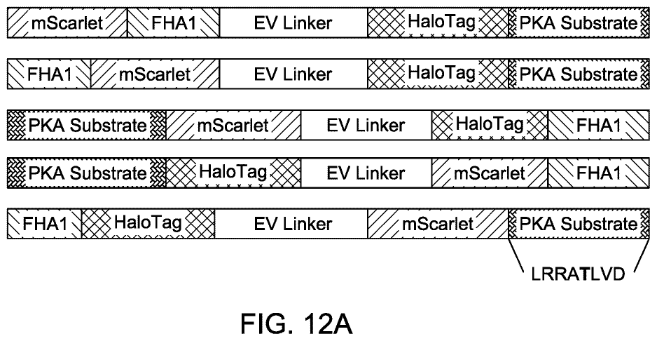

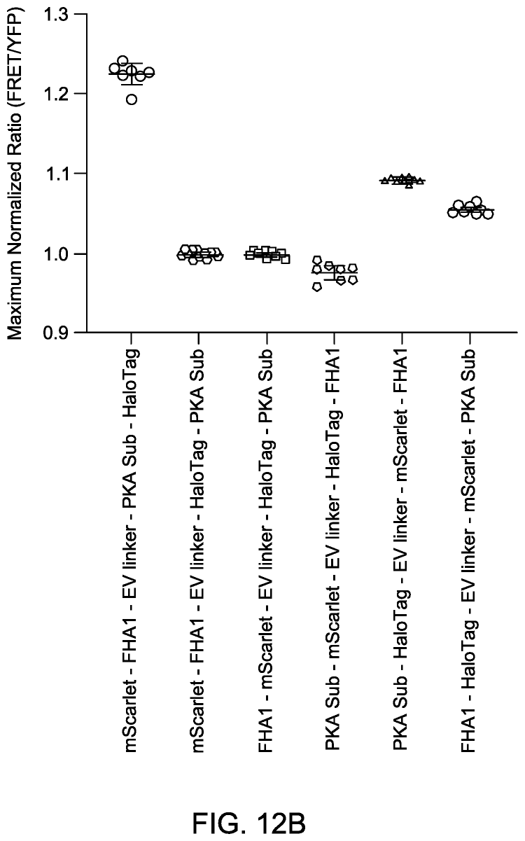

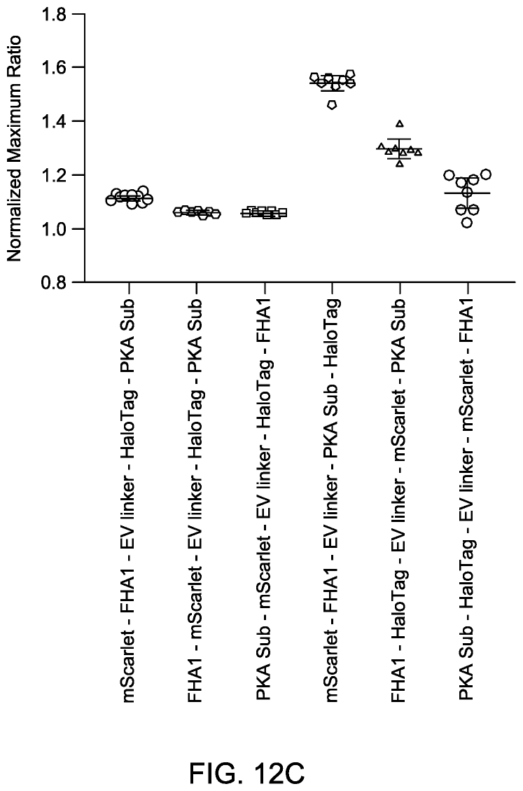

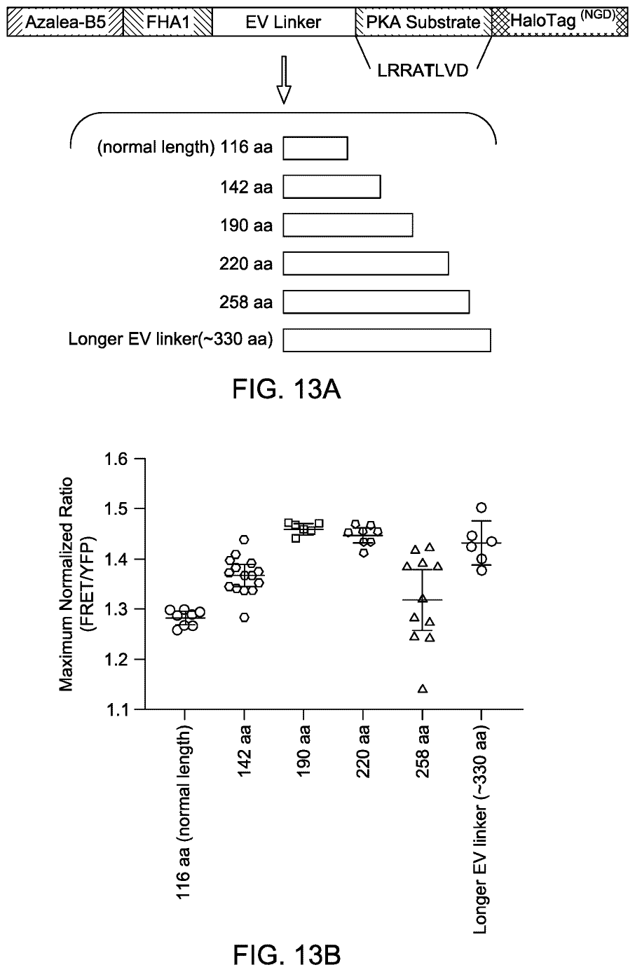

In order to generate KARs that are both bright and photostable, we replaced one of the FPs in the prototypic FRET-based A kinase activity reporter AKAR4 with the self-labeling protein tag HaloTag, which can be flexibly labeled with synthetic fluorophores in different spectral regions. Here, we used a specific green-yellow HaloTag fluorophore (NGD, Table 1)^{[12]}. To identify the best FRET acceptor for HaloTag^{(NGD)}, we selected a panel of 12 different RFPs with different excitation and emission properties. Given that the FRET efficiency depends not only on the distance between the donor and the acceptor fluorophore, but also on their relative orientation[¹³], we tested two different domain orientations (HaloTag-RFP or RFP-HaloTag) and two different linker lengths (flexible linker with 14 amino acids or EV linker with 116 amino acids^{[14]}, FIG. 1A). This resulted in a total of 48 yellow-red FRET-based AKARs which were tested to identify the biosensor with the largest dynamic range. The 48 biosensors were transfected into HeLa cells, the HaloTag labeled with NGD, and the cells stimulated with 50 i.iM forskolin (Fsk) and 100 i.iM 3-isobutyl-1-methylxanthine (IBMX, FIG. 1B). Upon stimulation, the signal in the FRET channel increased whereas the NGD signal decreased leading to an increase in the FRET/yellow emission ratio. Subsequently, with the addition of the PKA-specific inhibitor H89 after 8 minutes of stimulation, the FRET/yellow emission ratio of most biosensors decreased (FIG. 1C). We determined the maximum normalized ratio (dynamic range) of each biosensor after 8 minutes of stimulation, and found that the optimal orientation of the domains in the FRET-based biosensor is RFP-HaloTag. In addition, the use of the EV linker increased the dynamic range of most biosensors. The RFP Azalea-B5^{[15]} performed best showing a dynamic range of 26.9%±0.4% (n=21 cells) [mean±SEM]. (FIG. 1D). In order to further test the orientation dependence of the FRET efficiency we introduced a circularly permuted HaloTag (cpHaloTag)^{[16]} to four biosensors. We used NGD to label cpHaloTag and measured their response upon Fsk/IBMX stimulation. Measurement of the maximum normalized ratio after 8 min of stimulation revealed that cpHaloTag did not improve the dynamic range as compared to HaloTag. (FIGS. 11A-11C). Furthermore, we explored the option of further shuffling the domain order of RFP, HaloTag, FHA1, and PKA substrate under the constraint that the reporter domains must be separated by the EV linker. However, none of the 5 newly generated biosensors showed a higher dynamic range than the best candidate (mScarlet-FHA1-EV linker-PKA substrate-HaloTag^{(NGD)}, FIGS. 12A-12C). Next, we further tested if increasing the length of the EV linker between the sensing domains would lead to an increased dynamic range. Adding a flexible linker between the two halves of the sensing unit was reported to effectively reduce the basal emission ratio and therefore increase the dynamic range of the biosensor^{[14]}. Extending the current EV linker from 116 aa to 190 aa successfully increased the dynamic range of the Azalea-B5 containing biosensor to 46.2%±0.4% (n=6 cells) [mean±SEM] after stimulation (FIGS. 13A-13B) which is similar to the current best green-red FRET-based AKAR (44.6%)^{[17]}. We expect that this FRET-based AKAR will exhibit enhanced photostability over FP-based AKARs.

### FRET-Based AKARs Using HaloTag as FRET Acceptor

### Characterization of Red-Far Red FRET-Based AKARs

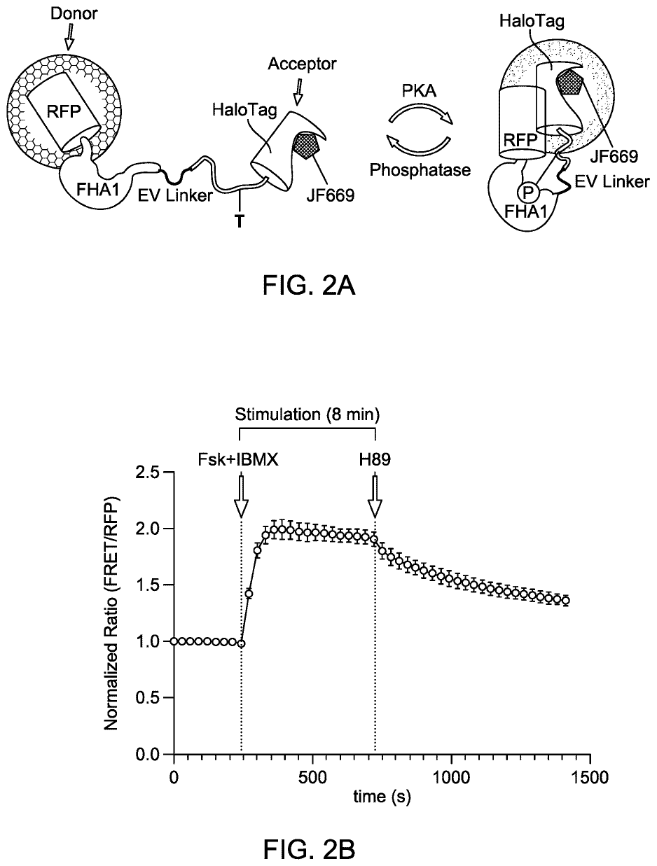

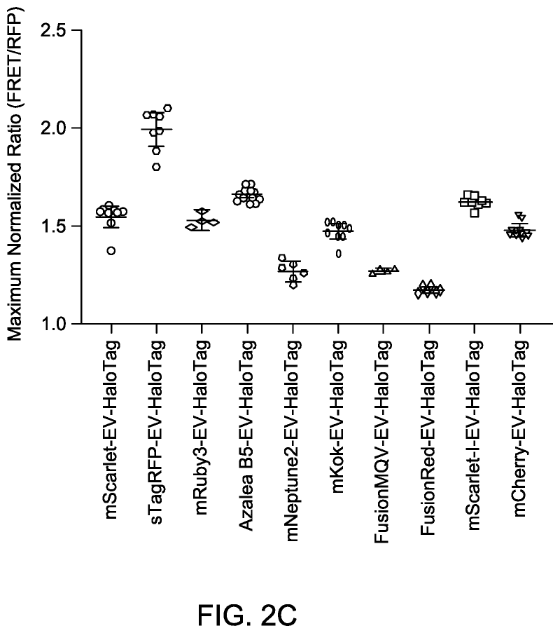

The HaloTag can be flexibly labeled with synthetic fluorophores with different spectral properties. We therefore tested if the HaloTag could serve as the FRET acceptor in our previously generated FRET-based AKARs. Instead of labeling the HaloTag with NGD (donor) we employed JF669^{[18]} (Table 1), a far-red fluorophore, which would then act as the FRET acceptor (FIG. 2A). To identify the best FRET donor for HaloTag labeled with JF669 we tested 10 RFPs of the previously identified (RFP-FAH1-EV linker-PKA substrate-HaloTag) domain orientation. Different RFP-HaloTag reporters were transfected into HeLa cells, labeled with JF669, and used to monitor changes in intracellular PKA activity after stimulation with 50 tM Fsk and 100 tM IBMX. Among the 10 different RFPs, the biosensor containing sTagRFP exhibited the largest dynamic range of 99.1%±3.4% (n=8 cells) [mean SEM](FIGS. 2B-2C). Moreover, to shift the biosensors even further to the far-red NIR region, we generated biosensors containing mRhubarb720 and miRFP720 for which HaloTag^{(JF669)} could once more act as the FRET donor (Table 2).

### Optimizing the Length of the EV Linker in FRET-Based AKARs

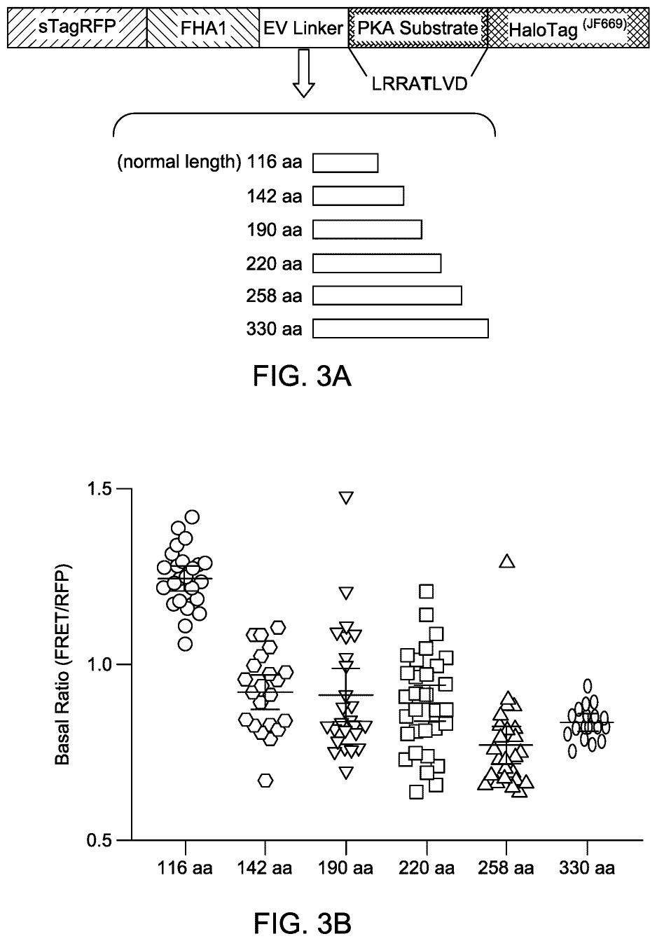

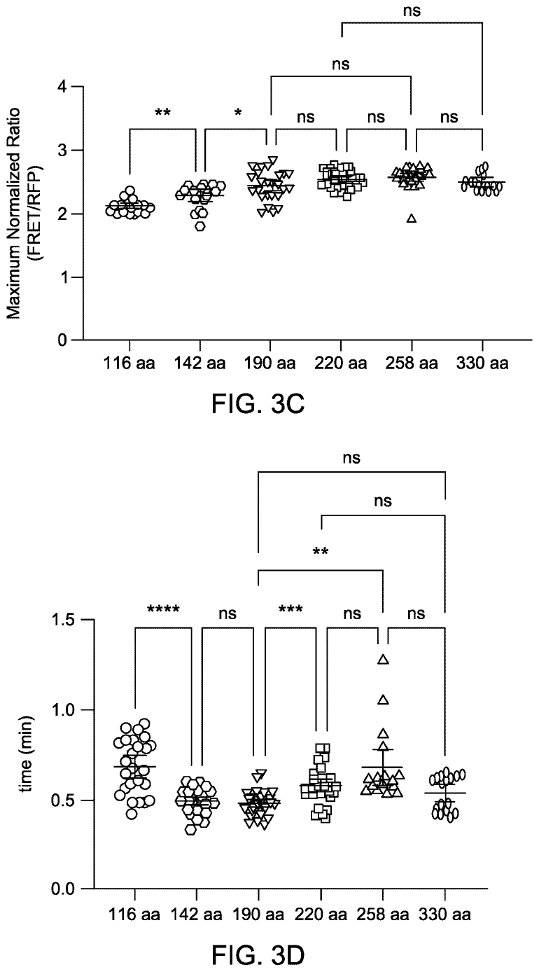

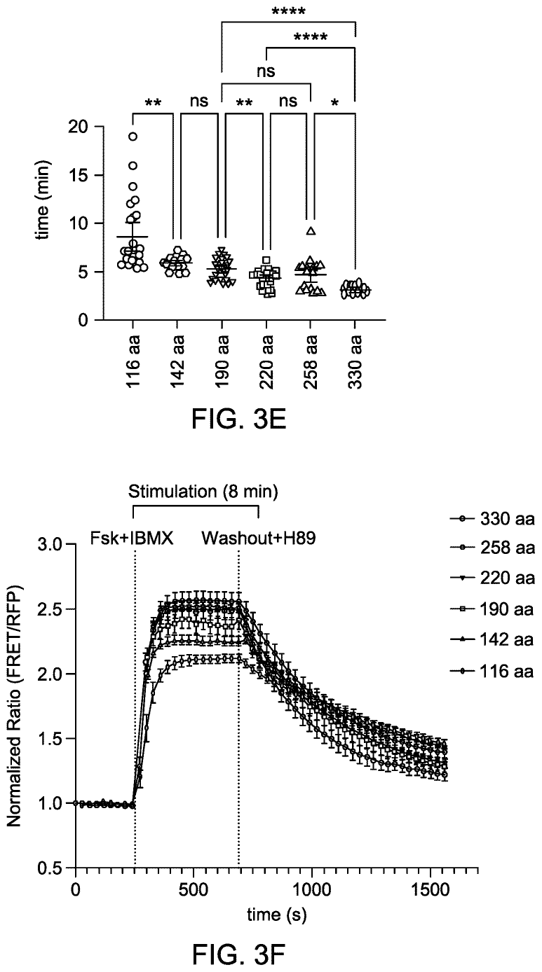

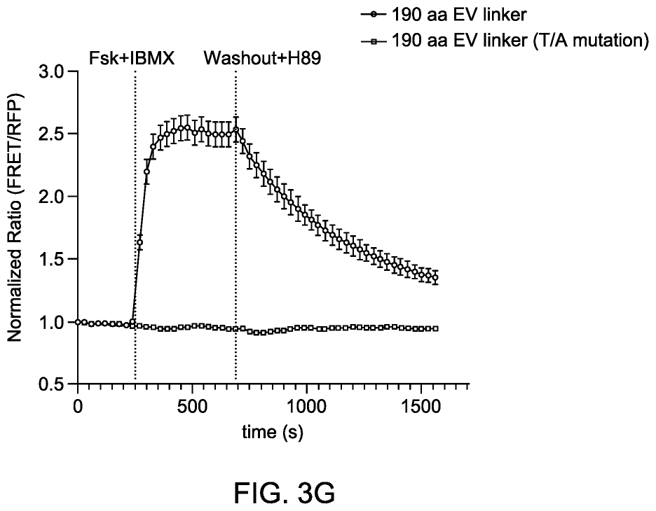

In order to further improve the dynamic range of the best red-far red FRET-based AKAR (sTagRFP-FHA1-EV linker-PKA substrate-HaloTag^{(JF669)}), we optimized the length of the flexible EV linker. Using molecular cloning techniques, 6 FRET-based AKARs containing different lengths of EV linkers were generated, ranging in length from a minimum of 116 aa to a maximum of 330 aa (FIG. 3A). We overexpressed the reporters in HeLa cells and monitored changes in PKA activity after stimulation with 50 tM Fsk and 100 tM IBMX. We found that the basal emission ratio decreased with increasing EV linker length while the dynamic range increased as expected (FIGS. 3B-3C). We additionally quantified the on and off kinetics of the biosensor. According to the dynamic range, the half-life of the on kinetics (Fsk/IBMX), and the half-life of the off kinetics (H89), sTagRFP-FHA1-258aa EV linker-PKA substrate-HaloTag^{(JF669)} showed the best overall performance with a dynamic range of 158.5%±2.3% (n=15 cells) [mean±SEM](FIGS. 3B-3F). In contrast, HeLa cells expressing a T/A mutant that cannot be phosphorylated showed no emission ratio response upon Fsk and IBMX treatment (FIG. 3G). This red-far red FRET AKAR is currently the biosensor with the highest reported dynamic range in this spectral region. It is hence a promising candidate for applications that require high sensitivity (e.g., high-throughput screening) or far red emission (e.g. multiplexing experiments or tissue/animal experiments).

Generalizing the Biosensor Design (sTagRFP-HaloTag(JF669)) to Other Kinase Activities and Small-Molecule Sensing

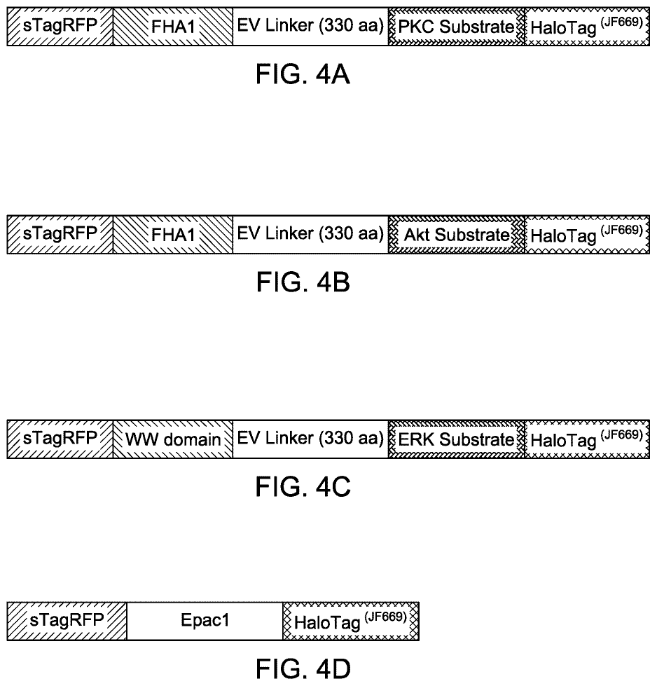

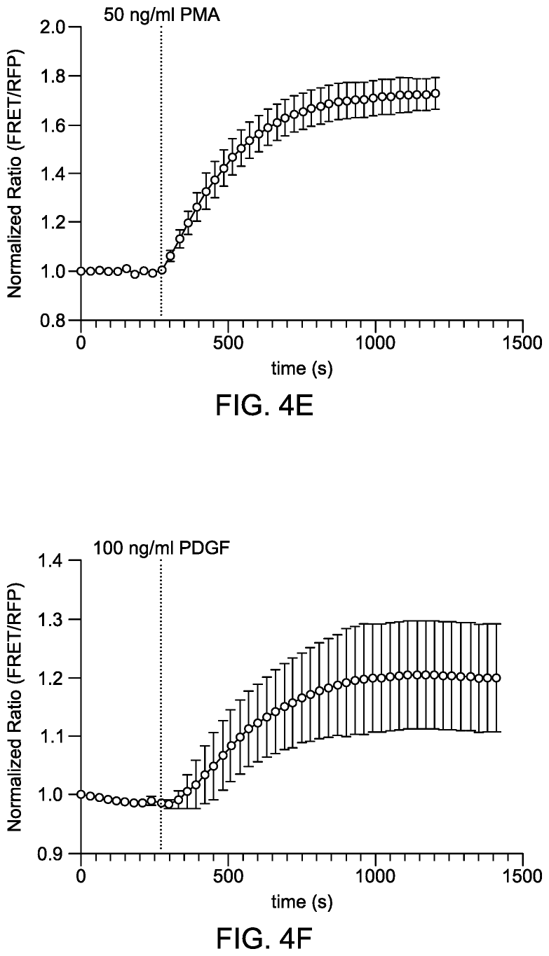

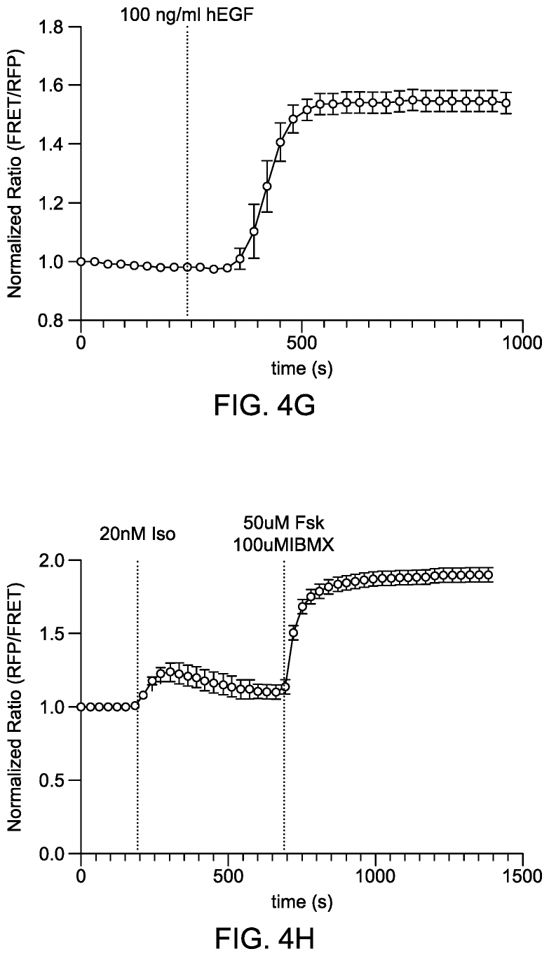

Due to the modular structure of FRET-based biosensors, which mainly consist of a reporting domain and a sensing domain, we generalized the biosensor design based on sTagRFP and HaloTag^{(JF669)} to other KARs. We replaced the PKA sensing domain in sTagRFP-FHA1-330 aa EV linker-PKA substrate-HaloTag^{(JF669)} by sensing domains for protein kinase C (PKC) and protein kinase B (PKB/Akt), generating FRET-based CKAR and AktAR using sTagRFP-HaloTag^{(JF669)} (FIGS. 4A-4B). The resulting reporters were expressed in HeLa and NIH3T3 cells. After stimulation by phorbol 12-myristate 13-acetate (PMA), the CKAR showed a 73.3%±2.8% (n=12 cells) [mean±SEM] increase in emission ratio (FIG. 4E). Similarly, AktAR showed a dynamic range of 20.7%±3.3% (n=6 cells) [mean±SEM] after stimulation with platelet-derived growth factor (PDGF) (FIG. 4F). We also generated enhanced reporters for extracellular signal-regulated kinase (ERK) and the second messenger cAMP (FIGS. 4C-4D). The former showed a dynamic range of 55.3% 1.4% (n=8 cells) [mean±SEM] after being stimulated by human epidermal growth factor (hEGF) in HEK293T cells (FIG. 4G), and the latter reached a 89.7%±2.0% (n=9 cells) [mean±SEM] positive response upon Fsk/IBMX stimulation in HEK293T cells (FIG. 411). This demonstrates that the sTagRFP-HaloTag^{(JF669)} FRET pair can successfully be used to generate other red-far red biosensors with high dynamic ranges.

### Characterization of Yellow-Far Red FRET-Based AKARs

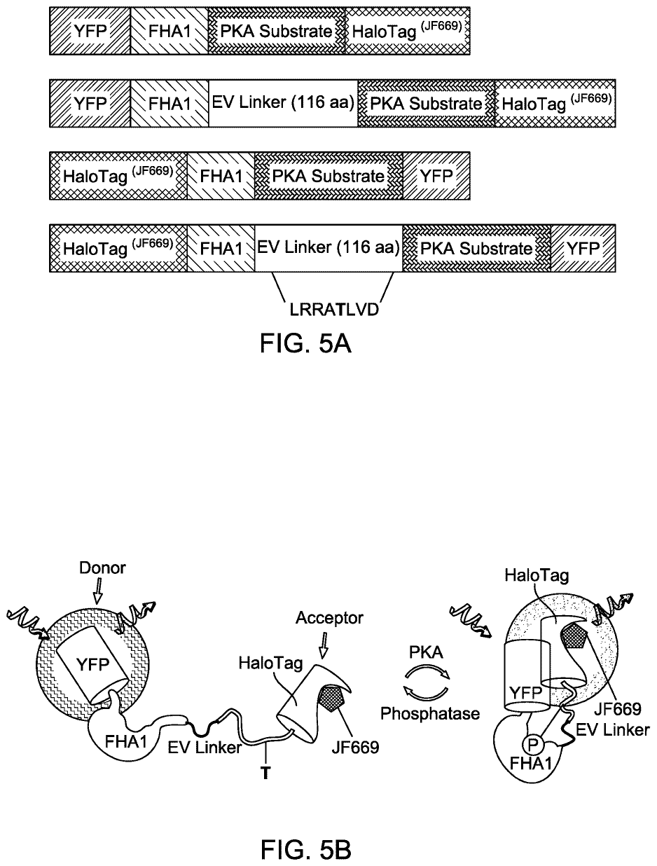

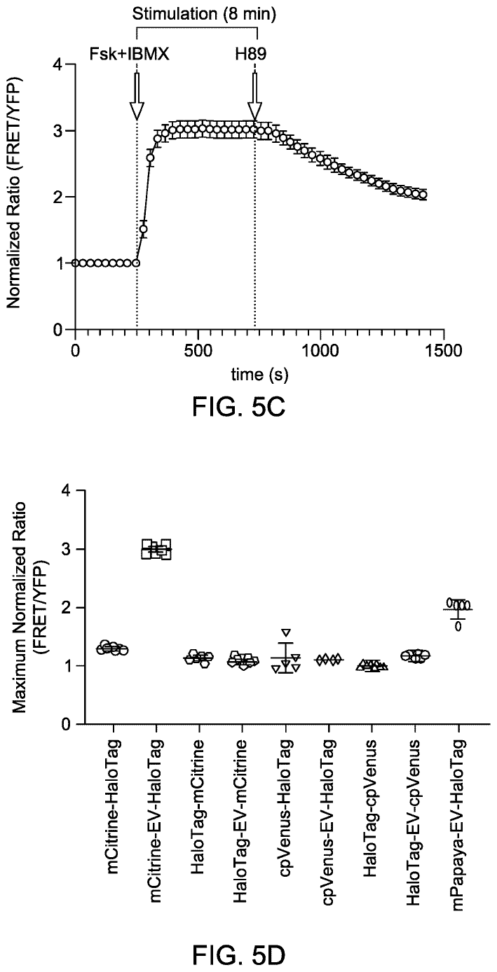

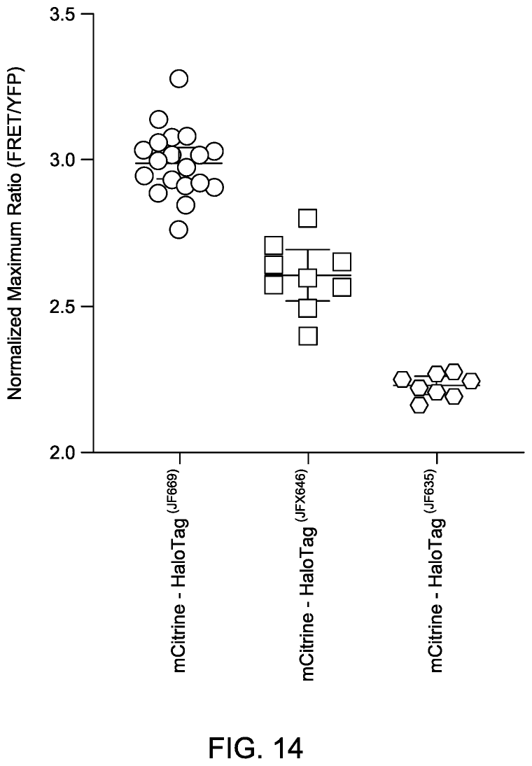

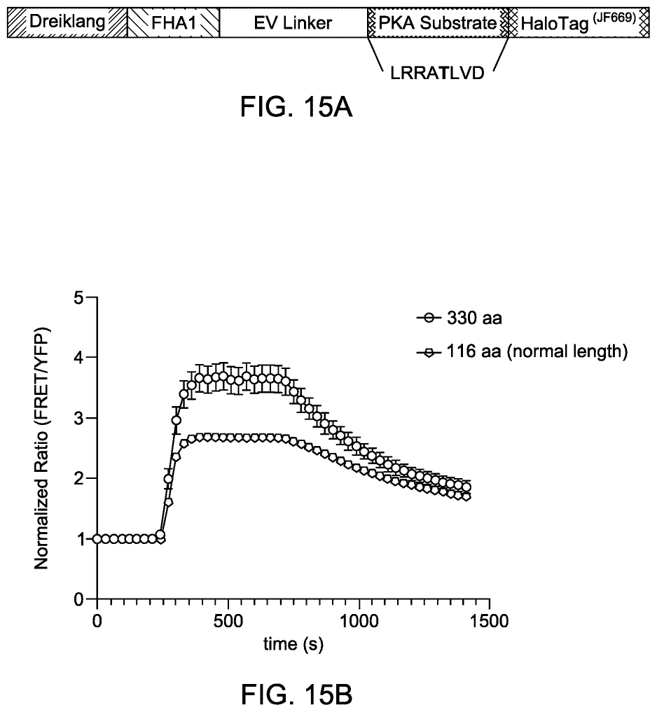

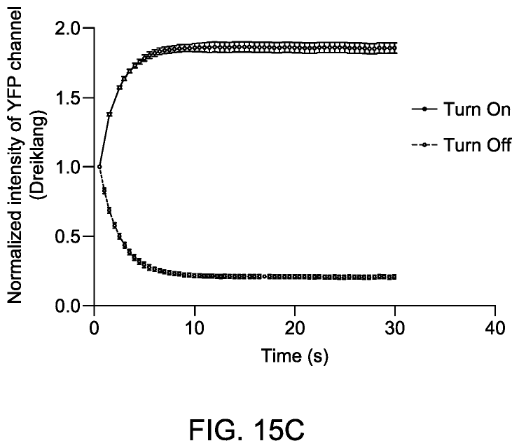

As it is challenging to measure subtle changes in PKA activity with current FRET-based biosensors, we aimed to generate FRET-based AKARs with even larger dynamic ranges. Since synthetic fluorophores have a larger extinction coefficient and higher brightness than FPs, using HaloTag and synthetic fluorophores as FRET acceptors could increase FRET efficiency. Besides, it was recently reported that HaloTags labeled with rhodamine based fluorophores and Aequorea victoria derived FPs form a favorable interface enhancing FRET efficiency^{[9]}. We therefore continued to employ HaloTag labeled with JF669 as the acceptor and set out to identify an optimal FP-based donor among different YFPs. Considering the distance between the two fluorophores and the effect of their orientation on FRET efficiency^{[13]}, we generated four different domain orientations for each of the different FPs (FIG. 5A). The different FRET-based AKARs were expressed in HeLa cells, the cells labeled with JF669 (FIG. 5B), and stimulated with 50 μM Fsk and 100 μM IBMX to induce maximal PKA activity. As expected, we observed a dramatic jump in the normalized FRET/donor ratio after stimulation and the response was reversible upon the addition of the PKA-specific inhibitor H89 (FIG. 5C). Compared with cpVenus173 and mPapaya, mCitrine-EV linker-HaloTag^{(JF669)} performed best with a dynamic range of 223.6%±4.6% (n=14 cells) [mean±SEM] for the ideal domain orientation (YFP-FHA1-EV linker-PKA substrate-HaloTag^{(JF669)} (FIG. 5D). We additionally tested if the dynamic range of this sensor could be further improved by using far-red acceptor fluorophores with slightly blue-shifted spectral properties. When testing the two fluorophores JF635 and JFX646 we found that labeling with JF669 led to the biosensor with the highest dynamic range (FIG. 14, Table 1)^{[19,20]} Furthermore, we tested if photoswitchable FPs could act as FRET donors. We generated a panel of biosensors using Dreiklang, ffDronpa, Skylan-S and rsEGFP2. Dreiklang-FHA1-EV linker-PKA substrate-HaloTag^{(JF669)} could successfully be switched on (365 nm) and off (405 nm) and showed a robust response upon stimulation with Fsk and IBMX (FIGS. 15A-15C).

We are anticipating that these biosensors will find applications in multiplexing strategies^{[21]} or super-resolution imaging.

### Optimizing the Length of the EV Linker in FRET-Based AKARs

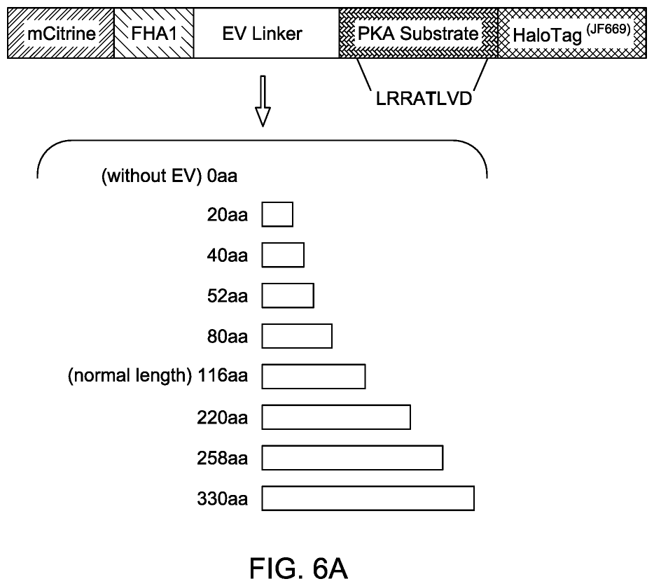

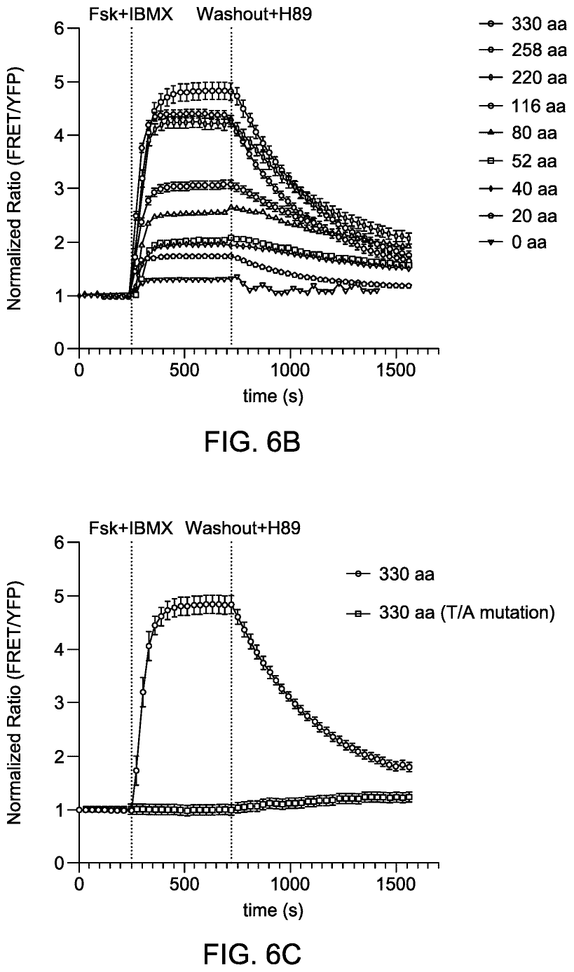

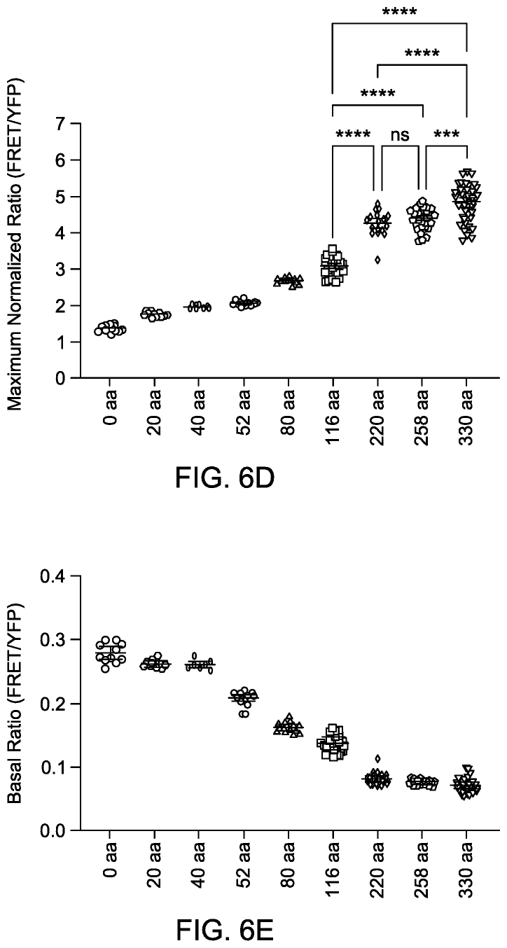

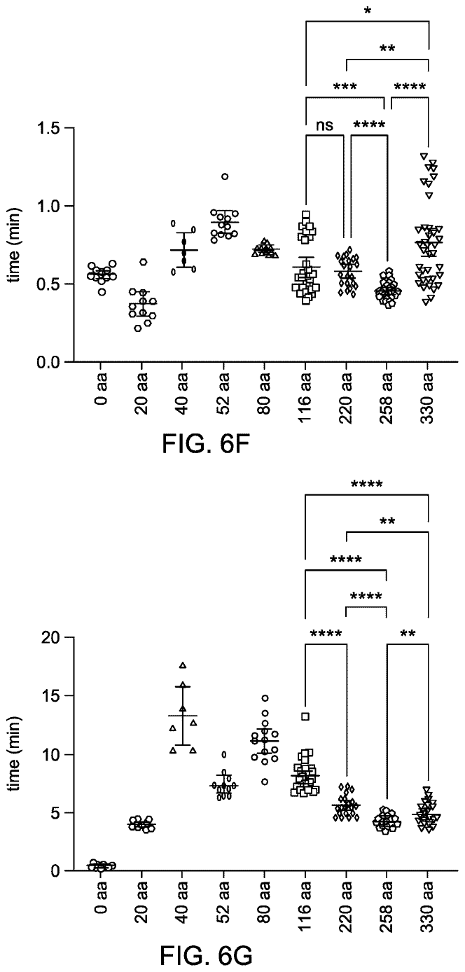

Given the improved dynamic ranges after optimization of the EV linker length for the sTagRFP-HaloTag(JF669) FRET pair, we also tested different lengths of EV linkers for mCitrine-HaloTag^{(JF669)} (FIG. 6A). After transfection of these reporters into HeLa cells and stimulation by 50 M Fsk and 100 μM IBMX rapid changes in emission ratios were visible (FIG. 6B). Among the biosensors, mCitrine-FHA1-330 aa EV linker-PKA substrate-HaloTag^{(JF669)} exhibited the best performance with a dynamic range of 386.2%±8.6% (n=38 cells) [mean±SEM](FIGS. 6D-6G). In contrast, HeLa cells expressing a T/A mutant showed no change in emission ratio upon Fsk and IBMX treatment (FIG. 6C), mCitrine-FHA1-330 aa EV linker-PKA substrate-HaloTag^{(JF669)} significantly outperforms existing FRET-based AKARs and shows the highest dynamic range of FRET-based AKARs reported so far. The enhanced sensitivity will enable us to monitor more subtle PKA activity changes and this biosensor is therefore ideally suited for high-throughput applications.

Generalizing the Biosensor Design (mCitrine-HaloTag^{(JF669)}) to Other Kinase Activities and Small-Molecule Sensing

mCitrine-HaloTag^{(JF669)} is a very promising FRET pair and we therefore tested if it could be used to generate more sensitive biosensors for other kinase activities. To do this, we replaced the PKA sensing domain in mCitrine-FHA1-330 aa EV linker-PKA substrate-HaloTag^{(JF669)} by PKC, Akt/PKB, ERK or cAMP sensing domains, generating FRET-based CKAR, FRET-based AktAR, FRET-based EKAR, and FRET-based ICUE using mCitrine.

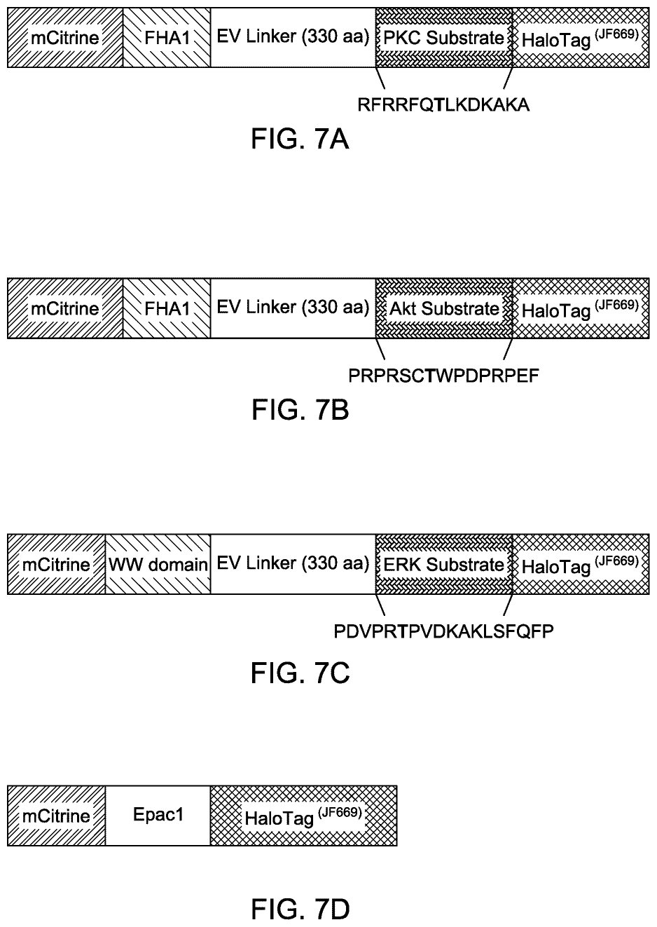

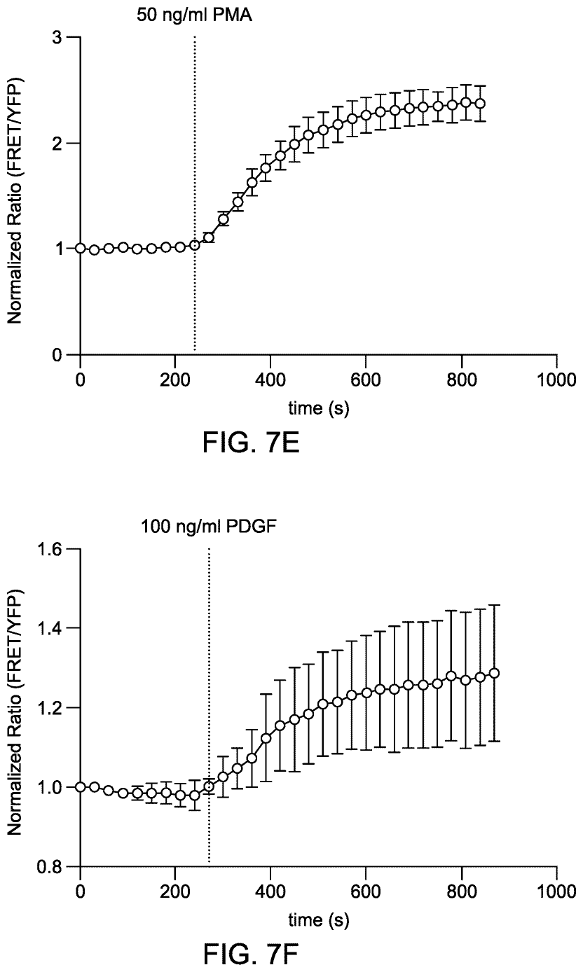

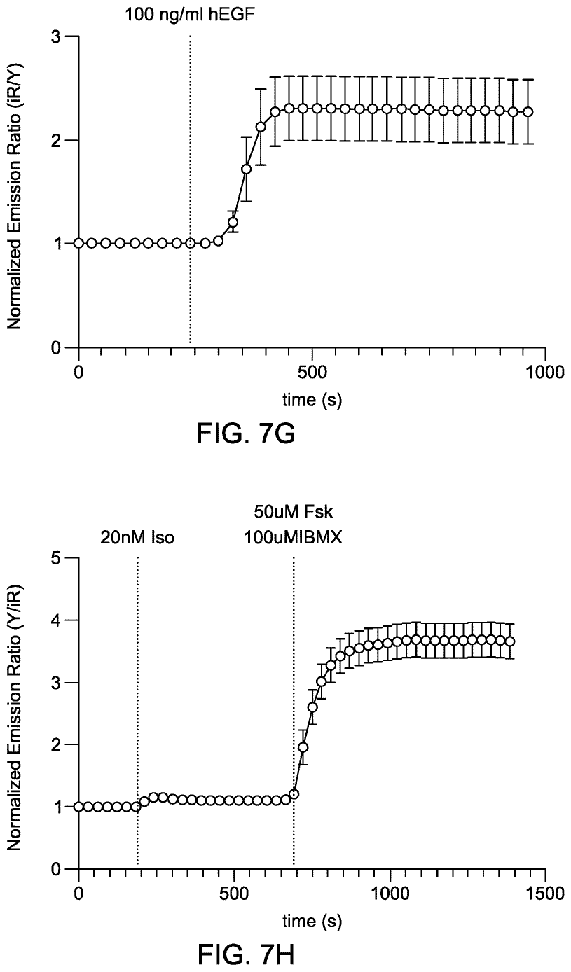

HaloTag^{(JF669)} (FIGS. 7A-7D). The CKAR reporter was expressed in HeLa cells and showed a dynamic range of 139.0%±4.2% (n=11 cells) [mean±SEM] after stimulation with PMA (FIG. 7E). The AktAR reporter was transfected in NIH3T3 cells and stimulated with PDGF. It showed a dynamic range of 29.2%±6.0% (n=6 cells) [mean±SEM](FIG. 7F), mCitrine-HaloTag^{(JF669)} based EKAR exhibited a dynamic range of 131.1%+±11.9% (n=8 cells) [mean±SEM] after the stimulation by hEGF in HEK293T cells (FIG. 7G). HaloTag based ICUE showed a dynamic range of 268.7%±11.0% (n=14 cells) [mean±SEM] after activation by Fsk and IBMX (FIG. 7H). According to these results, we expect that biosensors based on the mCitrine-HaloTag^{(JF669)} FRET pair will enable us to monitor multiple kinase activities as well as cAMP concentration with higher sensitivity.

### Enhanced FluoSTEPs Using HaloTag

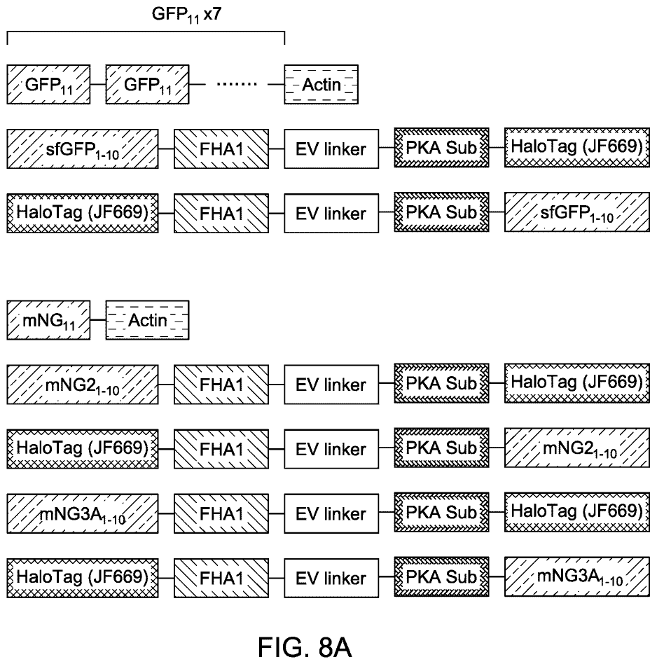

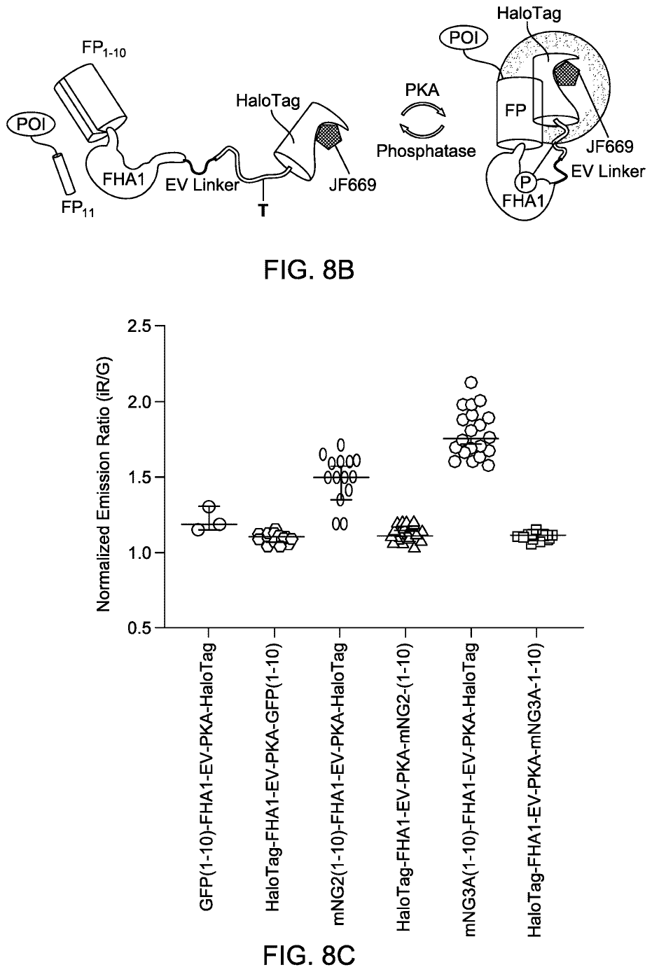

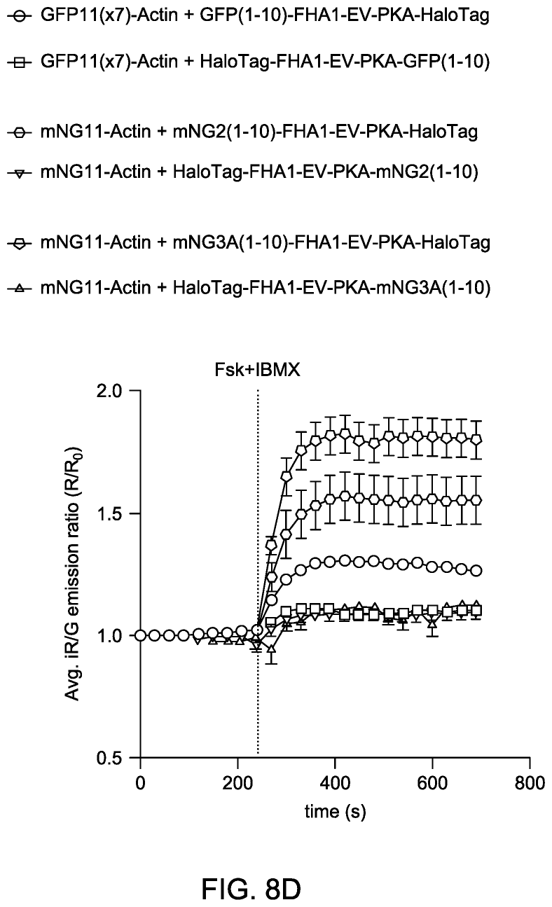

Recently, anew biosensor design was developed using self-complementing split green FP (GFP) as the FRET donor. The self-complementation allowed the biosensors to be recruited and reconstituted at proteins of interest (POI) endogenously tagged with the FPs eleventh-strand (FP₁₁). This allowed researchers to monitor compartmentalized signaling within endogenous microdomains without overexpressing the POI^{[11]}. However, this concept, fluorescent biosensors targeted to endogenous proteins (FluoSTEPs), suffers from two major limitations: low dynamic range and low brightness^{[11]}. Given the promising dynamic range achieved using HaloTag^{(JF669)} as a FRET acceptor in KARs, we decided to introduce HaloTag into FluoSTEP-AKAR. We replaced mRuby2 in FluoSTEP-AKAR with HaloTag and additionally introduced the EV linker to further increase the dynamic range. In order to address the limitation of low brightness of sfGFP as a FRET donor, we tested the use of two different split mNeonGreen (mNG) versions: split mNeonGreen2 (mNG2)^{[22]} and split mNeonGreen3A (mNG3A)[^{21]} in parallel (FIG. 8A). We co-expressed the generated biosensors with FP11-Actin in HeLa cells and used Fsk and IBMX to stimulate the reporters to monitor PKA signal. The best performing FluoSTEP, mNG3A₍₁₋₁₀₎-FHA1-EV linker-PKA substrate-HaloTag^{(JF669)} produced a 71.1%±3.1% (n=10 cells) [mean±SEM] increase when stimulated (FIGS. 8C-8D). Consistent with previous results biosensors with HaloTag at the C-terminus performed best. The dynamic range of this promising FluoSTEP is about 8 times that of the conventional one under the same experimental conditions[11].

### Variants of FluoSTEPs to Optimize Dynamic Range and Brightness

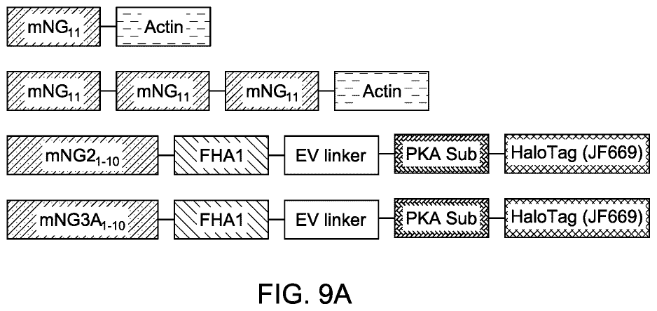

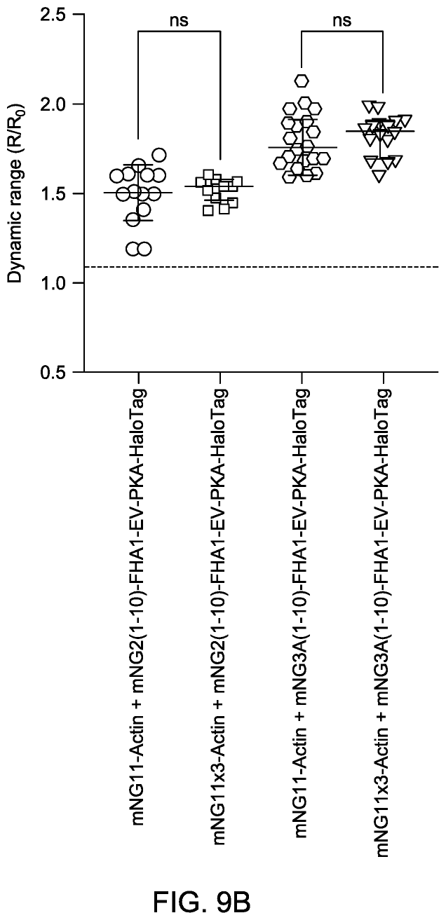

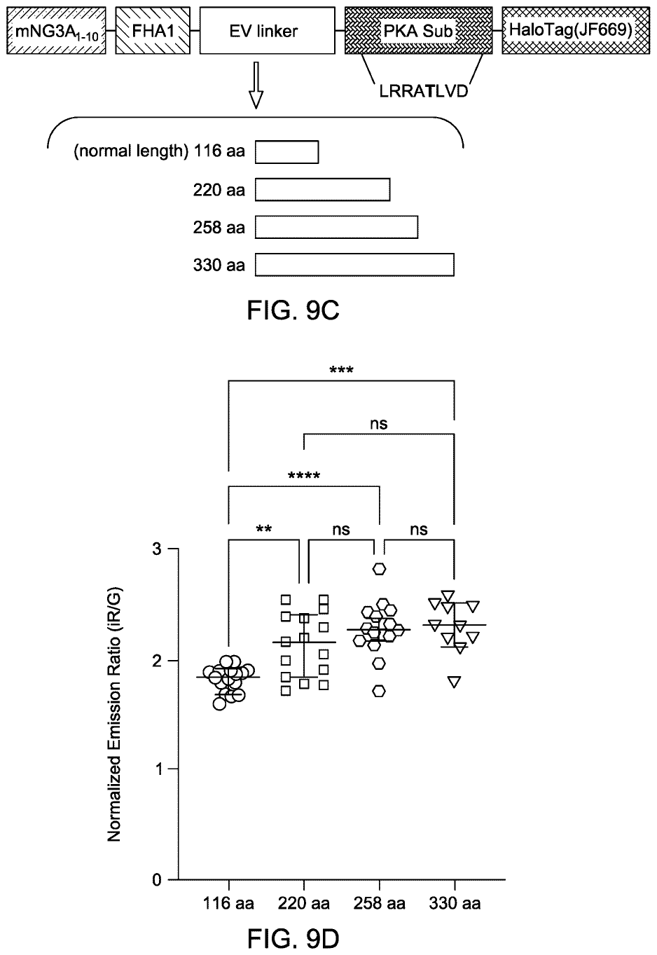

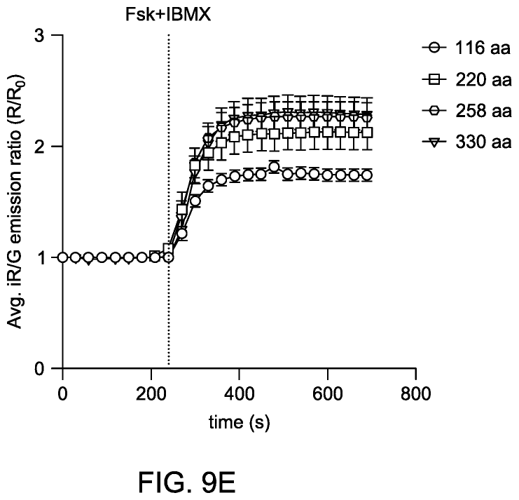

We further optimized the brightness of the enhanced FluoSTEP AKARs. To do this, we concatenated three mNG11 and fused them to actin, this method has effectively increase the brightness of the biosensor signal in previous studies^{[11]}. We tested the two best candidates, mNG2-FHA1-EV linker-PKA substrate-HaloTag, and mNG3A-FHA1-EV linker-PKA substrate-HaloTag (FIG. 9A). mNG11x3 did not show significant differences in terms of dynamic range when compared to a single mNG₁₁ for both mNG2 or mNG3A (FIG. 9B). Subsequently, we introduced longer EV linkers with 220 aa, 258 aa, and 330 aa linker length to decrease the basal emission ratio and thereby increase the dynamic range of the FluoSTEP-AKAR (FIG. 9C). Upon stimulation, the three FluoSTEP-AKARs containing different EV linkers showed significant higher dynamic range than the FluoSTEP-AKAR containing the normal length EV linker (116 aa) (FIGS. 9D-9E). We expect that the enhanced FluoSTEPs will allow us to monitor more subtle kinase activity changes on the endogenous level. We moreover anticipate that this concept will be generalizable to other kinase activities.

### Alternative HaloTag-Based FluoSTEP Design

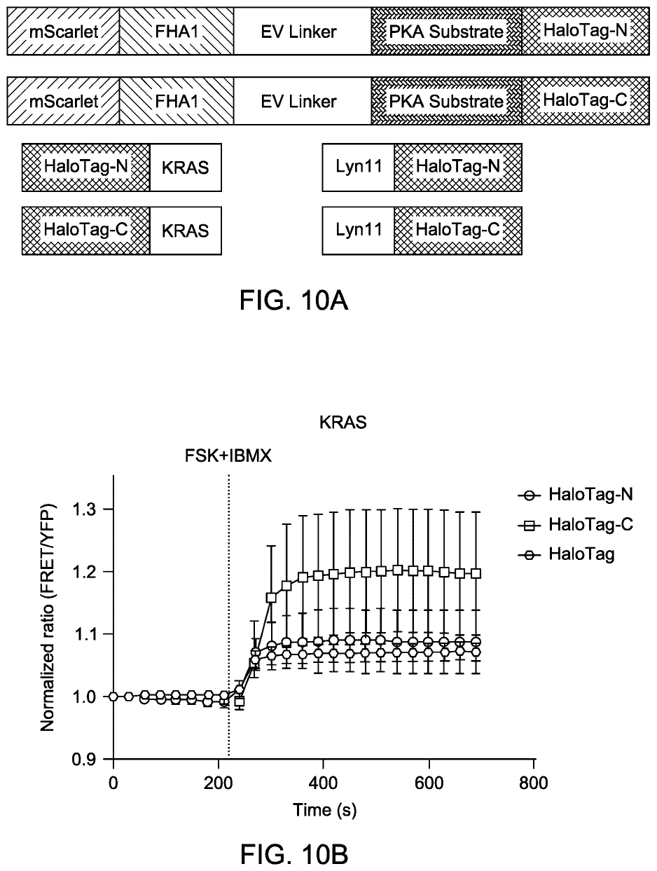

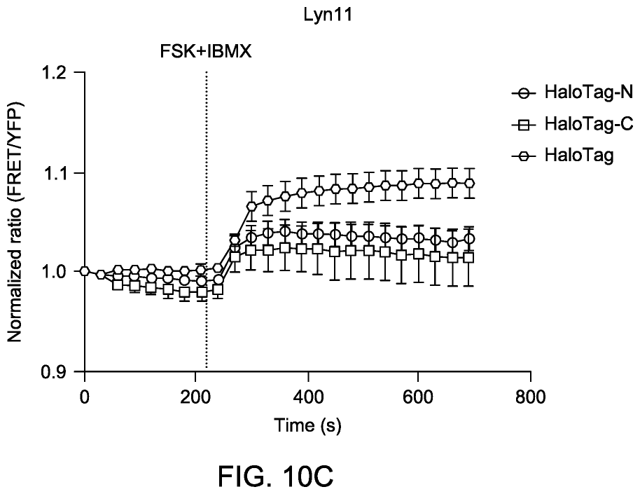

Instead of using a split FP HaloTag-based FluoSTEPs can also be generated using split HaloTag^{[24]}, which will then be used as the FRET donor. Based on mScarlet-FHA1-EV linker-PKA substrate-HaloTag we generated two FluoSTEP biosensors using either the N- or C-terminal part of split HaloTag (mScarlet-FHA1-EV linker-PKA substrate-HaloTag-N and mScarlet-FHA1-EV linker-PKA substrate-HaloTag-C, FIG. 10A). The biosensors were then co-expressed with HaloTag-C-KRAS or HaloTag-N-KRAS in HeLa cells. Both biosensors reconstituted successfully and were stimulated using Fsk and IBMX. Similar experiments were performed with a Lyn11 targeting sequences instead of KRAS (FIGS. 10B-10C). We additionally expect that replacement of mScarlet with Azalea-B5 and introduction of longer EV linkers will further improve the dynamic range of these FluoSTEPs.

### Methods

### Plasmid Generation

A pcDNA3.1 vector was used for transient expression in mammalian cells and all plasmids were cloned in this vector. Cloning was performed using standard molecular biology techniques including PCR amplification, Gibson cloning, restriction digest and ligation. Sequences were verified using Sanger sequencing. Entry plasmids were obtained from Addgene: mKOk: 98837, mRuby3 74252, FusionRed: 56103; mScarletI: 85066, mScarlet-H: 85067, Azalea-B5: 153521 mKate2: 37132, mCherry: 55102, mNeptune2: 41645, mNeptune2.5: 51310, mCitrin: 104839, mPapaya: 56396, miRFP720: 136574, mRhubarb720: 141201, rsEGFP2: 102879, Skylan-S: 86785, mNG3A: 157992, GFP1-10: 70219, GFP11: 70217HaloTag: 135444, cpHaloTag: 138327, Longer-EV-linker: 138373. Further entry plasmids were obtained from: ffDronpa: war provided by Peter Dedecker (University of Leuven), FusionMQV: was provided by Ralph Jimenez (University of Colorado Boulder), mNG, mNG2_1-10, and mNG11: were shared by Bo Huang (University of California, San Francisco).

### Cell Culture, Transfection, and HaloTag Labeling

Cell culture and transfection HeLa and HEK293T cells were cultured in Dulbecco's modified Eagle medium (DMEM; Gibco) containing glucose (1 g/L) and supplemented with 10% (v/v) fetal bovine serum (Sigma-Aldrich) and 1% (v/v) penicillin-streptomycin (Pen-Strep; Sigma-Aldrich). NIH3T3 cells were cultured in DMEM (Gibco) containing glucose (1 g/L) and supplemented with 10% (v/v) fetal calf serum and 1% (v/v) Pen-Strep (Sigma-Aldrich). All cells were maintained in a humidified incubator at 37° C. with a 5% CO2 atmosphere. Before transfection, cells were plated onto sterile quartered 35-mm glass-bottomed dishes and grown to 50 to 70% confluence. Cells were then transfected using Lipofectamine 2000 (Invitrogen, HeLa) or PolyJet (SignaGen Laboratories, HEK293T and NIH3T3) and grown an additional 48 hours (HeLa, HEK293T) before imaging. NIH3T3 cells were changed to serum-free DMEM immediately before transfection, medium was changed to complete medium after 6 h. NIH3T3 cells were then serum-starved for 24 hours before imaging. HaloTag labeling was performed 24 h before imaging using synthetic fluorophores (Fluorophore-CA 500 nM, 24 h, 37° C.) in the respective medium. Synthetic fluorophores were kindly provided by Luke Lavis (Janelia Research Campus, JF669-CA, JFX646-CA and JF635) or Kai Johnsson (Max Planck Institute for Medical Research (Germany) and EPFL (Switzerland), NGD-CA).

Cells were washed with the same medium (twice for 1 min, 37° C.). Cells were washed once with Hank's balanced salt solution (HBSS; Gibco) and subsequently imaged in HBSS in the dark at 37° C. Fsk (Calbiochem), IBMX (Sigma-Aldrich), H89 (Sigma-Aldrich), PMA (LC Laboratories), PDGF (Sigma-Aldrich), EGF (Sigma-Aldrich), and isoproterenol (Sigma) were added as indicated.

### Epifluorescence Microscopy

All epifluorescence imaging was performed on a AxioObserver Z1 Microscope equipped with a xenon/halogen lamp (XBO 75 Xenon arc lamp HAL100 Halogen lamp), a Sutter Lambda 10-3 that controls two separate filter wheels, a cooled EMCCD camera (Photometrics Evolve), a TempModule S (Zeiss), definite focus (fc12), and examined under a 40× oil immersion objective (Zeiss Plan Apochromate). FRET microscopy of biosensors was performed using the following excitation (EX) and emission (EM) filter combinations (maxima/bandwidth in nm):

### Image Analysis

Emission intensities of individual cells were background-subtracted, and the ratios between the donor and FRET channel were normalized to the starting point of the measurement (t=0 min).

### REFERENCES

1. Greenwald, E. C., S. Mehta, and J. Zhang, Genetically Encoded Fluorescent Biosensors Illuminate the Spatiotempora 1 Regulation of Signaling Networks. Chem Rev, 2018. 118(24): p. 11707-11794.
2. Kim, H., et al., Resource for FRET-Based Biosensor Optimization. Front Cell Dev Biol, 2022. 10: p. 885394.
3. Broch, F. and A. Gautier, Illuminating Cellular Biochemistry: Fluorogenic Chemogenetic Biosensors for Biologica 1 Imaging. Chempluschem. 2020. 85(7): p. 1487-1497.
4. Keppler, A., et al., A general method for the covalent labeling of fusion proteins with small molecules in vivo. Nat Biotechnol, 2003. 21(1): p. 86-9.
5. Los, G. V., et al., HaloTag: a novel protein labeling technology for cell imaging and protein analysis. ACS Chem Biol, 2008. 3(6): p. 373-82.
6. Lavis, L. D., Teaching Old Dyes New Tricks: Biological Probes Built from Fluoresceins and Rhodamines. Annu Rev Biochem, 2017. 86: p. 825-843.
7. Xue, L., et al., Imaging and manipulating proteins in live cells through covalent labeling. Nature Chemical Biology, 2015. 11(12): p. 917-923.
8. Matsu-ura, T., et al., Dual-FRET imaging of IP3 and Ca2-′-revealed Ca2-′-induced IP3 production maintains long lasting Ca2-′-oscillations in fertilized mouse eggs. Scientific Reports, 2019. 9(1): p. 4829.
9. Hellweg, L., et al., A general method for the development of multicolor biosensors with large dynamic ranges. bioRxiv, 2022: p. 2022.11.29.518186.
10. Vecchia, M. D., et al., Spectrally Tunable Förster Resonance Energy Transfer-Based Biosensors Using Organic Dye Grafting. ACS Sensors, 2022. 7(10): p. 2920-2927.
11. Tenner, B., et al., FluoSTEPs: Fluorescent biosensors for monitoring compartmentalized signaling within endogenous microdomains. Science Advances, 2021. 7(21): p. eabe4091.
12. Lardon, N., et al., Systematic Tuning of Rhodamine Spirocyclization for Super-resolution Microscopy. J Am Chem Soc, 2021. 143(36): p. 14592-14600.
13. Fritz, R. D., et al., A versatile toolkit to produce sensitive FRET biosensors to visualize signaling in time and space. Sci Signal, 2013. 6(285): p. rsl2.
14. Komatsu. N., et al., Development of an optimized backbone of TRET biosensors for kinases and GTPases. Mol Biol Cell, 2011. 22(23): p. 4647-56.
15. Ando, R., et al., Two new coral fluorescent proteins of distinct colors for sharp visualization of cell-cycle progression. bioRxiv, 2020: p. 2020.03.30.015156.
16. Deo, C., et al., The HaloTag as a general scaffold for far-red tunable chemigenetic indicators. Nature Chemical Biology, 2021. 17(6): p. 718-723.
17. Mo. G. C. H., et al., A rationally enhanced red fluorescent protein expands the utility of FRET biosensors. Nat Commun, 2020. 11(1): p. 1848.
18. Grimm, J. B., et al., A general method to optimize and functionalize red-shifted rhodamine dyes. Nat Methods, 2020. 17(8): p. 815-821.
19. Grimm. J. B., et al., A general method to fine-tune fluorophores for live-cell and in vivo imaging. Nat Methods, 2017. 14(10): p. 987-994.
20. Grimm, J. B., et al., A General Method to Improve Fluorophores Using Deuterated Auxochromes. JACS Au, 2021. 1(5): p. 690-696.
21. Roebroek, T., et al., Simultaneous readout of multiple FRET pairs using photochromism. Nat Commun, 2021. 12(1): p. 2005.
22. Feng. S., et al., Improved split fluorescent proteins for endogenous protein labeling. Nat Commun, 2017. 8(1): p. 370.
23. Zhou, S., et al., Improved yellow-green split fluorescent proteins for protein labeling and signal amplification. PLoS One, 2020. 15(11): p. e0242592.
24. Ishikawa, H., et al., Generation of a dual-functional split-reporter protein for monitoring membrane fusion using self-associating split GFP. Protein Eng Des Sel, 2012. 25(12): p. 813-20.

### Example 2: Enhanced FRET-Based Biosensors Using HaloTag and Synthetic Fluorophores

### 1. Multiplexed Biosensor Imaging Using HaloTag-Based FRET Biosensors.

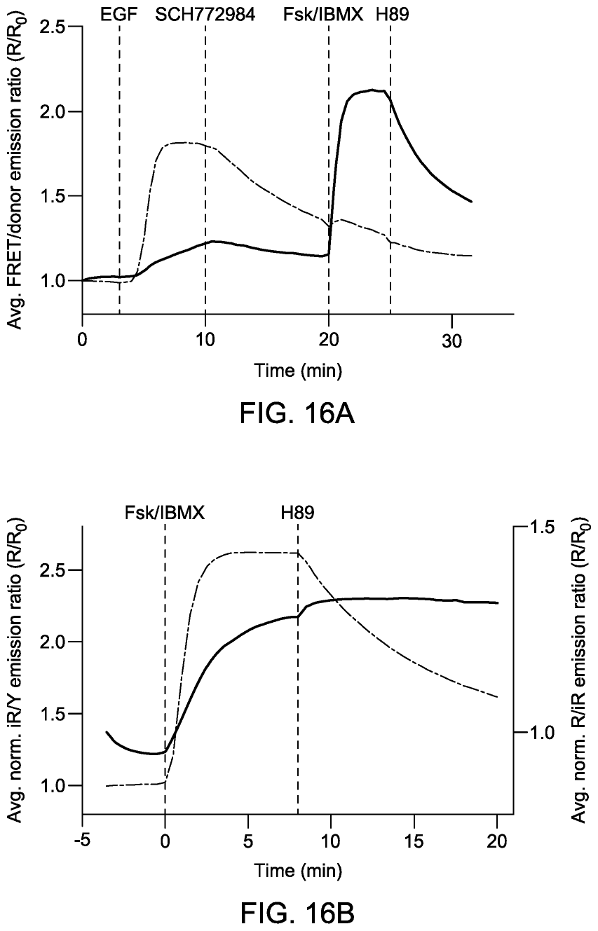

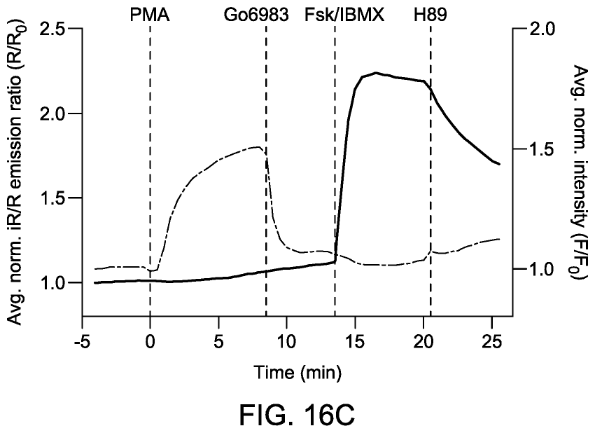

Combining biosensors based on the two most promising FRET-pairs mCitrine-HaloTag^{(JF669)} and sTagRFP-HaloTag^{(JF669)} allows us to perform multiplexing experiments as the two donors are spectrally well separated. Co-expression of mCitrine-WW domain-258 aa EV linker-ERK substrate-HaloTag^{(JF669)} (yellow/far-red HaloTag-EKAR) and sTagRFP-FHA1-314 aa EV linker-PKA substrate-HaloTag^{(JF669)} (red/far-red HaloTag-AKAR) in HEK293T cells allowed us to follow ERK activity and PKA response upon pharmacological stimulation. Stimulation with 100 ng/mL human epidermal growth factor (hEGF) led to an increase in both ERK and PKA activity, while treatment with the ERK inhibitor SCH772984 (1.7 M) only decreased ERK activity. Treatment with 50 M forskolin (Fsk) and 100 μM 3-isobutyl-1-methylxanthine (IBMX) led to a rapid increase in PKA activity and treatment with M H89 to a decrease in PKA activity (FIG. 16A)

Similarly, mCitrine-FHA1-258 aa EV linker-PKA substrate-HaloTag^{(JF669)} (yellow/far-red-HaloTag-AKAR) and sTagRFP-Epac-HaloTag^{(JF669)} (red/far-red HaloTag-ICUE) can be co-imaged to investigate the concentration of cAMP and the activity of PKA simultaneously (FIG. 16B). We could further combine our red/far-red biosensors with excitation ratiometric ExRai sensors in the green spectral region^{1,2}. Combination of ExRaiCKAR¹ with sTagRFP-FHA1-314 aa EV linker-PKA substrate-HaloTag^{(JF669)} (red/far-red HaloTag-AKAR) allowed us to co-image PKC and PKA activity in HeLa cells (FIG. 16C).

2. Measurements of Kinase Activity Using Enhanced FluoSTEPs Targeted to Proteins on their Endogenous Level.

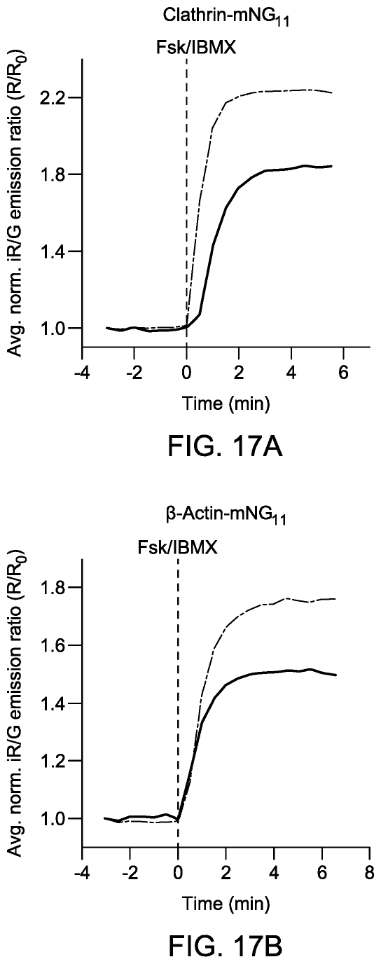

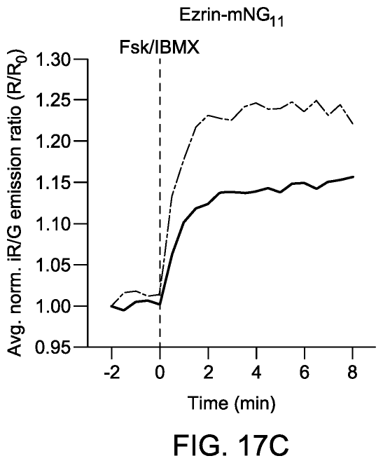

Using our enhanced HaloTag-based FluoSTEPs mNG3A₍₁₋₁₀₎-FHA1-xx aa EV linker-PKA substrate-HaloTag^{(JF669)} (xx=220 and 116 aa) we measured PKA activation upon treatment with 50 M Fsk and 100 μM IBMX in HeLa cells endogenously expressing mNG₁₁ at different locations (CRISPER-mediated knock-in). For all three cell lines (HeLa-mNG₁₁-β-Actin, HeLa-mNG₁₁-clathrin, and HeLa-mNG₁₁-ezrin) the biosensor with the 116 aa EV linker showed a diminished response compared to the sensor with the 220 aa EV linker (FIGS. 17A-17C).

### 3. Generalizing the Design of HaloTag-Based Chemigenetic Biosensors.

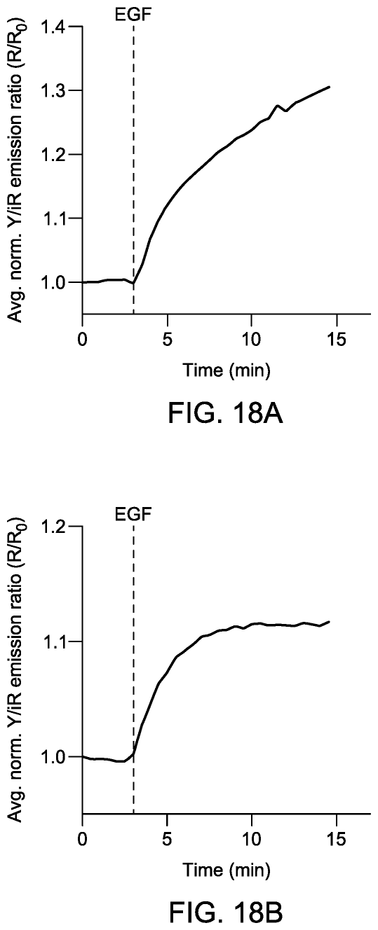

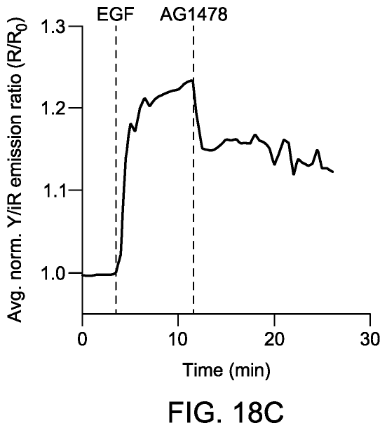

With the promising properties of HaloTag-based chemigenetic biosensors, the design can be generalized to a variety of cell signaling reporters. Thus, using the most promising FRET-pairs mCitrine-HaloTag^{(JF669)}, we further generated several FRET-based chemigenetic biosensors, they are: mCitrine-SH2-Src Sub-HaloTag^{(JF669)} (yellow/far-red HaloTag-SrcAR), mCitrine-SH2-Fyn Sub-HaloTag^{(JF669)} (yellow/far-red HaloTag-Fyn sensor), and mCitrine-RBD-PL-HaloTag^{(JF669)} (yellow/far-red HaloTag-RasAR) (FIGS. 18A-18C).

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B:” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together. A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. For example, the logic flows may include different and/or additional operations than shown without departing from the scope of the present disclosure. One or more operations of the logic flows may be repeated and/or omitted without departing from the scope of the present disclosure. Other implementations may be within the scope of the following claims.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B:” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. For example, the logic flows may include different and/or additional operations than shown without departing from the scope of the present disclosure. One or more operations of the logic flows may be repeated and/or omitted without departing from the scope of the present disclosure. Other implementations may be within the scope of the following claims.

TABLE 1
Structures and references of chemical fluorophores described.

| Name | Structure | Reference |
|---|---|---|
| NGD | 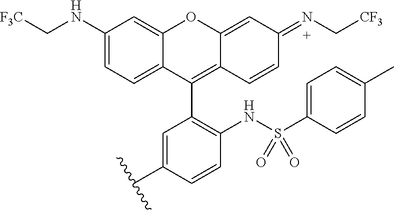 [SMILES: *c1ccc(NS(=O)(=O)c2ccc(C)cc2)c(-c2c3cc/c(=N\CC(F)(F)F)cc-3oc3cc(NCC(F)(F)F)ccc23)c1] | [12] |
| JF669 | 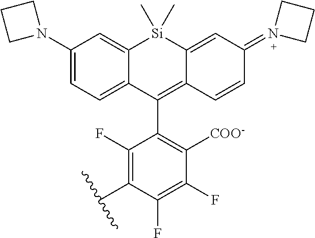 | [18] |
| JFX646 | 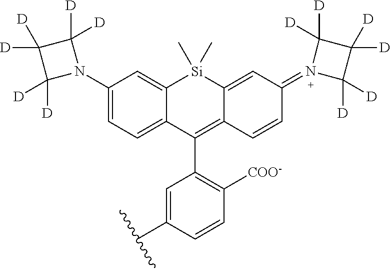 | [20] |
| JF635 | 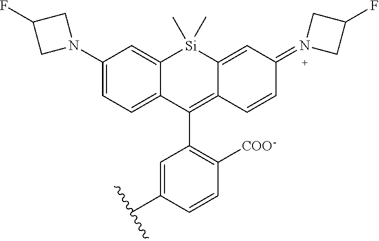 | [19] |

TABLE 2
Exemplary biosensor recombinant proteins provided herein.

| | | | | EV |
|---|---|---|---|---|
| | | | Sensing | linker |
| Name | Donor | Acceptor | domain | length |
| FIG. 1 | | | | |
| HaloTag - FHA1 - PKA | HaloTag^{(NGD)} | mScarlet | PKA | / |
| sub - mScarlet | | | | |
| mScarlet - FHA1 - PKA | HaloTag^{(NGD)} | mScarlet | PKA | / |
| sub - HaloTag | | | | |
| HaloTag - FHA1 - EV | HaloTag^{(NGD)} | mScarlet | PKA | 116 aa |
| linker - PKA sub - mScarlet | | | | |
| mScarlet - FHA1 - EV | HaloTag^{(NGD)} | mScarlet | PKA | 116 aa |
| linker - PKA sub - HaloTag | | | | |
| HaloTag - FHA1 - PKA | HaloTag^{(NGD)} | stagRFP | PKA | / |
| sub - stagRFP | | | | |
| stagRFP - FHA1 - PKA | HaloTag^{(NGD)} | stagRFP | PKA | / |
| sub - HaloTag | | | | |
| HaloTag - FHA1 - EV | HaloTag^{(NGD)} | stagRFP | PKA | 116 aa |
| linker - PKA sub - stagRFP | | | | |
| stagRFP - FHA1 - EV | HaloTag^{(NGD)} | stagRFP | PKA | 116 aa |
| linker - PKA sub - HaloTag | | | | |
| HaloTag - FHA1 - PKA | HaloTag^{(NGD)} | mKOk | PKA | / |
| sub - mKOk | | | | |
| mKOk - FHA1 - PKA sub - | HaloTag^{(NGD)} | mKOk | PKA | / |
| HaloTag | | | | |
| HaloTag - FHA1 - EV | HaloTag^{(NGD)} | mKOk | PKA | 116 aa |
| linker - PKA sub - mKOk | | | | |
| mKOk - FHA1 - EV linker - | HaloTag^{(NGD)} | mKOk | PKA | 116 aa |
| PKA sub - HaloTag | | | | |
| HaloTag - FHA1 - PKA | HaloTag^{(NGD)} | mRuby3 | PKA | / |
| sub - mRuby3 | | | | |
| mRuby3 - FHA1 - PKA | HaloTag^{(NGD)} | mRuby3 | PKA | / |
| sub - HaloTag | | | | |
| HaloTag - FHA1 - EV | HaloTag^{(NGD)} | mRuby3 | PKA | 116 aa |
| linker - PKA sub - mRuby3 | | | | |
| mRuby3 - FHA1 - EV | HaloTag^{(NGD)} | mRuby3 | PKA | 116 aa |
| linker - PKA sub - HaloTag | | | | |
| HaloTag - FHA1 - PKA | HaloTag^{(NGD)} | FusionMQV | PKA | / |
| sub - FusionMQV | | | | |
| FusionMQV - FHA1 - PKA | HaloTag^{(NGD)} | FusionMQV | PKA | / |
| sub - HaloTag | | | | |
| HaloTag - FHA1 - EV | HaloTag^{(NGD)} | FusionMQV | PKA | 116 aa |
| linker - PKA sub - FusionMQV | | | | |
| FusionMQV - FHA1 - EV | HaloTag^{(NGD)} | FusionMQV | PKA | 116 aa |
| linker - PKA sub - HaloTag | | | | |
| HaloTag - FHA1 - PKA | HaloTag^{(NGD)} | FusionRed | PKA | / |
| sub - FusionRed | | | | |
| FusionRed - FHA1 - PKA | HaloTag^{(NGD)} | FusionRed | PKA | / |
| sub - HaloTag | | | | |
| HaloTag - FHA1 - EV | HaloTag^{(NGD)} | FusionRed | PKA | 116 aa |
| linker - PKA sub - FusionRed | | | | |
| FusionRed - FHA1 - EV | HaloTag^{(NGD)} | FusionRed | PKA | 116 aa |
| linker - PKA sub - HaloTag | | | | |
| HaloTag - FHA1 - PKA | HaloTag^{(NGD)} | mScarlet-I | PKA | / |
| sub - mScarlet-I | | | | |
| mScarlet-I - FHA1 - PKA | HaloTag^{(NGD)} | mScarlet-I | PKA | / |
| sub - HaloTag | | | | |
| HaloTag - FHA1 - EV | HaloTag^{(NGD)} | mScarlet-I | PKA | 116 aa |
| linker - PKA sub - mScarlet-I | | | | |
| mScarlet-I - FHA1 - EV | HaloTag^{(NGD)} | mScarlet-I | PKA | 116 aa |
| linker - PKA sub - HaloTag | | | | |
| HaloTag - FHA1 - PKA | HaloTag^{(NGD)} | Azalea-B5 | PKA | / |
| sub - Azalea-B5 | | | | |
| Azalea-B5 - FHA1 - PKA | HaloTag^{(NGD)} | Azalea-B5 | PKA | / |
| sub - HaloTag | | | | |
| HaloTag - FHA1 - EV | HaloTag^{(NGD)} | Azalea-B5 | PKA | 116 aa |
| linker - PKA sub - Azalea-B5 | | | | |
| Azalea-B5 - FHA1 - EV | HaloTag^{(NGD)} | Azalea-B5 | PKA | 116 aa |
| linker - PKA sub - HaloTag | | | | |
| HaloTag - FHA1 - PKA | HaloTag^{(NGD)} | mKate2 | PKA | / |
| sub - mKate2 | | | | |
| mKate2- FHA1 - PKA sub - | HaloTag^{(NGD)} | mKate2 | PKA | / |
| HaloTag | | | | |
| HaloTag - FHA1 - EV | HaloTag^{(NGD)} | mKate2 | PKA | 116 aa |
| linker - PKA sub - mKate2 | | | | |
| mKate2- FHA1 - EV linker - | HaloTag^{(NGD)} | mKate2 | PKA | 116 aa |
| PKA sub - HaloTag | | | | |
| HaloTag - FHA1 - PKA | HaloTag^{(NGD)} | mCherry | PKA | / |
| sub - mCherry | | | | |
| mCherry - FHA1 - PKA | HaloTag^{(NGD)} | mCherry | PKA | / |
| sub - HaloTag | | | | |
| HaloTag - FHA1 - EV | HaloTag^{(NGD)} | mCherry | PKA | 116 aa |
| linker - PKA sub - mCherry | | | | |
| mCherry - FHA1 - EV | HaloTag^{(NGD)} | mCherry | PKA | 116 aa |
| linker - PKA sub - HaloTag | | | | |
| HaloTag - FHA1 - PKA | HaloTag^{(NGD)} | mNeptune2 | PKA | / |
| sub - mNeptune2 | | | | |
| mNeptune2- FHA1 - PKA | HaloTag^{(NGD)} | mNeptune2 | PKA | / |
| sub - HaloTag | | | | |
| HaloTag - FHA1 - EV | HaloTag^{(NGD)} | mNeptune2 | PKA | 116 aa |
| linker - PKA sub - mNeptune2 | | | | |
| mNeptune2- FHA1 - EV | HaloTag^{(NGD)} | mNeptune2 | PKA | 116 aa |
| linker - PKA sub - HaloTag | | | | |
| HaloTag - FHA1 - PKA | HaloTag^{(NGD)} | mNeptune2.5 | PKA | / |
| sub - Azalea-B5 | | | | |
| mNeptune2.5- FHA1 - PKA | HaloTag^{(NGD)} | mNeptune2.5 | PKA | / |
| sub - HaloTag | | | | |
| HaloTag - FHA1 - EV | HaloTag^{(NGD)} | mNeptune2.5 | PKA | 116 aa |
| linker - PKA sub - mNeptune2.5 | | | | |
| mNeptune2.5- FHA1 - EV | HaloTag^{(NGD)} | mNeptune2.5 | PKA | 116 aa |
| linker - PKA sub - HaloTag | | | | |
| FIG. 2 | | | | |
| mScarlet - FHA1 - EV | mScarlet | HaloTag^{(JF669)} | PKA | 116 aa |
| linker - PKA sub - HaloTag | | | | |
| stagRFP - FHA1 - EV | stagRFP | HaloTag^{(JF669)} | PKA | 116 aa |
| linker - PKA sub - HaloTag | | | | |
| mRuby3 - FHA1 - EV | mRuby3 | HaloTag^{(JF669)} | PKA | 116 aa |
| linker - PKA sub - HaloTag | | | | |
| Azalea-B5 - FHA1 - EV | Azalea-B5 | HaloTag^{(JF669)} | PKA | 116 aa |
| linker - PKA sub - HaloTag | | | | |
| mNeptune2- FHA1 - EV | mNeptune2 | HaloTag^{(JF669)} | PKA | 116 aa |
| linker - PKA sub - HaloTag | | | | |
| mKOk - FHA1 - EV linker - | mKOk | HaloTag^{(JF669)} | PKA | 116 aa |
| PKA sub - HaloTag | | | | |
| FusionMQV - FHA1 - EV | FusionMQV | HaloTag^{(JF669)} | PKA | 116 aa |
| linker - PKA sub - HaloTag | | | | |
| FusionRed - FHA1 - EV | FusionRed | HaloTag^{(JF669)} | PKA | 116 aa |
| linker - PKA sub - HaloTag | | | | |
| mScarlet-I - FHA1 - EV | mScarlet-I | HaloTag^{(JF669)} | PKA | 116 aa |
| linker - PKA sub - HaloTag | | | | |
| mCherry - FHA1 - EV | mCherry | HaloTag^{(JF669)} | PKA | 116 aa |
| linker - PKA sub - HaloTag | | | | |
| FIG. 3 | | | | |
| stagRFP - FHA1 - EV | stagRFP | HaloTag^{(JF669)} | PKA | 116 aa |
| linker - PKA sub - HaloTag | | | | |
| sTagRFP - FHA1 - 142aa | sTagRFP | HaloTag^{(JF669)} | PKA | 142 aa |
| EV - PKA sub - HaloTag | | | | |
| sTagRFP - FHA1 - 190aa | sTagRFP | HaloTag^{(JF669)} | PKA | 190 aa |
| EV - PKA sub - HaloTag | | | | |
| sTagRFP - FHA1 - 220aa | sTagRFP | HaloTag^{(JF669)} | PKA | 220 aa |
| EV - PKA sub - HaloTag | | | | |
| sTagRFP - FHA1 - 258aa | sTagRFP | HaloTag^{(JF669)} | PKA | 258 aa |
| EV - PKA sub - HaloTag | | | | |
| sTagRFP - FHA1 - 330aa | sTagRFP | HaloTag^{(JF669)} | PKA | 330 aa |
| EV - PKA sub - HaloTag | | | | |
| sTagRFP - FHA1 - 190 aa | sTagRFP | HaloTag^{(JF669)} | PKA | 190 aa |
| EV - PKA sub - HaloTag | | | | |
| (T/A mutation) | | | | |
| FIG. 4 | | | | |
| sTagRFP - FHA1 - 330 aa | sTagRFP | HaloTag^{(JF669)} | PKC | 330 aa |
| EV - PKC sub - HaloTag | | | | |
| sTagRFP - FHA1 - 330 aa | sTagRFP | HaloTag^{(JF669)} | Akt | 330 aa |
| EV - Akt sub - HaloTag | | | | |
| sTagRFP - WW domain - | sTagRFP | HaloTag^{(JF669)} | ERK | 330 aa |
| 330 aa EV - Erk sub - | | | | |
| HaloTag | | | | |
| sTagRFP - Epac1 - HaloTag | sTagRFP | HaloTag^{(JF669)} | cAMP | / |
| FIG. 5 | | | | |
| HaloTag - FHA1 - PKA | mCitrine | HaloTag^{(JF669)} | PKA | / |
| sub - mCitrine | | | | |
| mCitrine - FHA1 - PKA | mCitrine | HaloTag^{(JF669)} | PKA | / |
| sub - HaloTag | | | | |
| HaloTag - FHA1 - EV | mCitrine | HaloTag^{(JF669)} | PKA | 116 aa |
| linker - PKA sub - mCitrine | | | | |
| mCitrine - FHA1 - EV | mCitrine | HaloTag^{(JF669)} | PKA | 116 aa |
| linker - PKA sub - HaloTag | | | | |
| HaloTag - FHA1 - PKA | cpVenus | HaloTag^{(JF669)} | PKA | / |
| sub - cp Venus | | | | |
| cp Venus - FHA1 - PKA | cpVenus | HaloTag^{(JF669)} | PKA | / |
| sub - HaloTag | | | | |
| cp Venus - FHA1 - EV | cp Venus | HaloTag^{(JF669)} | PKA | 116 aa |
| linker - PKA sub - HaloTag | | | | |
| HaloTag - FHA1 - EV | cpVenus | HaloTag^{(JF669)} | PKA | 116 aa |
| linker - PKA sub - cpVenus | | | | |
| mPapaya - FHA1 - EV | mPapaya | HaloTag^{(JF669)} | PKA | 116 aa |
| linker - PKA sub - HaloTag | | | | |
| FIG. 6 | | | | |
| mCitrine - FHA1 - PKA | mCitrine | HaloTag^{(JF669)} | PKA | / |
| sub - Halo Tag | | | | |
| mCitrine - FHA1 - 20aa | mCitrine | HaloTag^{(JF669)} | PKA | 20 aa |
| EV - PKA sub - HaloTag | | | | |
| mCitrine - FHA1 - 40aa | mCitrine | HaloTag^{(JF669)} | PKA | 40 aa |
| EV - PKA sub - HaloTag | | | | |
| mCitrine - FHA1 - 52aa | mCitrine | HaloTag^{(JF669)} | PKA | 52 aa |
| EV - PKA sub - HaloTag | | | | |
| mCitrine - FHA1 - 80aa | mCitrine | HaloTag^{(JF669)} | PKA | 80 aa |
| EV - PKA sub - HaloTag | | | | |
| mCitrine - FHA1 - EV | mCitrine | HaloTag^{(JF669)} | PKA | 116 aa |
| linker - PKA sub - HaloTag | | | | |
| mCitrine - FHA1 - 220aa | mCitrine | HaloTag^{(JF669)} | PKA | 220 aa |
| EV - PKA sub - HaloTag | | | | |
| mCitrine - FHA1 - 258aa | mCitrine | HaloTag^{(JF669)} | PKA | 258 aa |
| EV - PKA sub - HaloTag | | | | |
| mCitrine - FHA1 - 330aa | mCitrine | HaloTag^{(JF669)} | PKA | 330 aa |
| EV - PKA sub - HaloTag | | | | |
| mCitrine - FHA1 - 330aa | mCitrine | HaloTag^{(JF669)} | PKA | 330 aa |
| EV - PKA sub - HaloTag | | | | |
| (T/A mutation) | | | | |
| FIG. 7 | | | | |
| mCitrine - FHA1 - 330aa | mCitrine | HaloTag^{(JF669)} | PKC | 330 aa |
| EV - PKC sub - HaloTag | | | | |
| mCitrine - FHA1 - 330aa | mCitrine | HaloTag^{(JF669)} | Akt | 330 aa |
| EV - Akt sub - HaloTag | | | | |
| mCitrine - WW domain - | mCitrine | HaloTag^{(JF669)} | ERK | 330 aa |
| 330aa EV - Erk sub - | | | | |
| HaloTag | | | | |
| mCitrine - Epac1 - HaloTag | mCitrine | HaloTag^{(JF669)} | cAMP | / |
| FIG. 8 | | | | |
| GFP11 (x7) - Actin | / | / | / | / |
| GFP (1-10) - FHA1 - 116aa | sfGFP | HaloTag^{(JF669)} | PKA | 116 aa |
| EV - PKA sub - HaloTag | | | | |
| HaloTag - FHA1 - 116aa | sfGFP | HaloTag^{(JF669)} | PKA | 116 aa |
| EV - PKA sub - GFP (1-10) | | | | |
| mNG11 - Actin | / | / | / | / |
| mNG2 (1-10) - FHA1 - | Split mNG2 | HaloTag^{(JF669)} | PKA | 116 aa |
| 116aa EV - PKA sub - | | | | |
| HaloTag | | | | |
| HaloTag - FHA1 - 116aa | Split mNG2 | HaloTag^{(JF669)} | PKA | 116 aa |
| EV - PKA sub - mNG2 | | | | |
| (1-10) | | | | |
| mNG3A (1-10) - FHA1 - | Split mNG3A | HaloTag^{(JF669)} | PKA | 116 aa |
| 116aa EV - PKA sub - | | | | |
| HaloTag | | | | |
| HaloTag - FHA1 - 116aa | Split mNG3A | HaloTag^{(JF669)} | PKA | 116 aa |
| EV - PKA sub - mNG3A | | | | |
| (1-10) | | | | |
| FIG. 9 | | | | |
| mNG11 - Actin | / | / | / | / |
| mNG11 (x3) - Actin | / | / | / | / |
| mNG2 (1-10) - FHA1 - | Split mNG2 | HaloTag^{(JF669)} | PKA | 116 aa |
| 116aa EV - PKA sub - | | | | |
| HaloTag | | | | |
| mNG3A (1-10) - FHA1 - | Split mNG3A | HaloTag^{(JF669)} | PKA | 116 aa |
| 116aa EV - PKA sub - | | | | |
| HaloTag | | | | |
| mNG3A (1-10) - FHA1 - | Split mNG3A | HaloTag^{(JF669)} | PKA | 220 aa |
| 220aa EV - PKA sub - | | | | |
| HaloTag | | | | |
| mNG3A (1-10) - FHA1 - | Split mNG3A | HaloTag^{(JF669)} | PKA | 258 aa |
| 258aa EV - PKA sub - | | | | |
| HaloTag | | | | |
| mNG3A (1-10) - FHA1 - | Split mNG3A | HaloTag^{(JF669)} | PKA | 330 aa |
| 330aa EV - PKA sub - | | | | |
| HaloTag | | | | |
| FIG. 10 | | | | |
| mScarlet - FHA1 - EV | HaloTag^{(NGD)} | mScarlet | PKA | 116 aa |
| linker - PKA sub - | | | | |
| HaloTag - KRAS | | | | |
| Lynn11-mScarlet - FHA1 - | HaloTag^{(NGD)} | mScarlet | PKA | 116 aa |
| EV linker - PKA sub - | | | | |
| HaloTag | | | | |
| mScarlet - FHA1 - EV | HaloTag^{(NGD)} | mScarlet | PKA | 116 aa |
| linker - PKA sub - HaloTag- | | | | |
| mScarlet - FHA1 - EV | HaloTag^{(NGD)} | mScarlet | PKA | 116 aa |
| linker - PKA sub - HaloTag- | | | | |
| C | | | | |
| HaloTag-N - KRAS | / | / | / | / |
| HaloTag-C - KRAS | / | / | / | / |
| Lyn11 - HaloTag-N | / | / | / | / |
| Lyn11 - HaloTag-C | / | / | / | / |
| FIG. 11 | | | | |
| HaloTag - FHA1 - PKA | HaloTag^{(NGD)} | mScarlet | PKA | / |
| sub - mScarlet | | | | |
| mScarlet - FHA1 - PKA | HaloTag^{(NGD)} | mScarlet | PKA | / |
| sub - HaloTag | | | | |
| HaloTag - FHA1 - EV | HaloTag^{(NGD)} | mScarlet | PKA | 116 aa |
| linker - PKA sub - mScarlet | | | | |
| mScarlet - FHA1 - EV | HaloTag^{(NGD)} | mScarlet | PKA | 116 aa |
| linker - PKA sub - HaloTag | | | | |
| mScarlet - FHA1 - PKA | cpHaloTag^{(NGD)} | mScarlet | PKA | / |
| sub - cpHaloTag | | | | |
| mScarlet - FHA1 - EV | cpHaloTag^{(NGD)} | mScarlet | PKA | 116 aa |
| linker - PKA sub - | | | | |
| cpHaloTag | | | | |
| cpHaloTag - FHA1 - PKA | cpHaloTag^{(NGD)} | mScarlet | PKA | / |
| sub - mScarlet | | | | |
| cpHaloTag - FHA1 - EV | cpHaloTag^{(NGD)} | mScarlet | PKA | 116 aa |
| linker - PKA sub - mScarlet | | | | |
| FIG. 12 | | | | |
| mScarlet - FHA1 - EV | HaloTag^{(NGD)} | mScarlet | PKA | 116 aa |
| linker - PKA sub - HaloTag | | | | |
| mScarlet - FHA1 - EV | cpHaloTag^{(NGD)} | mScarlet | PKA | 116 aa |
| linker - HaloTag - PKA sub | | | | |
| FHA1 - mScarlet - EV | cpHaloTag^{(NGD)} | mScarlet | PKA | 116 aa |
| linker - HaloTag - PKA sub | | | | |
| PKA sub - mScarlet - EV | cpHaloTag^{(NGD)} | mScarlet | PKA | 116 aa |
| linker - HaloTag - FHA1 | | | | |
| PKA sub - HaloTag - EV | cpHaloTag^{(NGD)} | mScarlet | PKA | 116 aa |
| linker - mScarlet - FHA1 | | | | |
| FHA1 - HaloTag - EV | cpHaloTag^{(NGD)} | mScarlet | PKA | 116 aa |
| linker - mScarlet - PKA sub | | | | |
| mScarlet - FHA1 - EV | mScarlet | HaloTag^{(JF669)} | PKA | 116 aa |
| linker - PKA sub - HaloTag | | | | |
| mScarlet - FHA1 - EV | mScarlet | HaloTag^{(JF669)} | PKA | 116 aa |
| linker - HaloTag - PKA sub | | | | |
| FHA1 - mScarlet - EV | mScarlet | HaloTag^{(JF669)} | PKA | 116 aa |
| linker - HaloTag - PKA sub | | | | |
| PKA sub - mScarlet - EV | mScarlet | HaloTag^{(JF669)} | PKA | 116 aa |
| linker - HaloTag - FHA1 | | | | |
| PKA sub - HaloTag - EV | mScarlet | HaloTag^{(JF669)} | PKA | 116 aa |
| linker - mScarlet - FHA1 | | | | |
| FHA1 - HaloTag - EV | mScarlet | HaloTag^{(JF669)} | PKA | 116 aa |
| linker - mScarlet - PKA sub | | | | |
| FIG. 13 | | | | |
| Azalea-B5 - FHA1 - EV | HaloTag^{(NGD)} | Azalea-B5 | PKA | 116 aa |
| linker - PKA sub - HaloTag | | | | |
| Azalea B5 - FHA1 - 142aa | Azalea B5 | HaloTag^{(JF669)} | PKA | 142 aa |
| EV - PKA sub - HaloTag | | | | |
| Azalea B5 - FHA1 - 190aa | Azalea B5 | HaloTag^{(JF669)} | PKA | 190 aa |
| EV - PKA sub - HaloTag | | | | |
| Azalea B5 - FHA1 - 220aa | Azalea B5 | HaloTag^{(JF669)} | PKA | 220 aa |
| EV - PKA sub - HaloTag | | | | |
| Azalea B5 - FHA1 - 258aa | Azalea B5 | HaloTag^{(JF669)} | PKA | 258 aa |
| EV - PKA sub - HaloTag | | | | |
| Azalea B5 - FHA1 - 330aa | Azalea B5 | HaloTag^{(JF669)} | PKA | 330 aa |
| EV - PKA sub - HaloTag | | | | |
| FIG. 14 | | | | |
| mCitrine - FHA1 - EV | mCitrine | HaloTag^{(JF669)} | PKA | 116 aa |
| linker - PKA sub - HaloTag | | | | |
| mCitrine - FHA1 - EV | mCitrine | HaloTag^{(JFX646)} | PKA | 116 aa |
| linker - PKA sub - HaloTag | | | | |
| mCitrine - FHA1 - EV | mCitrine | HaloTag^{(JF635)} | PKA | 116 aa |
| linker - PKA sub - HaloTag | | | | |
| FIG. 15 | | | | |
| Dreiklang - FHA1 - EV | Dreiklang | HaloTag^{(JF669)} | PKA | 116 aa |
| linker - PKA sub - HaloTag | | | | |

Additional biosensors generated not presented in the figures in this manuscript

| | | | | |
|---|---|---|---|---|
| Shuffling biosensors | | | | |
| PKA sub - cpHaloTag - | cpHaloTag^{(NGD)} | mScarlet | PKA | / |
| mScarlet - FHA1 | | | | |
| PKA sub - mScarlet - | cpHaloTag^{(NGD)} | mScarlet | PKA | / |
| cpHaloTag - FHA1 | | | | |
| mScarlet - FHA1 - EV | HaloTag^{(NGD)} | mScarlet | PKA | 116 aa |
| linker - cpHaloTag - PKA | | | | |
| FHA1 - mScarlet - EV | HaloTag^{(NGD)} | mScarlet | PKA | 116 aa |
| linker - cpHaloTag - PKA | | | | |
| sub | | | | |
| PKA sub - mScarlet - EV | HaloTag^{(NGD)} | mScarlet | PKA | 116 aa |
| linker - cpHaloTag - FHA1 | | | | |
| PKA sub - cpHaloTag - EV | HaloTag^{(NGD)} | mScarlet | PKA | 116 aa |
| linker - mScarlet - FHA1 | | | | |
| FHA1 - cpHaloTag - EV | HaloTag^{(NGD)} | mScarlet | PKA | 116 aa |
| linker - mScarlet - PKA sub | | | | |
| EV linker length variation | | | | |
| mScarlet - FHA1 - EV | HaloTag^{(NGD)} | mScarlet | PKA | 64 aa |
| linker 64aa - PKA sub - | | | | |
| HaloTag | | | | |
| mScarlet - FHA1 - EV | HaloTag^{(NGD)} | mScarlet | PKA | 32 aa |
| linker 32aa - PKA sub - | | | | |
| HaloTag | | | | |
| mScarlet - FHA1 - EV | HaloTag^{(NGD)} | mScarlet | PKA | 24 aa |
| linker 24aa - PKA sub - | | | | |
| HaloTag | | | | |
| Alternative fluorescent | | | | |
| proteins including rsFPs | | | | |
| miRFP720 - FHA1 -EV | HaloTag^{(JF669)} | miRFP720 | PKA | 116 aa |
| linker - PKA sub - HaloTag | | | | |
| HaloTag - FHA1 - EV | HaloTag^{(JF669)} | miRFP720 | PKA | 116 aa |
| linker - PKA sub - miRFP720 | | | | |
| mRhubarb720 - FHA1 - EV | HaloTag^{(JF669)} | mRhubarb720 | PKA | 116 aa |
| linker - PKA sub - HaloTag | | | | |
| HaloTag - FHA1 - EV | HaloTag^{(JF669)} | mRhubarb720 | PKA | 116 aa |
| linker - PKA sub - mRhubarb720 | | | | |
| mNG3A - FHA1 - 116aa | mNG3A | HaloTag^{(JF669)} | PKA | 116 aa |
| EV - PKA sub - HaloTag | | | | |
| Dreiklang - FHA1 - 330aa | Dreiklang | HaloTag^{(JF669)} | PKA | 330 aa |
| EV - PKA sub - HaloTag | | | | |
| Lyn - Dreiklang - FHA1 - | Dreiklang | HaloTag^{(JF669)} | PKA | 330 aa |
| 330aa EV - PKA sub - | | | | |
| HaloTag | | | | |
| Skylan-S - FHA1 - EV - | Skylan-S | HaloTag^{(JF669)} | PKA | 116 aa |
| PKA sub - HaloTag | | | | |
| HaloTag - FHA1 - EV - | Skylan-S | HaloTag^{(JF669)} | PKA | 116 aa |
| PKA sub - Skylan-s | | | | |
| HaloTag - FHA1 - EV - | ffDronpa | HaloTag^{(JF669)} | PKA | 116 aa |
| PKA sub - ffDronpa | | | | |
| rsEGFP2 - FHA1 - EV - | rsEGFP2 | HaloTag^{(JF669)} | PKA | 116 aa |
| PKA sub - HaloTag | | | | |
| HaloTag - FHA1 - EV - | rsEGFP2 | HaloTag^{(JF669)} | PKA | 116 aa |
| PKA sub - rsEGFP2 | | | | |
| Lyn - Skylan-S - FHA1 - | Skylan-S | HaloTag^{(JF669)} | PKA | 116 aa |
| 116 aa EV - PKA sub - | | | | |
| HaloTag | | | | |
| Lyn - HaloTag - FHA1 - 116 | ffDronpa | HaloTag^{(JF669)} | PKA | 116 aa |
| aa EV - PKA sub - ffDronpa | | | | |
| Lyn - HaloTag - FHA1 - 116 | rsEGFP2 | HaloTag^{(JF669)} | PKA | 116 aa |
| aa EV - PKA sub - rsEGFP2 | | | | |
| Alternative kinase targets | | | | |
| sTagRFP - FHA1 - 190 aa | sTagRFP | HaloTag^{(JF669)} | PKC | 190 aa |
| EV - PKC sub - HaloTag | | | | |
| sTagRFP - FHA1 - 190 aa | sTagRFP | HaloTag^{(JF669)} | Akt | 190 aa |
| EV - Akt sub - HaloTag | | | | |
| sTagRFP - WW domain - | sTagRFP | HaloTag^{(JF669)} | ERK | 116 aa |
| EV - Erk sub - HaloTag | | | | |
| sTagRFP - FHA1 - 116aa | sTagRFP | HaloTag^{(JF669)} | AMPK | 116 aa |
| EV - AMPK sub - HaloTag | | | | |
| mCitrine - FHA1 - EV | mCitrine | HaloTag^{(JF669)} | PKC | 116 aa |
| linker - PKC sub - HaloTag | | | | |
| mCitrine - FHA1 - EV | mCitrine | HaloTag^{(JF669)} | Akt | 116 aa |
| linker - Akt sub - HaloTag | | | | |
| mCitrine - WW domain - | mCitrine | HaloTag^{(JF669)} | ERK | 116 aa |
| EV linker - Erk sub - | | | | |
| HaloTag | | | | |
| mCitrine - FHA1 - 116aa | mCitrine | HaloTag^{(JF669)} | AMPK | 116 aa |
| EV - AMPK sub - HaloTag | | | | |
| mCitrine - FHA1 - 258aa | mCitrine | HaloTag^{(JF669)} | PKC | 258 aa |
| EV - PKC sub - HaloTag | | | | |
| mCitrine - FHA1 - 258aa | mCitrine | HaloTag^{(JF669)} | Akt | 258 aa |
| EV - Akt sub - HaloTag | | | | |
| mCitrine - WW domain - | mCitrine | HaloTag^{(JF669)} | ERK | 258 aa |
| 258aa EV - Erk sub - | | | | |
| HaloTag | | | | |
| mCitrine - 4EBP1 - HaloTag | mCitrine | HaloTag^{(JF669)} | mTOR | / |
| mClover3 - 4EBP1 - | mClover3 | HaloTag^{(JF669)} | mTOR | / |
| HaloTag | | | | |
| EGFP - 4EBP1 - HaloTag | EGFP | HaloTag^{(JF669)} | mTOR | / |
| mNG3A - 4EBP1 - HaloTag | mNG3A | HaloTag^{(JF669)} | mTOR | / |
| T/A | | | | |
| mCitrine - FHA1 - EV | mCitrine | HaloTag^{(JF669)}) | PKA | 116 aa |
| linker - PKA sub - HaloTag | | | | |
| (T/A mutation) | | | | |
| Azalea B5 - FHA1 - EV | HaloTag^{(NGD)} | Azalea B5 | PKA | 116 aa |
| linker - PKA sub (T to A | | | | |
| mutation) - HaloTag | | | | |
| mScarlet - FHA1 - EV | HaloTag^{(NGD)} | mScarlet | PKA | 116 aa |
| linker - PKA sub (T to A | | | | |
| mutation) - HaloTag | | | | |
| FluoSTEPs | | | | |
| mNG2 (1-10) - FHA1 - | Split mNG2 | HaloTag^{(JF669)} | PKA | 220 aa |
| 220aa EV - PKA sub - | | | | |
| HaloTag | | | | |
| mNG2 (1-10) - FHA1 - | Split mNG2 | HaloTag^{(JF669)} | PKA | 258 aa |
| 258aa EV - PKA sub - | | | | |
| HaloTag | | | | |
| mNG2 (1-10) - FHA1 - | Split mNG2 | HaloTag^{(JF669)} | PKA | 330 aa |
| 330aa EV - PKA sub - | | | | |
| HaloTag | | | | |

TABLE 3
Imaging settings.

| Combination | Channel | Excitation | DC | Emission | Exposure |
|---|---|---|---|---|---|
| HaloTag^{(NGD)}-RFP | Donor | HQ495/10 | 515dcxr | HQ535/25 | 500 ms |
| | FRET | HQ495/10 | ZT568rdc | ET650/100 | 500 ms |
| | Acceptor | ET555/25 | ZT568rdc | ET650/100 | 100 ms |
| RFP- HaloTag^{(JF669)} | Donor | ET555/25 | ZT568rdc | ET605/52 | 500 ms |
| | FRET | ET555/25 | ZT568rdc | ET700/75 | 500 ms |
| | Acceptor | ET640/30 | T6601pxr | ET700/75 | 100 ms |
| YFP- HaloTag^{(JF669)} | Donor | HQ495/10 | 515dcxr | HQ535/25 | 500 ms |
| | FRET | HQ495/10 | 515dcxr | ET700/75 | 100 ms |
| | Acceptor | ET640/30 | T6601pxr | ET700/75 | |
| Dreiklang | Deactivation | ET340 | 450dcxru | — | — |
| Dreiklang | Activation | ET405/40 | 450dcxru | — | — |

INFORMAL SEQUENCE LISTING

| SEQ ID NO: | Name of sequence | Sequence |
|---|---|---|
| 1. | mScarlet | MVSKGEAVIKEFMRFKVHMEGSMNGHEFEIEGEGEGRPY |
| | | EGTQTAKLKVTKGGPLPFSWDILSPQFMYGSRAFTKHPA |
| | | DIPDYYKQSFPEGFKWERVMNFEDGGAVTVTQDTSLEDG |
| | | TLIYKVKLRGTNFPPDGPVMQKKTMGWEASTERLYPEDG |
| | | VLKGDIKMALRLKDGGRYLADFKTTYKAKKPVQMPGAYN |
| | | VDRKLDITSHNEDYTVVEQYERSEGRHSTGGMDELYK |
| 2. | sTagRFP | MSELIKENMHMKLYMEGTVNNHHFKCTSEGEGKPYEGTQ |
| | | TMRIKVVEGGPLPFAFDILATSFMYGSRTFINHTQGIPDFF |
| | | KQSFPEGFTWERVTTYEDGGVLTATQDTSLQDGCLIYNV |
| | | KIRGVNFPSNGPVMQKKTLGWEANTEMLYPADGGLEGRT |
| | | VMALKLVGGGHLICNFKTTYRSKKPAKNLKMPGVYYVDH |
| | | RLERIKEADKETYVEQHEVAVARYCDLPSKLGHK |
| 3. | mKok | MVSKGEESVIKPEMKMRYYMDGSVNGHEFTIEGEGTGRP |
| | | YEGHQEMTLRVTMAEGGPMPFAFDLVSHVFCYGHRVFT |
| | | KYPEEIPDYFKQAFPEGLSWERSLEFEDGGSASVSAHISL |
| | | RGNTFYHKSKFTGVNFPADGPIMQNQSVDWEPSTEKITA |
| | | SDGVLKGDVTMYLKLEGGGNHKCQFKTTYKAAKEILEMP |
| | | GDHYIGHRLVRKTEGNITEQVEDAVAHS |
| 4. | mRuby3 | MVSKGEELIKENMRMKVVMEGSVNGHQFKCTGEGEGRP |
| | | YEGVQTMRIKVIEGGPLPFAFDILATSFMYGSRTFIKYPADI |
| | | PDFFKQSFPEGFTWERVTRYEDGGVVTVTQDTSLEDGEL |
| | | VYNVKVRGVNFPSNGPVMQKKTKGWEPNTEMMYPADG |
| | | GLRGYTDIALKVDGGGHLHCNFVTTYRSKKTVGNIKMPGV |
| | | HAVDHRLERIEESDNETYVVQREVAVAKYSNLGGGMDEL |
| | | YK |
| 5. | Fusion MQV | MVSELIKENMPMKLYMEGTVNNHHFKCTSEGEGKPYEGT |
| | | QTQRIKVVEGGPLPFAFDILATSFMYGSRTFIKHPPGIPDF |
| | | FKQSFPEGFTWERVTTYEDGGVLTATQDTSLQDGCLIYN |
| | | VKVRGVNFPANGPVMQKKTLGWEASTETMYPADGGLEG |
| | | AVDMALKLVGGGHLICNMETTYRSKKPATNLKMPGVYNV |
| | | DHRLERIKEADDETYVEQHEVAVARYSTGGAGDGGK |
| 6. | FusionRed | MVSELIKENMPMKLYMEGTVNNHHFKCTSEGEGKPYEGT |
| | | QTMRIKVVEGGPLPFAFDILATSFMYGSRTFIKHPPGIPDF |
| | | FKQSFPEGFTWERVTTYEDGGVLTATQDTSLQDGCLIYN |
| | | VKVRGVNFPANGPVMQKKTLGWEASTETMYPADGGLEG |
| | | ACDMALKLVGGGHLICNLETTYRSKKPATNLKMPGVYNV |
| | | DHRLERIKEADDETYVEQHEVAVARYSTGGAGDGGK |
| 7. | mScarlet-I | MVSKGEAVIKEFMRFKVHMEGSMNGHEFEIEGEGEGRPY |
| | | EGTQTAKLKVTKGGPLPFSWDILSPQFMYGSRAFIKHPAD |
| | | IPDYYKQSFPEGFKWERVMNFEDGGAVTVTQDTSLEDGT |
| | | LIYKVKLRGTNFPPDGPVMQKKTMGWEASTERLYPEDGV |
| | | LKGDIKMALRLKDGGRYLADFKTTYKAKKPVQMPGAYNV |
| | | DRKLDITSHNEDYTVVEQYERSEGRHSTGGMDELYK |
| 8. | Azalea-B5 | MENVRRKTGIQTEMKTKLHMDGMVNGHSFEIKGEGKGSP |
| | | YEGVQTMKLKVTKGAPLPFSIDILLPQCMYGSKPFIKYPEN |
| | | IPDYLKLSFPEGITWERTMTFEDGAVCDVSNDSRLVGNCF |
| | | IYTVKFQGVNFPLDGPVMQKKTRGWEPSTEVLYECDGW |
| | | MRGLVDIALKLENGGHYMCNFKTTYKSKKGLEVPPYHFV |
| | | DHKLDLLSHNTDGATFEEFEQGEIAHAHLSKLA |
| 9. | mKate2 | MVSELIKENMHMKLYMEGTVNNHHFKCTSEGEGKPYEGT |
| | | QTMRIKAVEGGPLPFAFDILATSFMYGSKTFINHTQGIPDF |
| | | FKQSFPEGFTWERVTTYEDGGVLTATQDTSLQDGCLIYN |
| | | VKIRGVNFPSNGPVMQKKTLGWEASTETLYPADGGLEGR |
| | | ADMALKLVGGGHLICNLKTTYRSKKPAKNLKMPGVYYVD |
| | | RRLERIKEADKETYVEQHEVAVARYCDLPSKLGHR |
| 10. | mCherry | MVSKGEEDNMAIIKEFMRFKVHMEGSVNGHEFEIEGEGE |
| | | GRPYEGTQTAKLKVTKGGPLPFAWDILSPQFMYGSKAYV |
| | | KHPADIPDYLKLSFPEGFKWERVMNFEDGGVVTVTQDSS |
| | | LQDGEFIYKVKLRGTNFPSDGPVMQKKTMGWEASSERM |
| | | YPEDGALKGEIKQRLKLKDGGHYDAEVKTTYKAKKPVQLP |
| | | GAYNVNIKLDITSHNEDYTIVEQYERAEGRHSTGGMDELY |
| | | K |
| 11. | mNeptune2 | MVSKGEELIKENMHMKLYMEGTVNNHHFKCTSEGEGKPY |
| | | EGTQTGRIKVVEGGPLPFAFDILATCFMYGSKTFINHTQGI |
| | | PDFFKQSFPEGFTWERVTTYEDGGVLTVTQDTSLQDGCLI |
| | | YNVKLRGVNFPSNGPVMQKKTLGWEASTETLYPADGGLE |
| | | GRCDMALKLVGGGHLHCNLKTTYRSKKPAKNLKMPGVYF |
| | | VDRRLERIKEADNETYVEQHEVAVARYCDLPSKLGHKLNG |
| | | MDELYK |
| 12. | mNeptune2.5 | MVSKGEELIKENMHTKLYMEGTVNNHHFKCTHEGEGKPY |
| | | EGTQTNRIKVVEGGPLPFAFDILATCFMYGSKTFINHTQGI |
| | | PDFFKQSFPEGFTWERVTTYEDGGVLTVTQDTSLQDGCLI |
| | | YNVKLRGVNFPSNGPVMQKKTLGWEASTETLYPADGGLE |
| | | GRCDMALKLVGGGHLHCNLKTTYRSKKPAKNLKMPGVYF |
| | | VDRRLERIKEADNETYVEQHEVAVARYCDLPSKLGHKLNG |
| | | MDELYK |
| 13. | mCitrine | MVSKGEELFTGWPILVELDGDVNGHKFSVSGEGEGDAT |
| | | YGKLTLKFICTTGKLPVPWPTLVTTFGYGLMCFARYPDHM |
| | | KQHDFFKSAMPEGYVQERTIFFKDDGNYKTRAEVKFEGD |
| | | TLVNRIELKGIDFKEDGNILGHKLEYNYNSHNVYIMADKQK |
| | | NGIKVNFKIRHNIEDGSVQLADHYQQNTPIGDGPVLLPDN |
| | | HYLSYQSKLSKDPNEKRDHMVLLEFVTAAGITLGMDELYK |
| 14. | cp Venus | GGVQLADHYQQNTPIGDGPVLLPDNHYLSYQSKLSKDPN |
| | | EKRDHMVLLEFVTAAGITLGMDELYKGGTGGSMVSKGEE |
| | | LFTGVVPILVELDGDVNGHKFSVSGEGEGDATYGKLTLKLI |
| | | CTTGKLPVPWPTLVTTLGYGLQCFARYPDHMKQHDFFKS |
| | | AMPEGYVQERTIFFKDDGNYKTRAEVKFEGDTLVNRIELK |
| | | GIDFKEDGNILGHKLEYNYNSHNVYITADKQKNGIKANFKI |
| | | RHNIE |
| 15. | mPapaya | MVSKGEGQSKHGLKEEMTVKYHMEGCVNGHKFVITGEGI |
| | | GNPFKGKQTANLCVIEGGPLPFSEDILSPGFKYGDRIFTEY |
| | | PQDIVDYFKNSCPAGYTWERSFLFEDGAVCRCNVDITVSE |
| | | KENCIYHKSIFRGVNFPADGPVMKKMTTNWEASTEKIVPV |
| | | PKQGILKGKVKMYLLLKDGGRYHCQFDTVYKAKSVPSKM |
| | | PEWHFIQHKLLREDRSDAKNQKWQLTEHAIAGMDELYK |
| 16. | mNG3A full length | MVSKGEEDNMASLPATHELHIFGSINGVDFDMVGQGTGN |
| | | PNDGYEELNLKSTKGDLQFSPWILVPHIGYGFHQYLPYPD |
| | | GMSPFQAAMVDGSGYQVHRTMQFEDGASLTVNYRYTYE |
| | | GSHIKGEAQVMGTGFPADGPVMTNTLTAADLCVSKMTYP |
| | | NDKTIISTFKWSYTTVNGKRYRSTARTTYTFAKPMAAKYLK |
| | | NQPMYVLRKTELKHSMTELNFKEWQKAFTDMM |
| 17. | miRFP720 | MAEGSVARQPDLLTCDDEPIHIPGAIQPHGLLLALAADMTI |
| | | VAGSDNLPELTGLAIGALIGRSAADVFDSETHNRLTIALAE |
| | | PGAAVGAPITVGFTMRKDAGFIGSWHRHDQLIFLELEPPQ |
| | | RDVAEPQAFFRRTNSAIRRLQAAETLESACAAAAQEVRKI |
| | | TGFDRVMIYRFASDFSGSVIAEDRCAEVESKLGLHYPASFI |
| | | PAQARRLYTINPVRIIPDINYRPVPVTPDLNPVTGRPIDLSF |
| | | AILRSVSPNHLEFMRNIGMHGTMSISILRGERLWGLIVCHH |
| | | RTPYYVDLDGRQACKRVAERLATQIGVMEE |
| 18. | mRhubarb720 | MAEGSVARQPDLLTCDDEPIHIPGAIQPHGLLLALAADMTI |
| | | VAGSDNLPELTGLAIGALIGRSAADVFDSETHNRLTIALAE |
| | | PGAAVGAPITVGFTMRKDAGFIGSWHRHDQLIFLELEPPQ |
| | | RDVAEPQAFFRHTNSAIRRLQAAETLESACAAAAQEVRKI |
| | | TGFDRVMIYRFASDFSGEVIAEDRCAEVESKLGQHYPASD |
| | | IPAQARRLYTINPVRIIPDINYRPVPVTPDLNPVTGRPIDLSF |
| | | AILRSVSPVHLEFMRNIGMHGTMSISILRGERLWGLIVCHH |
| | | RTPYYVDLDGRQACELVAQVLARAIGVMEE |
| 19. | Dreiklang | MVSKGEELFTGWVPILVELDGDVNGHKFSVSGEGEGDAT |
| | | YGKLTLKFICTTGKLPVPWPTLLTTIGYGLMCFARYPDHMK |
| | | QHDFFKSAMPEGYVQERTIFFKDDGNYKTRAEVKFEGDT |
| | | LVNRIELKGIDFKEDGNILGHKLEYNHDSHNVYIMADKQKN |
| | | GIKVNFKIRHNIEDGSVQLADHYQQNTPIGDGPVLLPDNHY |
| | | LSYQSKLSKDPNEKRDHMVLLEFVTAAGITLGMDELYK |
| 20. | Skylan-S | MSAIKPDMKIKLRMEGNVNGHHFVIDGDGTGKPFEGKQS |
| | | MDLEVKEGGPLPFAFDILTTAFSYGNRVFAKYPDNIQDYF |
| | | KQSFPKGYSWERSLTFEDGGICNARNDITMEGDTFYNKV |
| | | RFYGTNFPANGPVMQKKTLKWEPSTEKMYVRDGVLTGD |
| | | VEMALLLEGNAHYRCDFRTTYKAKEKGVKLPGAHFVDHCI |
| | | EILSHDKDYNKVKLYEHAVAHSGLPDNARR |
| 21. | ffDronpa | MSVIKPDMKIKLRMEGAVNGHPFAIEGVGLGKPFEGKQS |
| | | MDLKVKEGGPLPFAYDILTTAFCYGNRVFAKYPENIVDYFK |
| | | QSFPEGYSWERSMNYEDGGICNATNDITLDGDCYIYEIRF |
| | | DGVNFPANGPVMQKRTVKWEPSTEKLYVRDGVLKGDVN |
| | | MALSLEGGGHYRCDFKTTYKAKKVVQLPDYHFVDHHIEIK |
| | | SHDKDYSNVNLHEHAEAHSELPRQAK |
| 22. | rsEGFP2 | MVSKGEELFTGVVPILVELDGDVNGHKFSVSGEGEGDAT |
| | | YGKLTLKFICTTGKLPVPWPTLVTTLAYGVLCFSRYPDHM |
| | | KQHDFFKSAMPEGYVQERTIFFKDDGNYKTRAEVKFEGD |
| | | TLVNRIELKGIDFKEDGNILGHKLEYNYNSHNVYIMADKQK |
| | | NGIKSNFKIRHNIEDGSVQLADHYQQNTPIGDGPVLLPDN |
| | | HYLSTQSKLSKDPNEKRDHMVLLEFVTAAGITLGMDELYK |
| 23. | mClover3 | MVSKGEELFTGVVPILVELDGDVNGHKFSVRGEGEGDAT |
| | | NGKLTLKFICTTGKLPVPWPTLVTTFGYGVACFSRYPDHM |
| | | KQHDFFKSAMPEGYVQERTISFKDDGTYKTRAEVKFEGD |
| | | TLVNRIELKGIDFKEDGNILGHKLEYNFNSHYVYITADKQK |
| | | NCIKANFKIRHNVEDGSVQLADHYQQNTPIGDGPVLLPDN |
| | | HYLSHQSKLSKDPNEKRDHMVLLEFVTAALESGGEDP |
| 24. | EGFP | MVSKGEELFTGWVPILVELDGDVNGHKFSVSGEGEGDAT |
| | | YGKLTLKFICTTGKLPVPWPTLVTTLTYGVQCFSRYPDHM |
| | | KQHDFFKSAMPEGYVQERTIFFKDDGNYKTRAEVKFEGD |
| | | TLVNRIELKGIDFKEDGNILGHKLEYNYNSHNVYIMADKQK |
| | | NGIKANFKIRHNIEDGSVQLADHYQQNTPIGDGPVLLPDN |
| | | HYLSTQSALSKDPNEKRDHMVLLEFVTAAGITLGMDELYK |
| 25. | HaloTag | MGSEIGTGFPFDPHYVEVLGERMHYVDVGPRDGTPVLFL |
| | | HGNPTSSYVWRNIIPHVAPTHRCIAPDLIGMGKSDKPDLG |
| | | YFFDDHVRFMDAFIEALGLEEVVLVIHDWGSALGFHWAKR |
| | | NPERVKGIAFMEFIRPIPTWDEWPEFARETFQAFRTTDVG |
| | | RKLIIDQNVFIEGTLPMGVVRPLTEVEMDHYREPFLNPVDR |
| | | EPLWRFPNELPIAGEPANIVALVEEYMDWLHQSPVPKLLF |
| | | WGTPGVLIPPAEAARLAKSLPNCKAVDIGPGLNLLQEDNP |
| | | DLIGSEIARWLSTLEISG |
| 26. | cpHaloTag | ETFQAFRTTDVGRKLIIDQNVFIEGTLPMGVVRPLTEVEMD |
| | | HYREPFLNPVDREPLWRFPNELPIAGEPANIVALVEEYMD |
| | | WLHQSPVPKLLFWGTPGVLIPPAEAARLAKSLPNCKAVDI |
| | | GPGLNLLQEDNPDLIGSEIARWLSTLEISGGGTGGSGGTG |
| | | GSGGTGGSMAEIGTGFPFDPHYVEVLGERMHYVDVGPR |
| | | DGTPVLFLHGNPTSSYVWRNIIPHVAPTHRCIAPDLIGMGK |
| | | SDKPDLGYFFDDHVRFMDAFIEALGLEEVVLVIHDWGSAL |
| | | GFHWAKRNPERVKGIAFMEFIRPIPTWDEW |
| 27. | PKA sensing | LRRATLVDGGTGGSEL |
| | domain (PKA | |
| | substrate) | |
| 28. | PKA sensing | LRRAALVDGGTGGSEL |
| | domain-negative | |
| | control (PKA | |
| | substrateT-to-A) | |
| 29. | PKC sensing | RFRRFQTLKDKAKAGGSEL |
| | domain (PKC | |
| | substrate) | |
| 30. | Akt sensing domain | PRPRSCTWPDPRPEFGGSEL |
| | (Akt substrate) | |
| 31. | ERK sensing | PDVPRTPVDKAKLSFQFPGGSEL |
| | domain (ERK | |
| | substrate) | |
| 32. | AMPK sensing | MRRVATLVDLGTGGSEL |
| | domain (AMPK | |
| | substrate) | |
| 33. | Src sensing domain | WMEDYDYVHLQG |
| | (Src substrate) | |
| 34. | Fyn sensing | EKIEGTYGVV |
| | domain (Fyn | |
| | substrate) | |
| 35. | RBD (Ras, Rap1) | PSKTSNTIRVFLPNKQRTVVNVRNGMSLHDCLMKALKVR |
| | | GLQPECCAVFRLLHEHKGKKARLDWNTDAASLIGEELQV |
| | | DFL |
| 36. | CaM (Calcium) | DQLTEEQIAEFKEAFSLFDKDGDGTITTKELGTVMRSLGQ |
| | | NPTEAELQDMINEVDADGDGTIDFPEFLTMMARKMKDTD |
| | | SEEEIREAFRVFDKDGNGYISAAELRHVMTNLGEKLTDEE |
| | | VDEMIREADIDGDGQVNYEEFVQMMTAK |
| 37. | cAMP sensing | PVGTHEMEEELAEAVALLSQRGPDALLTVALRKPPGQRT |
| | domain (Epac) | DEELDLIFEELLHIKAVAHLSNSVKRELAAVLLFEPHSKAGT |
| | | VLFSQGDKGTSWYIIWKGSVNWTHGKGLVTTLHEGDDF |
| | | GELALVNDAPRAATIILREDNCHFLRVDKQDFNRIIKDVEA |
| | | KTMRLEEHGKVVLVLERASQGAGPSRPPTPGRNRYTVMS |
| | | GTPEKILELLLEAMGPDSSAHDQTETFLSDFLLTHRVFMPS |
| | | AQLCAALLHHFHVEPAGGSEQERSTYVCNKRQQILRLVS |
| | | QWVALYGSMLHTDPVATSFLQKLSDLVGRDTRLSNLLRE |
| | | QWPERRRCHRLENGCGNASPQMKARNLPVWLPNQDEP |
| | | LPGSSCAIQVGDKVPYDICRPDHSVLTLQLPVTASVREVM |
| | | AALAQEDGWTKGQVLVKVNSAGDAIGLQPDARGVATSLG |
| | | LNERLFWVNPQEAHELIPHPDQLGPTVGSAEGLDLVSAKD |
| | | LAGQLTDHDWSLFNSIHQVELIHYVLGPQHLRDVTTANLE |
| | | RFMRRFNELQYWVATELCLCPVPGPRAQLLRKFIKLAAHL |
| | | KEQKNLNSFFAVMFGLSNSAISHLAHTWERLPHKVRKLYS |
| | | ALERLLDPSWNHRVYRLALAKLSPPVIPFMPLLLKDMTFIH |
| | | EGNHTLVENLINFEKMRMMARVARMLHHCRSHNPVPLSP |
| | | LRSRVSHLHEDSQVARISTCSEQSLSTRSPASTWAYVQQ |
| | | LKVIDNQRELSRLSRELEP |
| 38. | mTOR sensing | MSGGSSCSQTPSRAIPATRRVVLGDGVQLPPGDYSTTPG |
| | domain (4EBP1) | GTLFSTTPGGTRIIYDRKFLMECRNSPVTKTPPRDLPTIPG |
| | | VTSPSSDEPPMEASQSHLRNSPEDKRAGGEESQFEMDI |
| 39. | FHA1 (PKA, Akt, | KFSQEQIGENIVCRVICTTGQIPIRDLSADISQVLKEKRSIKK |
| | AMPK) | VWTFGRNPACDYHLGNISRLSNKHFQILLGEDGNLLLNDIS |
| | | TNGTWLNGQKVEKNSNQLLSQGDEITVGVGVESDILSLVI |
| | | FINDKFKQCLEQNKVDR |
| 40. | WW (ERK) | MADEEKLPPGWEKRMSRSSGRVYYFNHITNASQWERPS |
| | | GNSSSGGKNGQGEPAR |
| 41. | SH2 (Src and Fyn) | WYFGKITRRESERLLLNPENPRGTFLVRESETTKGAYCLS |
| | | VSDFDNAKGLNVKHYKIRKLDSGGFYITSRTQFSSLQQLV |
| | | AYYSKHADGLCHRLTNVC |
| 42. | Ras sensing domain | QNHFVDEYDPTIGGSEL |
| | (PL) | |
| 43. | CaM-binding | SSRRKWNKTGHAVRAIGRLSS |
| | peptide of myosin | |
| | light-chain kinase | |
| | (M13) | |
| 44. | H-Ras | MTEYKLVVVGAGGVGKSALTIQLIQNHFVDEYDPTIEDSYR |
| | | KQWIDGETCLLDILDTAGQEEYSAMRDQYMRTGEGFLCV |
| | | FAINNTKSFEDIHQYREQIKRVKDSDDVPMVLVGNKCDLA |
| | | ARTVESRQAQDLARSYGIPYIETSAKTRQGVEDAFYTLVR |
| | | EIRQHKLRKLNG |
| 45. | Rap1 | MREYKLVVLGSGGVGKSALTVQFVQGIFVEKYDPTIEDSY |
| | | RKQVEVDCQQCMLEILDTAGTEQFTAMRDLYMKNGQGF |
| | | ALVYSITAQSTFNDLQDLREQILRVKDTEDVPMILVGNKCD |
| | | LEDERVVGKEQGQNLARQWCNCAFLESSAKSKINVNEIFY |
| | | DLVRQINRKTPVEKKKPKKKSCLLL |
| 46. | Original EV linker | SAGGSAGGSAGGSAGGSAGGSGSAGGSAGGSTSAGGS |
| | (116 aa) | AGGSAGGSAGGSAGGSGSAGGSAGGSTSAGGSAGGSA |
| | | GGSAGGSAGGSGSAGGSAGGSTSAGGSAGGSAGGSAG |
| | | GSAGG |
| 47. | Normal linker | SAGKPGSGEGSTKG |
| 48. | 20 aa EV linker | SAGGSAGGSAGGSAGGSAGG |
| 49. | 24 aa EV linker | GTSAGGSAGGSAGGSAGGSAGGSG |
| 50. | 32 aa EV linker | SAGGSAGGSAGGSAGGSAGGSGSAGGSAGGST |
| 51. | 40 aa EV linker | SAGGSAGGSGSAGGSAGGSTSAGGSAGGSAGGSAGGS |
| | | AGG |
| 52. | 52 aa EV linker | SAGGSAGGSAGGSAGGSAGGSGSAGGSAGGSTSAGGS |
| | | AGGSAGGSAGGSAGG |
| 53. | 64 aa EV linker | SAGGSAGGSAGGSAGGSAGGSGSAGGSAGGSTSAGGS |
| | | AGGSAGGSAGGSAGGSGSAGGSAGGST |
| 54. | 80 aa EV linker | SAGGSAGGSAGGSAGGSAGGSGSAGGSAGGSTSAGGS |
| | | AGGSAGGSAGGSGSAGGSAGGSTSAGGSAGGSAGGSA |
| | | GGSAGG |
| 55. | 142 aa EV linker | SAGGSAGGSAGGSAGGSAGGSGSAGGSAGGSTSAGGS |
| | | AGGSAGGSAGGSAGGSGSAGGSAGGSAGGSAGGSGSA |
| | | GGSAGGSTSAGGSAGGSAGGSAGGSAGGSGSAGGSAG |
| | | GSTSAGGSAGGSAGGSAGGSAGGSAGGSAGG |
| 56. | 190 aa EV linker | SAGGSAGGSAGGSAGGSAGGSGSAGGSAGGSTSAGGS |
| | | AGGSAGGSAGGSAGGSGSAGGSAGGSTSAGGSAGGSA |
| | | GGSAGGSAGGSGSAGGSAGGSTSAGGSAGGSAGGSAG |
| | | GSAGGSGSAGGSAGGSTSAGGSAGGSAGGSAGGSAGG |
| | | SGSAGGSAGGSTSAGGSAGGSAGGSAGGSAGGSGSAG |
| | | GSAGG |
| 57. | 220 aa EV linker | SAGGSAGGSAGGSAGGSAGGSGSAGGSAGGSTSAGGS |
| | | AGGSAGGSAGGSAGGSGSAGGSAGGSTSAGGSAGGSA |
| | | GGSAGGSAGGSGSAGGSAGGSTSAGGSAGGSAGGSAG |
| | | GSAGGSGSAGGSAGGSTSAGGSAGGSAGGSAGGSAGG |
| | | SGSAGGSAGGSTSAGGSAGGSAGGSAGGSAGGSGSAG |
| | | GSAGGSTSAGGSAGGSAGGSAGGSAGGSAGGSAGG |
| 58. | 258 aa EV linker | SAGGSAGGSAGGSAGGSAGGSGSAGGSAGGSTSAGGS |
| | | AGGSAGGSAGGSAGGSGSAGGSAGGSTSAGGSAGGSA |
| | | GGSAGGSAGGSGSAGGSAGGSTSAGGSAGGSAGGSAG |
| | | GSAGGSGSAGGSAGGSTSAGGSAGGSAGGSAGGSAGG |
| | | SGSAGGSAGGSTSAGGSAGGSAGGSAGGSTSAGGSAG |
| | | GSAGGSAGGSAGGSGSAGGSAGGSTSAGGSAGGSAGG |
| | | SAGGSGSAGGSAGGSTSAGGSAGGSAGGSAGGSAGG |
| 59. | 314 aa EV linker | GGTACCAGTGCTGGTGGTAGTGCTGGTGGTAGTGCTG |
| | | GTGGTTCCGGCAGTGCTGGTGGTAGTGCTGGTGGTAG |
| | | TACCAGTGCTGGTGGTAGTGCTGGTGGTAGTGCTGGT |
| | | GGTAGTGCTGGTGGTAGTGCTGGTGGTTCCGGCAGTG |
| | | CTGGTGGTAGTGCTGGTGGTAGTACCAGTGCTGGTGG |
| | | TAGTGCTGGTGGTAGTGCTGGTGGTAGTGCTGGTGGTA |
| | | GTGCTGGTGGTTCCGGCAGTGCTGGTGGTAGTGCTGG |
| | | TGGTAGTACCAGTGCTGGTGGTAGTGCTGGTGGTAGTG |
| | | CTGGTGGTAGTGCTGG |
| 60. | sfGFP(1-10) | MSKGEELFTGVVPILVELDGDVNGHKFSVRGEGEGDATIG |
| | | KLTLKFICTTGKLPVPWPTLVTTLTYGVQCFSRYPDHMKR |
| | | HDFFKSAMPEGYVQERTISFKDDGKYKTRAVVKFEGDTLV |
| | | NRIELKGTDFKEDGNILGHKLEYNFNSHNVYITADKQKNGI |
| | | KANFTVRHNVEDGSVQLADHYQQNTPIGDGPVLLPDNHY |
| | | LSTQTVLSKDPNEK |
| 61. | mNG2(1-10) | MVSKGEEDNMASLPATHELHIFGSINGVDFDMVGQGTGN |
| | | PNDGYEELNLKSTKGDLQFSPWILVPHIGYGFHQYLPYPD |
| | | GMSPFQAAMVDGSGYQVHRTMQFEDGASLTVNYRYTYE |
| | | GSHIKGEAQVMGTGFPADGPVMTNTLTAADWCMSKKTY |
| | | PNDKTIISTFKWSYTTVNGKRYRSTARTTYTFAKPMAANY |
| | | LKNQPMYVFRKTELKHSM |
| 62. | mNG3A(1-10) | MVSKGEEDNMASLPATHELHIFGSINGVDFDMVGQGTGN |
| | | PNDGYEELNLKSTKGDLQFSPWILVPHIGYGFHQYLPYPD |
| | | GMSPFQAAMVDGSGYQVHRTMQFEDGASLTVNYRYTYE |
| | | GSHIKGEAQVMGTGFPADGPVMTNTLTAADLCVSKMTYP |
| | | NDKTIISTFKWSYTTVNGKRYRSTARTTYTFAKPMAAKYLK |
| | | NQPMYVLRKTELKHSM |
| 63. | GFP11 | RDHMVLHEYVNAAGIT |
| 64. | mNG11 | TELNFKEWQKAFTDMM |
| 65. | GFP11 x7 | RDHMVLHEYVNAAGITGGSGSSGGGSGGGSSRDHMVLH |
| | | EYVNAAGITGSGGSGSGSGSGGSSRDHMVLHEYVNAAGI |
| | | TGSGSGSSGGGSSGSSRDHMVLHEYVNAAGITGSGGSG |
| | | SGSSGGSGSRDHMVLHEYVNAAGITGSGGSGGSGSGSG |
| | | GSRDHMVLHEYVNAAGITGGSSGGSSGSGGSSSRDHMV |
| | | LHEYVNAAGIT |
| 66. | mNG11 x3 | TELNFKEWQKAFTDMMGGSGSTELNFKEWQKAFTDMMG |
| | | SGGSTELNFKEWQKAFTDMM |
| 67. | splitHalo Tag-N | MGSEIGTGFPFDPHYVEVLGERMHYVDVGPRDGTPVLFL |
| | | HGNPTSSYVWRNIIPHVAPTHRCIAPDLIGMGKSDKPDLG |
| | | YFFDDHVRFMDAFIEALGLEEVVLVIHDWGSALGFHWAKR |
| | | NPERVKGIAFMEFIRPIPTWDEWPEFARETFQAFRTT |
| 68. | splitHalo Tag-C | MGSDVGRKLIIDQNVFIEGTLPMGWVRPLTEVEMDHYREP |
| | | FLNPVDREPLWRFPNELPIAGEPANIVALVEEYMDWLHQS |
| | | PVPKLLFWGTPGVLIPPAEAARLAKSLPNCKAVDIGPGLNL |
| | | LQEDNPDLIGSEIARWLSTLEISG |

### P Embodiments

P Embodiment 1. Methods, systems, and articles of manufacture for biosensors as substantially described and shown herein.

### EMBODIMENTS

Embodiment 1. A recombinant protein covalently bound to a chemical fluorophore, said recombinant protein comprising a fluorescent protein domain bound to a biosensing protein domain and a haloalkane dehalogenase domain bound to said biosensing protein domain, wherein: said chemical fluorophore is covalently bound to said haloalkane dehalogenase domain; and said chemical fluorophore and said fluorescent protein domain are a Forster Resonance Energy Transfer (FRET) pair.

Embodiment 2. The recombinant protein of embodiment 1, wherein said fluorescent protein domain is a red fluorescent protein (RFP) domain, an orange fluorescent protein (OFP) domain, a yellow fluorescent protein (YFP) domain, or a green fluorescent protein (GFP) domain.

Embodiment 3. The recombinant protein of embodiment 1 or 2, wherein said fluorescent protein domain is an mScarlet domain, a stagRFP domain, an mKOk domain, an mRuby3 domain, a FusionMQV domain, a FusionRed domain, an mScarlet-1 domain, an Azalea-B5 domain, an mKate2 domain, an mCherry domain, an mNeptune2 domain, an mNeptune2.5 domain, an mCitrine domain, a cpVenus domain, an mPapaya domain, a sfGFP domain, an mNG2 domain, an mNG3A domain, an miRFP720 domain, an mRhubarb720 domain, a Dreiklang domain, a Skylan-S domain, an ffDronpa domain, an rsEGFP2 domain, an mClover3 domain, or an EGFP domain.

Embodiment 4. The recombinant protein of any one of embodiments 1-3, wherein said fluorescent protein domain comprises the amino acid sequence of any one of SEQ ID NO:1 to SEQ ID NO:24.

Embodiment 5. The recombinant protein of any one of embodiments 1-4, wherein said haloalkane dehalogenase domain comprises the amino acid sequence of SEQ ID NO:25 or SEQ ID NO:26.

Embodiment 6. The recombinant protein of any one of embodiments 1-5, wherein said chemical fluorophore is NGD, JF669, JFX646, or JF635.

Embodiment 7. The recombinant protein of any one of embodiments 1-6, wherein said chemical fluorophore comprises the formula of

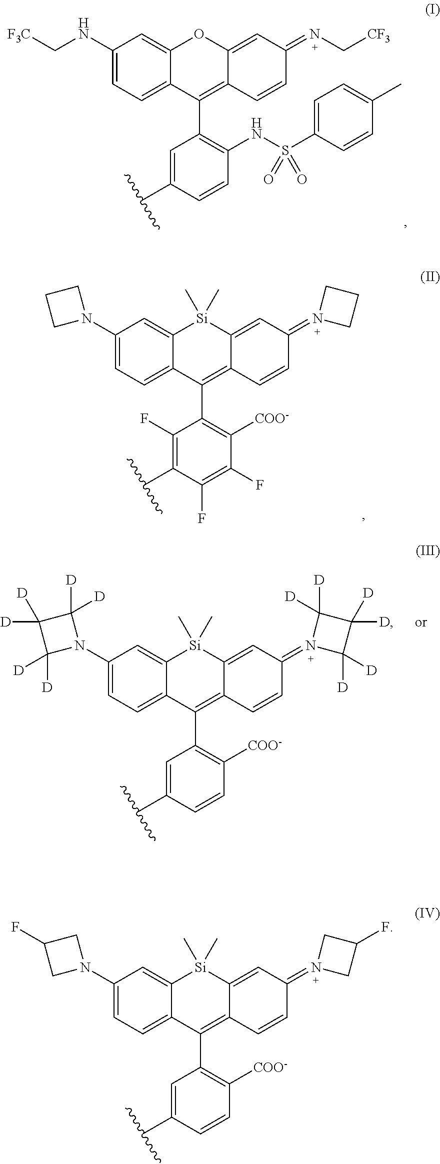

Embodiment 8. The recombinant protein of any one of embodiments 1-7, wherein said biosensing protein domain comprises: (a) a target biomolecule binding domain: or (b) a target biomolecule substrate domain, a linker domain, and a target biomolecule activity sensing domain: or (c) a target biomolecule domain, a linker domain, and a target biomolecule substrate domain.

Embodiment 9. The recombinant protein of embodiment 8, wherein said target biomolecule is a protein kinase, a second messenger molecule, or a GTPase.

Embodiment 10. The recombinant protein of embodiment 9, wherein said protein kinase is a protein kinase A (PKA), a protein kinase C (PKC), a protein kinase B (PKB), an extracellular signal-regulated kinase (ERK), a 5′ adenosine monophosphate-activated protein kinase (AMPK), a mammalian target of rapamycin (mTOR), a Fyn kinase, or a Src kinase.

Embodiment 11. The recombinant protein of embodiment 9, wherein said second messenger molecule is cyclic adenosine monophosphate (cAMP), cyclic guanosine monophosphate (cGMP), inositol triphosphate (IP₃), diacylglycerol (DAG), or calcium (Ca²⁺).

Embodiment 12. The recombinant protein of embodiment 9, wherein said GTPase is a Ras GTPase or a Rap1 GTPase.

Embodiment 13. The recombinant protein of any one of embodiments 8-12, wherein said biomolecule substrate domain comprises the amino acid sequence of any one of SEQ ID NO:27 to SEQ ID NO:36.

Embodiment 14. The recombinant protein of any one of embodiments 8-13, wherein said target biomolecule substrate domain further comprises a phosphorylatable amino acid residue.

Embodiment 15. The recombinant protein of embodiment 14, wherein said phosphorylatable amino acid residue is capable of being phosphorylated by said protein kinase.

Embodiment 16. The recombinant protein of any one of embodiments 8-15, wherein said target biomolecule domain comprises the amino acid sequence of SEQ ID NO:44 or SEQ ID NO:45.

Embodiment 17. The recombinant protein of any one of embodiments 8-15, wherein said target biomolecule activity sensing domain comprises a phosphoamino acid binding domain.

Embodiment 18. The recombinant protein of any one of embodiments 17, wherein said phosphoamino acid binding domain comprises the amino acid sequence of SEQ ID NO:39, SEQ ID NO:40, or SEQ ID NO:41.

Embodiment 19. The recombinant protein of any one of embodiments 8-18, wherein said linker domain is a peptide linker.

Embodiment 20. The recombinant protein of any one of embodiments 8-19, wherein said linker domain is between about 1 to about 500 amino acids in length.

Embodiment 21. The recombinant protein of any one of embodiments 8-20, wherein said linker domain comprises the amino acid sequence of any one of SEQ ID NO:46 to SEQ ID NO:59.

Embodiment 22. The recombinant protein of any one of embodiments 8-12, wherein said target biomolecule binding domain comprises the amino acid sequence of SEQ ID NO:37 or SEQ ID NO:38.

Embodiment 23. The recombinant protein of any one of embodiments 1-21, wherein said fluorescent protein is attached to the N-terminus of said biosensing protein domain and said biosensing protein domain is attached to the N-terminus of said haloalkane dehalogenase domain.

Embodiment 24. The recombinant protein of any one of embodiments 1-21, wherein said haloalkane dehalogenase domain is attached to the N-terminus of said biosensing protein domain and said biosensing protein domain is attached to the N-terminus of said fluorescent protein.

Embodiment 25. A recombinant protein covalently bound to a chemical fluorophore, said recombinant protein comprising a split fluorescent protein domain bound to a biosensing protein domain and a haloalkane dehalogenase domain bound to said biosensing protein domain, wherein: said chemical fluorophore is covalently bound to said haloalkane dehalogenase domain; said split fluorescent protein domain is one part of a fluorescent protein split pair that forms a reconstituted fluorescent protein upon binding of a cognate split fluorescent protein; and said chemical fluorophore and said reconstituted fluorescent protein domain are a Forster Resonance Energy Transfer (FRET) pair.

Embodiment 26. The recombinant protein of embodiment 25, wherein said recombinant protein is non-covalently bound to a second recombinant protein comprising the cognate split fluorescent protein domain bound to a protein of interest, wherein the split fluorescent protein domain is non-covalently bound to the cognate split fluorescent protein thereby forming said reconstituted fluorescent protein.

Embodiment 27. The recombinant protein of embodiment 25 or 26, wherein said reconstituted fluorescent protein is a reconstituted red fluorescent protein (RFP), a reconstituted orange fluorescent protein (OFP), a reconstituted yellow fluorescent protein (YFP), or a reconstituted green fluorescent protein (GFP).

Embodiment 28. The recombinant protein of any one of embodiment 25-27, wherein said reconstituted fluorescent protein is a reconstituted mScarlet, a reconstituted stagRFP, a reconstituted mKOk, a reconstituted mRuby3, a reconstituted FusionMQV, a reconstituted FusionRed, a reconstituted mScarlet-1, a reconstituted Azalea-B5, a reconstituted mKate2, a reconstituted mCherry, a reconstituted mNeptune2, a reconstituted mNeptune2.5, a reconstituted mCitrine, a reconstituted cpVenus, a reconstituted mPapaya, a reconstituted sfGFP, a reconstituted mNG2, a reconstituted mNG3A, a reconstituted miRFP720, a reconstituted mRhubarb720, a reconstituted Dreiklang, a reconstituted Skylan-S, a reconstituted ffDronpa, a reconstituted rsEGFP2, a reconstituted mClover3, or a reconstituted EGFP.

Embodiment 29. The recombinant protein of any one of embodiments 25-28, wherein said split fluorescent protein domain comprises the amino acid sequence of SEQ ID NO:60, SEQ ID NO:61, or SEQ ID NO:62.

Embodiment 30. The recombinant protein of any one of embodiments 25-29, wherein said cognate split fluorescent protein comprises the amino acid sequence of SEQ ID NO:63, SEQ ID NO:64, SEQ ID NO:65, or SEQ ID NO:66.

Embodiment 31. The recombinant protein of any one of embodiments 25-30, wherein said haloalkane dehalogenase domain comprises the amino acid sequence of SEQ ID NO:25 or SEQ ID NO:26.

Embodiment 32. The recombinant protein of any one of embodiments 25-31, wherein said chemical fluorophore is NGD. JF669, JFX646, or JF635.

Embodiment 33. The recombinant protein of any one of embodiments 25-32, wherein said chemical fluorophore comprises the formula of

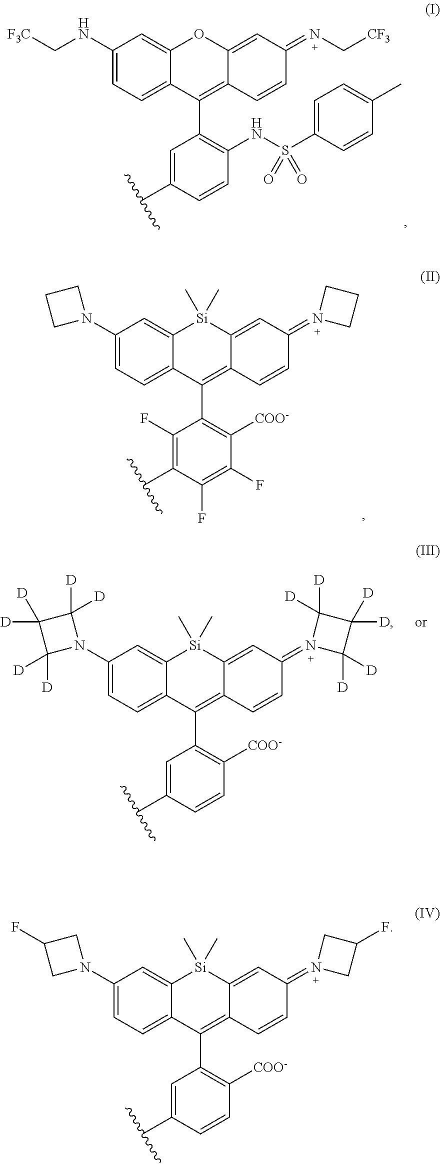

Embodiment 34. The recombinant protein of any one of embodiments 25-33, wherein said biosensing protein domain comprises: (a) a target biomolecule binding domain; or (b) a target biomolecule substrate domain, a linker domain, and a target biomolecule activity sensing domain; or (c) a target biomolecule domain, a linker domain, and a target biomolecule substrate domain.

Embodiment 35. The recombinant protein of embodiment 34, wherein said target biomolecule is a protein kinase, a second messenger molecule, or a GTPase.

Embodiment 36. The recombinant protein of embodiment 35, wherein said protein kinase is a protein kinase A (PKA), a protein kinase C (PKC), a protein kinase B (PKB), an extracellular signal-regulated kinase (ERK), a 5′ adenosine monophosphate-activated protein kinase (AMPK), a mammalian target of rapamycin (mTOR), a Fyn kinase, or a Src kinase.

Embodiment 37. The recombinant protein of embodiment 35, wherein said second messenger molecule is cyclic adenosine monophosphate (cAMP), cyclic guanosine monophosphate (cGMP), inositol triphosphate (IP₃), diacylglycerol (DAG), or calcium (Ca²⁺).

Embodiment 38. The recombinant protein of embodiment 35, wherein said GTPase is a Ras GTPase or a Rap1 GTPase.

Embodiment 39. The recombinant protein of any one of embodiments 34-38, wherein said biomolecule substrate domain comprises the amino acid sequence of any one of SEQ ID NO:27 to SEQ ID NO:36.

Embodiment 40. The recombinant protein of any one of embodiments 34-39, wherein said target biomolecule substrate domain further comprises a phosphorylatable amino acid residue.

Embodiment 41. The recombinant protein of embodiment 40, wherein said phosphorylatable amino acid residue is capable of being phosphorylated by said protein kinase.

Embodiment 42. The recombinant protein of any one of embodiments 34-41, wherein said target biomolecule domain comprises the amino acid sequence of SEQ ID NO:44 or SEQ ID NO:45.

Embodiment 43. The recombinant protein of any one of embodiments 34-41, wherein said target biomolecule activity sensing domain comprises a phosphoamino acid binding domain.

Embodiment 44. The recombinant protein of any one of embodiments 43, wherein said phosphoamino acid binding domain comprises the amino acid sequence of SEQ ID NO:39, SEQ ID NO:40, or SEQ ID NO:41.

Embodiment 45. The recombinant protein of any one of embodiments 34-44, wherein said linker domain is a peptide linker.

Embodiment 46. The recombinant protein of any one of embodiments 34-45, wherein said linker domain is between about 1 to about 500 amino acids in length.

Embodiment 47. The recombinant protein of any one of embodiments 34-46, wherein said linker domain comprises the amino acid sequence of any one of SEQ ID NO:46 to SEQ ID NO:59.

Embodiment 48. The recombinant protein of any one of embodiments 34-38, wherein said target biomolecule binding domain comprises the amino acid sequence of SEQ ID NO:37 or SEQ ID NO:38.

Embodiment 49. The recombinant protein of any one of embodiments 25-48, wherein said split fluorescent protein domain is attached to the N-terminus of said biosensing protein domain and said biosensing protein domain is attached to the N-terminus of said haloalkane dehalogenase domain.

Embodiment 50. The recombinant protein of any one of embodiments 25-48, wherein said haloalkane dehalogenase domain is attached to the N-terminus of said biosensing protein domain and said biosensing protein domain is attached to the N-terminus of said split fluorescent protein domain.

Embodiment 51. A kit comprising the recombinant protein of any one of embodiments 25-50 and a second recombinant protein, wherein said second recombinant protein comprises a protein of interest domain bound to said cognate split fluorescent protein.

Embodiment 52. A recombinant protein comprising a fluorescent protein domain bound to a biosensing protein domain and a split haloalkane dehalogenase domain, wherein: said split haloalkane dehalogenase domain is one part of a haloalkane dehalogenase split pair that forms a reconstituted haloalkane dehalogenase protein upon binding of a cognate haloalkane dehalogenase split protein domain.

Embodiment 53. The recombinant protein of embodiment 52, wherein said split haloalkane dehalogenase domain is bound to a second recombinant protein comprising said cognate haloalkane dehalogenase split protein domain bound to a protein of interest, wherein said cognate haloalkane dehalogenase split protein domain is covalently bound to a chemical fluorophore, wherein said chemical fluorophore and said fluorescent protein domain are a Forster Resonance Energy Transfer (FRET) pair.

Embodiment 54. The recombinant protein of embodiment 51 or 53, wherein said fluorescent protein domain is a red fluorescent protein (RFP), an orange fluorescent protein (OFP), a yellow fluorescent protein (YFP), or a green fluorescent protein (GFP).

Embodiment 55. The recombinant protein of any one of embodiment 52-54, wherein said fluorescent protein domain is an mScarlet domain, a stagRFP domain, an mKOk domain, an mRuby3 domain, a FusionMQV domain, a FusionRed domain, an mScarlet-1 domain, an Azalea-B5 domain, an mKate2 domain, an mCherry domain, an mNeptune2 domain, an mNeptune2.5 domain, an mCitrine domain, a cpVenus domain, an mPapaya domain, a sfGFP domain, an mNG2 domain, an mNG3A domain, an miRFP720 domain, an mRhubarb720 domain, a Dreiklang domain, a Skylan-S domain, an ffDronpa domain, an rsEGFP2 domain, an mClover3 domain, or an EGFP domain.

Embodiment 56. The recombinant protein of any one of embodiments 52-55, wherein said fluorescent protein domain comprises the amino acid sequence of any one of SEQ ID NO:1 to SEQ ID NO:24.

Embodiment 57. The recombinant protein of any one of embodiments 52-56, wherein said split haloalkane dehalogenase domain comprises the amino acid sequence of SEQ ID NO:67 or SEQ ID NO:68

Embodiment 58. The recombinant protein of any one of embodiments 52-57, wherein said cognate haloalkane dehalogenase split protein domain comprises the amino acid sequence of SEQ ID NO:67 or SEQ ID NO:68.

Embodiment 59. The recombinant protein of any one of embodiments 52-58, wherein said chemical fluorophore is NGD, JF669, JFX646, or JF635.

Embodiment 60. The recombinant protein of any one of embodiments 52-59, wherein said chemical fluorophore comprises the formula of

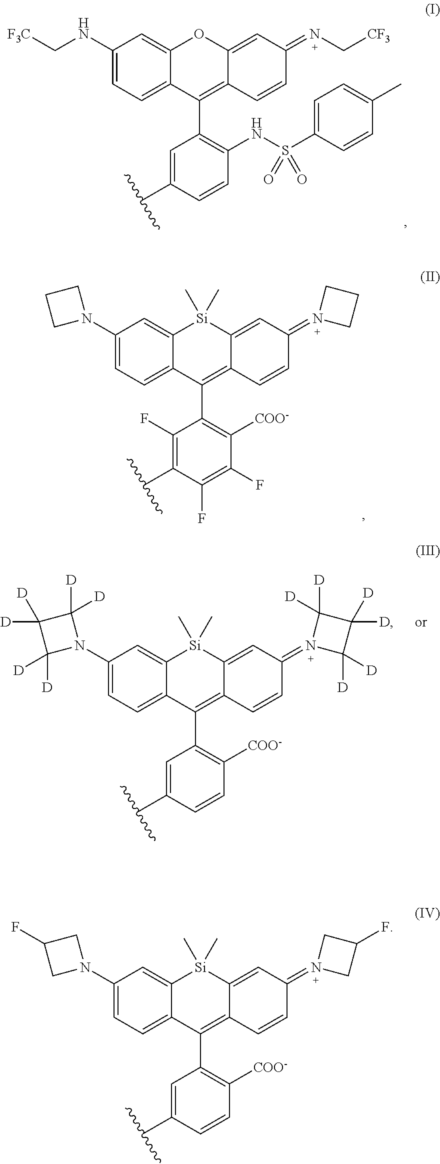

Embodiment 61. The recombinant protein of any one of embodiments 52-60, wherein said biosensing protein domain comprises: (a) a target biomolecule binding domain; (b) a target biomolecule substrate domain, a linker domain, and a target biomolecule activity sensing domain; or (c) a target biomolecule domain, a linker domain, and a target biomolecule substrate domain.

Embodiment 62. The recombinant protein of embodiment 61, wherein said target biomolecule is a protein kinase, a second messenger molecule, or a GTPase.

Embodiment 63. The recombinant protein of embodiment 62, wherein said protein kinase is a protein kinase A (PKA), a protein kinase C (PKC), a protein kinase B (PKB), an extracellular signal-regulated kinase (ERK), a 5′ adenosine monophosphate-activated protein kinase (AMPK), a mammalian target of rapamycin (mTOR), a Fyn kinase, or a Src kinase.

Embodiment 64. The recombinant protein of embodiment 62, wherein said second messenger molecule is cyclic adenosine monophosphate (cAMP), cyclic guanosine monophosphate (cGMP), inositol triphosphate (IP₃), diacylglycerol (DAG), or calcium (CaW).

Embodiment 65. The recombinant protein of embodiment 62, wherein said GTPase is a Ras GTPase or a Rap1 GTPase.

Embodiment 66. The recombinant protein of any one of embodiments 61-65, wherein said biomolecule substrate domain comprises the amino acid sequence of any one of SEQ ID NO:27 to SEQ ID NO:36.

Embodiment 67. The recombinant protein of any one of embodiments 61-66, wherein said target biomolecule substrate domain further comprises a phosphorylatable amino acid residue.

Embodiment 68. The recombinant protein of embodiment 67, wherein said phosphorylatable amino acid residue is capable of being phosphorylated by said protein kinase.

Embodiment 69. The recombinant protein of any one of embodiments 61-68, wherein said target biomolecule domain comprises the amino acid sequence of SEQ ID NO:44 or SEQ ID NO:45.

Embodiment 70. The recombinant protein of any one of embodiments 61-68, wherein said target biomolecule activity sensing domain comprises a phosphoamino acid binding domain.

Embodiment 71. The recombinant protein of any one of embodiments 70, wherein said phosphoamino acid binding domain comprises the amino acid sequence of SEQ ID NO:39, SEQ ID NO:40, or SEQ ID NO:41.

Embodiment 72. The recombinant protein of any one of embodiments 61-71, wherein said linker domain is a peptide linker.

Embodiment 73. The recombinant protein of any one of embodiments 61-72, wherein said linker domain is between about 1 to about 500 amino acids in length.

Embodiment 74. The recombinant protein of any one of embodiments 61-73, wherein said linker domain comprises the amino acid sequence of any one of SEQ ID NO:46 to SEQ ID NO:59.

Embodiment 75. The recombinant protein of any one of embodiments 61-65, wherein said target biomolecule binding domain comprises the amino acid sequence of SEQ ID NO:37 or SEQ ID NO:38.

Embodiment 76. The recombinant protein of any one of embodiments 52-75, wherein said fluorescent protein domain is attached to the N-terminus of said biosensing protein domain and said biosensing protein domain is attached to the N-terminus of said split haloalkane dehalogenase domain.

Embodiment 77. The recombinant protein of any one of embodiments 52-75, wherein said split haloalkane dehalogenase domain is attached to the N-terminus of said biosensing protein domain and said biosensing protein domain is attached to the N-terminus of said fluorescent protein domain.

Embodiment 78. A kit comprising the recombinant protein of any one of embodiments 52-77 and a second recombinant protein, wherein said second recombinant protein comprises said cognate haloalkane dehalogenase split protein domain bound to a protein of interest.

Embodiment 79. An isolated nucleic acid encoding the recombinant protein of any one of embodiments 1-50 or 52-77.

Embodiment 80. An expression vector comprising the isolated nucleic acid of embodiment 79.

Embodiment 81. The expression vector of embodiment 80, wherein the expression vector is a viral vector.

Embodiment 82. The expression vector of embodiment 81, wherein the viral vector is an Adeno-associated viral (AAV) vector, an Adenovirus vector, or a lentiviral vector.

Embodiment 83. A method for detecting a target biomolecule in a cell, said method comprising: (a) contacting said cell with an expression vector comprising a nucleic acid, wherein said nucleic acid encodes a recombinant protein of any one of embodiments 1-24; (b) transducing said cell with said expression vector: (c) allowing said cell to express said recombinant protein and interact with said target biomolecule in said cell; and (d) detecting a change in fluorescent signal from said recombinant protein, thereby detecting said target biomolecule.

Embodiment 84. The method of embodiment 83, wherein the cell is imaged at a first time point to generate a first image.

Embodiment 85. The method of embodiment 83 or 84, wherein the cell is imaged at a second time point to generate a second image.

Embodiment 86. The method of any one of embodiment 85, wherein step (d) comprises comparing said first image to said second image to determine activity of said target biomolecule at said first time point compared to said second time point.

Embodiment 87. The method of any one of embodiments 83-86, wherein said cell is a living cell.

Embodiment 88. The method of any one of embodiments 83-87, wherein said cell is a mammalian cell.

Embodiment 89. The method of any one of embodiments 83-88, wherein said cell is imaged using a fluorescence microscope.

Embodiment 90. A method for detecting a target biomolecule in a cell, said method comprising: (a) contacting said cell with an expression vector comprising a nucleic acid, wherein said nucleic acid encodes a recombinant protein of any one of embodiments 25-50; (b) transducing said cell with said expression vector; (c) allowing said cell to express said recombinant protein and interact with said target biomolecule in said cell; and (d) detecting a change in fluorescent signal from said recombinant protein, thereby detecting said target biomolecule.

Embodiment 91. The method of embodiment 90, wherein said expression vector further comprises a second isolated nucleic acid encoding said second recombinant protein of any one of embodiments 26-50.

Embodiment 92. The method of embodiment 90, wherein step (a) further comprises contacting said cell with a second expression vector comprising a second isolated nucleic acid, wherein said second isolated nucleic acid encodes said second recombinant protein of any one of embodiments 26-50; wherein step (b) further comprises transducing said cell with said second expression vector; and wherein step (c) further comprises allowing said cell to express said second recombinant protein.

Embodiment 93. The method of any one of embodiments 90-92, wherein the cell is imaged at a first time point to generate a first image.

Embodiment 94. The method of any one of embodiments 90-93, wherein the cell is imaged at a second time point to generate a second image.

Embodiment 95. The method of any one of embodiment 94, wherein step (d) comprises comparing said first image to said second image to determine activity of said target molecule at said first time point compared to said second time point.

Embodiment 96. The method of any one of embodiments 90-95, wherein said cell is a living cell.

Embodiment 97. The method of any one of embodiments 90-96, wherein said cell is a mammalian cell.

Embodiment 98. The method of any one of embodiments 90-97, wherein said cell is imaged using a fluorescence microscope.

Embodiment 99. A method for detecting a target biomolecule in a cell, said method comprising: (a) contacting said cell with an expression vector comprising a nucleic acid, wherein said nucleic acid encodes a recombinant protein of any one of embodiments 52-77; (b) transducing said cell with said expression vector; (c) allowing said cell to express said recombinant protein and interact with said target biomolecule in said cell; and (d) detecting a change in fluorescent signal from said recombinant protein, thereby detecting said target biomolecule.

Embodiment 100. The method of embodiment 99, wherein said expression vector further comprises a second isolated nucleic acid encoding said second recombinant protein of any one of embodiments 53-77.

Embodiment 101. The method of embodiment 99, wherein step (a) further comprises contacting said cell with a second expression vector comprising a second isolated nucleic acid, wherein said second isolated nucleic acid encodes said second recombinant protein of any one of embodiments 53-77; wherein step (b) further comprises transducing said cell with said second expression vector; and wherein step (c) further comprises allowing said cell to express said second recombinant protein.

Embodiment 102. The method of any one of embodiments 99-101, wherein the cell is imaged at a first time point to generate a first image.

Embodiment 103. The method of any one of embodiments 99-102, wherein the cell is imaged at a second time point to generate a second image.

Embodiment 104. The method of any one of embodiment 103, wherein step (d) comprises comparing said first image to said second image to determine activity of said target biomolecule at said first time point compared to said second time point.

Embodiment 105. The method of any one of embodiments 99-104, wherein said cell is a living cell.

Embodiment 106. The method of any one of embodiments 99-105, wherein said cell is a mammalian cell.

Embodiment 107. The method of any one of embodiments 99-106, wherein said cell is imaged using a fluorescence microscope.

What is claimed is:

1. A recombinant protein covalently bound to a chemical fluorophore, said recombinant protein comprising a fluorescent protein domain bound to a biosensing protein domain and a haloalkane dehalogenase domain bound to said biosensing protein domain, wherein:
said chemical fluorophore is covalently bound to said haloalkane dehalogenase domain; and
said chemical fluorophore and said fluorescent protein domain are a Forster Resonance Energy Transfer (FRET) pair.

2. The recombinant protein of claim 1, wherein said fluorescent protein domain is a red fluorescent protein (RFP) domain, an orange fluorescent protein (OFP) domain, a yellow fluorescent protein (YFP) domain, or a green fluorescent protein (GFP) domain.

3. The recombinant protein of claim 1, wherein said fluorescent protein domain is an mScarlet domain, a stagRFP domain, an mKOk domain, an mRuby3 domain, a FusionMQV domain, a FusionRed domain, an mScarlet-1 domain, an Azalea-B5 domain, an mKate2 domain, an mCherry domain, an mNeptune2 domain, an mNeptune2.5 domain, an mCitrine domain, a cpVenus domain, an mPapaya domain, a sfGFP domain, an mNG2 domain, an mNG3A domain, an miRFP720 domain, an mRhubarb720 domain, a Dreiklang domain, a Skylan-S domain, an ffDronpa domain, an rsEGFP2 domain, an mClover3 domain, or an EGFP domain.

4. The recombinant protein of claim 1, wherein said fluorescent protein domain comprises the amino acid sequence of any one of SEQ ID NO:1 to SEQ ID NO:24.

5. The recombinant protein of claim 1, wherein said haloalkane dehalogenase domain comprises the amino acid sequence of SEQ ID NO:25 or SEQ ID NO:26.

6. The recombinant protein of claim 1, wherein said chemical fluorophore is NGD, JF669, JFX646, or JF635.

7. The recombinant protein of claim 1, wherein said chemical fluorophore comprises the formula of

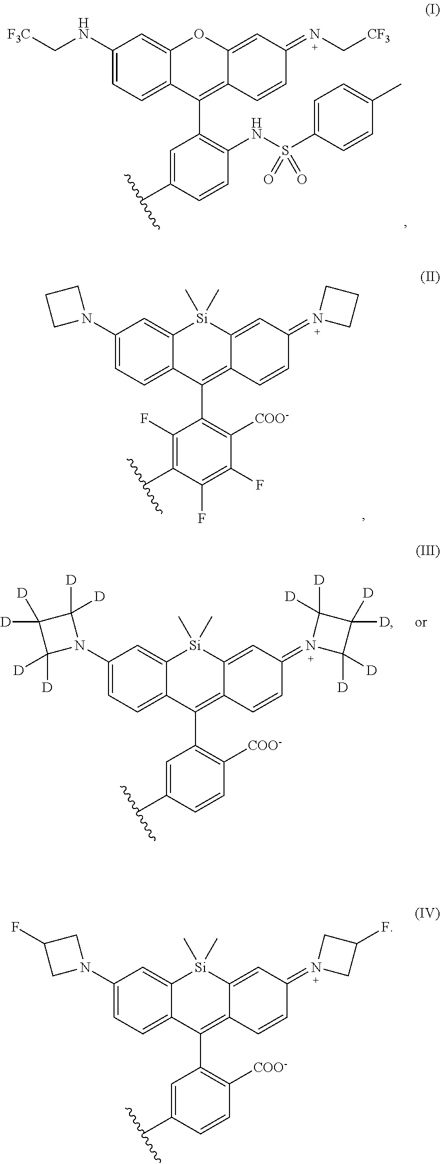

8. The recombinant protein of claim 1, wherein said biosensing protein domain comprises:
(a) a target biomolecule binding domain;
(b) a target biomolecule substrate domain, a linker domain, and a target biomolecule activity sensing domain; or
(c) a target biomolecule domain, a linker domain, and a target biomolecule substrate domain.

9. The recombinant protein of claim 8, wherein said target biomolecule is a protein kinase, a second messenger molecule, or a GTPase.

10. The recombinant protein of claim 9, wherein said protein kinase is a protein kinase A (PKA), a protein kinase C (PKC), a protein kinase B (PKB), an extracellular signal-regulated kinase (ERK), a 5′ adenosine monophosphate-activated protein kinase (AMPK), a mammalian target of rapamycin (mTOR), a Fyn kinase, or a Src kinase.

11. The recombinant protein of claim 9, wherein said second messenger molecule is cyclic adenosine monophosphate (cAMP), cyclic guanosine monophosphate (cGMP), inositol triphosphate (IP₃), diacylglycerol (DAG), or calcium (Ca²⁺).

12. The recombinant protein of claim 9, wherein said GTPase is a Ras GTPase or a Rap1 GTPase.

13. The recombinant protein of claim 8, wherein said biomolecule substrate domain comprises the amino acid sequence of any one of SEQ ID NO:27 to SEQ ID NO:36.

14. The recombinant protein of claim 8, wherein said target biomolecule substrate domain further comprises a phosphorylatable amino acid residue.

15. The recombinant protein of claim 14, wherein said phosphorylatable amino acid residue is capable of being phosphorylated by said protein kinase.

16. The recombinant protein of claim 8, wherein said target biomolecule domain comprises the amino acid sequence of SEQ ID NO:44 or SEQ ID NO:45.

17. The recombinant protein of claim 8, wherein said target biomolecule activity sensing domain comprises a phosphoamino acid binding domain.

18. The recombinant protein of claim 17, wherein said phosphoamino acid binding domain comprises the amino acid sequence of SEQ ID NO:39, SEQ ID NO:40, or SEQ ID NO:41.

19. The recombinant protein of claim 8, wherein said linker domain is a peptide linker.

20. The recombinant protein of claim 8, wherein said linker domain is between about 1 to about 500 amino acids in length.

21. The recombinant protein of claim 8, wherein said linker domain comprises the amino acid sequence of any one of SEQ ID NO:46 to SEQ ID NO:59.

22. The recombinant protein of claim 8, wherein said target biomolecule binding domain comprises the amino acid sequence of SEQ ID NO:37 or SEQ ID NO:38.

23. The recombinant protein of claim 1, wherein said fluorescent protein is attached to the N-terminus of said biosensing protein domain and said biosensing protein domain is attached to the N-terminus of said haloalkane dehalogenase domain.

24. The recombinant protein of claim 1, wherein said haloalkane dehalogenase domain is attached to the N-terminus of said biosensing protein domain and said biosensing protein domain is attached to the N-terminus of said fluorescent protein.

25. A recombinant protein covalently bound to a chemical fluorophore, said recombinant protein comprising a split fluorescent protein domain bound to a biosensing protein domain and a haloalkane dehalogenase domain bound to said biosensing protein domain, wherein:
said chemical fluorophore is covalently bound to said haloalkane dehalogenase domain;
said split fluorescent protein domain is one part of a fluorescent protein split pair that forms a reconstituted fluorescent protein upon binding of a cognate split fluorescent protein; and
said chemical fluorophore and said reconstituted fluorescent protein domain are a Förster Resonance Energy Transfer (FRET) pair.

26. The recombinant protein of claim 25, wherein said recombinant protein is non-covalently bound to a second recombinant protein comprising the cognate split fluorescent protein domain bound to a protein of interest, wherein the split fluorescent protein domain is non-covalently bound to the cognate split fluorescent protein thereby forming said reconstituted fluorescent protein.

27. The recombinant protein of claim 25, wherein said reconstituted fluorescent protein is a reconstituted red fluorescent protein (RFP), a reconstituted orange fluorescent protein (OFP), a reconstituted yellow fluorescent protein (YFP), or a reconstituted green fluorescent protein (GFP).

28. The recombinant protein of any one of claim 25, wherein said reconstituted fluorescent protein is a reconstituted mScarlet, a reconstituted stagRFP, a reconstituted mKOk, a reconstituted mRuby3, a reconstituted FusionMQV, a reconstituted FusionRed, a reconstituted mScarlet-1, a reconstituted Azalea-B5, a reconstituted mKate2, a reconstituted mCherry, a reconstituted mNeptune2, a reconstituted mNeptune2.5, a reconstituted mCitrine, a reconstituted cpVenus, a reconstituted mPapaya, a reconstituted sfGFP, a reconstituted mNG2, a reconstituted mNG3A, a reconstituted miRFP720, a reconstituted mRhubarb720, a reconstituted Dreiklang, a reconstituted Skylan-S, a reconstituted ffDronpa, a reconstituted rsEGFP2, a reconstituted mClover3, or a reconstituted EGFP.

29. The recombinant protein of claim 25, wherein said split fluorescent protein domain comprises the amino acid sequence of SEQ ID NO:60, SEQ ID NO:61, or SEQ ID NO:62.

30. The recombinant protein of claim 25, wherein said cognate split fluorescent protein comprises the amino acid sequence of SEQ ID NO:63, SEQ ID NO:64, SEQ ID NO:65, or SEQ ID NO:66.

31. The recombinant protein of claim 25, wherein said haloalkane dehalogenase domain comprises the amino acid sequence of SEQ ID NO:25 or SEQ ID NO:26.

32. The recombinant protein of claim 25, wherein said chemical fluorophore is NGD, JF669, JFX646, or JF635.

33. The recombinant protein of claim 25, wherein said chemical fluorophore comprises the formula of

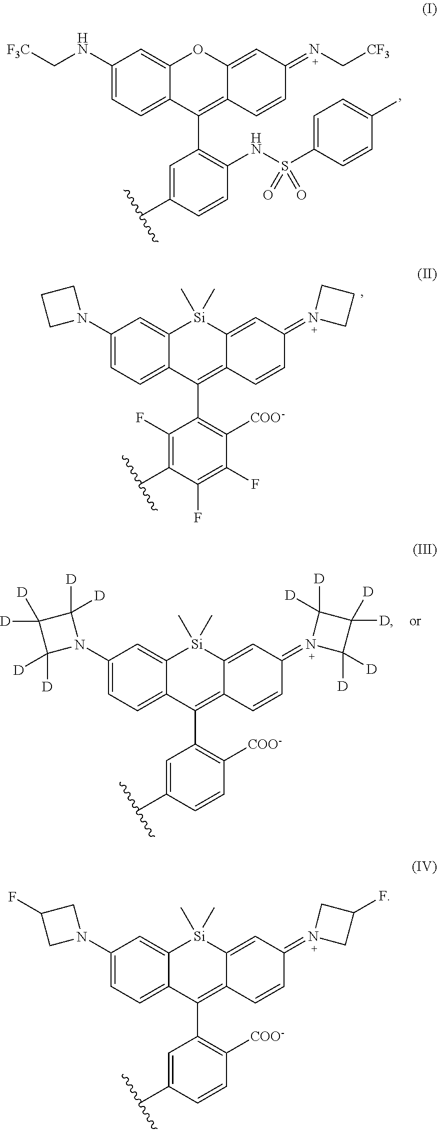

34. The recombinant protein of claim 25, wherein said biosensing protein domain comprises:
(a) a target biomolecule binding domain;
(b) a target biomolecule substrate domain, a linker domain, and a target biomolecule activity sensing domain; or
(c) a target biomolecule domain, a linker domain, and a target biomolecule substrate domain.

35. The recombinant protein of claim 34, wherein said target biomolecule is a protein kinase, a second messenger molecule, or a GTPase.

36. The recombinant protein of claim 35, wherein said protein kinase is a protein kinase A (PKA), a protein kinase C (PKC), a protein kinase B (PKB), an extracellular signal-regulated kinase (ERK), a 5′ adenosine monophosphate-activated protein kinase (AMPK), a mammalian target of rapamycin (mTOR), a Fyn kinase, or a Src kinase.

37. The recombinant protein of claim 35, wherein said second messenger molecule is cyclic adenosine monophosphate (cAMP), cyclic guanosine monophosphate (cGMP), inositol triphosphate (IP₃), diacylglycerol (DAG), or calcium (Ca²⁺).

38. The recombinant protein of claim 35, wherein said GTPase is a Ras GTPase or a Rap1 GTPase.

39. The recombinant protein of claim 34, wherein said biomolecule substrate domain comprises the amino acid sequence of any one of SEQ ID NO:27 to SEQ ID NO:36.

40. The recombinant protein of claim 34, wherein said target biomolecule substrate domain further comprises a phosphorylatable amino acid residue.

41. The recombinant protein of claim 40, wherein said phosphorylatable amino acid residue is capable of being phosphorylated by said protein kinase.

42. The recombinant protein of claim 34, wherein said target biomolecule domain comprises the amino acid sequence of SEQ ID NO:44 or SEQ ID NO:45.

43. The recombinant protein of claim 34, wherein said target biomolecule activity sensing domain comprises a phosphoamino acid binding domain.

44. The recombinant protein of claim 43, wherein said phosphoamino acid binding domain comprises the amino acid sequence of SEQ ID NO:39, SEQ ID NO:40, or SEQ ID NO:41.

45. The recombinant protein of claim 34, wherein said linker domain is a peptide linker.

46. The recombinant protein of claim 34, wherein said linker domain is between about 1 to about 500 amino acids in length.

47. The recombinant protein of claim 34, wherein said linker domain comprises the amino acid sequence of any one of SEQ ID NO:46 to SEQ ID NO:59.

48. The recombinant protein of claim 34, wherein said target biomolecule binding domain comprises the amino acid sequence of SEQ ID NO:37 or SEQ ID NO:38.

49. The recombinant protein of claim 25, wherein said split fluorescent protein domain is attached to the N-terminus of said biosensing protein domain and said biosensing protein domain is attached to the N-terminus of said haloalkane dehalogenase domain.

50. The recombinant protein of claim 25, wherein said haloalkane dehalogenase domain is attached to the N-terminus of said biosensing protein domain and said biosensing protein domain is attached to the N-terminus of said split fluorescent protein domain.

51. A kit comprising the recombinant protein of claim 25 and a second recombinant protein, wherein said second recombinant protein comprises a protein of interest domain bound to said cognate split fluorescent protein.

52. A recombinant protein comprising a fluorescent protein domain bound to a biosensing protein domain and a split haloalkane dehalogenase domain, wherein:
said split haloalkane dehalogenase domain is one part of a haloalkane dehalogenase split pair that forms a reconstituted haloalkane dehalogenase protein upon binding of a cognate haloalkane dehalogenase split protein domain.

53. The recombinant protein of claim 52, wherein said split haloalkane dehalogenase domain is bound to a second recombinant protein comprising said cognate haloalkane dehalogenase split protein domain bound to a protein of interest, wherein said cognate haloalkane dehalogenase split protein domain is covalently bound to a chemical fluorophore, wherein said chemical fluorophore and said fluorescent protein domain are a Forster Resonance Energy Transfer (FRET) pair.

54. The recombinant protein of claim 51, wherein said fluorescent protein domain is a red fluorescent protein (RFP), an orange fluorescent protein (OFP), a yellow fluorescent protein (YFP), or a green fluorescent protein (GFP).

55. The recombinant protein of any one of claim 52, wherein said fluorescent protein domain is an mScarlet domain, a stagRFP domain, an mKOk domain, an mRuby3 domain, a FusionMQV domain, a FusionRed domain, an mScarlet-1 domain, an Azalea-B5 domain, an mKate2 domain, an mCherry domain, an mNeptune2 domain, an mNeptune2.5 domain, an mCitrine domain, a cpVenus domain, an mPapaya domain, a sfGFP domain, an mNG2 domain, an mNG3A domain, an miRFP720 domain, an mRhubarb720 domain, a Dreiklang domain, a Skylan-S domain, an ffDronpa domain, an rsEGFP2 domain, an mClover3 domain, or an EGFP domain.

56. The recombinant protein of claim 52, wherein said fluorescent protein domain comprises the amino acid sequence of any one of SEQ ID NO:1 to SEQ ID NO:24.

57. The recombinant protein of claim 52, wherein said split haloalkane dehalogenase domain comprises the amino acid sequence of SEQ ID NO:67 or SEQ ID NO:68.

58. The recombinant protein of claim 52, wherein said cognate haloalkane dehalogenase split protein domain comprises the amino acid sequence of SEQ ID NO:67 or SEQ ID NO:68.

59. The recombinant protein of claim 52, wherein said chemical fluorophore is NGD, JF669, JFX646, or JF635.

60. The recombinant protein of claim 52, wherein said chemical fluorophore comprises the formula of

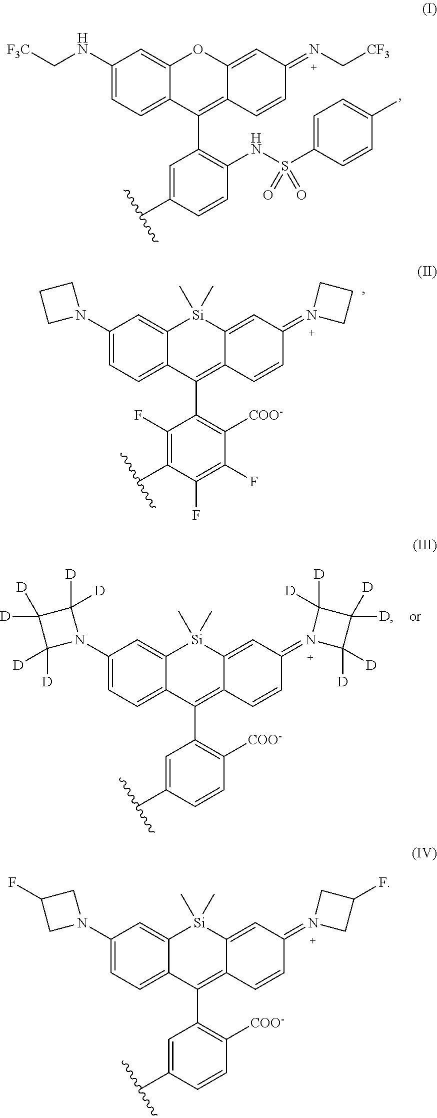

61. The recombinant protein of claim 52, wherein said biosensing protein domain comprises:
(a) a target biomolecule binding domain;
(b) a target biomolecule substrate domain, a linker domain, and a target biomolecule activity sensing domain; or
(c) a target biomolecule domain, a linker domain, and a target biomolecule substrate domain.

62. The recombinant protein of claim 61, wherein said target biomolecule is a protein kinase, a second messenger molecule, or a GTPase.

63. The recombinant protein of claim 62, wherein said protein kinase is a protein kinase A (PKA), a protein kinase C (PKC), a protein kinase B (PKB), an extracellular signal-regulated kinase (ERK), a 5′ adenosine monophosphate-activated protein kinase (AMPK), a mammalian target of rapamycin (mTOR), a Fyn kinase, or a Src kinase.

64. The recombinant protein of claim 62, wherein said second messenger molecule is cyclic adenosine monophosphate (cAMP), cyclic guanosine monophosphate (cGMP), inositol triphosphate (IP₃), diacylglycerol (DAG), or calcium (Ca²⁺).

65. The recombinant protein of claim 62, wherein said GTPase is a Ras GTPase or a Rap1 GTPase.

66. The recombinant protein of claim 61, wherein said biomolecule substrate domain comprises the amino acid sequence of any one of SEQ ID NO:27 to SEQ ID NO:36.

67. The recombinant protein of claim 61, wherein said target biomolecule substrate domain further comprises a phosphorylatable amino acid residue.

68. The recombinant protein of claim 67, wherein said phosphorylatable amino acid residue is capable of being phosphorylated by said protein kinase.

69. The recombinant protein of claim 61, wherein said target biomolecule domain comprises the amino acid sequence of SEQ ID NO:44 or SEQ ID NO:45.

70. The recombinant protein of claim 61, wherein said target biomolecule activity sensing domain comprises a phosphoamino acid binding domain.

71. The recombinant protein of claim 70, wherein said phosphoamino acid binding domain comprises the amino acid sequence of SEQ ID NO:39, SEQ ID NO:40, or SEQ ID NO:41.

72. The recombinant protein of claim 61, wherein said linker domain is a peptide linker.

73. The recombinant protein of claim 61, wherein said linker domain is between about 1 to about 500 amino acids in length.

74. The recombinant protein of claim 61, wherein said linker domain comprises the amino acid sequence of any one of SEQ ID NO:46 to SEQ ID NO:59.

75. The recombinant protein of claims 61-65, wherein said target biomolecule binding domain comprises the amino acid sequence of SEQ ID NO:37 or SEQ ID NO:38.

76. The recombinant protein of claim 52, wherein said fluorescent protein domain is attached to the N-terminus of said biosensing protein domain and said biosensing protein domain is attached to the N-terminus of said split haloalkane dehalogenase domain.

77. The recombinant protein of claim 52, wherein said split haloalkane dehalogenase domain is attached to the N-terminus of said biosensing protein domain and said biosensing protein domain is attached to the N-terminus of said fluorescent protein domain.

78. A kit comprising the recombinant protein of claim 52 and a second recombinant protein, wherein said second recombinant protein comprises said cognate haloalkane dehalogenase split protein domain bound to a protein of interest.

79. An isolated nucleic acid encoding the recombinant protein of claim 1.

80. An expression vector comprising the isolated nucleic acid of claim 79.

81. The expression vector of claim 80, wherein the expression vector is a viral vector.

82. The expression vector of claim 81, wherein the viral vector is an Adeno-associated viral (AAV) vector, an Adenovirus vector, or a lentiviral vector.

83. A method for detecting a target biomolecule in a cell, said method comprising:
(a) contacting said cell with an expression vector comprising a nucleic acid, wherein said nucleic acid encodes a recombinant protein of claim 1;
(b) transducing said cell with said expression vector;
(c) allowing said cell to express said recombinant protein and interact with said target biomolecule in said cell; and
(d) detecting a change in fluorescent signal from said recombinant protein, thereby detecting said target biomolecule.

84. The method of claim 83, wherein the cell is imaged at a first time point to generate a first image.

85. The method of claim 83, wherein the cell is imaged at a second time point to generate a second image.

86. The method of any one of claim 85, wherein step (d) comprises comparing said first image to said second image to determine activity of said target biomolecule at said first time point compared to said second time point.

87. The method of claim 83, wherein said cell is a living cell.

88. The method of claim 83, wherein said cell is a mammalian cell.

89. The method of claim 83, wherein said cell is imaged using a fluorescence microscope.

90. A method for detecting a target biomolecule in a cell, said method comprising:
(a) contacting said cell with an expression vector comprising a nucleic acid, wherein said nucleic acid encodes a recombinant protein of claim 25;
(b) transducing said cell with said expression vector;
(c) allowing said cell to express said recombinant protein and interact with said target biomolecule in said cell; and
(d) detecting a change in fluorescent signal from said recombinant protein, thereby detecting said target biomolecule.

91. The method of claim 90, wherein said expression vector further comprises a second isolated nucleic acid encoding said second recombinant protein of claim 26.

92. The method of claim 90, wherein step (a) further comprises contacting said cell with a second expression vector comprising a second isolated nucleic acid, wherein said second isolated nucleic acid encodes said second recombinant protein of claim 26-50; wherein step (b) further comprises transducing said cell with said second expression vector; and wherein step (c) further comprises allowing said cell to express said second recombinant protein.

93. The method of claim 90, wherein the cell is imaged at a first time point to generate a first image.

94. The method of claim 90, wherein the cell is imaged at a second time point to generate a second image.

95. The method of any one of claim 94, wherein step (d) comprises comparing said first image to said second image to determine activity of said target molecule at said first time point compared to said second time point.

96. The method of claim 90, wherein said cell is a living cell.

97. The method of claim 90, wherein said cell is a mammalian cell.

98. The method of claim 90, wherein said cell is imaged using a fluorescence microscope.

99. A method for detecting a target biomolecule in a cell, said method comprising:
(a) contacting said cell with an expression vector comprising a nucleic acid, wherein said nucleic acid encodes a recombinant protein of claim 52;
(b) transducing said cell with said expression vector;
(c) allowing said cell to express said recombinant protein and interact with said target biomolecule in said cell; and
(d) detecting a change in fluorescent signal from said recombinant protein, thereby detecting said target biomolecule.

100. The method of claim 99, wherein said expression vector further comprises a second isolated nucleic acid encoding said second recombinant protein of claim 53.

101. The method of claim 99, wherein step (a) further comprises contacting said cell with a second expression vector comprising a second isolated nucleic acid, wherein said second isolated nucleic acid encodes said second recombinant protein of claim 53-77; wherein step (b) further comprises transducing said cell with said second expression vector; and wherein step (c) further comprises allowing said cell to express said second recombinant protein.

102. The method of claim 99, wherein the cell is imaged at a first time point to generate a first image.

103. The method of claim 99, wherein the cell is imaged at a second time point to generate a second image.

104. The method of any one of claim 103, wherein step (d) comprises comparing said first image to said second image to determine activity of said target biomolecule at said first time point compared to said second time point.

105. The method of claim 99, wherein said cell is a living cell.

106. The method of claim 99, wherein said cell is a mammalian cell.

107. The method of claim 99, wherein said cell is imaged using a fluorescence microscope.
